(19) United States
(12) Patent Application Publication
(10) Pub. No.: US 2007/0227971 A1
(43) Pub. Date: Oct. 4, 2007

(54) Flocculation Method and Flocculated Organism

(75) Inventor: Larry W. Denney, Loxahatchee, FL (US)

Correspondence Address: BARNES & THORNBURG LLP, 11 SOUTH MERIDIAN, INDIANAPOLIS, IN, 46204, US

(73) Assignee: Biomass Processing Technology, Inc., West Palm Beach, FL (US)

(21) Appl. No.: 11/596,694

(22) PCT Filed: May 16, 2005
(86) PCT No.: PCT/US05/17053

§ 371 Date: Nov. 16, 2006

Related U.S. Application Data
(60) Provisional application No. 60/572,166, filed on May 18, 2004.
(60) Provisional application No. 60/572,179, filed on May 18, 2004.
(60) Provisional application No. 60/572,187, filed on May 18, 2004.
(60) Provisional application No. 60/572,206, filed on May 18, 2004.
(60) Provisional application No. 60/572,226, filed on May 18, 2004.
(60) Provisional application No. 60/571,959, filed on May 18, 2004.
(60) Provisional application No. 60/571,996, filed on May 18, 2004.

(51) Int. Cl. C02F 3/34; C12N 1/38

(52) U.S. Cl. 210/606; 435/244

(57) ABSTRACT

A method for flocculating microorganisms is described. The method comprises the steps of contacting the microorganisms with a cationic flocculating agent, contacting the microorganisms with an anionic flocculating agent, and flocculating the microorganisms. A method for sedimenting microorganisms is also described. The method comprises the steps of contacting the microorganisms with a cationic flocculating agent, contacting the microorganisms with an anionic flocculating agent, flocculating the microorganisms, and sedimenting the microorganisms. Also described are the flocculated microorganisms produced by the described methods and an animal feed supplement and a feed composition containing these flocculated microorganisms. The flocculated microorganisms can be, for example, yeast, bacteria, fungi, mycoplasma, and combinations thereof.

### CROSS REFERENCE TO RELATED APPLICATIONS

This application claims the benefit under 35 U.S.C. 119(e) of U.S. Provisional Applications Ser. Nos. 60/572,206, 60/572,187; 60/572,226;, 60/572,166; 60/572,179; 60/571,996; and 60/571,959; filed May 18, 2004, each of which is expressly incorporated herein by reference.

### FIELD OF THE INVENTION

The invention relates to a flocculation method and a flocculated organism. More particularly, the invention relates to a method for catalyzed flocculation and an organism produced by catalyzed flocculation.

### BACKGROUND

Many industrial processes and technical procedures used in research laboratories, among other processes, require the separation of microorganisms, such as yeast or bacteria, from bulk fluids as one step in the process. Microorganisms can be separated from bulk fluids by sedimentation, for example, by using such methods as centrifugation or flocculation, followed by settling, if the microorganisms are naturally flocculating. These naturally flocculating microorganisms can be separated from bulk fluids by flocculation with minimal disruption of the microorganisms. Microorganisms can also be separated from bulk fluids by filtration.

U.S. Pat. No. 4,060,456 discloses the stabilization of microorganisms having an associated enzyme activity by treating the microorganisms with a cationic flocculating agent such as a polyethylenimine or polyvinylpyrrolidone. U.S. Pat. No. 3,989,596 discloses the use of polyelectrolytes, such as polyamines, and cationic polyacrylamides in the stabilization of microorganisms having associated enzyme activities. U.S. Pat. No. 4,212,943 discloses a bacterial cell aggregate having increased particle hardness which is produced by contacting bacterial cells with a cross-linked reaction product of glutaraldehyde or cyanuric halide and a cationic polymer obtained by polymerization of an epihalohydrin and an alkylenepolyamine. U.S. Pat. No. 4,337,313 discloses the immobilization of the bacteria P. rubrum by treatment with tannin and an adduct of glutaraldehyde and an epihalohydrin/polyamine copolymer. None of these patents discloses a method of flocculation of microorganisms using a cationic and an anionic flocculating agent for the purpose of separating microorganisms from bulk fluids.

There is a need for methods that are capable of increasing the rate and extent of flocculation of naturally flocculating microorganisms. For microorganisms that are not naturally flocculating, methods are needed to artificially flocculate these microorganisms for use in separating the microorganisms from bulk fluids. There is also a need for methods that can be used to catalyze flocculation of microorganisms and to control the rate and extent of flocculation during separation of microorganisms from bulk fluids.

### SUMMARY

The present invention comprises one or more of the features, or combinations thereof, outlined in the attached claims and in the following paragraphs. The present invention is based, in part, on the discovery of a method useful for catalyzed flocculation of microorganisms. The method comprises contacting the microorganisms with a cationic flocculating agent, contacting the microorganisms with an anionic flocculating agent, and flocculating the microorganisms. The rate and the extent of flocculation of microorganisms that are naturally flocculating, or are not naturally flocculating, can be controlled using this method. Thus, this method results in the catalyzed flocculation of microorganisms whereby the rate and extent of flocculation of naturally or non-naturally flocculating microorganisms can be controlled.

In one embodiment, the method is useful for, for example, sedimenting microorganisms that cannot be naturally flocculated and for increasing the rate and the extent of sedimentation of microorganisms that cannot be naturally flocculated. In another embodiment, the method is useful for sedimenting microorganisms that can be naturally flocculated and for increasing the rate and the extent of sedimentation of microorganisms that can be naturally flocculated. In another embodiment, the method can be used for controlling the rate and/or extent of flocculation of naturally or non-naturally flocculating microorganisms.

Flocculated microorganisms made according to this method are also provided. In one embodiment, the flocculated microorganisms made according to the method can be used, for example, to treat a biomaterial waste stream to remove pollutants in the biomaterial waste stream during fermentation by converting the pollutants to a valuable product as described more fully below. The flocculated microorganisms can also be used for any other suitable purpose including, but not limited to, the manufacture of products that serve as a nutrient source such as an animal feed, an animal feed supplement, a fertilizer, a fertilizer ingredient, a soil conditioner, and the like.

As described above, in one embodiment, a method for flocculating microorganisms is provided comprising the steps of contacting the microorganisms with a cationic flocculating agent, contacting the microorganisms with an anionic flocculating agent, and flocculating the microorganisms. In one embodiment the microorganisms are naturally flocculating, and, in another embodiment, the microorganisms are not naturally flocculating. In alternate embodiments the microorganisms are partially or completely flocculated.

Cationic flocculating agents useful in the compositions and methods described herein are positively charged molecules or molecules capable of carrying one or more positive charges under predetermined conditions, and include but are not limited to salt counterions, such as metal cations and salts thereof, including iron, chromium, cobalt, nickel, copper, manganese, and the like, and including multivalent metal cations, such as divalent and trivalent metal cations; small molecules such as di-, tri-, and tetraamines; polymeric materials, such as polyamines and salts thereof; and combinations thereof. Anionic flocculating agents useful in the compositions and methods described herein are negatively charged molecules or molecules capable of carrying one or more negative charges under predetermined conditions, and include but are not limited to salt counterions such as carbonates, sulfates, phosphates, and the like; small molecules such as di-, tri-, and tetracarboxylic acids, di-, tri-, and tetrasulfinic and sulfonic acids, di-, tri-, and tetraphosphinic and phosphonic acids; polymeric materials that carry or can carry a negative charge, such as polyols, polythiols, polyacids, polysulfonates, polycarboxylates, polyphosphonates, and salts thereof; and combinations thereof.

Exemplary flocculating agents of the cationic type include ferrous chloride, ferrous sulphate, ferric chloride, ferric sulphate, chlorinated ferric sulphate, aluminum sulphates, chlorinated basic aluminum sulphates, magnesium chloride, magnesium sulphate, and combinations thereof. Exemplary flocculating agents of the anionic type are polyacrylamides, polyacrylates, polymethacrylates, polycarboxylates, partially hydrolyzed polyacrylamides, polyacrylates, polymethacrylates, and polycarboxylates, polysaccharides, including xanthan gum, partially hydrolyzed guar gum, gum Arabic, or alginates and partially hydrolyzed alginates, chitosan, celluloses, hemicelluloses, polypeptides and proteins, and combinations thereof. It is appreciated that polyacrylamides are weakly anionic; however, similar to polyacrylates, polymethacrylates, polycarboxylates, polysaccharides, and the like, partial hydrolysis of the side chains will also provide anionic flocculating agents that may be used in the compositions and methods described herein.

In one illustrative embodiment the flocculating agent of the cationic type is ferric chloride and the flocculating agent of the anionic type is xanthan gum. Alternatively, the microorganisms can be flocculated using a mixture of flocculating agents of the cationic and anionic types. In one embodiment, the concentration of the flocculating agent of the cationic type can be from about 0.01 ppm to about 300 ppm. In one embodiment, the concentration of the flocculating agent of the anionic type can be from about 0.001 g/L to about 10 g/L.

In one embodiment, the polymeric cationic or anionic flocculating agents have a relatively low charge density, illustratively, as a function of molecular weight, such that there is a large charge separation or large distance between sequential charged functionalities.

The microorganisms that can be flocculated using the method described herein can be any type of microorganism capable of being flocculated using flocculating agents of the cationic and anionic type. For example, the microorganisms can be yeast, bacteria, fungi, mycoplasma, and the like. Exemplary yeast species that can be used are Sacchromyces species, Candida species, Kluyveromyces species, Pichia species, Debaromyces species, and combinations thereof, but any other suitable species of yeast can be used. Gram negative or gram positive bacteria can be flocculated using the method described herein, and fungi including, but not limited to, fungi that grow as molds or are yeastlike can also be flocculated using the presently described method. Mixtures of yeast, bacteria, fungi, mycoplasma, and the like are also suitable for use in the method described herein.

The microorganisms can be thermophilic or not thermophilic. In one embodiment, the microorganisms can be isolated microorganisms, or a mixture thereof. In another embodiment, the microorganisms can be microorganisms that have not been previously isolated. In another embodiment, the microorganisms can be naturally present in a sample and the microorganisms can be flocculated in the sample. In another embodiment, the microorganisms can be flocculated and inoculated into a sample, such as, for example, a biomaterial waste stream, or another bulk fluid.

In one embodiment, the microorganisms flocculated according to the method described herein can be preserved using any method known in the art for preventing degradation of a microorganism or its protein components. In alternate embodiments, the microorganisms can be pasteurized or the microorganisms can be refrigerated or frozen to preserve the microorganisms. In another embodiment, the microorganism can be partially degraded.

The flocculated microorganisms can be in any form, such as, for example, a paste, another aqueous mixture, a dry powder, or any other suitable form. In one embodiment, the paste, aqueous mixture, or dry powder can be added to, for example, an animal feed or fertilizer.

### BRIEF DESCRIPTION OF THE DRAWINGS

FIG. 1 is a block diagram of one illustrative embodiment of a system for processing a biomaterial waste stream.

FIG. 2A is a front elevational view of one illustrative embodiment of the sand separation unit forming part of the waste stream pre-treatment system in the biomaterial waste stream processing system of FIG. 1.

FIG. 2B is a side elevational view of the sand separation unit of FIG. 2A.

FIG. 3 is a schematic diagram of one illustrative embodiment of a control system for controlling the sand separation unit of FIGS. 1-2B.

FIGS. 4A and 4B show a flowchart of one illustrative embodiment of a software control algorithm for controlling the sand separation unit of FIGS. 1-2B via the control system of FIG. 3.

FIG. 5A is a side elevational view of one illustrative embodiment of the liquid/solid separation unit forming part of the waste stream pre-treatment system in the biomaterial waste stream processing system of FIG. 1.

FIG. 5B is a front elevational view of the liquid solid separation unit of FIG. 5A.

FIG. 6 is a schematic diagram of one illustrative embodiment of a control system for controlling the liquid/solid separation unit of FIGS. 1 and 5A-5B.

FIG. 7 is a flowchart of one illustrative embodiment of a software control algorithm for controlling the liquid/solid separation unit of FIGS. 1 and 5A-5B via the control system of FIG. 6.

FIG. 8A is a schematic diagram of one illustrative embodiment of the pH adjustment unit and corresponding control system that forms part of the waste stream pre-treatment system in the biomaterial waste stream processing system of FIG. 1.

FIG. 8B is a schematic diagram of another illustrative embodiment of the pH adjustment unit and corresponding control system that forms part of the waste stream pre-treatment system in the biomaterial waste stream processing system of FIG. 1.

FIG. 8C is a diagrammatic representation of one illustrative embodiment of the settling tank forming part of the pH adjustment unit of FIG. 8B.

FIG. 8D is a cross-sectional view of the settling tank of FIG. 8C viewed along section lines 8D-8D.

FIG. 8E is a diagrammatic representation of the settling tank of FIGS. 8C and 8D illustrating operation thereof.

FIG. 9 is a flowchart of one illustrative embodiment of a software control algorithm for controlling the pH adjustment unit of FIG. 1 via the control system of either of FIGS. 8A and 8B.

FIG. 10 is a schematic diagram of one illustrative embodiment of the air system and corresponding control system that forms part of the biomaterial waste stream processing system of FIG. 1.

FIG. 11 is a schematic diagram of one illustrative embodiment of the water system and corresponding control system that forms part of the biomaterial waste stream processing system of FIG. 1.

FIG. 12 is a block diagram of one illustrative embodiment of the waste fermentation system forming part of the biomaterial waste processing system of FIG. 1.

FIG. 13A is a schematic diagram of one illustrative embodiment of the sterilization unit and corresponding control system that forms part of the waste fermentation system of FIG. 12.

FIG. 13B is a schematic diagram of another illustrative embodiment of the sterilization unit and corresponding control system that forms part of the waste fermentation system of FIG. 12.

FIG. 13C is a cross-sectional view of one illustrative embodiment of either of the settling tanks forming part of the sterilization system of FIG. 13B.

FIG. 13D is a diagrammatic representation of one of the number of truncated cone-topped cylinders positioned within the settling tank of FIG. 13B.

FIG. 13E is a magnified cross-sectional view a portion of the settling tank of FIG. 13C illustrating operation thereof.

FIG. 13F is a magnified cross-sectional view of another portion of the settling tank of FIG. 13C illustrating operation thereof.

FIGS. 14A-14C show a flowchart of one illustrative embodiment of a software control algorithm for controlling the sterilization unit of either of FIGS. 13A and 13B.

FIG. 15 is a schematic diagram of one illustrative embodiment of the steam unit and corresponding control system that forms part of the waste fermentation system of FIG. 12.

FIG. 16 is a flowchart of one illustrative embodiment of a software control algorithm for controlling the steam unit of FIG. 15.

FIG. 17 is a schematic diagram of one illustrative embodiment of the cooling tower unit and corresponding control system that forms part of the waste fermentation system of FIG. 12.

FIGS. 18A-18B show a flowchart of one illustrative embodiment of a software control algorithm for controlling the cooling tower unit of FIG. 17.

FIG. 19 is a diagrammatic representation of one illustrative embodiment of the fermentation unit forming part of the waste fermentation system of FIG. 12.

FIG. 20 is a diagrammatic illustration of the general operation of either of the fermentation tanks of FIG. 19 in a normal, continuous flow operational mode.

FIG. 21 is a diagrammatic illustration of the operation of the air spargers and fermenting organism collection cone in either of the fermentation tanks of FIG. 19 in a fermenting organism reduction operational mode.

FIG. 22 is a diagrammatic illustration of the operation of the air spargers and fermenting organism collection cone in either of the fermentation tanks of FIG. 19 in the normal, continuous flow operational mode.

FIG. 23A is a front elevational view of one illustrative embodiment of the first fermentation tank of FIG. 19.

FIG. 23B is a magnified front elevational view of the lower portion of the first fermentation tank of FIG. 23A illustrating some of the structural details of the air spargers and fermenting organism collection cone.

FIG. 23C is a cross-sectional view of the lower portion of the first fermentation tank of FIG. 23B viewed along section lines 23C-23C.

FIG. 24A is a front elevational view of one illustrative embodiment of the second fermentation tank of FIG. 19.

FIG. 24B is a cross sectional view of the lower portion of the second fermentation tank of FIG. 24A viewed along section lines 24B,C-24B,C and illustrating some of the structural details of the outer air sparger.

FIG. 24C is a cross sectional view of the lower portion of the second fermentation tank of FIG. 24A viewed along section lines 24B,C-24B,C and illustrating some of the structural details of the inner air sparger.

FIG. 25 is a schematic diagram of one illustrative embodiment of a control system for controlling the fermentation unit of FIGS. 12 and 19-24C.

FIGS. 26A-26H show a flowchart of one illustrative embodiment of a software control algorithm for controlling the fermentation unit of FIGS. 12 and 19-24C via the control system of FIG. 25.

FIG. 27A is a schematic diagram of one illustrative embodiment of the pasteurization unit and corresponding control system that forms part of the waste fermentation system of FIG. 12.

FIG. 27B is a schematic diagram of another illustrative embodiment of the pasteurization unit and corresponding control system that forms part of the waste fermentation system of FIG. 12.

FIG. 28 is a flowchart of one illustrative embodiment of a software control algorithm for controlling the pasteurization unit of either of FIGS. 27A and 27B.

FIG. 29 is a schematic diagram of one illustrative embodiment of the residual liquid processing unit and corresponding control system that forms part of the biomaterial waste processing system of FIG. 1.

FIG. 30 is a flowchart of one illustrative embodiment of a software control algorithm for controlling the residual liquid processing unit of FIG. 29.

FIG. 31 shows the catalysis of flocculation of Pichia stipitis in the presence of 0.125 g/L of xanthan gum and increasing amounts of iron in ppm (x-axis). Flocculation was measured by allowing the yeast to settle for 4 minutes, taking samples from the supernatant, and counting the cells using a hemocytometer.

FIG. 32 shows the catalysis of flocculation of Pichia stipitis in the presence of various xanthan gum (see legend) and iron concentrations (x-axis). Flocculation was measured as described in the description of FIG. 31 above.

FIG. 33 shows the iron concentrations in ppm in the supernatant (y-axis) for Pichia stipitis flocculated in the presence of 0.0125 g/L of xanthan gum and increasing amounts in ppm of iron (x-axis).

FIG. 34 shows the catalysis of flocculation of Saccharomyces cerevisiae in the presence of increasing amounts in ppm of iron (x-axis) and under control conditions (diamonds), in the presence of 0.5 g/L of magnesium (triangles), at pH 7.11 (crosses), or in the presence of 2.5 g/L of NaCl (pluses).

FIG. 35 shows the iron concentration in the supernatant in ppm (y-axis) during catalyzed flocculation of Saccharomyces cerevisiae with 0.025 g/L of xanthan gum and increasing concentrations of iron (x-axis) in the presence of 0.5 g/L of magnesium (squares), at pH 7.11 (triangles), or in the presence of 2.5 g/L of NaCl (crosses).

FIG. 36 shows the percentage of yeast in the flocculating form (y-axis) versus the percentage of dilution of the sample (x-axis) for Saccharomyces cerevisiae (diamonds), Pichia stipitis (triangles), and Candida utilis (squares) flocculated in the presence of 0.025 g/L of xanthan gum and 15 ppm (S. cerevisiae and C. utilis) or 20 ppm (P. stipitis) of iron.

FIG. 37 shows the settling rate (inches/minute) (centimeters/minute) for Saccharomyces cerevisiae, Pichia stipitis, Kluyveromyces lactis, and Candida utilis flocculated in the presence of 0.025 g/L of xanthan gum (0.025 g/L) and 15 ppm of iron.

FIG. 38 shows the catalysis of flocculation of Saccharomyces cerevisiae flocculated in the presence of 0.025 g/L of xanthan gum and in the presence of 5 ppm (diamonds), 10 ppm (squares), or 15 ppm (triangles) of iron at increasing pH (x-axis).

FIG. 39 shows the catalysis of flocculation of Saccharomyces cerevisiae flocculated in the presence of 0.025 g/L of xanthan gum and in the presence of 5 ppm of iron and 2 g/L of xylitol (diamonds), 10 ppm of iron and 4 g/L of xylitol (squares), or 15 ppm of iron and 6 g/L of xylitol (triangles) at increasing pH (x-axis).

FIG. 40 shows the catalysis of flocculation of E. coli flocculated in the presence of 0.025 g/L of xanthan gum and in the presence of increasing concentrations of iron (x-axis) at a pH of 5 (diamonds) or 9 (squares).

FIG. 41 shows the catalysis of flocculation of Bacillus sp. flocculated in the presence of 0.025 g/L of xanthan gum and in the presence of increasing concentrations of iron (x-axis) at a pH of 5 (diamonds) or 9 (squares).

FIG. 42 shows the catalysis of flocculation of E. coli flocculated in the presence of 0.025 g/L of xanthan gum and in the presence of increasing concentrations of iron (x-axis) at pH's of 3, 5, 7, 9, and 11.

FIG. 43 shows the catalysis of flocculation of Bacillus sp. flocculated in the presence of 0.025 g/L of xanthan gum and in the presence of increasing concentrations of iron (x-axis) at pH's of 3, 5, 7, 9, and 11.

FIGS. 44A and 44B show an illustrative system for treating a ruminant waste stream.

FIG. 44C shows an illustrative system for removing lignin.

FIG. 44D shows an illustrative system for acid hydrolysis.

FIG. 45A shows an illustrative system for treating a swine waste stream.

FIG. 45B shows an illustrative embodiment of a swine waste receptacle.

FIG. 46 shows an illustrative system for treating a cheese processing waste stream.

FIG. 47 shows an illustrative correlation between conductivity and pH.

FIG. 48 shows an illustrative system for treating fat and oil waste.

FIG. 49 shows an illustrative system for removing dissolved and/or undissolved solids from an aqueous solution.

FIGS. 50A and 50B show a front view and a top view, respectively, of an illustrative aggregation tank for removing dissolved and/or undissolved solids from an aqueous solution.

FIG. 51 shows an illustrative process for disposing of a biomaterial waste stream, including an optional pre-processing step for treating solids removed from the biomaterial waste stream, and an optional post-processing step for removing dissolved and undissolved solids from a biomaterial waste stream.

FIG. 52 shows chromatographic traces for various samples of barn flush liquid waste based on changes in pH, added aluminum, heating, and spiking with Bovine Carbonic Anhydrase.

### DETAILED DESCRIPTION

### Illustrative Embodiments of a System for Processing a Biomaterial Waste Stream

For the purpose of promoting an understanding of the principles of this disclosure, reference will now be made to one or more embodiments illustrated in the drawings and specific language will be used to describe the same. It will nevertheless be understood that no limitation of the scope of the claims appended hereto is thereby intended.

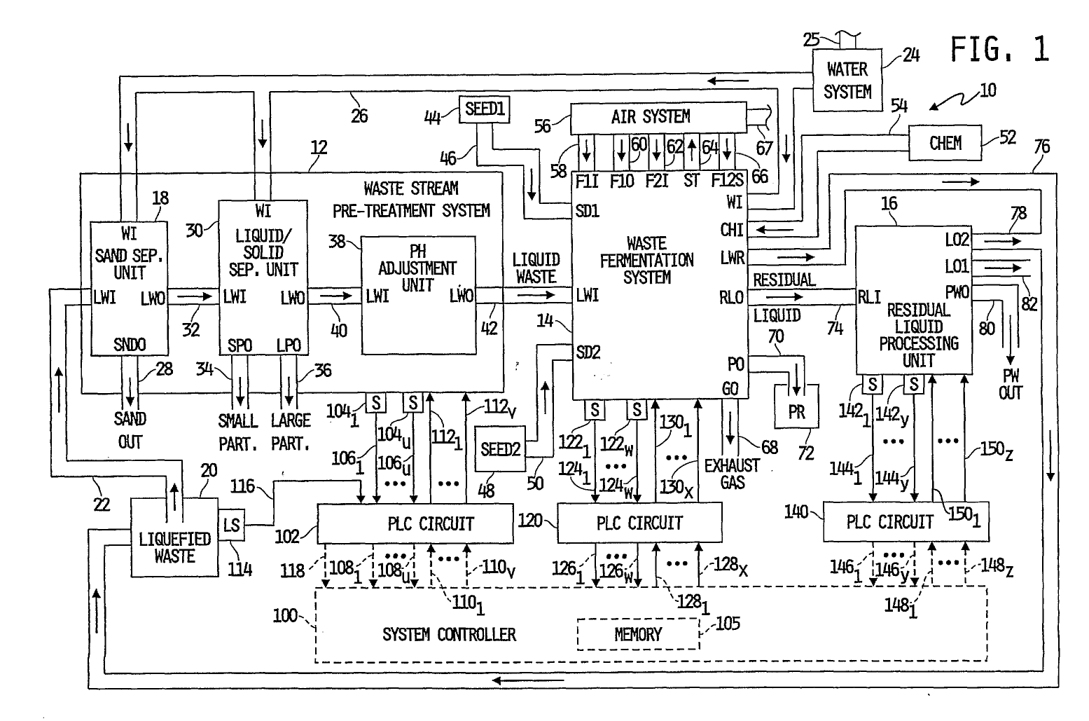

Referring to FIG. 1, a block diagram of one illustrative embodiment 10 of a system for processing a biomaterial waste stream is shown. The system 10 illustrated in FIG. 1 will be described in detail herein as being operable to process a continuous stream or flow of liquefied biomaterial waste, having dilute, and/or variable, nutrient content, in a manner that converts the biomaterial waste stream to a fermenting organism, such as yeast, and water. In the following description of system 10 and its various components, illustrative embodiments will be shown and described with particular emphasis on processing a biomaterial waste stream in the form of animal waste, such as that produced by livestock, although it will be understood that system 10 is operable to process liquid or liquefied biomaterial waste streams produced by other sources such as food processing plants, slaughterhouses and other animal or fish processing facilities, agricultural sources, such as livestock and poultry feeding operations, human waste processing facilities, and other sources of organic waste. In any case, the fermenting organism produced by this process may have value, such as a food supplement for livestock or other animals, and the water produced by the process is generally safe for disposal as ground water.

System 10 includes a waste stream pre-treatment system 12 configured to pre-treat liquefied biomaterial waste, and to supply a resulting liquid biomaterial waste stream to a waste fermentation system 14 via conduit 42. The waste stream pre-treatment system 12 includes a sand separation unit 18 having a liquefied waste inlet, LWI, for receiving liquefied biomaterial waste from a liquefied waste source 20 via conduit 22. In the illustrated embodiment, the liquefied biomaterial waste source 20 may be an animal waste storage lagoon or other animal waste storage arrangement having liquefied animal waste stored therein, or may instead be another waste processing system configured to process animal waste in a manner that produces liquefied animal waste and that supplies a stream of such liquefied animal waste to the waste stream pre-treatment system 12. One example of such a waste processing system may be a processing system configured to receive animal waste in the form of a dry or semi-dry composition of sand and animal waste, and to hydrate and separate the composition into bulk sand and liquefied animal waste in a manner that produces a continuous stream of the liquefied animal waste. One embodiment of such a sand and animal waste composition processing system is disclosed in PCT/US2005/______, entitled SAND AND ANIMAL WASTE SEPARATION SYSTEM (attorney docket no. 35479-77857), which is assigned to the assignee of the present invention, and incorporated herein by reference.

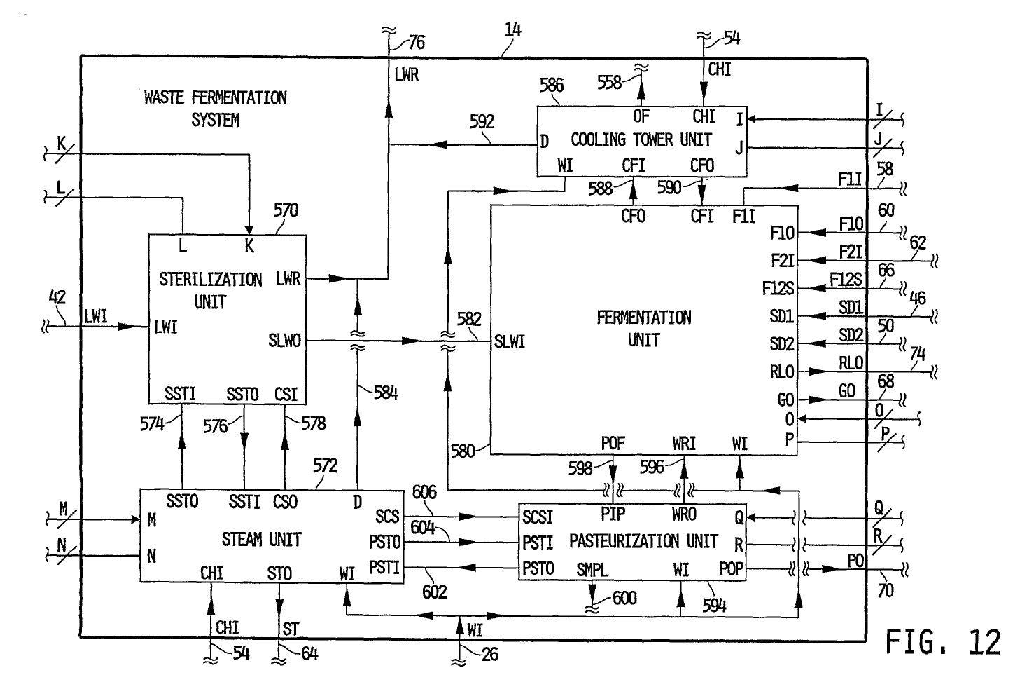

The sand separation unit 18 further includes a water inlet, WI, receiving fresh water from a water system 24 via water inlet conduit 26, a sand outlet, SNDO, producing bulk sand via sand outlet conduit 28, and a liquefied waste outlet, LWO, supplying liquefied waste to a liquefied waste conduit 32. In the illustrated embodiment, the water system 24 is a water processing system operable to receive tap water from a conventional tap water source (not shown) via conduit 25, to condition the water via conventional water conditioning; e.g., softening, techniques, and to supply the conditioned water to water conduit 26, and one illustrative embodiment of such a water system will be described in detail hereinafter with respect to FIG. 11. Alternatively, the water system 24 may be a conventional source of tap water, wherein such tap water may or may not be conditioned.

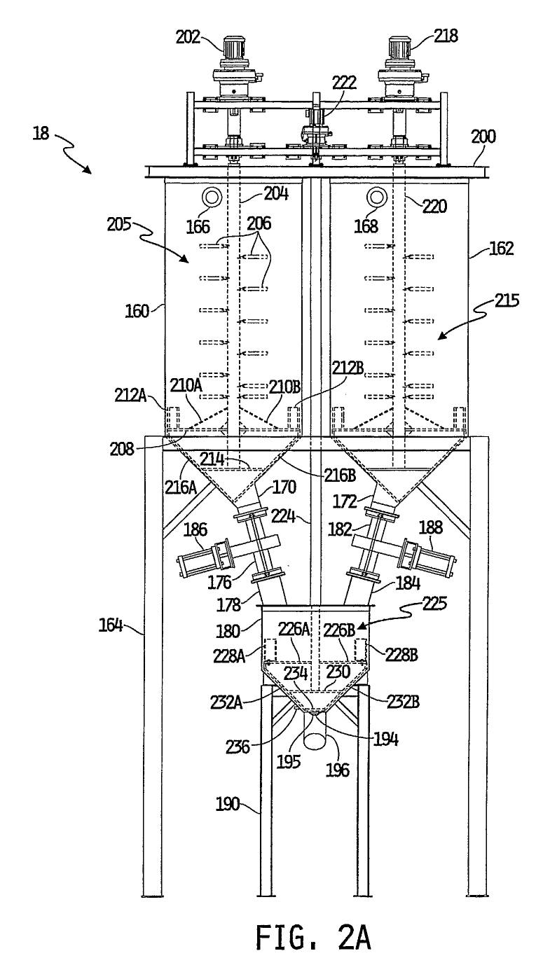

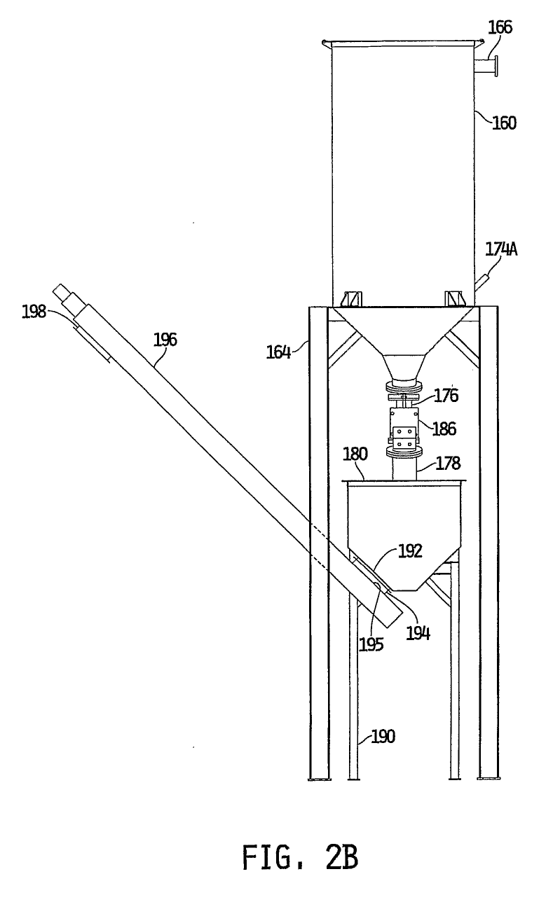

In the illustrated embodiment, the sand separation unit 18 is operable to separate sand from the liquefied waste supplied by the liquefied waste source 20, and to supply the resulting liquefied waste to a liquefied waste conduit 32. It will be understood that in embodiments of system 10 wherein the liquefied waste source 20 is a sand and waste composition processing system of the type described hereinabove, the sand separation unit 18 may also be included within some implementations of the waste stream pre-treatment system 12 to act as a secondary sand separation unit, or may in other implementations be omitted from the waste stream pre-treatment system 12. Whether to include the sand separation unit 18 in such embodiments will depend on a number of factors including the volume of sand, or sand to waste ratio in the sand and animal waste composition, the sand extraction capacity of the sand and waste composition processing system, the volume of sand in, or sand to waste ratio of, the liquefied waste supplied to the sand separation unit 18, the maximum allowable sand volume in, or sand to waste ratio of, the liquefied waste stream provided to the remaining components of the biomaterial waste processing system 10, and the like. In any case, further details relating to one illustrative structure, control system and control strategy for the sand separation unit 18 will be described in detail hereinafter with respect to FIGS. 2A-4B.

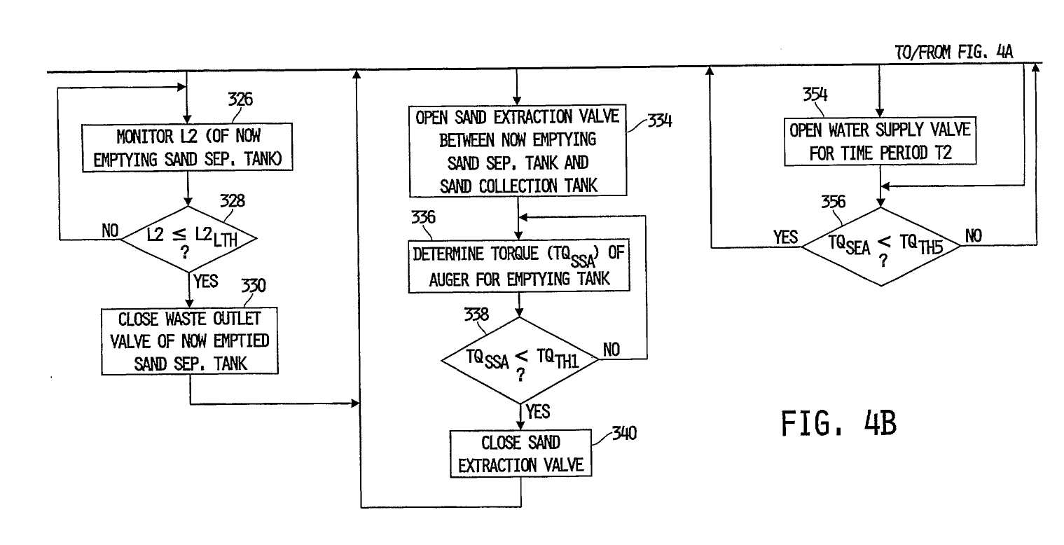

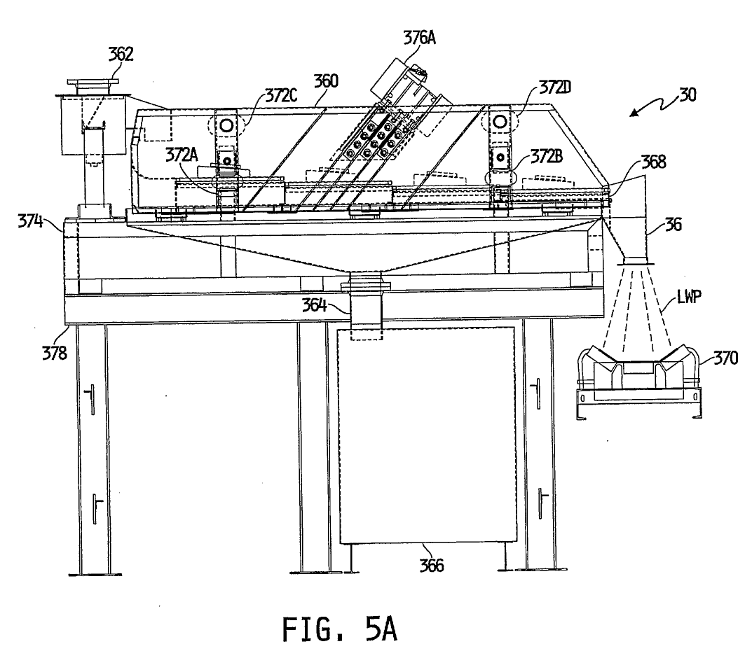

The waste stream pre-treatment system 12 further includes a liquid/solid separation unit 30 having a liquefied waste inlet, LWI, for receiving the liquefied biomaterial waste stream from the sand separation unit 18, a water inlet, WI, receiving fresh water from water system 24 via the water inlet conduit 26, a small particle outlet, SPO, coupled to a small particle outlet conduit 34, a large particle outlet, LPO, coupled to a large particle outlet conduit 36, and a liquid waste outlet, LWO, supplying liquid waste to a liquid waste conduit 40. In the illustrated embodiment, the liquid/solid separation unit 30 is operable to separate waste particles larger than a predefined size from the liquefied waste stream supplied by the sand separation unit 18, and to produce a resulting liquid waste, from which small waste particles are further extracted, and to supply the resulting liquid waste to the liquid waste conduit 40. Further details relating to one illustrative structure, control system and control strategy for the liquid/solid separation unit 30 will be described in detail hereinafter with respect to FIGS. 5A-7.

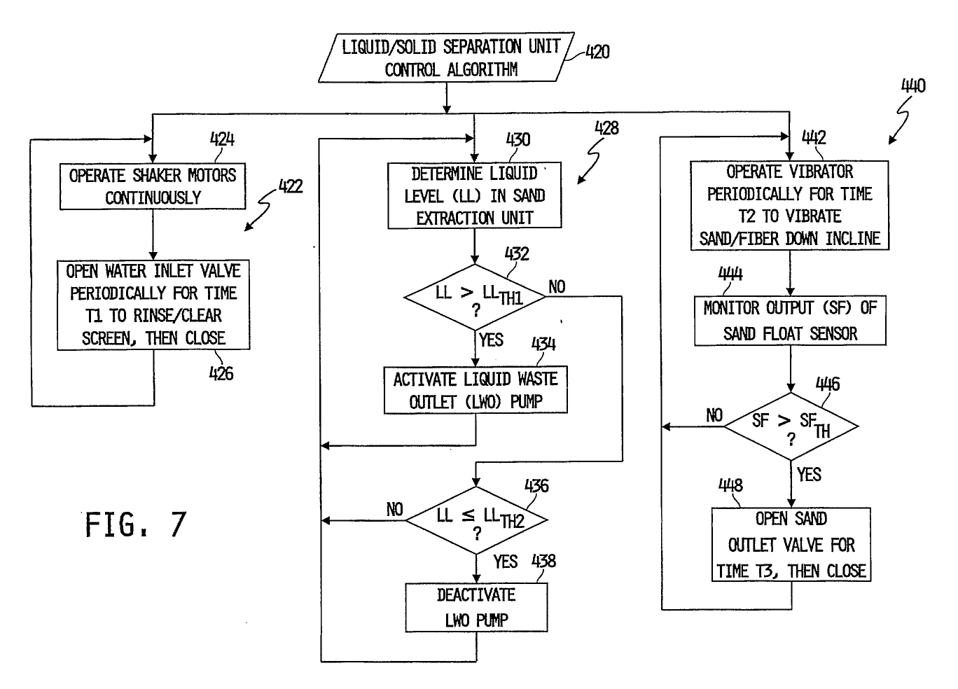

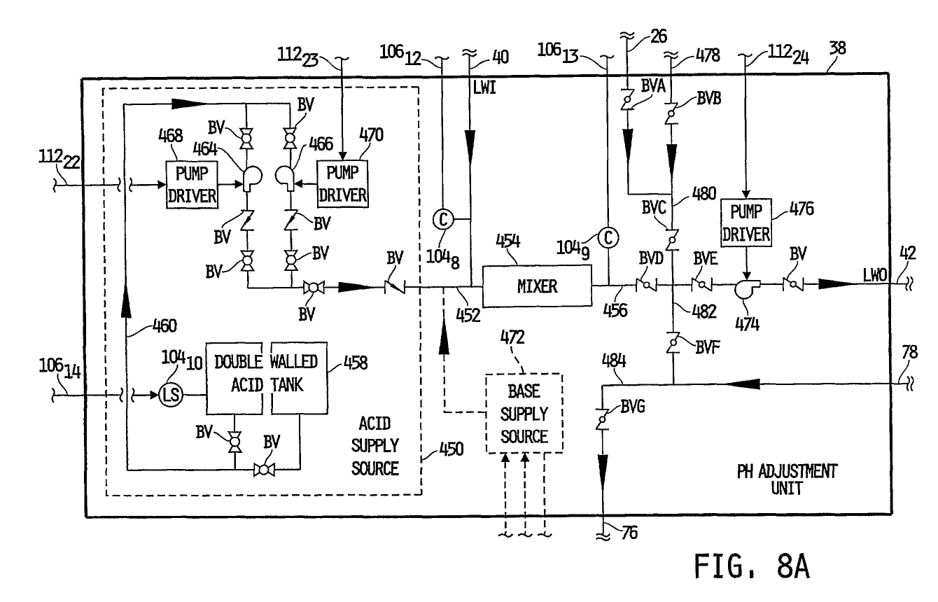

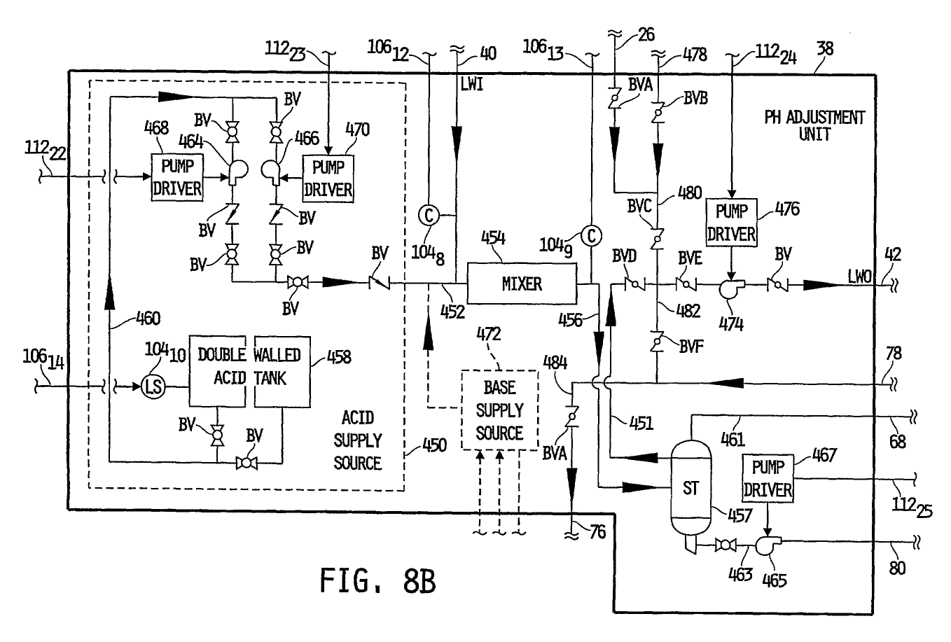

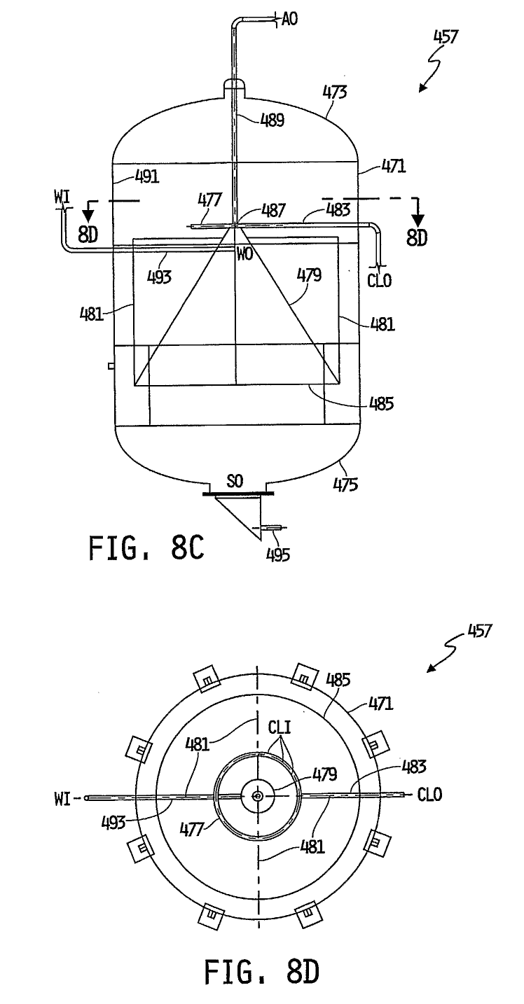

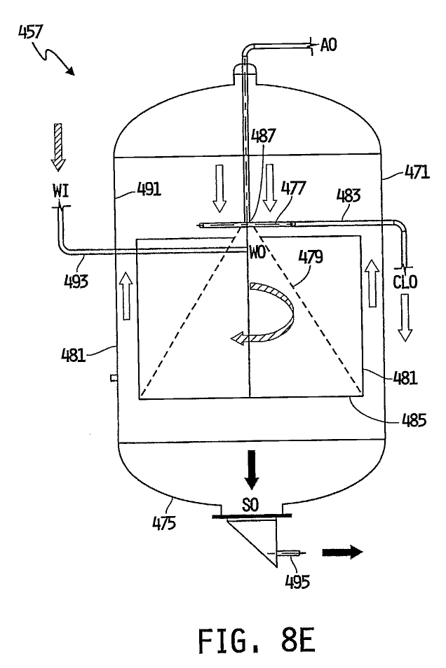

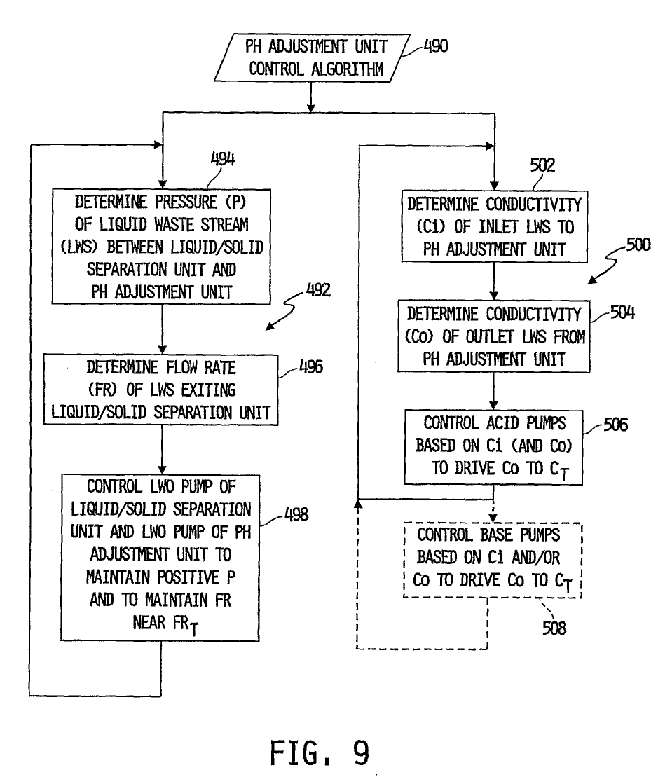

The waste stream pre-treatment system 12 further includes a pH adjustment unit 38 having a liquid waste inlet, LWI, for receiving the liquid biomaterial waste stream from the liquid/solid separation unit 30 and a liquid waste outlet, LWO, supplying liquid waste to a liquid waste conduit 42. In the illustrated embodiment, the pH adjustment unit 38 is operable to selectively adjust the pH level of the liquid waste stream supplied to conduit 42 to a target pH level. Further details relating to one illustrative structure, control system and control strategy for the pH adjustment unit 38 will be described in detail hereinafter with respect to FIGS. 8A-9.

The liquid biomaterial waste stream exiting the waste stream pre-treatment system 12 is supplied to a liquid waste inlet, LWI, of the waste fermentation system 14 via conduit 42. It will be understood that one or more of the components of the waste stream pretreatment system 12 just described may not be strictly required in some embodiments of the biomaterial waste processing system 10 for effective operation the waste fermentation system 14. However, inclusion of the components of the pre-treatment system 12 illustrated in FIG. 1 provide for optimization of the some of the physical properties of the liquid waste stream supplied to the waste fermentation system 14 in embodiments wherein the liquefied biomaterial waste is liquefied animal waste. In any case, the waste fermentation system 14 further includes a first seed inlet, SD1, fluidly coupled to a first seed source 44 via conduit 46, and a second seed inlet, SD2, fluidly coupled to a second seed source 48 via conduit 50. Seed inlet ports SD1 and SD2 are each configured to receive a microorganism seed from a corresponding seed source to begin fermentation within the waste fermentation system 14 as will be described in greater detail hereinafter. The waste fermentation system 14 further includes a chemical inlet, CHI, fluidly coupled to a chemical source 52 via conduit 54, wherein the chemical inlet, CHI, is configured to receive a chemical solution for conditioning water used by one or more of the components of the waste fermentation system 14.

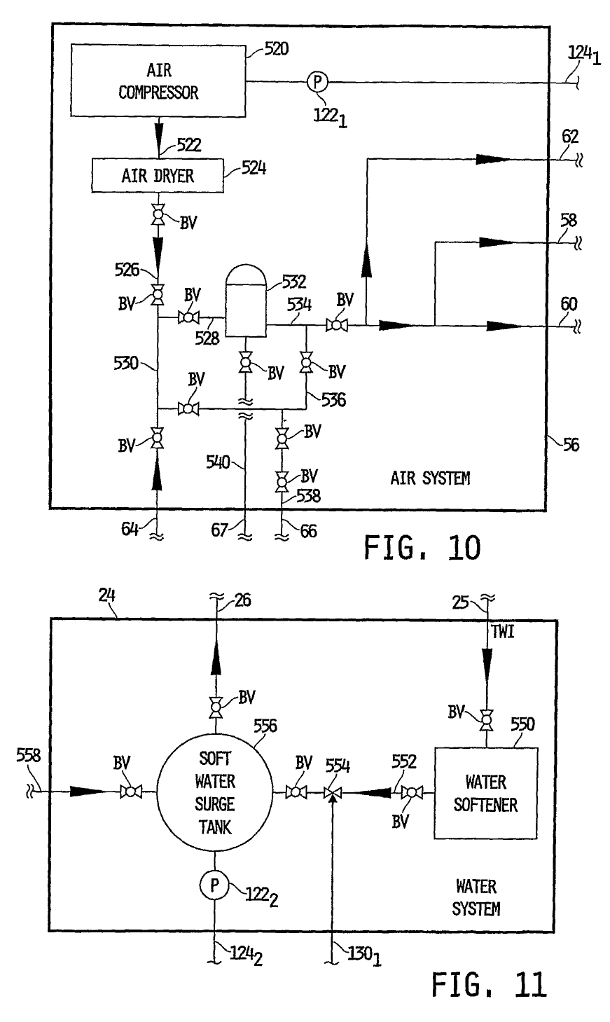

An air system 56 is coupled to the waste fermentation system 14 via a number of conduits, and is configured to supply pressurized air for use by one or more components of the waste fermentation system 14. In the illustrated embodiment, the waste fermentation system 14 includes a first inner air sparger inlet, F1I, receiving pressurized air from the air system 56 via conduit 58, a first outer air sparger inlet, F1O, receiving pressurized air from the air system 56 via conduit 60, a second inner air sparger inlet, F2I, receiving pressurized air from air system 56 via conduit 62, a steam outlet, ST, providing steam to the air system 56 via conduit 64, and a seed steam inlet, F12S, receiving pressurized steam from the air system 56 via conduit 66. The air system 56 further includes a drain outlet to allow draining of condensed water via a drain conduit 67. One illustrative embodiment of the air system 56 will be described in detail hereinafter with respect to FIG. 10.

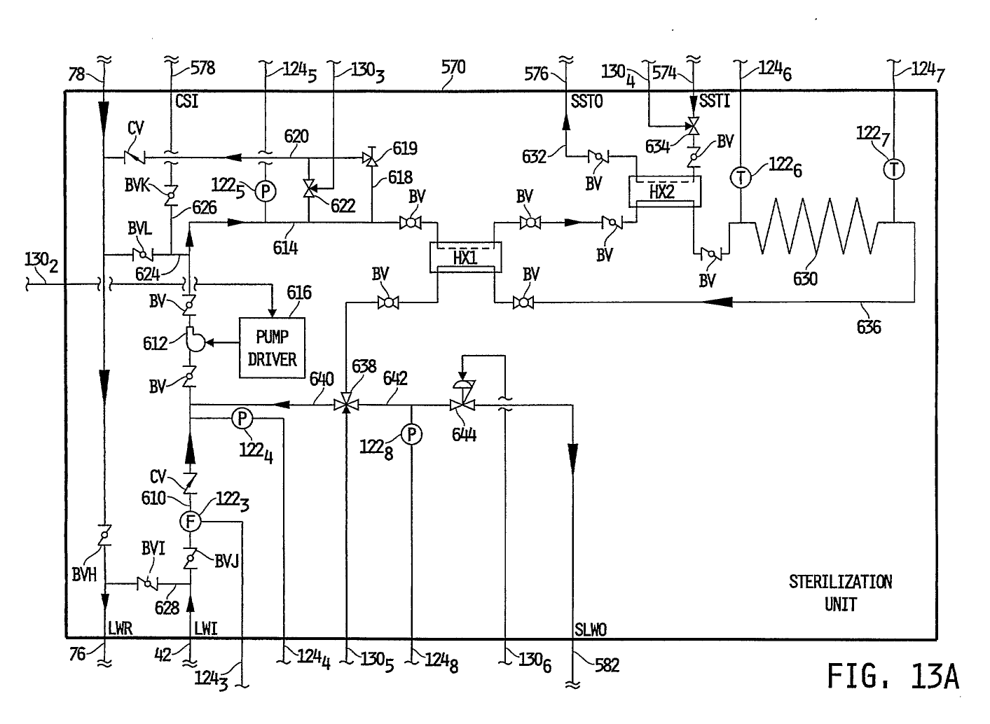

The waste fermentation system 14 further includes a gas outlet, GO, fluidly coupled to a gas outlet conduit 68, wherein the waste fermentation system 14 is operable to expel exhaust gases; e.g., exhaust air, resulting from the waste fermentation process. A product outlet port, PO, of the waste fermentation system 14 is fluidly coupled to a product outlet conduit 70, and the fermenting organism resulting from the fermentation process within the waste fermentation system 14 may be extracted from the waste fermentation system 14 via conduit 70 and collected in a suitable product receiving container 72. A residual liquid outlet, RLO, of the waste fermentation system 14 is fluidly coupled to a residual liquid conduit 74, and the waste fermentation system 14 is configured to expel residual liquid resulting from the fermentation process therein via conduit 74. A liquid waste return outlet, LWR, of the waste fermentation system 14 is fluidly coupled to a liquid waste return conduit 76, and the waste fermentation system 14 is configured to expel waste water resulting from the operation of the waste fermentation system 14 via conduit 76. One illustrative embodiment of the waste fermentation system 14 will be described in detail hereinafter with respect to FIGS. 12-28.

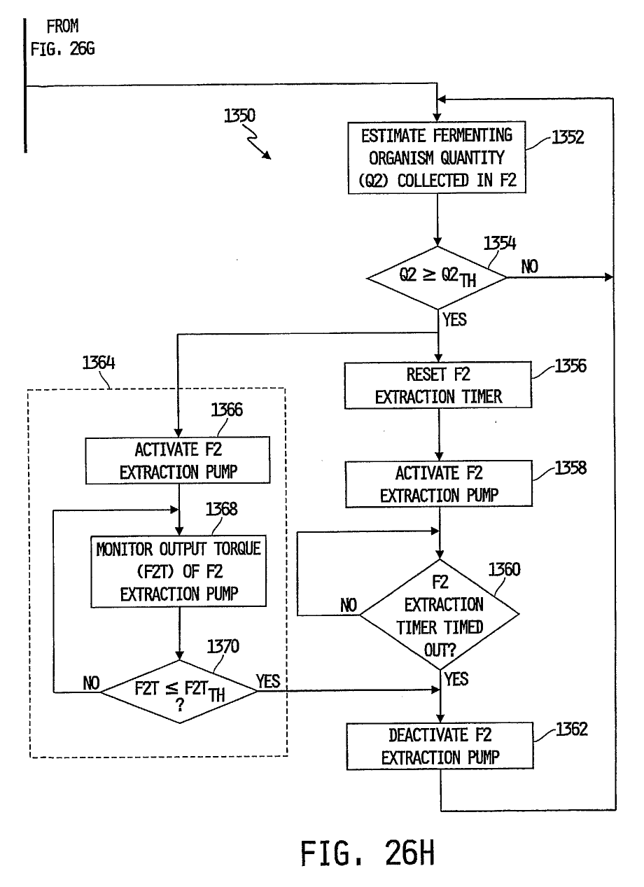

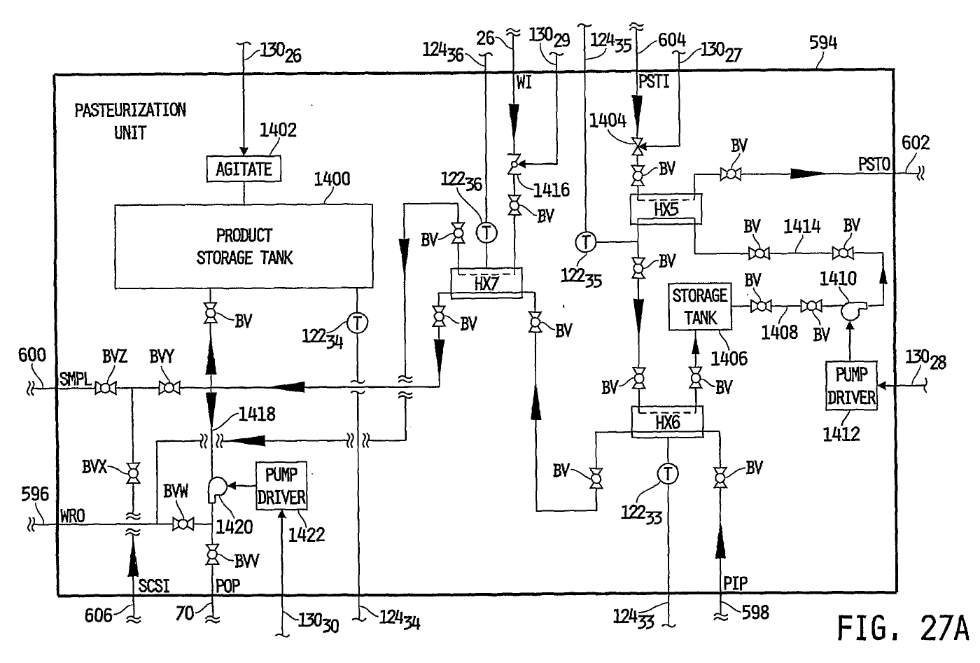

The biomaterial waste processing system 10 further includes a residual liquid post-processing unit 16 having a residual liquid inlet, RLI, receiving via conduit 74 the residual liquid produced by the waste fermentation system 14, a first liquid outlet, LO1, fluidly coupled to a liquid outlet conduit 82, a second liquid outlet, LO2, fluidly connected to the liquid waste return conduit 76 via conduit 78, and a precipitated waste outlet, PWO, fluidly coupled to a precipitated waste outlet conduit 80. In the illustrated embodiment, the residual liquid produced by the waste fermentation system 14 is the residual liquid resulting from fermentation of the liquid biomaterial waste stream. As such, this residual liquid may include a variable residual waste content, and the residual liquid processing unit 16 is configured to precipitate at least a substantial portion of the residual waste from the residual liquid and expel the resulting substantially waste-free, cleaned water via the liquid waste conduit 82 in the form of ground water. In cases where the liquid resulting from the precipitation process is not sufficiently clean to expel from the residual liquid processing unit in the form of ground water, it may be routed back to the liquefied waste source 20 via the liquid waste return conduit 76. One illustrative embodiment of the residual liquid processing unit 16 will be described in detail hereinafter with respect to FIGS. 29-30.

At least some of the operational aspects of the biomaterial waste processing system 10 are electronically controlled, and system 10 may accordingly include any number of control circuits for executing such control. In one embodiment, for example, electronic control of the biomaterial waste processing system 10 is accomplished via a number of conventional programmable logic circuits (PLCs) distributed throughout the system 10, wherein such PLCs have a number of inputs for receiving sensory data produced by one or more sensors and a number of outputs configured to control one or more system actuators. The number of PLCs include microprocessor-based controllers and on-board memory, and may be configured to communicate with each other yet operate independently. In one illustrative embodiment, such PLCs are commercially available through ControLLogix, Inc.

In the embodiment of system 10 illustrated in FIG. 1, three such programmable logic circuits are shown; a first PLC 102 configured to control the operation of the waste stream pre-treatment system 12, a second PLC 120 configured to control operation of the waste fermentation system 14 and a third PLC 140 configured to control operation of the residual liquid processing unit 16. It will be understood that only three such PLC circuits are shown in FIG. 1 for ease of illustration and subsequent description of the operation of the system 10, and that a practical implementation of the system 10 may include any number of PLCs distributed throughout the waste stream pre-treatment system 12, the waste fermentation system 14 and the residual liquid processing unit 16 to effectuate electronic control of the biomaterial waste processing system 10.

In the illustrated embodiment, the waste stream pre-treatment system 12 includes a number, u, of sensors 104₁-104ᵤ operable to sense a corresponding number of physical operating conditions of the various components 18, 30 and 38 of the waste stream pretreatment system 12, and to supply such sensory information in the form of analog sensor signals to corresponding sensor inputs of the PLC circuit 102 via corresponding signal paths 106₁-106ᵤ, wherein “u” may be any positive integer. In embodiments of system 10 wherein the liquefied waste source 20 is a liquefied waste storage arrangement, such as a liquefied waste storage lagoon, the liquefied waste source 20 includes a level sensor 114 operable to sense the level of liquefied waste in the liquefied waste source 20, and to supply this sensory information in the form of another analog sensor signal to PLC circuit 102 via signal path 118. The PLC circuit 102 is, in turn, operable to process the sensory data provided by the number of sensors 104₁-104ᵤ and 114, and produce corresponding analog actuator signals, which are then provided via signal paths 112₁-112ᵥ to corresponding actuators associated with the various components 18, 30 and 38 of the waste stream pre-treatment system 12 to effectuate control of the various components 18, 30 and 38, wherein “v” maybe any positive integer.

The waste fermentation system 14 includes a number, w, of sensors 122₁-122_{w} operable to sense one or more physical operating conditions of the waste fermentation system 14, and to supply such sensory information to the PLC circuit 120 via corresponding signal paths 124₁-124_{w}, wherein “w” may be any positive integer. The PLC circuit 120 is, in turn, operable to process the sensory data provided by the one or more sensors 122₁-122_{w} and produce one or more resulting analog actuator signals, which are then provided via signal paths 130₁-130ₓ to corresponding actuators associated with the waste fermentation system 14 to effectuate control of the fermentation process within the waste fermentation system 14 wherein “x” may be any positive integer.

The residual liquid processing unit 16 includes a number, y, of sensors 142₁-142_{y} operable to sense one or more physical operating conditions of the residual liquid processing unit 16, and to supply such sensory information to the PLC circuit 140 via corresponding signal paths 144₁-144_{y}, wherein “y” may be any positive integer. The PLC circuit 140 is, in turn, operable to process the sensory data provided by the one or more sensors 142₁-142_{y} and produce one or more resulting analog actuator signals, which are then provided via signal paths 150₁-150_{z} to corresponding actuators associated with the excess nutrient precipitation unit 16 to effectuate control of the excess nutrient precipitation process within unit 16, wherein “z” may be any positive integer.

In an alternate embodiment, the PLC circuits 102, 120 and 140 may each be configured to include a number of analog-to-digital and a number of digital-to-analog converters. In this embodiment, a system controller 100, as illustrated in phantom in FIG. 1, is operable to control the operation of the biomaterial waste processing system 10. The system controller 100 in this alternate embodiment is microprocessor-based, and includes a memory 105 having stored therein a number of software control algorithms, wherein the microprocessor portion of the system controller 100 is configured to execute such software algorithms to control operation of the biomaterial waste processing system 10. The system controller 100 includes a number of digital inputs and outputs (I/O) each electrically connected to corresponding I/Os of any number of programmable logic controllers; e.g., PLCs 102, 120 and 140. The PLC circuits 102, 120 and 140, in this embodiment, are configured to digitize analog signals provided by sensors associated with the biomaterial waste processing system 10 to the system controller 100, and to convert digital output signals from the system controller 100 to corresponding analog control signals for controlling actuators associated with the biomaterial waste processing system 10.

In the embodiment illustrated in phantom in FIG. 1, the waste stream pretreatment system 12 includes a number, u, of sensors 104₁-104ᵤ operable to sense a corresponding number of physical operating conditions of the various components 18, 30 and 38 of the waste stream pretreatment system 12, and to supply such sensory information in the form of analog sensor signals to corresponding sensor inputs of the PLC circuit 102 via corresponding signal paths 106₁-106ᵤ, wherein “u” may be any positive integer. In embodiments of system 10 wherein the liquefied waste source 20 is a liquefied waste storage arrangement, such as a liquefied waste storage lagoon, the liquefied waste source 20 includes a level sensor 114 operable to sense the level of liquefied waste in the liquefied waste source 20, and to supply this sensory information in the form of another analog sensor signal to PLC circuit 102 via signal path 118. The PLC circuit 102 is, in turn, operable convert the analog sensor signals to corresponding digital signals, and to supply the converted digital signals to the system controller 100 via signal paths 108₁-108ᵤ and signal path 118. The system controller 100 is operable, in this embodiment, to process the sensory data provided by the number of sensors 104₁-104ᵤ and 114, and produce corresponding digital actuator signals on any of a number, v, of signal paths 110₁-110ᵥ, wherein “v” may be any positive integer. The digital actuator signals are converted by the PLC circuit 102 to corresponding analog actuator signals, which are then provided via signal paths 112₁-112ᵥ to corresponding actuators associated with the various components 18, 30 and 38 of the waste stream pretreatment system 12 to effectuate control of the various components 18, 30 and 38.

The waste fermentation system 14 further includes a number, w, of sensors 122₁-122_{w} operable to sense one or more physical operating conditions of the waste fermentation system 14, and to supply such sensory information to the PLC circuit 120 via corresponding signal paths 124₁-124_{w}, wherein “w” may be any positive integer. The PLC circuit 120 is, in turn, operable to supply the sensory information to the system controller 100 via signal paths 126₁-126_{w}. PLC circuit 120 may include any number of PLC subcircuits and is in any case operable to convert the analog sensor data to one or more digital signals, and to supply the converted signals to the system controller 100. The system controller 100 is operable, in this embodiment, to process the sensory data provided by the one or more sensors 122₁-122_{w} and produce one or more resulting digital actuator signals on a number, x, of signal paths 128₁-128ₓ, wherein “x” may be any positive integer. The one or more corresponding digital actuator signals are converted by the PLC circuit 120 to corresponding analog actuator signals, which are then provided via signal paths 130₁-130ₓ to corresponding actuators associated with the waste fermentation system 14 to effectuate control of the fermentation process within the waste fermentation system 14.

The residual liquid post-processing system 16 includes a number, y, of sensors 142₁-142_{y} operable to sense one or more physical operating conditions of the excess nutrient precipitation unit 80, and to supply such sensory information to the PLC circuit 140 via corresponding signal paths 144₁-144_{y}, wherein “y” may be any positive integer. The PLC circuit 140 is, in turn, operable to supply the sensory information to the system controller 100 via signal paths 146₁-146_{y}. PLC circuit 140 may include any number of PLC subcircuits and is in any case operable to convert the analog sensor data to one or more digital signals, and to supply the converted signals to the system controller 100. The system controller 100 is operable, in this embodiment, to process the sensory data provided by the one or more sensors 142₁-142_{y} and produce one or more resulting digital actuator signals on a number, z, of signal paths 148₁-148_{z}, wherein “z” may be any positive integer. The one or more corresponding digital actuator signals are converted by the PLC circuit 140 to corresponding analog actuator signals, which are then provided via signal paths 150₁-150_{z} to corresponding actuators associated with the excess nutrient precipitation unit 16 to effectuate control of the excess nutrient precipitation process within unit 16.

For ease of illustration and description, electronic control of the various components of the biomaterial waste processing system 10 will be described herein as being accomplished via the three illustrated PLC circuits 102, 120 and 140, it being understood that alternate forms of such control may alternatively or additionally be implemented.

Referring now to FIGS. 2A and 2B, front and side elevational views respectively of one illustrative embodiment of the sand separation unit 18 forming part of the waste stream pre-treatment system 12 is shown. It will be appreciated that some of the details illustrated in FIG. 2A are not duplicated in FIG. 2B for brevity and ease of illustration. In the illustrated embodiment, the sand separation unit 10 includes a first separation tank 160 and a second separation tank 162 elevated above the ground or other support structure by support frame 164. The liquefied waste supplied by the liquefied waste source 20 via conduit 22 enters liquefied waste inlets 166 and 168 of tanks 160 and 162 respectively, wherein the inlets 166 and 168 are each positioned adjacent to the tops of tanks 160 and 162. At the bottom of tanks 160 and 162, sand outlets 170 and 172 respectively are defined, and liquefied waste outlets are defined through lower portions of the sidewalls of the tanks 160 and 162. Only one liquefied waste outlet 174A (of tank 160) is shown in the side elevational view of unit 18 in FIG. 2B, although it will be understood that tank 162 defines an identically positioned liquefied waste outlet.

The sand outlet 170 of the sand separation tank 160 is connected via a sand conduit 176 to a sand inlet 178 defined through the top of a sand collection tank 180 positioned below each of the sand separation tanks 160 and 162. The sand outlet 172 of the sand separation tank 162 is likewise connected via a sand conduit 182 to another sand inlet 184 defined through the top of the sand collection tank 180. Sand extraction valves 186 and 188 provide selective control of sand flow through sand conduits 176 and 182 respectively. The sand collection tank 180 is supported above the ground or other support structure by a support frame 190, and the bottom of the sand collection tank 180 defines a sand outlet 192 fluidly coupled to a sand inlet 195 of a sand transport device 196 via a sand conduit structure 194. In the illustrated embodiment, the sand transport device 196 is a conventional 45-degree elongated auger defining an auger outlet 198 near an end opposite the sand inlet port 195, wherein the auger 196 is operable in a known manner to receive sand expelled from the sand outlet 192 of the sand collection tank 180 via the sand conduit structure 194, and to transport the sand to the opposite end 198 of the sand extraction auger 196 where it may be collected and stored and/or transported to a convenient location using conventional machinery. Alternatively, the sand transport device 196 may be provided in the form of an auger positioned other than 45 degrees relative to unit 18 (or relative to the ground or other support surface supporting unit 18), a conventional sand conveyor, or the like.

Adjacent to the tops of the sand separation tanks 160 and 162 a support frame 200 supports a number of rotational auger motors 202, 218 and 222. Auger motor 202 is rotatably coupled to an auger shaft 204 extending into the sand separation tank 160. Adjacent to the interface between the cylindrical and conical portions of tank 160, a bar or rod 208 extends laterally away from auger shaft 204, which is connected adjacent free ends thereof to angled support bars or rods 210A and 210B extending generally upwardly toward, and connected to, the auger shaft 204. The opposite ends of bar or rod 208 carry upwardly extending plates 212A and 212B positioned adjacent the sidewalls of the tank 160, and the liquefied waste outlet 174A defined through the sidewall of the tank 160 is positioned between the bar or rod 208 and the tops of plates 212A and 212B. Another bar or rod 214 is affixed to a bottom end of the auger shaft 204, and opposite ends of the bar or rod 214 are connected to angled bars or rods 216A and 216B extending downwardly from opposite ends respectively of bar or rod 208 toward the bottom of the conical tank bottom. The ends of the angled bars or rods 216A and 216B at the bottom of the conical tank bottom are connected together. Between the angled support bars or rods 210A and 210B and the top of the tank 160, a number of mixing tines 206 extend transversely from the auger shaft 206. The auger shaft 204 and structures 206-216B extending from the auger shaft 204 define a first sand separation auger 205 rotatable within the sand separation tank 160 to separate sand from the liquefied waste entering the sand separation unit 18.

Auger motor 218 is rotatably coupled to an auger shaft 220 extending into the sand separation tank 162, and an auger structure identical to that just described with respect to the sand separation tank 160 extends from auger shaft 220 to define a second sand separation auger 215. The structures of the sand separation tanks 160 and 162, and of the sand separation augers 205 and 215, are configured to create a lateral flow of the liquefied waste about the tanks 160 and 162 when the augers 205 and 215 are rotatably driven while sand resident in the liquefied waste matter extracted from the liquefied waste source 20 drops out of the remaining liquefied waste and collects in the conical bottom portion of the sand separation tanks 160 and 162.

The auger motor 222 is rotatably coupled to an auger shaft 224 extending into the sand collection tank 160. Adjacent to the interface between the cylindrical and conical portions of the tank 160, a pair of bars or rods 226A and 226B extend laterally away from the auger shaft 224 in opposite directions, which are connected adjacent free ends thereof to ends of angled bars or rods 232A and 2132B extending generally downwardly toward the bottom of the conical bottom portion of the sand collection tank 180. The ends of bars or rods 226A and 226B adjacent to the sidewalls of the tank 180 carry upwardly extending plates 228A and 228B. Another bar or rod 230 is affixed to a bottom end of the auger shaft 224, and opposite ends of the bar or rod 230 are connected to angled bars or rods 232A and 232B extending downwardly toward the bottom of the conical portion of the tank 180. The ends of the angled bars or rods 232A and 232B at the bottom of the conical tank bottom are connected together via another bar or rod 234. The auger shaft 224 and structures 226A-234 extending from the auger shaft 224 define a sand extraction auger 225 rotatable within the sand collection tank 180 to agitate the sand collected from the sand separation tanks 160 and 162 and expel the collected sand from the sand collection tank 180. A water inlet 236 is defined through the sidewall of the sand collection tank 180 adjacent to the bottom of conical bottom portion of the tank 180.

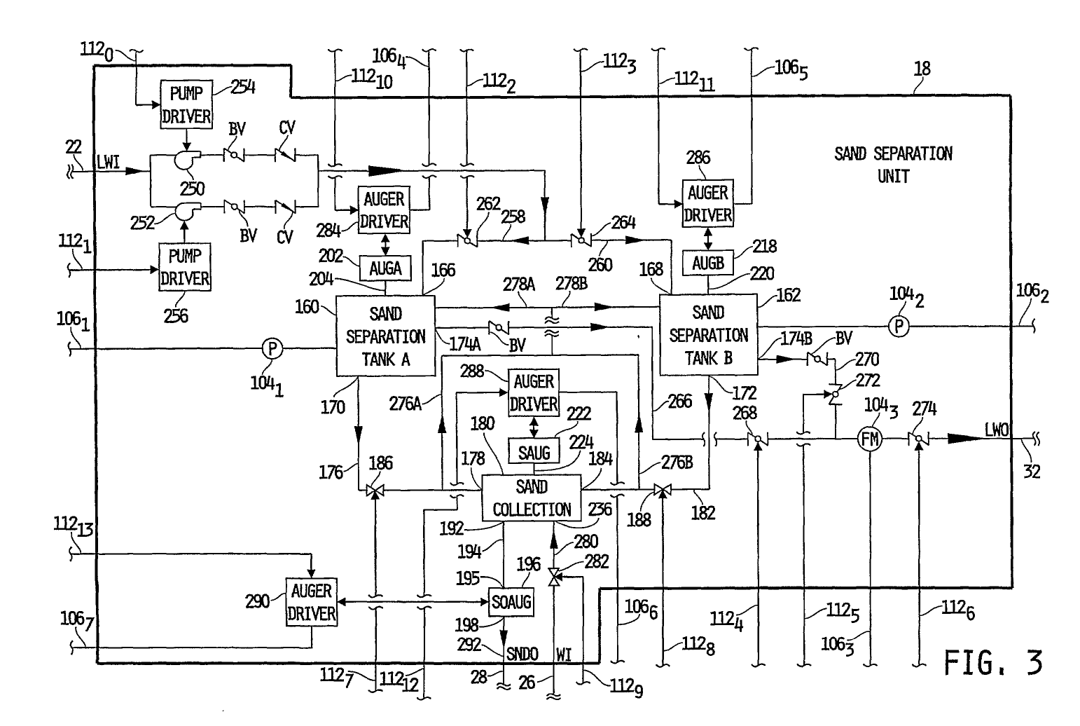

Referring now to FIG. 3, a schematic diagram of one illustrative embodiment of a control system for controlling the sand separation unit 18 of FIGS. 1-2B is shown. In the illustrated embodiment, the liquid waste inlet, LWI, of the sand separation unit 18 is fluidly connected to a pair of liquefied waste pumps 250 and 252. Pump 250 is electrically connected to a conventional pump driver 254 that is electrically connected to an actuator control output of PLC circuit 102 via signal path 112₀, and pump 252 is likewise electrically connected to a conventional pump driver 256 that is electrically connected to an actuator control output of PLC circuit 102 via signal path 112₁. The outlets of pumps 250 and 252 are each passed through mechanical butterfly and check valves BV and CV respectively, and are fluidly coupled to the liquefied waste inlets 166 and 168 of the sand separation tanks 160 and 162 respectively via conduits 258 and 260 respectively. In the illustrated embodiment, the pumps 250 and 252 are sized, as are the pump drivers 254 and 256, to provide for the pumping of liquefied waste from the liquefied waste source at a predefined liquefied waste flow rate; e.g., 100 gallons (379 liters) per minute (gpm) (lpm). Overworking of the pumps 250 and 252 is avoided by alternately activating each pump 250 and 252 for a predefined time period while the other is deactivated, thereby allowing for periodic cooling of the pumps 250 and 252 and pump drivers 254 and 256. It will be understood, however, that the pumps 250 and 252 and pump drivers 254 and 256 may alternatively be replaced with a single pump and single pump driver sized for continuous operation to at the predefined liquefied waste flow rate. In either case, a continuous flow of liquefied waste at the predefined liquefied waste flow rate is supplied to conduits 258 and 260.

A liquefied waste inlet valve 262 is disposed in-line with conduit 258, and is electrically connected to an actuator output of PLC circuit 102 via signal path 112₂. Likewise another liquefied waste inlet valve 264 is disposed in-line with conduit 260, and is electrically connected to another actuator output of PLC circuit 102 via signal path 112₃. The liquefied waste inlet valves 262 and 264 are controlled to selectively direct the liquefied waste provided by pumps 250 and 252 to the sand separation tanks 160 and 162.

The liquefied waste outlet 174A of the sand separation tank 160 is passed through a butterfly valve, BV, and fluidly connected to the liquefied waste outlet, LWO, of the sand separation unit 18 via conduit 266. A liquefied waste outlet valve 268 is disposed in-line with conduit 266, and is electrically connected to an actuator output of PLC circuit 102 via signal path 112₄. The liquefied waste outlet 174B of the sand separation tank 162 is also passed through a butterfly valve, BV, and is fluidly connected to conduit 266, downstream of the liquefied waste outlet valve 268, via conduit 270. Another liquefied waste outlet valve 272 is disposed in-line with conduit 270, and is electrically connected to another actuator output of PLC circuit 102 via signal path 112₅. Yet another liquefied waste outlet valve 274 is disposed in-line with conduit 266, downstream of valve 268 and of the junction of conduit 270 with conduit 266, and is electrically connected to yet another actuator output of PLC 102 via signal path 112₆. Conduit 266 is fluidly connected to conduit 32 supplying liquefied waste to the liquid/solid separation unit 30. The liquefied waste control valves 268, 272 and 274 are controlled to selectively extract liquefied waste from the sand separation tanks 160 and 162.

The sand extraction valve 186 disposed in-line with sand extraction conduit 176 is electrically connected to an actuator output of PLC circuit 102 via signal path 112₇, and the sand extraction valve 188 disposed in-line with sand extraction conduit 182 is electrically connected to another actuator output of PLC circuit 102 via signal path 112₈. An overflow conduit 276A is fluidly connected at one end to the sand extraction conduit 176 between the sand extraction valve 186 and the sand inlet 178 of the sand collection tank 180, and another overflow conduit 276B is fluidly connected at one end to the sand extraction conduit 182 between the sand extraction valve 188 and the sand inlet 184 of the sand collection tank 180. The opposite ends of overflow conduits 276A and 276B are both fluidly connected to another overflow conduit 278A fluidly connected to an overflow inlet of the sand separation tank 160, and to yet another overflow conduit 278B fluidly connected to an overflow inlet of the sand separation tank 162. The sand extraction valves 186 and 188 are controlled to selectively extract sand from the sand separation tanks 160 and 162 and collect the extracted sand in the sand collection tank.

The water inlet, WI, of the sand separation unit 18 is fluidly connected to the water inlet 236 of the sand collection tank 180 via a water conduit 280, wherein conduit 280 is fluidly connected to water inlet conduit 26. A water inlet valve 282 is disposed in-line with conduit 280, and is electrically connected to an actuator output of the PLC circuit 102 via signal path 112₉. The water inlet valve 282 is controlled to selectively supply water to the sand collection tank 180.

The control system illustrated in FIG. 3 also includes a number of sensors producing sensory information relating to operation of the sand separation unit 18. For example, a pressure sensor 104₁ is fluidly coupled to the sand separation tank 160, and is electrically connected to a sensor input of the PLC circuit 102 via signal path 106₁. Another pressure sensor 104₂ is fluidly coupled to the sand separation tank 162, and is electrically connected to another sensor input of the PLC circuit 102 via signal path 106₂. The pressure sensors 104₁ and 104₂ provide the PLC circuit 102 with information relating to the pressures within the sand separation tanks 160 and 162 respectively, and the PLC circuit 102 is operable in a known manner to process this pressure information and determine the levels of liquid or liquefied matter within the separation tanks 160 and 162 respectively. Alternatively, each tank 160 and 162 may include one or more other conventional level sensors configured to provide the PLC circuit 102 with information relating to one or more liquid or liquefied matter thresholds within tanks 160 and 162. In any case, a conventional flow meter or flow sensor 104₃ is disposed in-line with the liquefied waste outlet conduit 266 downstream of the liquefied waste control valve 268 and of the junction of conduit 270 with conduit 268, and upstream of the liquefied waste control valve 274, and is electrically connected to another sensor input of the PLC circuit 102 via signal path 106₃. The PLC circuit 102 is responsive to the sensory information provided by sensors 104₁-104₃ to control one or more operational features of the sand separation unit 18.

The auger motor 202 is electrically connected to an auger driver 284 that is electrically connected to another actuator output of the PLC 102 via signal path 112₁₀, and also electrically connected to a sensor input of the PLC circuit 102 via signal path 106₄. The auger driver 284 is responsive to an actuator control signal supplied by the PLC 102 to drive auger motor 202, and the auger driver 284 and/or auger motor 202 further includes a “sensor” for determining and monitoring the operating torque of the auger motor 202. Such a “sensor” may be a conventional strain-gauge type torque sensor operatively coupled to a rotating drive shaft of the auger motor 202 and operable to produce a sensor signal corresponding to the operating torque of the auger motor 202, or may alternatively be a so-called virtual sensor implemented in the form of one or more software algorithms resident within the PLC circuit 102 and responsive to one or more measurable operating parameters associated with the auger driver 284 and/or auger motor 202 to derive or infer the operating torque value. For example, the auger driver 284 may include a current sensor producing a current sensor signal indicative of drive current being drawn by the driver 284, and/or the auger motor 202 may include a position and/or speed sensor producing a signal corresponding to the rotational speed and/or position of the auger shaft 204. The PLC circuit 102 may be responsive to any such sensor signals, and/or to other information relating to the operation of the auger driver 284 and/or auger motor 202, to estimate the operating torque of the auger motor 202 as a known function thereof. In any case, the signal path 1064 carries one or more torque feedback signals to the PLC circuit 102 from which the operating torque of the auger motor 202 may be determined directly or estimated.

The auger motor 218 is likewise electrically connected to an auger driver 286 that is electrically connected to another actuator output of the PLC 102 via signal path 112₁₁, and also electrically connected to another sensor input of the PLC circuit 102 via signal path 106₅. The auger driver 284 is responsive to an actuator control signal supplied by the PLC 102 on signal path 112₁₁ to drive auger motor 218, and to provide a torque feedback signal to the PLC circuit 102, using any of the techniques just described, corresponding to the operating torque of the auger motor 218 or from which the operating torque of the auger motor 218 may be estimated.

The auger motor 222 is also electrically connected to an auger driver 288 that is electrically connected to another actuator output of the PLC 102 via signal path 112₁₂, and also electrically connected to another sensor input of the PLC circuit 102 via signal path 106₆. The auger driver 288 is responsive to an actuator control signal supplied by the PLC 102 on signal path 112₁₂ to drive auger motor 222, and to provide a torque feedback signal to the PLC circuit 102, using any of the techniques described hereinabove, corresponding to the operating torque of the auger motor 222 or from which the operating torque of the auger motor 222 may be estimated.

The sand extraction auger 196 also includes an auger motor electrically connected to an auger driver 290 that is electrically connected to another actuator output of the PLC 102 via signal path 112₁₃, and also electrically connected to another sensor input of the PLC circuit 102 via signal path 106₇. The auger driver 290 is responsive to an actuator control signal supplied by the PLC 102 on signal path 112₁₃ to drive the motor of the sand extraction auger 196, and to provide a torque feedback signal to the PLC circuit 102, using any of the techniques described hereinabove, corresponding to the operating torque of the auger 196 or from which the operating torque of the auger 196 may be estimated. The sand outlet of the sand collection tank 180 is fluidly coupled to the sand inlet of the sand extraction auger 196 via conduit structure 194. The outlet 198 of the sand extraction auger 196 defines the sand output, SNDO, of the sand separation unit 18 and is fluidly coupled to the sand extraction conduit 28 via conduit 292.

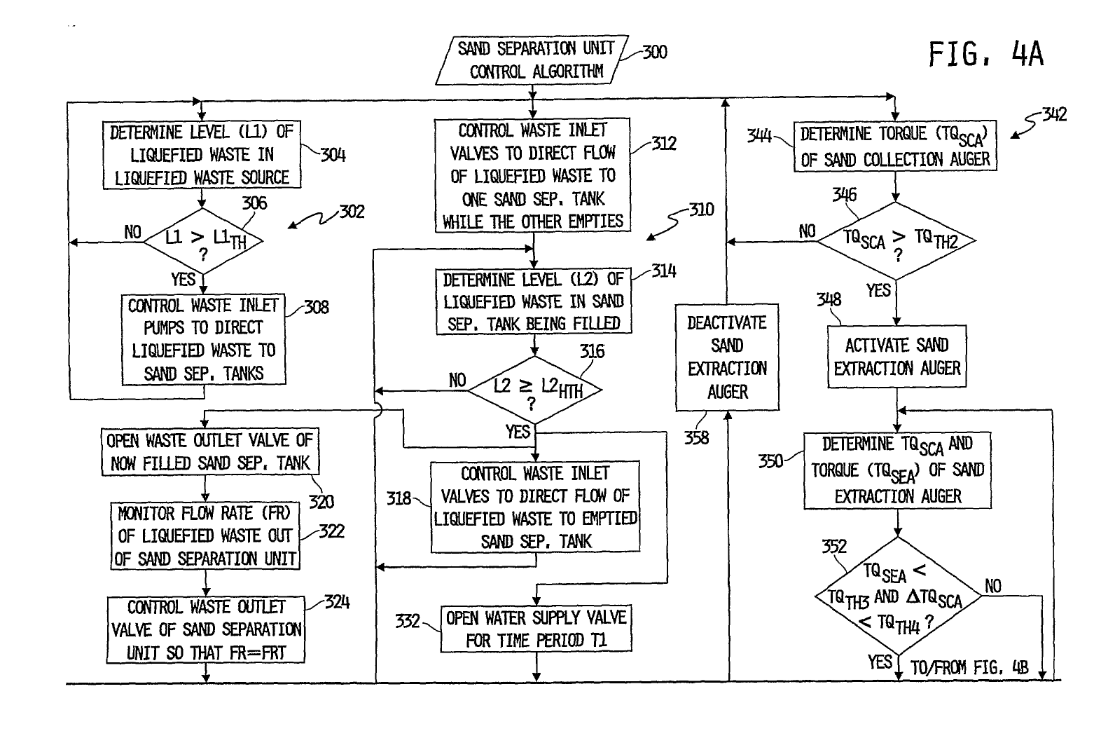

Referring now to FIGS. 4A and 4B, a flowchart of one illustrative embodiment of a software control algorithm 300 for controlling the sand separation unit 18 via the control system of FIG. 3 is shown. Control algorithm 300 is stored within, or programmed into, the PLC circuit 102, and the PLC circuit 102 is operable to execute algorithm 300 to control the operation of the sand separation unit 18. The control algorithm 300 includes a number of different and independently executing control routines, and each of these different control routines will be described separately. Throughout each of the different control routines of control algorithm 300, it will be understood that the PLC circuit 102 is operable to continually operate the sand separation augers 205 and 215, as well as the sand extraction auger 225. In any case, the control algorithm 300 includes a first control routine 302 for controlling the operation of the liquefied waste pumps 250 and 252, and routine 302 begins at step 304 where the PLC circuit 102 is operable to determine the level, L1, of the liquefied waste in the liquefied waste source. In the illustrated embodiment of the system 10 of FIG. 1, the PLC circuit 102 is operable to execute step 304 by monitoring the output of the level sensor 114. Following step 304, the PLC circuit 102 is operable at step 306 to compare L1 to a threshold level value, L1_{TH}, wherein L1_{TH} corresponds to a minimum allowable level of liquefied waste in the liquefied waste source 20. If the PLC circuit 102 determines at step 306 that L1 is less than or equal to L1_{TH}, execution of the control routine 302 loops back to step 304 with no further action. If, however, the PLC circuit 102 determines at step 306 that L1 is greater than L1_{TH}, execution of the control routine 302 advances to step 308 where the PLC circuit 102 is operable as described hereinabove to control the waste inlet pumps to direct liquefied waste from the liquefied waste source 20 to the sand separation tanks 160 and 162.

From step 308, execution of the control routine 302 loops back to step 304. The control routine 302 is thus operable to control the waste inlet pumps 250 and 252 to provide liquefied waste to the sand separation system 18 only as long as the liquefied waste source 20 has stored therein a sufficient quantity of liquefied waste. It will be understood that in embodiments of the biomaterial waste processing system 10 wherein the liquefied waste source is another waste processing system, the control routine 302 may be omitted, or may instead be modified to operate pumps 250 and 252 only when such a waste processing system is supplying a sufficient quantity of liquefied waste. Any such modifications to the control routine 302 would be a mechanical step for a skilled artisan.

The sand separation unit control algorithm 300 includes another control routine 310 for controlling the filling and emptying of the sand separation tanks 160 and 162. Control routine 310 begins at step 312 where the PLC circuit 102 is operable to control opening and closing of the liquefied waste inlet valves 262 and 264 to direct the flow of liquefied waste provided by pumps 250 and 252 to one of the sand separation tanks 160, 162 while the other tank 160, 162 is being emptied. Thereafter at step 314, the PLC circuit 102 is operable to determine the level, L2, of liquefied waste in the sand separation tank 160, 162 that is being filled as a result of step 312. In the illustrated embodiment of the system 10 of FIG. 1, the PLC circuit 102 is operable to execute step 314 by monitoring the output of an appropriate one of the pressure sensors 104₁ and 104₂, and determining the level of liquefied waste in the corresponding tank as a known function of the pressure signal. Following step 314, the PLC circuit 102 is operable at step 316 to compare L2 to a high threshold level value, L2_{HTH}, wherein L2_{HTH} corresponds to a maximum allowable level of liquefied waste in either tank 160 or 162. If the PLC circuit 102 determines at step 316 that L2 is less than L2_{HTH}, execution of the control routine 310 loops back to step 314 with no further action. If, however, the PLC circuit 102 determines at step 316 that L2 is greater than or equal to L2_{HTH}, indicating that the filling sand separation tank 160 or 162 is now full, execution of the control routine 310 advances to each of a number of control branches. For example, the “yes” branch of step 316 advances to step 318 where the PLC circuit 102 is operable to control opening and closing of the liquefied waste inlet valves 262 and 264 to direct the flow of liquefied waste provided by pumps 250 and 252 to the opposite (now empty) sand separation tank 160, 162 to commence filling that tank. Execution of the control routine 310 loops from step 318 back to step 314 to monitor the level, L2, of the sand separation tank 160, 162 now being filled as a result of step 318.

The “yes” branch of step 316 also advances to step 320 where the PLC circuit 102 is operable to open the liquefied waste outlet valve 268, 272 of the now filled sand separation tank 160, 162. While the sand separation tank 160, 162 was being filled with liquefied waste via steps 312-316, the corresponding sand separation auger 205, 215 was continuously rotating to create a lateral flow of the liquefied waste about the sand separation tank 160, 162 to thereby suspend the waste solids in the circulating fluid while the sand in the tank 160, 162 dropped out of the lateral flow and was collected in the bottom of the tank 160, 162. By the time the sand separation tank 160, 162 is filled at step 316, a substantial amount of the sand present in the tank 160,162 will have dropped out of the lateral flow, and the resulting liquefied waste may begun to be removed at step 320 by opening a corresponding liquefied waste outlet valve 268, 272. Following step 320, the PLC circuit 102 is operable at step 322 to monitor the flow rate, FR, of the liquefied waste exiting the sand separation unit 18 via conduit 266. In the illustrated embodiment, the PLC circuit 102 is operable to execute step 322 by monitoring the flow signal produced by the flow meter 104₃. Thereafter at step 324, the PLC circuit 102 is operable to adjust or modulate the opening of the liquefied waste outlet valve 268, 270 so that the flow rate, FR, of the liquefied waste out of the sand separation tank 160, 162 is maintained near a target flow rate, FRT; e.g., 100 gpm.

Following step 324, the PLC circuit 102 is operable at step 326 to monitor the level, L2, of liquefied waste in the sand separation tank 160, 162 from which the liquefied waste is being removed. Thereafter at step 328 the PLC circuit 102 is operable to compare L2 to a low threshold level; L2_{LTH}, wherein L2_{LTH} corresponds in the illustrated embodiment to a level at which the liquefied waste may be considered to have been substantially removed from the sand separation tank 160, 162. If, at step 328, the PLC circuit 102 determines that L2 is greater than L2_{LTH}, execution of the control routine 310 loops back to step 326. If, on the other hand, the PLC circuit 102 determines at step 328 that L2 is less than or equal to L2_{LTH}, the liquefied waste is considered to have been sufficiently removed from the sand separation tank 160, 162 and the PLC circuit 102 is operable thereafter at step 330 to close the waste outlet valve 268, 272 of the now emptied sand separation tank 160, 162. Execution of the control routine 310 loops from step 330 back to step 314.

The “yes” branch of step 316 also advances to step 332 where the PLC circuit 102 is operable to open the water supply valve 282 to the sand collection tank 180 for a predefined time period, T1. In the illustrated embodiment, T1 is selected such that water entering the sand collection tank 180 will fill the sand collection tank 180 and travel up the sand extraction conduit 176, 182 up to the outlet of the sand extraction valve 186, 188. Any excess water flows up the overflow conduits 276A, 276B and 278A, 278B, and is spilled into the sand separation tank 160, 162. Step 332 is included within the control routine 310 to provide a flow medium within the sand extraction conduit 176, 182 between the sand collection tank 180 and the sand extraction valve 186, 188 to facilitate the transfer of sand from the sand separation tank 160, 162 into the sand collection tank 180 via control of the sand extraction valve 186, 188. Following step 332, the PLC circuit 102 is operable at step 334 to open the sand extraction valve 186, 188 between the now emptying sand separation tank 160, 162 and the sand collection tank 180 to allow sand collected in the bottom of the sand separation tank 160, 162 to flow through the sand extraction conduit 176, 182 and into the sand collection tank 180.

Following step 334, the PLC circuit 102 is operable at step 336 to determine the operating torque, TQ_{SSA}, of the sand separation auger 205, 215 of the sand separation tank 160, 162 being emptied. In the illustrated embodiment, the PLC circuit 102 is operable to execute step 336 using any of the feedback torque monitoring techniques described hereinabove. Following step 336, the PLC circuit 102 is operable at step 338 to compare the operating torque, TQ_{SSA}, of the auger 205, 215 to a torque threshold, TQ_{TH1}. As sand is transferred from the sand separation tank 160, 162 to the sand collection tank 180, the operating torque of the sand separation auger 205, 215 will decrease due to the diminishing sand quantity in the bottom of the sand separation tank 160, 162. The torque threshold TQ_{TH1} corresponds to an operating torque of the sand separation auger 205, 215 below which the sand separation tank 160, 162 may be considered to be sufficiently emptied of sand. Thus, if the PLC circuit 102 determines at step 338 that TQ_{SSA} is greater than or equal to TQ_{TH1}, the sand separation tank 160, 162 still holds a quantity of sand that may be removed, and execution of the control routine 310 thus loops back to step 336. If, on the other hand, the PLC circuit 102 determines at step 338 that TQ_{SSA} is less than TQ_{TH1}, enough sand has been extracted from the sand separation tank 160, 162 to consider it emptied of sand, and execution of the control routine 310 advances to step 340 where the PLC circuit 102 is operable to close the sand extraction valve 176, 182. From step 340, execution of the control routine 310 loops back to step 314 where the PLC circuit 102 is operable to monitor the liquefied waste level of the opposite sand separation tank 160, 162 now being filled.

The sand separation unit control algorithm 300 further includes another control routine 342 for controlling emptying of the sand collection tank 180. Control routine 342 begins at step 344 where the PLC circuit 102 is operable to determine the operating torque, TQ_{SCA}, of the sand collection auger 225 rotating within the sand collection tank 180. In the illustrated embodiment, the PLC circuit 102 is operable to execute step 344 using any of the feedback torque monitoring techniques described hereinabove. Following step 344, the PLC circuit 102 is operable at step 346 to compare the operating torque, TQ_{SCA}, of the auger 225 to a torque threshold, TQ_{TH2}. As sand is transferred from the sand separation tanks 160 and 162 to the sand collection tank 180, the operating torque of the sand collection auger 225 will increase due to the increasing sand quantity in the sand collection tank 180. The torque threshold TQ_{TH2} corresponds to an operating torque of the sand collection auger 225 above which the sand collection tank 180 may be considered to have a quantity of sand collected therein that merits removal. Thus, if the PLC circuit 102 determines at step 346 that TQ_{SCA} is less than or equal to TQ_{TH2}, the sand collection tank 180 does not hold a sufficient quantity of sand that merits removal, and execution of the control routine 342 thus loops back to step 344. If, on the other hand, the PLC circuit 102 determines at step 344 that TQ_{SCA} is greater than TQ_{TH2}, the sand collection tank 180 holds a sufficient quantity of sand to merit removal of the collected sand, and execution of the control routine 342 advances to step 348 where the PLC circuit 102 is operable to activate the sand extraction auger 196. With the sand collection auger 180 constantly rotating, activation of the sand extraction auger 196 at step 348 will cause sand collected in the sand collection tank 180 to flow out of the sand outlet 192 and through the conduit structure 194 into the sand inlet 195 of the sand extraction auger. Operation of the sand extraction auger 196 transfers the sand from the sand inlet 195 to the sand outlet 198 of the auger 196, where the extracted sand may be stored and/or transported via conventional machinery to a convenient location.

Following step 348, the PLC circuit 102 is operable at step 350 to again determine the operating torque, TQ_{SCA}, of the sand collection auger 225 rotating within the sand collection tank 180, and to also determine the operating torque, TQ_{SEA}, of the sand extraction auger 196, using any of the feedback torque monitoring techniques described hereinabove. Thereafter at step 352, the PLC circuit 102 is operable to compare TQ_{SEA} to a torque threshold value, TQ_{TH3}, and to compare the change in TQ_{SCA} to another torque threshold value, TQ_{TH4}. The torque thresholds TQ_{TH3} and TQ_{TH4} are selected to allow detection of whether sand contained within the sand collection tank 180 is sufficiently loose to allow it to be extracted from the sand collection tank 180. In this regard, the PLC circuit 102 is operable at step 352 to determine whether the operating torque, TQ_{SEA}, of the sand extraction auger 196 has dropped below TQ_{TH3} while the change in the operating torque, ΔTQ_{SCA}, of the sand collection auger 225 over a recent time interval is less than TQ_{TH4}. If so, this indicates that the sand within the sand collection tank 180 has become too tightly packed, and rehydration of is necessary to facilitate extraction of the sand from the sand collection tank 180. In this case, execution of the control routine 342 advances to step 354 where the PLC circuit 102 is operable to open the water supply valve 282 for a time period T2 to supply water from the water source 24 to the sand collection tank 180. The time period T2 is selected to allow sufficient rehydration of the sand within the sand collection chamber 180 so that it may be subsequently removed via the sand removal valve 194 and sand extraction auger 196. If, however, the PLC circuit 102 determines at step 352 that TQ_{SEA} is greater than or equal to TQ_{TH3}, and/or ΔTQ_{SCA} is greater than or equal to TQ_{TH4}, this indicates that the sand within the sand collection tank 180 is sufficiently hydrated to allow extraction thereof via the sand removal valve 194 and sand extraction auger 196.

Execution of the control routine 342 advances from the “no” branch of step 352 and from step 354 to step 356 where the PLC circuit 102 is operable to compare the operating torque, TQ_{SEA}, of the sand extraction auger 196 to another torque threshold value, TQ_{TH5}. The torque threshold, TQ_{TH5}, is set to an operating torque value below which the sand extraction auger 196 is not transferring a sufficient quantity of sand to warrant operation of the sand extraction auger 196. Thus, if the PLC circuit 102 determines at step 356 that TQ_{SEA} is greater than or equal to TQ_{TH5}, execution of the control routine 342 loops back to step 350. If, on the other hand, the PLC circuit 102 determines at step 356 that TQ_{SEA} is less than TQ_{TH5}, execution of the control routine 342 advances to step 358 where the PLC circuit 102 is operable to deactivate the sand extraction auger 196. Thereafter, execution of the control routine 342 loops back to step 344.

For continuous flow operation of the sand separation unit 18, control routine 310 is coordinated in the timing of its various execution branches so that one sand separation tank 160 or 162 is being filled with liquefied waste according to steps 312-318 while the other sand separation tank 160 or 162 is being simultaneously emptied of liquefied waste and sand according to steps 320-340. In such a continuous flow system, steps 318, 330 and 340 thus loop directly back to step 314 of control routine 310. For non-continuous flow operation, control routine 310 may require one or more delay steps to coordinate the filling of one sand separation tank 160 or 162 with the emptying of the other sand separation tank 160 or 162, and/or control algorithm 300 may require an additional control routine to control the feed rate of the liquefied waste from the liquefied waste source 20 to the sand separation tanks 160 and 162. In either case, control routine 342 operates independently of control routine 310 such that sand is extracted from the sand collection tank 180 only when the operating torque of the sand collection auger 225 exceeds a specified torque threshold.

Referring now to FIGS. 5A and 5B, side and front elevational views respectively of one illustrative embodiment of the liquid/solid separation unit 30 forming part of the waste stream pre-treatment system 12 is shown. In the illustrated embodiment, the liquid/solid separation unit 30 includes a screen shaker in the form of a shaker table 360 having a liquefied waste inlet 362 configured to receive liquefied waste from the sand separation unit 18 and supply the liquefied waste through the top of the shaker table 360 to an interior of the table 360. The shaker table 362 includes a conventional screen, mesh fabric or the like (not shown) positioned over a liquid waste outlet 364 and configured to trap waste solid waste particles larger than a predefined particle size while allowing liquid waste and solid waste particles smaller than the predefined particle size to pass through the screen or mesh to a small particle extraction unit 366 via the liquid waste outlet 364. The shaker table 362 also has a large waste particle outlet 368 coupled to the large waste particle outlet conduit 36. In one illustrative embodiment, the screen or mesh is configured to trap waste and other particles; e.g., straw, hay, bedding and the like, that are approximately 20 microns and larger, while passing liquid waste and waste particles less than 20 microns in size to the liquid waste outlet 364. It will be understood, however, that the screen or mesh may be configured to trap waste particles having any desired minimum size without detracting from the scope of the claims appended hereto. In any case, a conventional conveyor system 370 or other conventional transport device is positioned beneath the large waste particle outlet conduit 36, and is configured to receive large waste particles, LWP, removed from the shaker table 360 via conduit 36, and to transport the removed large waste particles to a convenient location for storage, disposal or farther processing.

In the illustrated embodiment, the shaker table 360 is movably connected to a support frame 374 via four conventional shaker table supports 372A-372D, although more or fewer such supports may alternatively be used. In any case, the liquid/solid separation system 30 includes a number of shaker motors configured to shake, vibrate or otherwise rapidly move the shaker table relative to the support frame 374. In the illustrated embodiment, system 30 includes two such motors 376A and 376B mounted to the table 360 at approximately a 45-degree angle relative to a longitudinal plane of the support frame 374. Thusly mounted, the shaker motors 376A and 376B are controlled to shake the shaker table 360 in both the vertical and horizontal directions to cause the liquid portion and small particles carried by of the liquefied waste to pass through the screen or mesh while the large waste particles carried by the liquefied waste are trapped by the screen or mesh.

The shaker table 360 and support frame 374 are elevated above the small particle extraction unit 366 via another support frame 378 such that the liquid outlet 364 of the shaker table, which in the illustrated embodiment is defined centrally through the bottom of the shaker table 360, extends into the top of the small particle extraction unit 366 that is positioned under the shaker table 360.

Referring specifically to FIG. 5B, details relating to one illustrative embodiment of the small particle extraction unit 366 are shown. In the embodiment shown in FIG. 5B, the large waste particle outlet 368, large waste particle outlet conduit 36 and large waste particle transport device 370 have been omitted for ease of illustration and to more clearly illustrate details of the small particle extraction unit 366. In the illustrated embodiment, the small particle extraction unit 366 includes a first wall 380 extending from the top of the unit 366 downwardly into the interior of the unit 366 adjacent to the liquid outlet 364 of the shaker table 360. The first wall 380 is included in the illustrated embodiment to confine the liquid waste entering the small particle extraction unit 366 between the outer wall 366A of the unit 366 and the first wall 380 so as to direct the entering liquid waste to a small particle collection area 382A defined by the bottom floor 366C of the unit 366. A small particle outlet 396 is defined through the small particle collection area 382A of the bottom or floor 366C of the small particle extraction unit 366. An incline or ramp 384 extends upwardly away from the small particle collection area 382A toward an opposite wall 366B of the small particle extraction unit 366 at a predefined angle relative to the bottom or floor 366C of the unit 366; e.g., 15 degrees, and terminates at a second wall 386 extending upwardly from the bottom floor 366C of the small particle extraction unit 366. A first spill plate 388A extends away from the top of the second wall 386 downwardly and toward the opposite wall 366B of the small particle collection unit 366, and a second spill plate 388B extends away from the opposite wall 366B of the small particle extraction unit 366 below the first plate 386, and downwardly and toward the second wall 386. The bottom of the small particle extraction tank 366 between the second wall 386 and the opposite sidewall 366B defines a liquid waste collection area 382B having a liquid waste outlet 390 coupled to a liquid waste extraction pump 392 via conduit 410. Attached to the underside of the inclined or ramped floor 384 is a vibrator 394 that may be selectively operated to urge small particles collected or settled on the inclined or ramped floor 384 downwardly toward the small particle collection area 382A.

In the operation of the liquid/solid separation unit 30, the shaker table 360 receives liquefied waste via the liquefied waste inlet 362, and is controllably shaken to force the liquid waste and small waste particle portion downwardly toward the liquid waste outlet 364 while trapping the large waste particles carried by the liquefied waste and directing the collected large waste particles, LWP, toward the large waste particle outlet, LPO, and onto the large waste particle transport device 370. The screen or mesh (not shown) will require periodic backwashing with pressurized water to remove trapped large waste particles, and the shaker table accordingly includes a water inlet although this is not specifically shown in FIGS. 5A and 5B. In any case, the liquid waste stream exiting the shaker table 360 via the liquid waste outlet 364 enters the small particle extraction unit 366 and is confined by walls 380 and 366A, which direct the liquid waste toward the small particle collection area 382A of the bottom or floor 366C of the small particle extraction unit 366. As more liquid waste enters the small particle extraction unit 366, it rises up the inclined or ramped floor 384, up the vertical wall 386, and spills over plates 388A and 388B into the liquid waste collection area 382B of the bottom or floor of the small particle extraction unit 366. The configuration of the inclined or ramped floor 384, vertical wall 386 and spill plate 388A creates a very low liquid flow region, and small particles, including residual sand, in the liquid waste entering the small particle extraction unit 366 thus settle out of the liquid waste in this area onto the top surface of the inclined or ramped floor 384. The vibrator 394 is controllably operated to urge the settled small particles back toward the small particle collection area 382A for subsequent extraction via small particle outlet 396. The resulting liquid waste is removed from the liquid waste collection area 382B via the liquid waste outlet 390 by the liquid waste extraction pump 392.

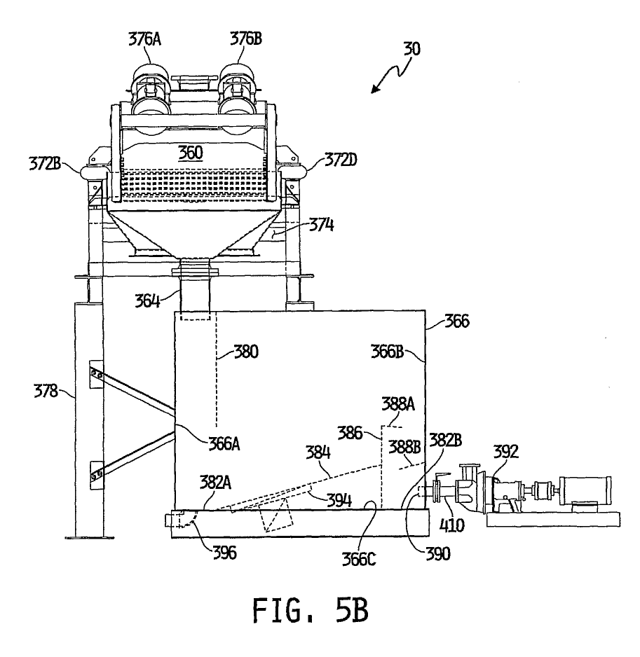

Referring now to FIG. 6, a schematic diagram of one illustrative embodiment of a control system for controlling the liquid/solid separation unit 30 is shown. In the illustrated embodiment, the shaker motor 376A is electrically connected to a conventional motor driver 400 that is electrically connected to an actuator output of the PLC circuit 102 via signal path 112₁₅. The shaker motor 376B is likewise electrically connected to a conventional motor driver 402 that is electrically connected to another actuator output of the PLC circuit 102 via signal path 112₁₆. The PLC circuit 102 is configured to control the operation of the shaker motors 376A and 376B to shake the shaker table 360 as described hereinabove to cause the liquid and small particle portion of the liquefied waste supplied by the sand separation unit 18 to separate from the large waste particles carried by the liquefied waste.

The water supply line 26 is coupled to a water inlet of the shaker table 360 via a water inlet valve 404 that is electrically connected to another actuator output of the PLC circuit 102 via signal path 112₁₇. The PLC circuit 102 is operable to control the water inlet valve to selectively supply pressurized water; e.g., 40 psi, to the shaker table 360 to rinse and clear the shaker table screen of trapped large waste particles. The large waste particle transport 370 is driven by a conventional motor 406 that is electrically connected to a conventional motor driver 408. The motor driver 408 is electrically connected to another actuator output of the PLC circuit 102 via signal path 112₁₈.

The liquid waste outlet 390 of the small particle extraction unit 366 is fluidly coupled via a conduit 410 to an inlet of the liquid waste extraction pump 392. The liquid waste extraction pump 392 is driven by a conventional pump driver 412 that is electrically connected to another actuator output of the PLC circuit 102 via signal path 112₂₀. The outlet of the pump 392 is fluidly connected to the liquid waste outlet, LWO, of the liquid/solid separation unit 30 by conduit 414, and a pair of mechanically actuated butterfly valves, BV, are disposed in-line with conduits 412 and 414 on either side of the liquid waste extraction pump 392 to allow for maintenance or replacement of pump 392 as needed. The small particle outlet port 396 of the small particle extraction unit 366 is fluidly coupled via a conduit 416 to the small particle outlet, SPO, of the liquid/solid separation unit 30. A small particle outlet valve 418 is disposed in-line with conduit 416 and is electrically connected to another actuator outlet of the PLC circuit 102 via signal path 112₂₁. The vibrator 394 is electrically connected to a further actuator output of the PLC circuit 102 via signal path 112₁₉, and the PLC circuit 102 is configured to control operation of the small particle extraction unit 366 as described hereinabove via control of the liquid waste extraction pump 392, the small particle outlet valve 418 and the vibrator 394.

The liquid/solid separation unit 30 further includes a number of sensors providing sensory information to the PLC circuit 102 relating to various operational conditions of unit 30. For example, the small particle extraction unit 366 includes a level sensor 104₄ in fluid communication therewith, which in the illustrated embodiment is implemented as a pressure sensor disposed in fluid communication with the interior of the small particle extraction unit along the incline or ramp 384. Alternatively, the level sensor 104₄ could be implemented using one or more other known level sensors. In any case, the level sensor 104₄ is electrically connected to a sensor input of the PLC circuit 102 via signal path 106₄, and the PLC circuit 102 is configured to determine the liquid waste level within the small particle extraction unit 366 via the sensor signal produced by the level sensor 104₄. Unit 30 further includes a conventional flow meter or other known flow rate sensor 104₅ disposed in-line with the liquid waste outlet conduit 414 and electrically connected to another sensor input of the PLC circuit 102 via signal path 106₉. The flow sensor 104₅ produces a sensor signal from which the PLC circuit 102 may determine the flow rate of liquid waste flowing out of the liquid waste outlet, LWO, of the liquid/solid separation unit 30. A pressure sensor 104₆ is also disposed in fluid communication with the liquid waste outlet conduit 414, and is electrically connected to another sensor input of the PLC circuit 102 via signal path 106₁₀. The pressure sensor 104₆ produces a sensor signal from which the PLC circuit 102 may determine the pressure of the liquid waste flowing out of the liquid waste outlet, LWO, of the unit 30.

Additionally, the small particle extraction unit 366 includes a conventional small particle float 398 positioned proximate or adjacent to the small particle collection area 382A of unit 366, and an associated small particle float sensor 104₇ that is electrically connected to another sensor input of the PLC circuit 102 via signal path 106₁₁. The position of the small particle float 398 varies with the quantity of small particles collected in the small particle collection area 382A, and in the illustrated embodiment the small particle float sensor 104₇ is a switch that changes state when the small particle float 398 reaches a predefined height as the result of a sufficient quantity of small particles collected in the small particle collection area 382A of unit 366. The small particle float sensor 104₇ may alternatively be implemented as an analog or other sensor producing a signal indicative of the position of the small particle float 398 relative to a reference position. In any case, the small particle float sensor 104₇ produces a signal from which the PLC circuit 102 may determine whether the quantity or level of small particles in the small particle collection area 382A of the small particle extraction unit 366 has reached a quantity or level that merits removal of the collected small particles.

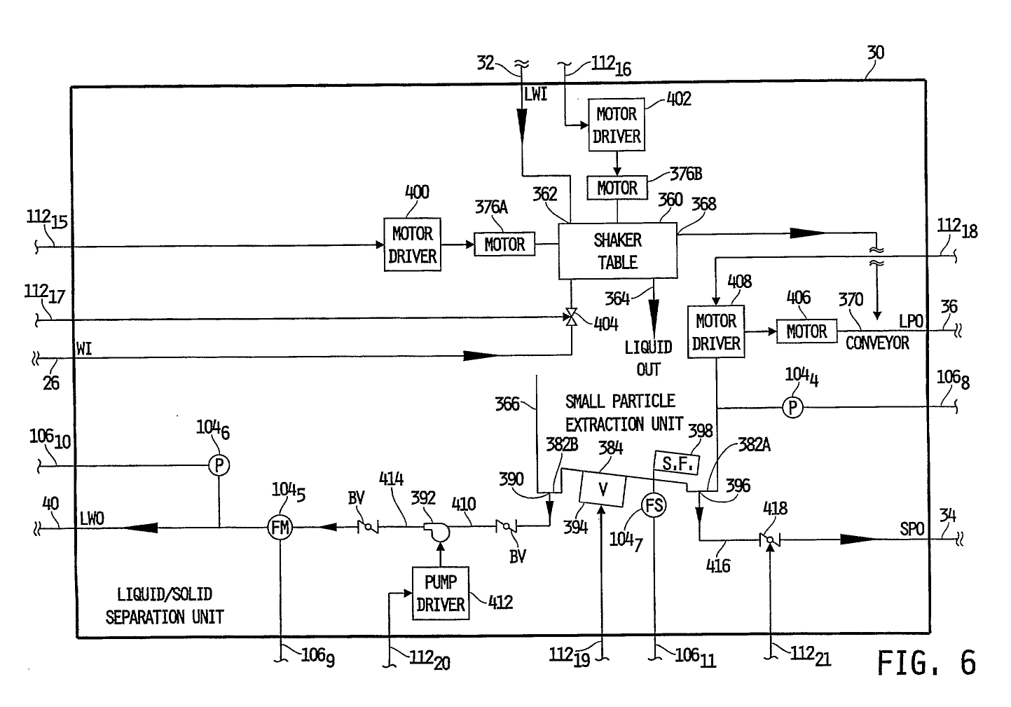

Referring now to FIG. 7, a flowchart of one illustrative embodiment of a software control algorithm 420 for controlling the liquid/solid separation unit 30 via the control system illustrated in FIG. 6 is shown. Control algorithm 420 is stored within, or programmed into, the PLC circuit 102, and the PLC circuit 102 is operable to execute algorithm 420 to control the operation of the liquid/solid separation unit 30. The control algorithm 420 includes a number of different and independently executing control routines, and each of these different control routines will be described separately. For example, the control algorithm 420 includes a first control routine 422 for controlling the operation of the shaker table 360. Control routine 424 begins at step 424 where the PLC circuit 102 is operable to continuously operate the shaker motors 376A and 376B, as liquefied waste is supplied to the shaker table 360 via the liquefied waste inlet 362, by controlling the motor drivers 400 and 402 respectively. Thereafter at step 426, the PLC circuit 102 is operable to periodically open the water supply valve 404 for a time period, T1, to rinse and clear the shaker table screen, and then to close the water inlet valve 404. The time period T1 is selected to allow for the clearing of large waste particles trapped on the top screen surface, and will depend upon the amount, size and density of the large waste particles carried by the liquefied waste, the porosity of the screen or mesh structure mounted within the shaker table 360 and other factors. In any case, execution of the control routine 422 loops from step 426 back to step 424.

The liquid/solid separation unit control algorithm 420 further includes another control routine 428 for controlling removal of liquid waste from the small particle extraction unit 366. Control routine 428 begins at step 430 where the PLC circuit 102 is operable to determine the liquid level (LL) in the small particle extraction unit 366. In the illustrated embodiment, the PLC circuit 102 is operable to execute step 430 by processing the pressure signal produced by the pressure sensor 104₄ in a known manner to determine the liquid waste level within the small particle extraction unit 366. Thereafter at step 432, the PLC circuit 102 is operable to compare LL to a first liquid level threshold, LL_{TH1}, where LL_{TH1} corresponds to a predefined liquid level above which it is desirable to remove liquid waste from the small particle extraction unit 366. Thus, if the PLC circuit 102 determines at step 432 that LL is greater than LL_{TH}, execution of the control routine 428 advances to step 434 where the PLC circuit 102 is operable to activate the liquid waste outlet pump 392. From step 434, execution of the control routine 428 loops back to step 430.

If, at step 432, the PLC circuit 102 determines that LL is not greater than LL_{TH1}, execution of the control routine 428 advances to step 436 where the PLC circuit is operable to compare LL to a second liquid level threshold, LL_{TH2}, where LL_{TH2} corresponds to a predefined liquid level at or below which it is desirable to cease removing liquid waste from the small particle extraction unit 366. Thus, if the PLC circuit 102 determines at step 436 that LL is less than or equal to LL_{TH2}, execution of the control routine 428 advances to step 438 where the PLC circuit 102 is operable to deactivate the liquid waste outlet pump 392. From step 438 and from the “no” branch of step 436, execution of the control routine 428 loops back to step 430.

The liquid/solid separation unit control algorithm 420 includes yet another control routine 440 for controlling the removal of collected small particles from the small particle extraction unit 366. Control routine 440 begins at step 442 where the PLC circuit 102 is configured to periodically operate the vibrator 394 for a time period, T2, to urge small particles settled on the inclined or ramped floor 384 downwardly toward the small particle collection area 382A of the small particle extraction unit 366. The time period T2 is selected to allow a substantial amount of the small particles settled onto the top surface of the inclined or ramped floor 384 to move down the inclined or ramped floor 384 and into the small particle collection area 382A, and will depend upon the amount, size and density of the small particles present in the liquid waste, the vibrating strength of the vibrator 394 and other factors.

In any case, execution of the control routine 440 advances from step 442 to step 444 where the PLC circuit 102 is operable to monitor the output, SF, of the small particle float sensor 104₇. Thereafter at step 446, the PLC circuit 102 is operable to compare SF to a threshold sensor value, SF_{TH}. If, at step 446, SF is greater than or equal to SF_{TH}, execution of control routine 440 advances to step 448, and otherwise loops back to step 442. In embodiments of the liquid/solid separation unit 30 wherein the small particle float sensor 104₇ is provided in the form of a switch, the threshold sensor value SF_{TH} corresponds to one of two switch states; e.g., high or low, and the PLC circuit 102 is operable to execute step 446 by determining whether SF is equal to the switch state triggered by the small particle float 398 when the small particle collection area 382A has a predefined quantity of small particles collected therein. In embodiments wherein the small particle float sensor 104₇ is provided in the form of a conventional small particle float position sensor, the threshold sensor value SF_{TH} corresponds to a position of the small particle float 398, relative to a reference position, when the small particle collection area 382A has the predefined quantity of small particles collected therein. In any case, the PLC circuit 102 is operable at step 448 to open the small particle outlet valve 418 for a time period T3, and then to close the small particle outlet valve 418. The time period T3 is selected to allow for removal of a substantial portion of the small particles collected in the small particle collection area 382A, and will depend upon the trigger height of the small particle float 398, the dimensions of the small particle collection area 382A and other factors. In any case, execution of the control routine 440 loops back to step 442. The sensory information provided by the flow sensor or meter 104₅ and the pressure sensor 104₆ is used to control the speed of the liquid waste outlet pump 392 in relation to the speed of another liquid waste outlet pump (474) in the pH adjustment stage 38 to provide for proper pump operation and a specified liquid waste flow rate as will be described in greater detail hereinafter with respect to FIGS. 8A, 8B and 9.

Referring now to FIG. 8A, a schematic diagram of one illustrative embodiment of the pH adjustment unit 38 and corresponding control system that forms part of the waste stream pre-treatment system 12 is shown. In the illustrated embodiment, the pH adjustment unit 38 includes an acid supply source 450 fluidly coupled to a liquid inlet of a liquid mixer 454 via a conduit 452. The liquid waste conduit 40 defining the liquid waste inlet, LWI, of the pH adjustment unit 38 is also fluidly coupled to conduit 452 between the acid supply source 450 and the liquid mixer 454. In one embodiment, the liquid mixer 454 is implemented in the form of a length of conduit configured with a number of sharp turns to facilitate mixing of the liquid waste as it flows therethrough. Alternatively, the liquid mixer 454 may be a tank or other conventional liquid mixing structure that may include one or more conventional agitators operable to mix the liquid waste. In any case, the liquid mixer 454 has a liquid outlet in fluid communication with a liquid outlet conduit 456.

The acid supply source 450 includes an acid storage tank, and in the illustrated embodiment the acid storage tank is provided in the form of a double-walled acid tank 458 having a pair of acid outlets each coupled via a ball valve, BV, to a conduit 460. In one embodiment, the acid tank 458 is filled with a sulfuric acid solution, although tank 458 may alternatively be filled with other acidic solutions or dry mixtures including, but not limited to, solutions or dry mixtures of inorganic or mineral acids such as hydrochloric acid, hydrobromic acid, nitric acid, sulfuric acid, and acidic salts thereof, phosphoric acid, and acidic salts thereof, perchloric acid, and the like; and organic acids such as carbonic acid, formic acid, acetic acid, and the like; and combinations thereof. In the illustrated embodiment, the acid tank 458 includes an ultrasonic or other suitable level sensor 104₁₀ in fluid communication therewith and electrically connected to a sensor input of the PLC circuit 102 via signal path 106₁₄. The PLC circuit 102 is operable to monitor the acid solution level within the acid tank 458 by monitoring the signal produced by the level sensor 104₁₀, and activate a conventional indicator when the acid solution level drops below a threshold acid level to prompt a technician to add acid solution to the acid tank 458.

The conduit 460 is fluidly coupled through a pair of ball valves, BV, to acid solution inlets of a pair of acid solution pumps 464 and 466. A first conventional pump driver 468 is electrically connected to the pump 464, and is also electrically connected to an actuator output of the PLC circuit 102 via signal path 112₂₂. A second conventional pump driver 470 is electrically connected to the pump 466, and is also electrically connected to another actuator output of the PLC circuit 102 via signal path 112₂₃. Acid solution outlets of the acid solution pumps 464 and 466 are fluidly coupled through a series of butterfly and ball valves, BV, to conduit 452. The PLC circuit 102 is operable to control the acid solution pumps 464 and 466 to controllably supply the acid solution stored in the acid tank 458 to the inlet of the mixer 454 via conduit 452. The various ball and butterfly valves, BV, are mechanically actuated valves, and are included to allow for maintenance and/or replacement of the acid tank 458 and acid pumps 464 and 466, and/or to isolate the acid tank 458 from the remainder of the acid supply source 450 or to isolate the acid supply source 450 from the liquid mixer 454.

As shown in phantom in FIG. 8A, the pH adjustment unit 38 may alternatively or additionally include a base supply source 472 having a base solution outlet fluidly coupled to the inlet of the mixer 454 via conduit 452. In embodiments including the base supply source 472, it may be configured identical to the acid supply source 450 except that the acid tank 458 will be replaced with a base tank filled with a suitable base in solution or as a dry mixture including, but not limited to, inorganic bases such as hydroxides such as sodium, potassium, cesium, ammonium, and like hydroxides; carbonates such as sodium, potassium, ammonium, and like carbonates; bicarbonates such as sodium, potassium, ammonium, and like bicarbonates, phosphates such as sodium, potassium, ammonium, and like phosphates; organic bases such as amines, substituted amines such as alkyl, dialkyl, and trialkylamines, tetraalkylammonium salts, heteroaryls such as pyridines, pyridazines, pyrimidines, and pyrazines, and combinations thereof.

The outlet conduit 456 is fluidly coupled through a series of butterfly valves, BV, to the inlet of a liquid waste outlet pump 474 having a pump outlet defining the liquid waste outlet, LWO, of the pH adjustment unit 38 and fluidly coupled to conduit 42. A conventional pump driver 476 is electrically connected to the pump 474, and is also electrically connected to another actuator output of the PLC circuit 102 via signal path 112₂₄. The PLC circuit 102 is operable to control the liquid waste outlet pump 474 to controllably supply liquid waste to the waste fermentation system 14 via conduit 42.

The pH adjustment unit 38 may further include a number of conduits and associated butterfly valves, BV, coupled to the liquid waste outlet conduit 456 between the outlet of the liquid mixer 454 and the inlet of the liquid waste outlet pump 474 to allow for the cleaning/sterilization of the outlet liquid waste outlet of the pH adjustment unit 38 and the liquid waste inlet of the waste fermentation system 14. Because such conduits are used only for the purpose of cleaning and sterilizing portions of the pH adjustment unit 38 and waste fermentation system 14, and are generally not used during the normal, continuous flow operating mode of the biomaterial waste processing system 10, the inlets and outlets of such conduits to and from the pH adjustment unit 38 are not shown in FIG. 1 for ease of illustration, but are shown in FIG. 8A to illustrate the cleaning/sterilization flow paths relative to the pH adjustment unit 38. In the illustrated embodiment, for example, the water inlet conduit 26 is fluidly coupled through a butterfly valve, BVA, to a conduit 480 coupled through another butterfly valve, BVC, to the liquid waste outlet conduit 456. A cleaning agent conduit 478 is coupled through another butterfly valve, BVB, to conduit 480, and a pair of butterfly valves, BVD and BVE, are disposed in-line with conduit 456; one, BVD, between the junction with conduit 480 and the outlet of the liquid mixer 454 and the other, BE, between the junction with conduit 480 and the inlet of the pump 474. The conduit 78 fluidly connected to the liquid outlet, LO, of the residual liquid processing unit 16 may further be coupled to the junction of conduits 456 and 480 through another butterfly valve, BVF, and yet another conduit 484 may be coupled to conduit 78 downstream of the butterfly valve, BVF. The conduit 484 may be coupled to the liquid waste return conduit 76 through yet another butterfly valve, BVG. In a cleaning/sterilization mode, valves BVA, BVB, BVE and BVF may be opened while valve BVG is closed, and a cleaning agent may be added to conduit 478 such that a mixture of cleaning agent and water is circulated through a portion of conduit 456, through the liquid waste outlet pump 474, through the liquid waste outlet conduit 42 and at least a portion of the waste fermentation system 14. When these conduits and pump 474 have been sufficiently cleaned/sterilized, valves BVB and BVF may be closed and valve BVG opened to flush the cleaning path with clean water. Thereafter, valve BVA may be closed, and valves BVD may be opened to resume normal, continuous flow operation of the biomaterial waste processing system 10.

The pH adjustment unit 38 further includes a number of sensors providing sensory information to the PLC circuit 102 relating to various operational conditions of the pH adjustment unit 38. For example, unit 366 includes an inlet conductivity sensor 104₈ in fluid communication with the liquid waste inlet conduit 40, and electrically connected to a sensor input of the PLC circuit 102 via signal path 106₁₂. The inlet conductivity sensor 104₈ produces an inlet conductivity signal, Ci, corresponding to the electrical conductivity of the liquid waste stream entering the liquid mixer 454, and the PLC circuit 102 is configured to process Ci in a known manner to determine the pH level of the liquid waste stream entering the liquid mixer 454. The pH adjustment unit 38 may further include an outlet conductivity sensor 104₉ in fluid communication with the liquid waste outlet conduit 456, and electrically connected to another sensor input of the PLC circuit 102 via signal path 106₁₃. The outlet conductivity sensor 104₉ produces an outlet conductivity signal, Co, corresponding to the electrical conductivity of the liquid waste exiting the liquid mixer 454, and the PLC circuit 102 is configured to process Co in a known manner to determine the pH level of the liquid waste stream exiting the liquid mixer 454. The PLC circuit 102 is configured to adjust the pH level of the liquid waste stream passing through the liquid mixer 454 to a target pH level by controlling the amount of acid solution entering conduit 452 (and/or the amount of base solution entering conduit 452) based on the inlet conductivity signal, Ci, alone, or alternatively based on the inlet conductivity signal, Ci, and the outlet conductivity signal, Co.

In embodiments of the biomaterial waste processing system 10 configured to process liquefied animal waste, the pH level of the liquid waste stream entering the pH adjustment unit 38 will generally be at least slightly basic, whereas optimal liquid waste processing conditions in the subsequent waste fermentation system 14 are often generally acidic; for example, fermenting organisms such as yeasts exhibit higher fermentation rates at pH levels less than about 7, and illustratively less than about 5. It is appreciated that the fermenting organism or organisms selected for inclusion in the fermentation system 14 will have a pH level that is optimum for fermentation. It is further appreciated that many organisms have a range of pH levels that might be used for fermentation. For those organisms, the pH may be adjusted to near optimum levels for fermentation in order to satisfy other criteria, such as diminishing the proliferation or growth of a competing organism. In the illustrated embodiment, the pH adjustment unit 38 is accordingly controlled by the PLC circuit 102 to selectively add acid solution to the liquid waste stream entering the liquid mixer 454 to adjust the pH level of the liquid waste exiting the liquid mixer 454 to a target acidic pH level. In embodiments of the biomaterial waste processing system 10 configured to process animal waste, the target pH level may be, for example, 4.0. In other embodiments, the biomaterial waste stream may be too acidic for optimal processing by the waste fermentation system 14, and in such embodiments the pH adjustment unit 38 may include the base supply source 472, and the PLC circuit 102 may be controlled in such embodiments to selectively add base solution to the liquid waste entering the liquid mixer 454 to adjust the pH level of the liquid waste exiting the liquid mixer 454 to the target pH level. In still other embodiments, regardless of the pH level of the incoming liquid waste stream, the pH adjustment unit 38 may include both of the acid and base supply sources 450 and 472 to provide for pH adjustment of the incoming liquid waste stream in either pH direction.

Referring now to FIG. 8B, a schematic diagram of another illustrative embodiment of the pH adjustment unit 38′ and corresponding control system that forms part of the waste stream pre-treatment system 12 is shown. The pH adjustment unit 38′ and associated control system illustrated in FIG. 8B is identical in many respects to the pH adjustment unit 38 and associated control system illustrated in FIG. 8A, and like numbers are therefore used to identify like components. In the embodiment illustrated in FIG. 8B, a biomaterial waste settling tank 457 is interposed between the mixer 454 and the pump 474. More particularly, the biomaterial waste outlet of the mixer 454 is fluidly coupled to a biomaterial waste inlet of the settling tank 457 via a conduit 456, and a biomaterial waste outlet of the settling tank 457 is fluidly coupled to the junction of the conduits 480 and 482. An air or gas outlet of the settling tank 457 is fluidly coupled to the gas outlet 68 of the waste fermentation system 14. A solid waste outlet of the settling tank 457 is fluidly coupled to an inlet of a solid waste outlet pump 465 having a pump outlet fluidly coupled to the precipitated waste outlet conduit 80. A conventional pump driver 467 is electrically connected to the pump 465, and is electrically connected to another actuator output of the PLC circuit 102 via signal path 112₂₅. The PLC circuit 102 is operable to control the solid waste outlet pump 465 to controllably pump solid waste from the settling tank 457 via the conduit 80.

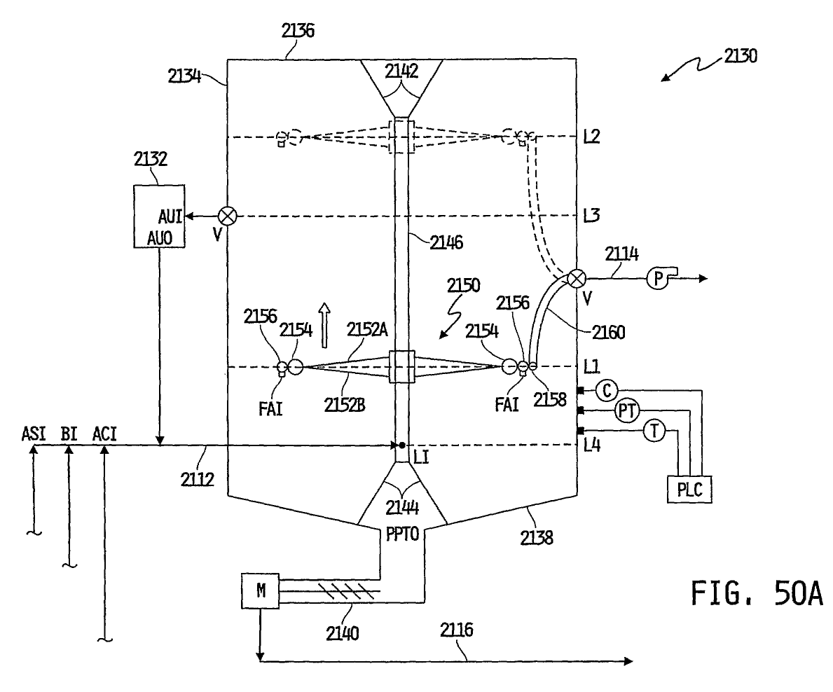

In another embodiment, a settling system is described (see, for example, the illustrative settling system 457 shown in FIGS. 8B-8E, and a system containing the same shown in FIG. 8A). The settling system is generally designed to remove particulates, including fine particulates from a biomaterial waste stream. In one aspect, the particulates or fine particulates include sand, straw, fibers, and the like. It is appreciated that the settling system may be advantageously used to remove small amounts of particulates from a biomaterial waste stream that has already been treated by another separation process, including the separation processes described herein, such as an illustrative liquid solid separation unit 30 (see FIG. 6), and the like. In another aspect, the settling system is used as a separation process prior to an additional separation process, including the separation processes described herein, such as illustrative aggregation unit 2110 designed to remove dissolved solids form aqueous solutions (see FIG. 49), and the like. In another aspect, the settling system is an independent or stand-alone separation system.

FIG. 8B shows an illustrative configuration of this alternate solid separation unit 457 following a pH adjustment unit. FIGS. 8C and 8D show side and top views, respectively, of an illustrative cylindrical embodiment of settling tank 457 defined by an outer wall 471, a sloping top 473, such as a domed top, and a sloping bottom 475, such as a domed bottom. It is appreciated that domed or sloping top 473, and domed or sloping bottom 475 may each facilitate the removal of material from settling tank 457. Settling tank 457 is fitted with a biomaterial waste stream inlet WI entering outer wall 471, a clarified liquid outlet CLO exiting outer wall 471, an air outlet AO exiting sloping top 473, and a solids outlet SO exiting sloping bottom 475. Solids outlet SO is a circular opening in sloped bottom 475, and is illustratively large compared to inlet WI, air outlet AO, and clarified liquid outlet CLO. Solids outlet SO may operate solely by gravity feed, or may be optionally fitted with a pump and/or auger attached at conduit 495 to facilitate removal of precipitated solids from settling tank 475. Removed solids may be transported to other optional processes by a conveyer system, enclosed pipe, and the like, depending upon the nature, viscosity, water content, and other properties of the removed solids, as appropriate. Settling tank 457 is supported above ground by supports (not shown), each being long enough to accommodate solids outlet SO and any other optional system for transporting solids removed from settling tank 457.

The interior of settling tank 457 is fitted with liquid sparger 477, cone 479, and four vertical plates 481. The top edge of liquid sparger 477 includes clarified liquid inlets CLI in fluid communication with the liquid contents of settling tank 457, and also in fluid communication with conduit 483 connected to clarified liquid outlet CLO. Cone 479 is radially centered on the vertical axis of settling tank 457, and is vertically positioned in tank 457, illustratively about midway in the cylindrical portion of settling tank 457, or slightly lower. The height of cone 479 is in the range from about 55% to about 75%, and illustratively about 60%, of the height of the cylindrical portion of settling tank 457. In one aspect, bottom edge 485 of cone 479 spans most of the horizontal dimension of the interior space of settling tank 457. In another aspect, bottom edge 485 of cone 479 spans the majority of the diameter of settling tank 457, such as in the range from about 75% to about 90%, or from about 80% to about 85% of the diameter of settling tank 457. Apex 487 of cone 479 is in fluid communication with air outlet AO via conduit 489, allowing trapped air to escape. Waste stream inlet WI extends into the interior of cone 479 to biomaterial waste stream outlet WO. Waste stream inlet WI is positioned near apex 487, but sufficiently below the opening to conduit 489 to allow trapped air to escape without simultaneously aspirating significant amounts of liquid phase.

Vertical plates 481, in the shape of right triangles are attached to the outer surface of cone 479 at 90 degree intervals when viewed from the top (see FIG. 8D). Vertical plates 481 extend to or nearly to the bottom edge 485 of cone 479, and to or nearly to the apex 487 of cone 479. In one illustrative variation, vertical plates 481 extend to bottom edge 485 and nearly to the apex 487 of cone 479.

In another illustrative variation, the cylindrical portion of settling tank 457 has a medium-sized or nearly equal aspect ratio, such as an aspect ratio in the range from about 1.1 to about 1.5.

In one illustrative embodiment, the cylindrical portion of settling tank 457 is about 12 feet (3.7 m) in height and 11 feet (3.4 m) in diameter, cone 479 is about 7.5 feet (2.3 m) in height and about 9.5 feet (2.9 m) in diameter, and cone 479 is positioned about 2 feet (0.6 m) from the bottom of the cylindrical portion of, and about 1 foot (0.3 m) from inner wall 491 of settling tank 157. Solids outlet SO is about 30 inches (0.8 m) in diameter.

FIG. 8E shows the flow of liquid and solid components of the biomaterial waste stream entering settling tank 457 via waste stream inlet WI. Referring to FIG. 8E, liquid biomaterial waste (hashed arrow) enters inlet WI and proceeds to the interior of cone 479. The configuration of cone 479 will naturally create a vortex in the liquid moving down cone 479, under gravity flow and optionally some residual pressure, creating thereby a Coriolis, centrifugal, or centripetal force directly radially outward to the sides of cone 479 (hashed arcing arrow). Net velocity within cone 479 is vertically downward (hashed arrow), allowing substantial settling of particulates and other solid components of the biomaterial waste stream. Contributing to the generated radially outward velocity is the movement of liquid away from the outer and bottom edge of cone 479. Because the amount of mass removed via clarified liquid outlet CLO is greater, illustratively as high as ten-fold greater, than the amount of mass removed via solids outlet SO, net velocity outside cone 479 and above bottom edge 485 is vertically upward (open arrows). Conversely, net velocity outside cone 479 and below bottom edge 485 is vertically downward (solid arrows). Circular rotation of liquid outside cone 479 and above bottom edge 485 may be opposite that of circular rotation of liquid inside cone 479 and/or outside cone 479 and below bottom edge 485. Vertical plates 481 are positioned to decrease or limit the circular rotation of liquid outside cone 479 and above bottom edge 485 to reduce, minimize, or preclude the generation of turbulence at the interface between mass moving vertically upward and mass moving vertically downward. It is appreciated that the circular rotation of liquid outside cone 479 and below bottom edge 485 may create a sweeping effect to facilitate movement of settling solid components to solids outlet SO, and also facilitated by sloping bottom 475. It is further appreciated that other particulates or solids that are less dense than the bulk liquid biomaterial waste stream entering cone 479 via conduit 493 will float on top of the entering biomaterial waste stream at apex 487, be trapped thereby, and be effectively separated from the entering biomaterial waste stream to produce clarified liquid.

Liquid sparger 477 is positioned sufficiently high in settling tank 457 to maximize the laminar flow of clarified liquid into clarified liquid inlets CLI positioned on the top face of sparger 477, thus maximizing the clarity of liquid exiting settling tank 457 via conduit 483 and clarified liquid outlet CLO.

Referring now to FIG. 9, a flowchart of one illustrative embodiment of a software control algorithm 490 for controlling the pH adjustment unit 38 is shown. Control algorithm 490 is stored within, or programmed into, the PLC circuit 102, and the PLC circuit 102 is operable to execute algorithm 490 to control the operation of the pH adjustment unit 38. The control algorithm 490 includes a number of different and independently executing control routines, and each of these different control routines will be described separately. For example, the control algorithm 490 includes a first control routine 492 for controlling the flow of liquid waste out of the liquid waste outlet, LWO, of the pH adjustment unit 38. Control routine 492 begins at step 494 where the PLC circuit 102 is operable to determine the pressure, P, of the liquid waste stream between the liquid/solid separation unit 30 and the pH adjustment unit 38; i.e., the pressure signal produced by the pressure sensor 104₆ of the liquid/solid separation unit 30 of FIG. 6. Thereafter at step 496, the PLC circuit 102 is operable to determine the flow rate, FR, of the liquid waste stream exiting the liquid/solid separation unit 30; i.e., the flow rate signal produced by the flow rate sensor or meter 104₅. Thereafter at step 498, the PLC circuit 102 is operable to control the operation of the liquid waste outlet pump of the liquid/solid separation unit 30 of FIG. 6; i.e., pump 392, and the operation of the liquid waste outlet pump of the pH adjustment unit 38 of FIG. 8A or 8B; i.e., pump 474 to maintain positive pressure within conduits 414 and 40, and to maintain a flow rate of the liquid waste stream through the pH adjustment system 38 and into liquid waste conduit 42 near a target flow rate, FRT; e.g., 100 gpm. The PLC circuit 102 is operable to execute step 498 by controlling the relative speeds of pumps 392 and 474 to prevent pump cavitation by maintaining a positive pressure therebetween, while also controlling the speeds of both pumps 392 and 474 to maintain FR near FRT. From step 498, execution of the control routine 492 loops back to step 494.

The pH adjustment unit control algorithm 490 includes another control routine 500 for continuously adjusting the pH level of the liquid waste stream flowing through the pH adjustment unit 38. In the illustrated embodiment, the control routine 500 begins at step 502 where the PLC circuit 102 is operable to determine the inlet conductivity, Ci, corresponding to the conductivity of the liquid waste stream entering the liquid mixer 454. The PLC circuit 102 is operable to execute step 502 by monitoring the signal produced by the conductivity sensor 104₈. Thereafter at step 504, the PLC circuit 102 is operable to determine the outlet conductivity, Co, corresponding to the conductivity of the liquid waste stream exiting the liquid mixer 454. The PLC circuit 102 is operable to execute step 504 by monitoring the signal produced by the conductivity sensor 104₉. Following step 504, the PLC circuit 102 is operable at step 506 to control the acid pumps 464 and 466 based on Ci alone, or on Ci and Co, to drive Co to a target conductivity value, C_{T}. In one embodiment, the PLC circuit 102 may be configured to determine the flow rate of acid solution required to change Co to C_{T} as a function of the inlet conductivity, Ci, of the liquid waste stream entering the liquid mixer 454 and the flow rate, FR, of the liquid waste stream entering the liquid mixer 454, and to control the acid pumps 464 and 466 to supply the acid solution to the liquid mixer 454 at the required acid solution flow rate. Alternatively, the PLC circuit 102 may be configured to determine the flow rate of acid solution required to change Co to C_{T} as a function of the conductivity differential; e.g., Co-Ci, across the liquid mixer 454. In either case, execution of the control routine 500 loops back to step 502 in embodiments of the pH adjustment unit 38 that do not include a base supply source 472. In embodiments of the pH adjustment unit 38 including the base supply source 472, however, the control routine 500 may additionally or alternatively to step 506 include step 508, as shown in phantom in FIG. 9, wherein the PLC circuit 102 is operable similarly as just described with respect to step 506 to control base solution pumps contained within the base supply source 472 based on Ci, or on Ci and Co, to drive Co to C_{T}. Execution of the control routine 500 loops from step 508 back to step 502.

Referring now to FIG. 10, a schematic diagram of one illustrative embodiment of the air system 56 and corresponding control system that forms part of the biomaterial waste stream processing system 10 is shown. In the illustrated embodiment, the air system 56 includes a conventional air compressor 520 fluidly coupled to a conventional air dryer 524 via a conduit 522. A pressure sensor 122₁ is disposed in fluid communication with the air compressor 520, and is electrically connected to a sensor input of the PLC circuit 120. The air dryer 524 is operable in a known manner to dry the pressurized air supplied by the air compressor 520, and to supply the dried air to an air conduit 526 having a pair of ball valves, BV, disposed in-line therewith. Another air conduit 530 extends from air conduit 426 and through another ball valve to an air inlet of a conventional pressure regulator 532. An air outlet of the pressure regulator 532 is fluidly coupled via another air conduit 534, through another ball valve, BV, to air outlet conduits 58, 60 and 62.

Another conduit 530 is fluidly coupled to the junction of air conduits 526 and 528, and is also coupled to the steam inlet conduit 64 through another ball valve, BV. Conduit 530 is also coupled to conduit 534 through a pair of ball valves, BV, and another conduit 538 is coupled to conduit 536 between the pair of ball valves, BV. The conduit 528 is also coupled to steam outlet conduit 66 through another pair of ball valves, BV. Another conduit 540 couples a drain outlet of the pressure regulator 532 to the drain conduit 67 through another ball valve, BV.

In the illustrated embodiment, the pressure regulator 532 may be manually set to regulate the pressurized air supplied by the air compressor 520 and air dryer 524 to a desired air pressure, wherein the air regulated to the desired air pressure is supplied by the pressure regulator to air outlets 58, 60 and 62. The various ball valves, BV, may be selectively opened to allow a combination of steam and pressurized air to flow out of the steam conduit 66.

Referring now to FIG. 11, a schematic diagram of one illustrative embodiment of the water system 24 and corresponding control system that forms part of the biomaterial waste stream processing system 10 is shown. In the illustrated embodiment, tap water; e.g., 40 psi, is supplied via water conduit 25 to a tap water inlet, TWI, of the water system 24 that is coupled through a ball valve, BV, to an inlet of a conventional water softener 550. An outlet of the water softener 550 is coupled through a pair of ball valves, BV, and a control valve 554 disposed therebetween to a soft water surge tank 556 having a pressure sensor 122₂ or other suitable fluid level sensor in fluid communication therewith and electrically connected to a sensor input of the PLC circuit 120 via signal path 124₂. The control valve 554 is electrically connected to an actuator output of the PLC circuit 120 via signal path 130₁. The PLC circuit 120 is operable to maintain a sufficient amount of water within the soft water surge tank 556 by monitoring the signal produced by the pressure sensor 122₂, processing this signal to determine a level of water within the soft water surge tank 556, and controlling the control valve 554 to supply soft water from the water softener 550 to the soft water surge tank 556 when the water level within the soft water surge tank 556 is below a threshold water level. A water outlet of the soft water surge tank 556 is coupled through another ball valve, BV, to the water outlet conduit 26.

In embodiments including the water system 24 illustrated in FIG. 11, the soft water surge tank 556 also includes an overflow inlet coupled through another ball valve, BV, to an overflow conduit 558 extending from the waste fermentation system 14. In an alternative embodiment, the water system 24 may be omitted, and the tap water supplied via conduit 25 may be instead used as the water source. In such embodiments, the overflow conduit 558 may be routed to a suitable overflow container or may instead be configured to spill overflow water to the ground.

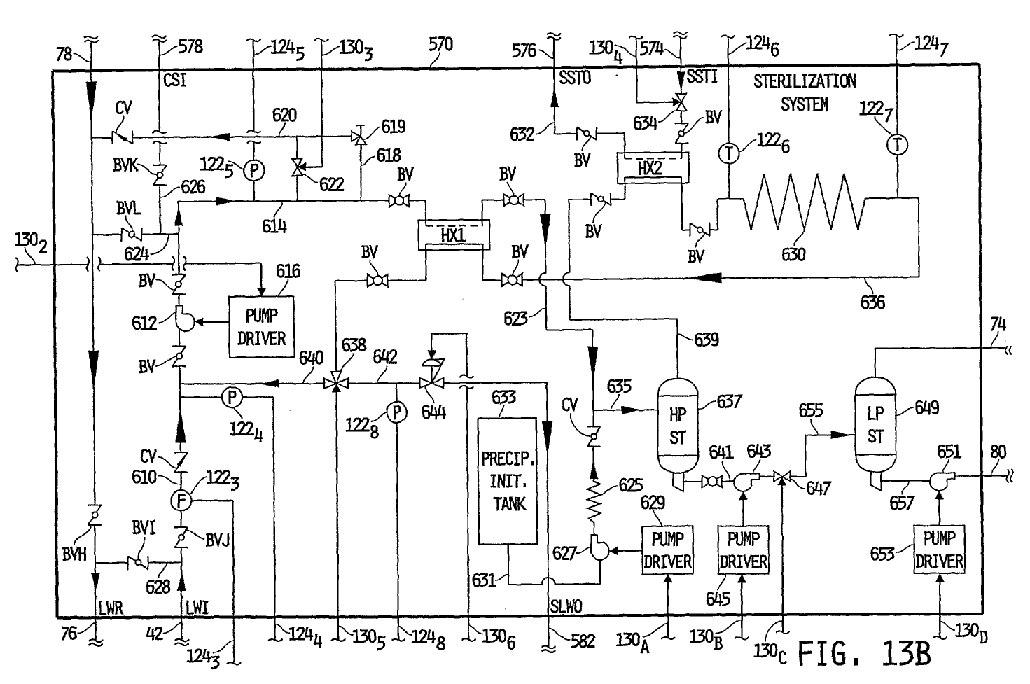

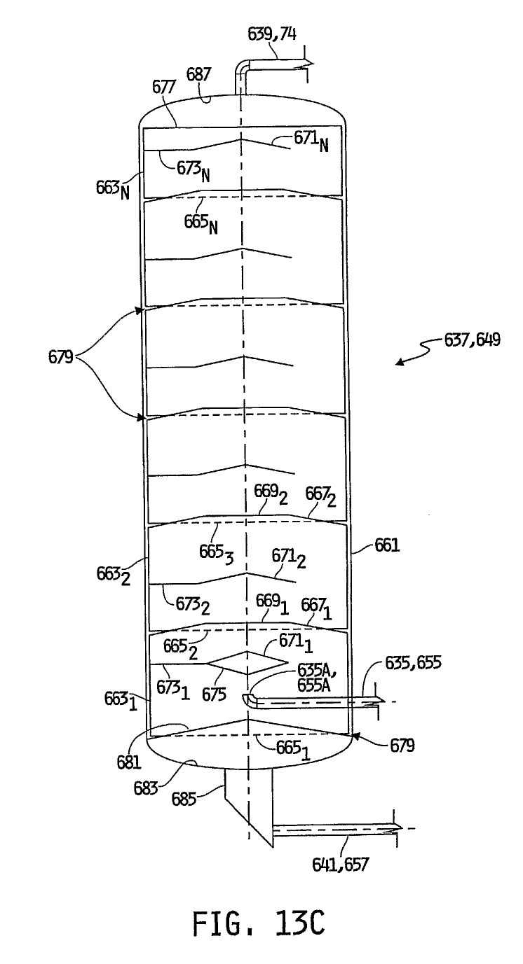

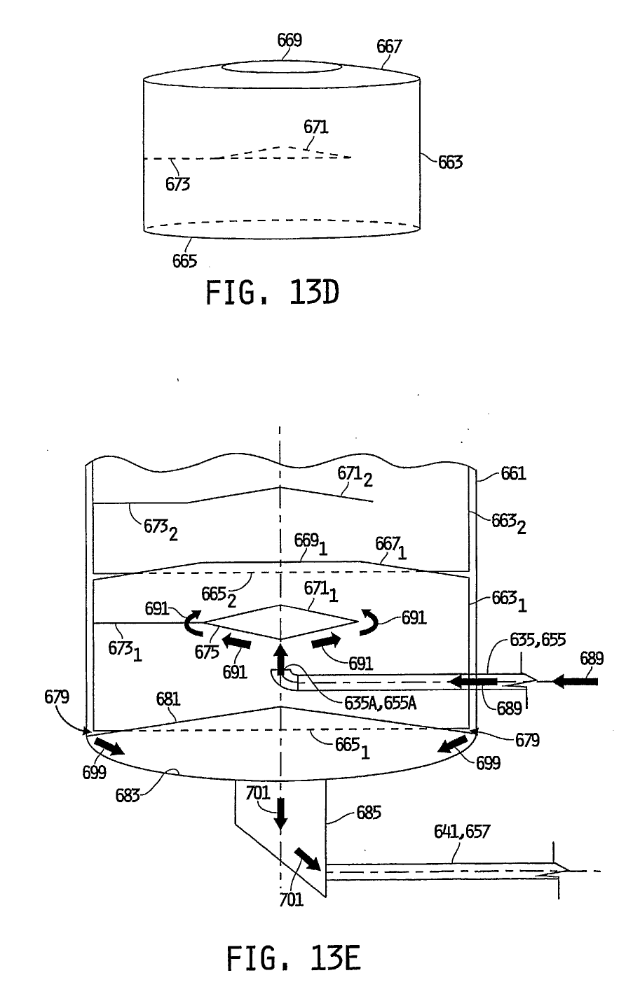

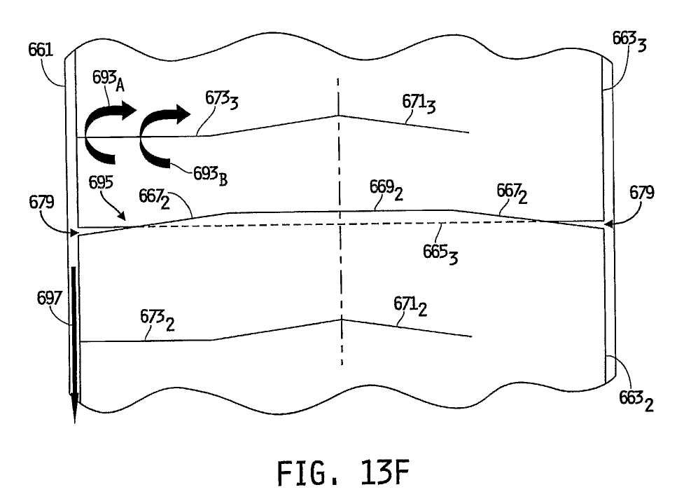

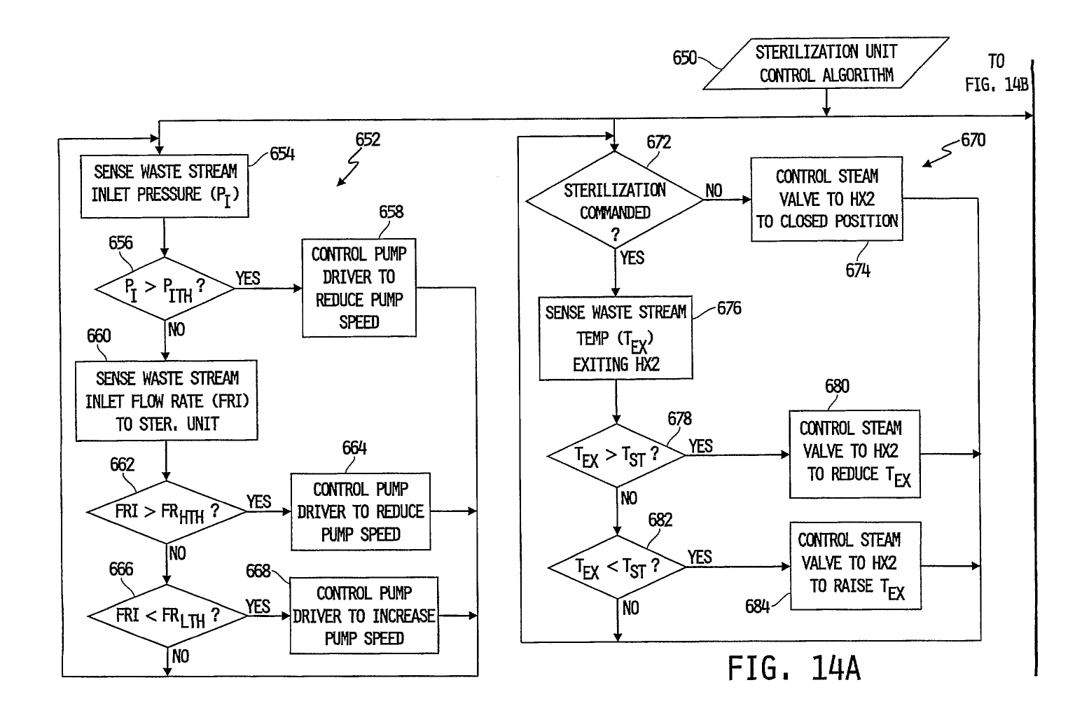

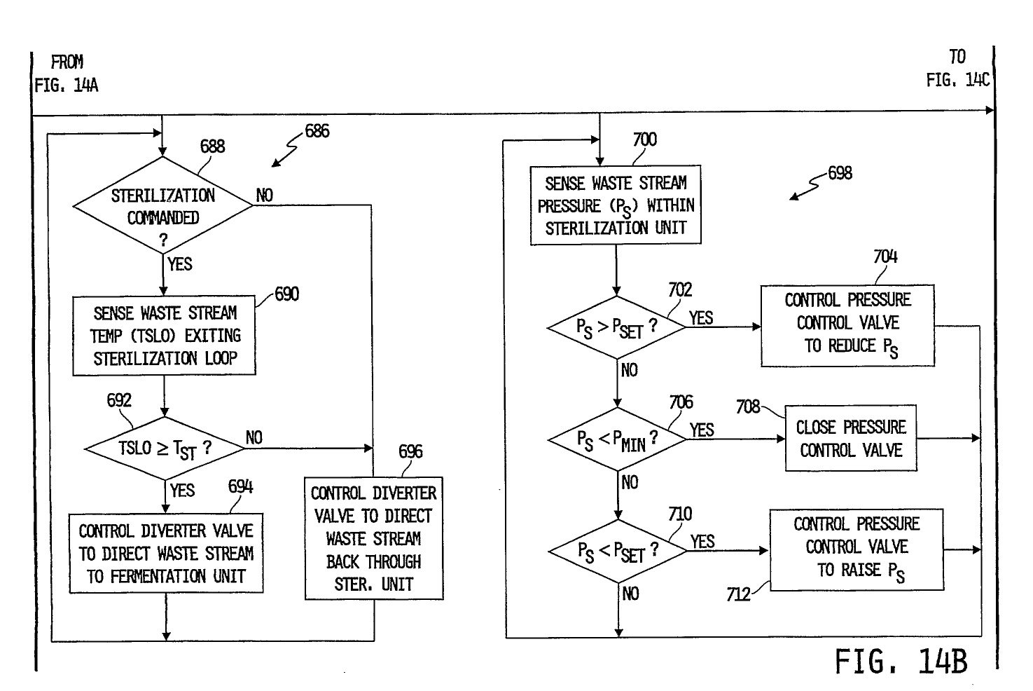

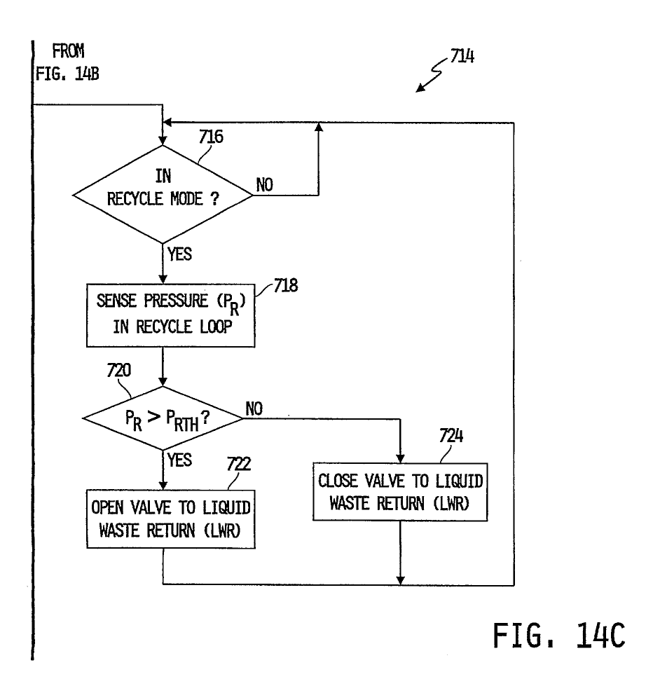

Referring now to FIG. 12 a block diagram of one illustrative embodiment of the waste fermentation system 14 forming part of the biomaterial waste processing system 10 is shown. In the illustrated embodiment, the waste fermentation system 14 includes a sterilization unit 570 having a liquid waste inlet, LWI, fluidly coupled to the liquid waste inlet, LWI, of the waste fermentation system 14 and receiving the liquid biomaterial waste stream via conduit 42, a sterilized liquid waste outlet, SLWO, supplying a stream of sterilized liquid biomaterial waste to a sterilized liquid waste inlet, SLWI, of a fermentation unit 580 via conduit 582 and a liquid waste return outlet, LWR, fluidly coupled to the liquid waste return outlet, LWR, of the waste fermentation system 14. The sterilization unit 570 further includes a sterilization steam inlet, SSTI, fluidly coupled to a sterilization steam outlet, SSTO, of a steam unit 572 via conduit 576, a sterilization steam outlet, SSTO, fluidly coupled to a sterilization steam inlet, SSTI, of the steam unit 572 via conduit 576 and a cleaning steam inlet, CSI, fluidly coupled to a cleaning steam outlet, CSO, of the steam unit 572 via conduit 578. The sterilization unit 570 further includes a number, L, of sensors each producing a sensor signal indicative of a corresponding operating condition of the sterilization unit 570, wherein L may be any positive integer. The “L” sensor signals are supplied to the PLC circuit 120 via a corresponding number of signal paths as illustrated in FIG. 1. The sterilization unit 570 further includes a number, K, of actuators each responsive to a corresponding actuator control signal supplied by the PLC circuit 120 to control a corresponding operating parameter of the sterilization unit 570. The sterilization unit 570 is generally operable, as will be described in greater detail hereinafter with respect to FIGS. 13A-14C, to sterilize the liquid biomaterial waste stream supplied thereto via conduit 42 and provide a sterilized liquid biomaterial waste stream to the fermentation unit 580 via conduit 582.

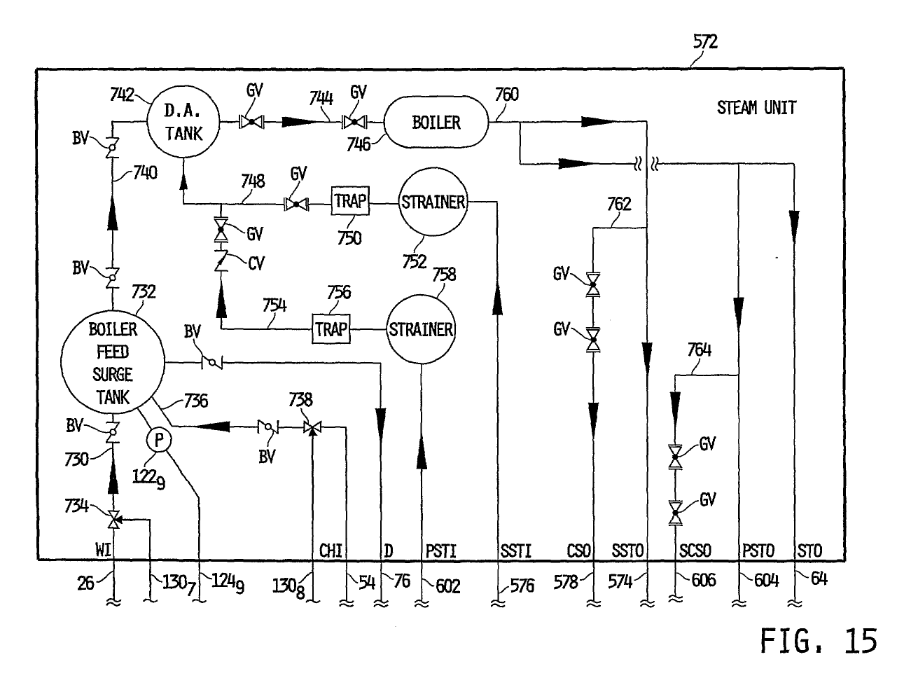

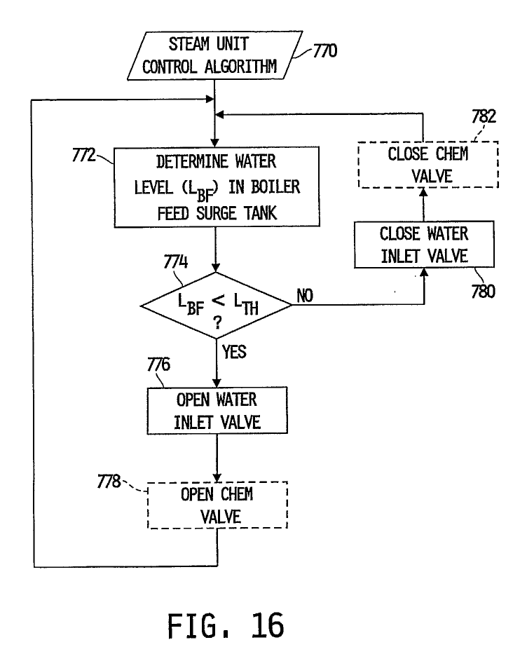

The temperature of the sterilization process performed by the sterilization unit 570 is controlled by the steam unit 572 configured to controllably circulate steam through the sterilization unit 570 via conduits 574 and 576. The steam unit 572 further includes a water inlet, WI, fluidly coupled to the water inlet, WI, of the waste fermentation system 14 via conduit 26, and a chemical inlet, CHI, fluidly coupled to the chemical inlet, CHI, of the waste fermentation system via conduit 54. A pasteurization steam outlet, PSTO, of the steam unit 572 is fluidly coupled to a pasteurization steam inlet, PSTI, of a pasteurization unit 594 via conduit 604, and a pasteurization steam inlet, PSTI, of the steam unit 572 is fluidly coupled to a pasteurization steam outlet, PSTO, of the pasteurization unit 594 via conduit 602. The steam unit 572 further includes a sample clean steam outlet, SCSO, fluidly coupled to a sample clean steam inlet, SCSI, of the pasteurization unit 594 via conduit 606. A drain outlet, D, of the steam unit 570 is fluidly connected to the liquid waste return outlet, LWR, of the waste fermentation system 14 via conduit 584, and another steam outlet, STO, of the steam unit 570 is fluidly connected to the steam outlet, ST, of the waster fermentation system 14 via conduit 64. The steam unit 572 further includes a number, M, of sensors each producing a sensor signal indicative of a corresponding operating condition of the steam unit 572, wherein M may be any positive integer. The “M” sensor signals are supplied to the PLC circuit 120 via a corresponding number of signal paths as illustrated in FIG. 1. The steam unit 572 further includes a number, N, of actuators each responsive to a corresponding actuator control signal supplied by the PLC circuit 120 to control a corresponding operating parameter of the steam unit 572. The steam unit 572 is generally operable, as will be described in greater detail hereinafter with respect to FIGS. 15-16 to provide for the circulation of steam through the sterilization unit 570 and the steam unit 572 via conduits 574, 476 and 578, and also to provide for the circulation of steam through the pasteurization unit 594 and the steam unit 572 via conduits 602 and 604.

In addition to the sterilized liquid waste inlet, SLWI, the fermentation unit 580 further includes a first inner air sparger air inlet, F1I, a first outer air sparger inlet, F1O, a second inner air sparger air inlet, F2I, and a seed steam inlet, F12S, fluidly coupled to the air system 56 via conduits 58, 60, 62 and 66 respectively. First and second seed inlets, SD1 and SD2, of the fermentation unit 580 are fluidly coupled to conduits 46 and 50 respectively. A residual liquid outlet, RLO of the fermentation unit 580 is fluidly coupled to conduit 74, and a gas outlet, GO, of the fermentation unit 580 is fluidly coupled to conduit 68. The fermentation unit 580 further includes a product outlet, POF, fluidly coupled to a product inlet, PIP, of the pasteurization unit 594 via conduit 598, a waste return inlet fluidly coupled to a waste return outlet, WRO, of the pasteurization unit 594 and a water inlet, WI, fluidly coupled to the fresh water conduit 26.

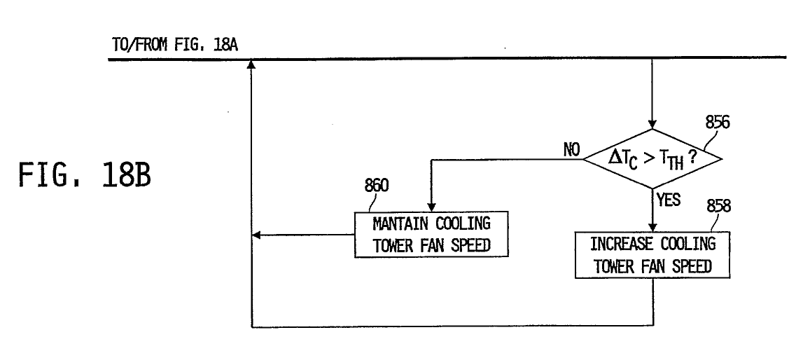

The fermentation unit 580 further includes a coolant flow outlet, CFO, fluidly coupled to a coolant flow inlet, CFI, of a cooling tower unit 586 via conduit 588, and a coolant flow inlet, CFI, fluidly coupled to a coolant flow outlet, CFO, of the cooling tower unit 586 via conduit 590. The temperature of the sterilized liquid biomaterial waste stream supplied to the fermentation unit 580 via conduit 582 is controlled to a target temperature by the cooling tower unit 586 configured to controllably circulate coolant fluid; e.g., water, through the fermentation unit 580 via conduits 588 and 590. The fermentation unit 580 further includes a number, P, of sensors each producing a sensor signal indicative of a corresponding operating condition of the fermentation unit 580, wherein P may be any positive integer. The “P” sensor signals are supplied to the PLC circuit 120 via a suitable number of signal paths as illustrated in FIG. 1. The fermentation unit 580 further includes a number, 0, of actuators each responsive to a corresponding actuator control signal supplied by the PLC circuit 120 to control a corresponding operating parameter of the fermentation unit 580. The fermentation unit 580 is operable, as will be described in greater detail hereinafter with respect to FIGS. 19-26B, to process the incoming sterilized biomaterial waste stream in a manner that produces fermenting organism and residual liquid. The residual liquid stream exits the fermentation unit 580 via the residual liquid outlet, RLO, and the fermenting organism product is supplied to the pasteurization unit 594 via the product outlet port, POF.

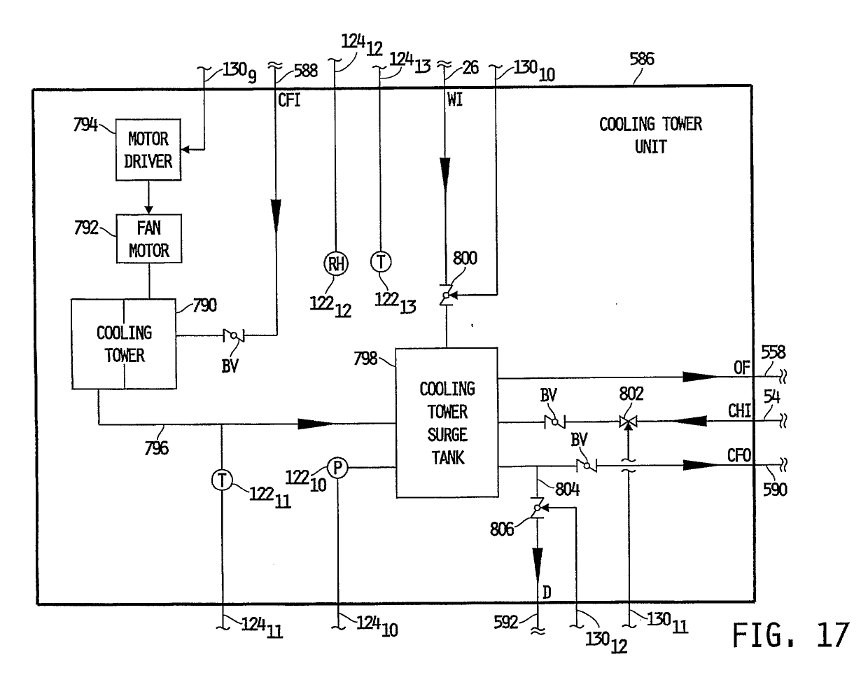

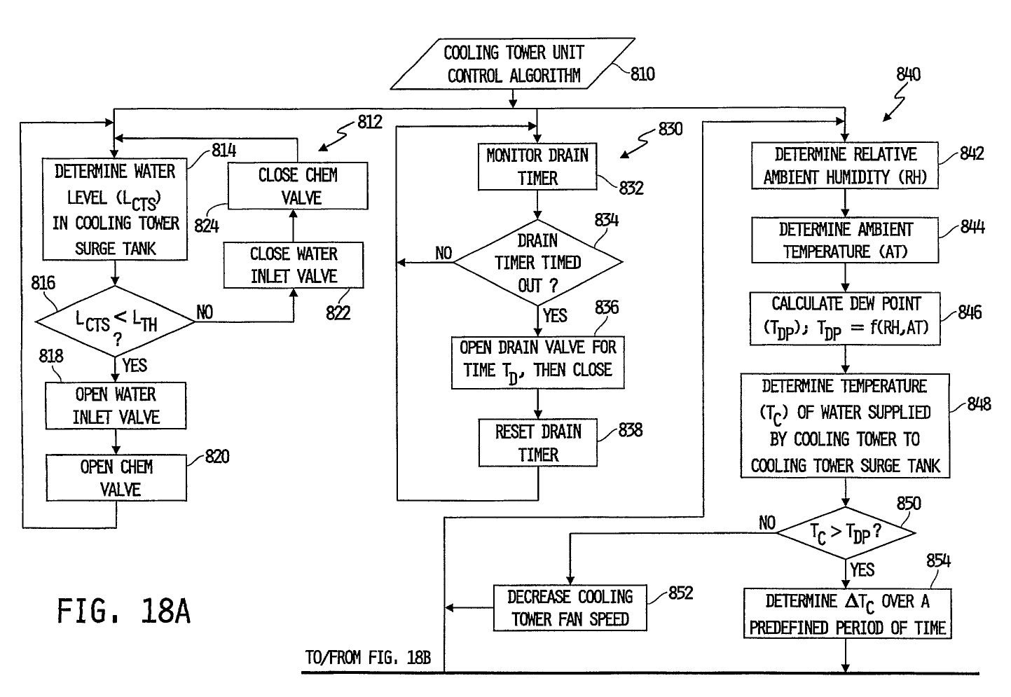

The cooling tower unit 586 further includes a chemical inlet, CHI, fluidly coupled to conduit 54, and an overflow outlet, OF, fluidly coupled to conduit 558. As described hereinabove, in embodiments of the biomaterial waste processing system 10 including a water system 24 of the type illustrated in FIG. 11, the overflow conduit 558 is fluidly coupled to the soft water surge tank 556 for recovery of any overflow water produced by the cooling tower unit 596. In embodiments of the biomaterial waste processing system 10 that do not include a water system 24 of the type illustrated in FIG. 11, and alternatively receive tap water directly from a conventional water source, the overflow conduit 558 may be fluidly coupled to a suitable collection container or system, fluidly coupled to the liquid waste return conduit 76 or allowed to drain to the ground. In any case, the cooling tower unit 586 further includes a drain outlet, D, fluidly coupled to the liquid waste return outlet, LWR, of the waste fermentation system 14 via conduit 592. The cooling tower unit 586 further includes a number, J, of sensors each producing a sensor signal indicative of a corresponding operating condition of the cooling tower unit 586, wherein J may be any positive integer. The “J” sensor signals are supplied to the PLC circuit 120 via a suitable number of signal paths as illustrated in FIG. 1. The cooling tower unit 586 further includes a number, I, of actuators each responsive to a corresponding actuator control signal supplied by the PLC circuit 120 to control a corresponding operating parameter of the fermentation unit 580. The cooling tower unit 586 is operable, as will be described in greater detail hereinafter with respect to FIGS. 17-18B, to controllably circulate coolant fluid; e.g., water, to a portion of the fermentation unit 580 via conduits 588 and 590 to control the temperature of the incoming sterilized liquid biomaterial waste stream to a target temperature.

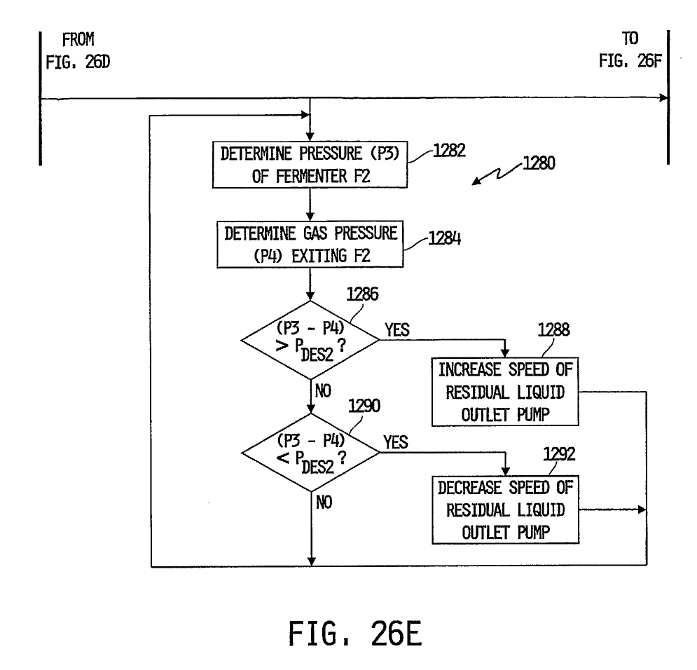

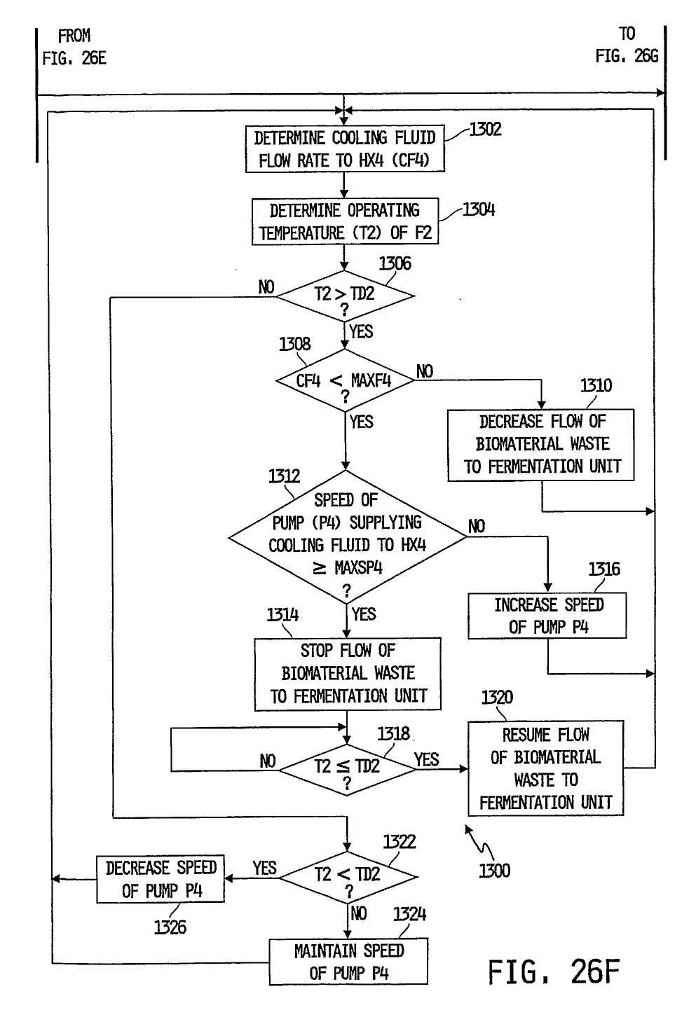

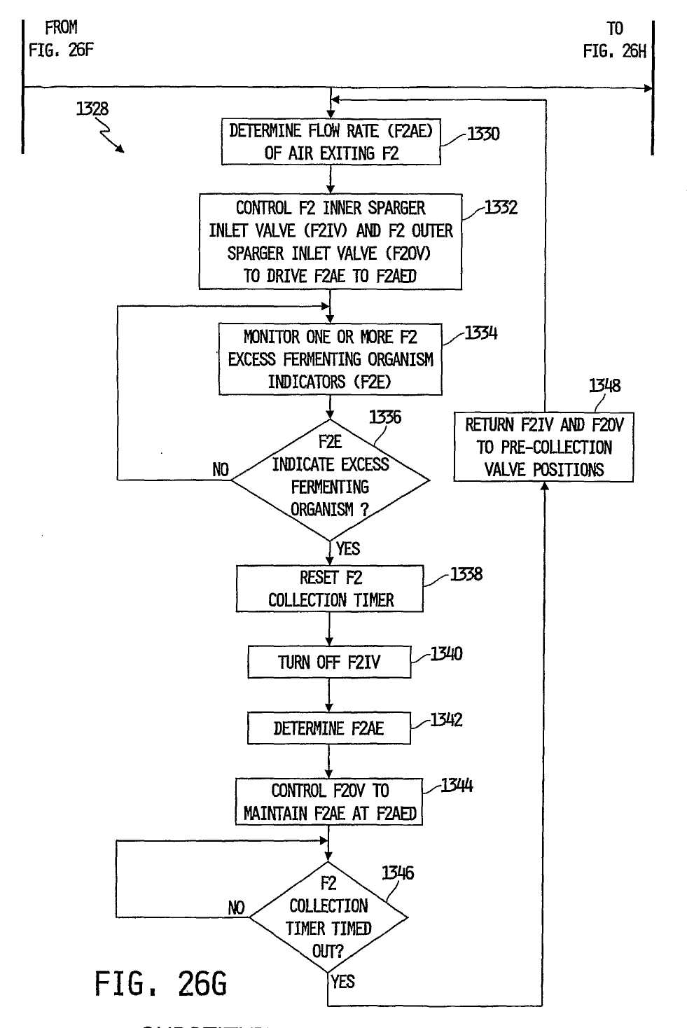

The pasteurization unit 594 further includes a water inlet, WI, fluidly connected to the water inlet conduit 26, and a sample outlet, SMPL, fluidly coupled to a sample outlet conduit 600. The pasteurization unit 594 further includes a number, R, of sensors each producing a sensor signal indicative of a corresponding operating condition of the pasteurization unit 594, wherein R may be any positive integer. The “R” sensor signals are supplied to the PLC circuit 120 via a suitable number of signal paths as illustrated in FIG. 1. The pasteurization unit 594 further includes a number, I, of actuators each responsive to a corresponding actuator control signal supplied by the PLC circuit 120 to control a corresponding operating parameter of the pasteurization unit 594. The pasteurization unit 594 is operable, as will be described in greater detail hereinafter with respect to FIGS. 27-28, to pasteurize and store for later use the fermenting organism produced by the fermentation unit 580.

Referring now to FIG. 13A, a schematic diagram of one illustrative embodiment of the sterilization unit 570 forming part of the waste fermentation stage 14 of FIG. 12 is shown. In the illustrated embodiment, the liquid waste inlet, LWI, is fluidly coupled to the waste stream inlet conduit 42 and to one end of another conduit 610 having an opposite end fluidly coupled through a butterfly valve, BVJ, a check valve, CV and another butterfly valve, BV, to an inlet of a liquid waste pump 612 having a pump outlet fluidly coupled through another butterfly valve, BV, to one end of yet another conduit 614. The liquid waste pump 612 is electrically connected to a conventional pump driver circuit 616 that is also electrically connected to an actuator output of the PLC circuit 120 via one of the “K” signal paths 1302. The PLC circuit 120 is configured to control the liquid waste pump 612 via the pump driver 616 to control the flow of the liquid biomaterial waste stream through the sterilization unit 570.

One of the “L” sensors included within the sterilization unit 570 is a conventional flow rate sensor or flow meter 1223 disposed in-line with conduit 610 between the butterfly valve BVJ and the check valve, CV, and electrically connected to the PLC circuit 120 via signal path 124₃. The flow rate sensor 122₃ is operable to produce a signal on signal path 124₃ indicative of a flow rate of the liquid biomaterial waste stream flowing into the liquid waste inlet, LWI, of the sterilization unit 570. Another one of the “L” sensors included within the sterilization unit 570 is a conventional pressure sensor 122₄ disposed in fluid communication with conduit 610 between the check valve, CV, and the butterfly valve, BV, and electrically connected to the PLC circuit 120 via signal path 124₄. The pressure sensor 122₄ is operable to produce a signal on signal path 124₄ indicative of the pressure of the liquid biomaterial waste stream entering the inlet of the liquid waste pump 612.

Downstream of the outlet of the liquid waste pump 612, conduit 614 passes through another ball valve, BV, a first fluid passageway of a post-sterilization heat exchanger HX1 of known construction, another ball valve, BV, a butterfly valve, BV, and then through a first fluid passageway of a pre-sterilization heat exchanger HX2 also of known construction. After passing through another butterfly valve, BV, conduit 616 is fluidly connected to an inlet of a sterilization loop 630. Between the butterfly valve, BV, adjacent the outlet of the liquid waste pump 612 and the ball valve leading to the heat exchanger HX1, another conduit 618 fluidly connects conduit 614 to one inlet of a pressure relief valve 619 having an outlet coupled through a check valve, CV, to the liquid outlet conduit 78 via conduit 620. A control valve 622 is fluidly connected at one end to the conduit 614 between the intersection of conduit 614 with conduit 618 and the butterfly valve, BV, adjacent to the outlet of the liquid waste pump 612, and at an opposite end to the conduit 620 between the pressure relief valve 619 and the check valve, CV. The control valve 622 is electrically connected to another actuator output of the PLC circuit 120 via another one of the “K” signal paths 130₃, and the PLC circuit 120 is operable to control liquid flow between conduits 614 and 618 via control of the control valve 622. Between the control valve 622 and the butterfly valve, BV, adjacent to the outlet of the liquid waste pump 612, a pressure sensor 122₅ is disposed in fluid communication with conduit 614 and electrically connected to a sensor input of the PLC circuit 120 via another one of the “L” signal paths 124₅.

Another conduit 624 is fluidly connected at one end to conduit 614 between the butterfly valve, BV, adjacent to the outlet of the liquid waste pump 612 and the pressure sensor 122₅, and is coupled through another butterfly valve, BVL, to the liquid outlet conduit 78. Yet another conduit 626 is fluidly connected at one end to conduit 624 between the intersection of conduit 614 and 624 and the butterfly valve, BVL, and is coupled through another butterfly valve, BVK, to the cleaning steam inlet, CSI, of the sterilization unit 570 which is fluidly coupled to conduit 578. Still another conduit 628 is fluidly connected at one end to the inlet conduit 612 between the liquid waste inlet, LWI, of the sterilization system 570 and the butterfly valve, BVJ, and is coupled through another butterfly valve, BVI, to the liquid waste return conduit 76. The junction of conduits 618 and 76 is coupled through yet another butterfly valve, BVH, to the junction of the liquid outlet conduit 78 and conduit 624.

The sterilization steam inlet, SSTI, of the sterilization unit 570 that is fluidly coupled to conduit 574 is also coupled through a control valve 634 and a butterfly valve, BV, to one end of a second fluid passageway defined through the pre-sterilization heat exchanger HX2. An opposite end of the second fluid passageway of HX2 is fluidly coupled to a conduit 632 that is coupled through another butterfly valve, BV, to the sterilization steam outlet, SSTO, of the sterilization unit 570 and also to conduit 576. The control valve 634 is electrically connected to another actuator output of the PLC circuit 120 via another one of the “K” signal paths 130₄. The PLC circuit 120 is configured to controllably circulate steam or other temperature-controlled liquid from the steam unit 572 through the pre-sterilization heat exchanger HX2, via control of the control valve 634, to controllably transfer heat therefrom via the pre-sterilization heat exchanger HX2 to the liquid biomaterial waste stream flowing through conduit 614 to elevate the temperature of the biomaterial waste stream to a sterilization temperature.

The sterilization loop 630 is illustratively provided as a conduit formed in a serpentine, looped or other suitable configuration, wherein the length of the loop 630 and the cross-sectional flow area through the loop 630 define its volumetric capacity, and this volumetric capacity, in turn, defines the sterilization time of the loop 630. In general, the sterilization time of the liquid waste, and the liquid waste temperature required to for such sterilization, is a function of the pH level of the liquid waste passing through the sterilization system 570. By lowering the pH level of the liquid waste stream to an acidic level; e.g., pH 4.0, the combination of time and temperature required for sterilization of the liquid waste stream is also lowered below what would otherwise be required at more neutral pH levels; e.g., pH 7.0. It is appreciated that the optimum pH level for sterilization of the liquid waste is dependent upon the competing organisms that are present in the liquid waste. Therefore, in variations of the sterilization process described herein, a pH level other than e.g. 4.0 is used to shorten the time required to sterilize the liquid waste.

Another one of the “L” sensors included within the sterilization unit 570 is a conventional temperature sensor 122₆ disposed in fluid communication with conduit 614 between the ball valve, BV, disposed in-line with the conduit 614 downstream of the liquid waste outlet of the pre-sterilization heat exchanger HX2 and the inlet of the sterilization loop 630, and electrically connected to a sensor input of the PLC circuit 120 via another one of the “L” signal paths 124₆. The temperature sensor 122₆ may be alternatively positioned relative to the waste stream outlet of the heat exchanger HX2 and the inlet of the sterilization loop 630, and is in any case operable to produce a signal on signal path 124₆ indicative of the temperature of the liquid biomaterial waste stream exiting the waste stream outlet of the pre-sterilization heat exchanger 630 and entering the sterilization loop 630. Yet another of the “L” sensors included within the sterilization unit 570 is a sterilization loop outlet temperature sensor 122₇ of known construction and disposed in fluid communication with a conduit 636 fluidly coupled to the outlet of the sterilization loop 630, and electrically connected to another sensor input of the PLC circuit 120 via another one of the “L” signal paths 124₇. The temperature sensor 122₄ is operable to produce a signal on signal path 127₄ indicative of the temperature of the liquid biomaterial waste stream exiting the outlet of the sterilization loop 630.

The fluid outlet of the sterilization loop 630 is fluidly coupled through another ball valve, BV, through a second fluid passageway of the post-sterilization heat exchanger HX1, and then through another ball valve, BV, to an inlet of a diverter valve 638. Heat from the sterilized biomaterial waste stream exiting the sterilization loop 630 and flowing through conduit 636 is transferred via the post-sterilization heat exchanger HX1 to the biomaterial waste stream flowing through conduit 614 in order to pre-heat the biomaterial waste stream prior to entering the pre-sterilization heat exchanger HX2. Inclusion of the post-sterilization heat exchanger HX1 thus allows for recovery of some of the heat transferred by the pre-sterilization heat exchanger HX2 to the biomaterial waste stream, and thereby reduces the temperature requirements of the steam or other temperature-controlled liquid supplied to the pre-sterilization heat exchanger HX2 via control valve 634 below what would otherwise be required in the absence of the post-sterilization heat exchanger HX1.

One outlet of the diverter valve 638 is fluidly coupled to the liquid waste inlet conduit 610 between the check valve, CV, and the butterfly valve, BV, adjacent to the inlet of the liquid waste pump 612. Another outlet of the diverter valve 638 is fluidly coupled via conduit 642 to an inlet of a pressure control valve 644, and the outlet of the pressure control valve 644 defines the sterilized liquid waste outlet, SLWO, of the sterilization unit 570 and is fluidly coupled to conduit 582. The diverter valve 638 represents another one of the “K” actuators of the sterilization unit 570, and is electrically connected to another actuator output of the PLC circuit 120 via another one of the “K” signal paths 130₆. The PLC circuit 120 is configured to control operation of the diverter valve 638 to control the flow direction of the liquid waste flowing through conduit 636. Under certain operating conditions, the PLC circuit 120 is operable to control the diverter valve 638 to direct the biomaterial waste stream exiting the post-sterilization heat exchanger HX1 back to the inlet of the liquid waste pump 612 for recirculation of the liquid waste through the sterilization unit 570. Otherwise, the PLC circuit 120 is operable to control the diverter valve 638 to direct the biomaterial waste stream out of the sterilization unit 570 and to the fermentation unit 580.

Yet another one of the “L” sensors included within the sterilization unit 570 is a conventional outlet pressure sensor 122₈ disposed in fluid communication with conduit 642 between one outlet of the diverter valve 638 and the inlet of the pressure control valve 644, and electrically connected to another sensor input of the PLC circuit 120 via another of the “L” signal paths 124s. The pressure sensor 122₈ is operable to produce a signal on signal path 124₈ indicative of the pressure of the liquid biomaterial waste stream entering the inlet of the pressure control valve 644, which corresponds to the pressure of the biomaterial waste stream within the sterilization unit 570. The pressure control valve 644 represents yet another one of the “K” actuators of the sterilization unit 570, and is electrically connected to another actuator output of the PLC circuit 120 via another of the “K” signal paths 130₆. The PLC circuit 120 is configured to control operation of the pressure control valve 644 by providing an appropriate actuator control signal on signal path 130₆ and based on the signal produced by the pressure sensor 122₈ to maintain the liquid waste within the sterilization unit 570 near a desired liquid waste pressure.

Some of the conduits and butterfly valves just described are included to allow for the cleaning/sterilization of the sterilization unit 570. For example, in the cleaning/sterilization process described hereinabove with respect to the pH adjustment unit 38, butterfly valves BVJ and BVH may be closed and the butterfly valve BVI opened to provide a cleaning/sterilization path back to the pH adjustment unit 38. During normal, continuous flow operation of the sterilization unit 570, the butterfly valves BVJ and BVH are opened and the butterfly valve BVH is closed. Similarly, butterfly valve BVL may be closed and butterfly valve BVK may be opened to allow steam provided by the steam system 572 via conduit to be supplied to conduit 614 for circulation throughout the sterilization unit 570 when the diverter valve 636 is controlled by the PLC 120 to recirculate the liquid, in this case water, flowing through conduit 634 back through the pump 612 via conduits 638 and 610. When such cleaning/sterilization is complete, the butterfly valve BVK may be closed and the butterfly valve BVL opened to direct the liquid circulating through the sterilization system 470 to the liquid waste return conduit 76 via butterfly valve BVL. During normal, continuous flow operation, the butterfly valves BVK and BVL are both closed. In addition to the manual butterfly valves just discussed, the sterilization unit 570 further includes a number of additional manual valves as illustrated in FIG. 13A. Some of these manual valves are check valves, CV, that are positioned in a number of locations to ensure one-way liquid flow. Others of the manual valves are butterfly or ball valves, BV, and are included within the sterilization unit 570 at various locations to allow for bypassing of, and maintenance or replacement of, various components of the sterilization unit 570.

In another embodiment, a separation process is described where proteins, enzymes, peptides, and the like are removed from the biomaterial waste stream. This separation process may used as a stand-alone treatment process, or as a component of a purification system, treatment system, or fermentation system, such as those described herein. In one aspect, the proteins, enzymes, peptides, and the like are removed by a process that includes the steps of treating the biomaterial waste stream with heat, and removing the proteins, enzymes, peptides, and the like on the basis of density. In another aspect, the heating step is adapted to cause the precipitation, polymerization, or aggregation of the proteins, enzymes, peptides, and the like to form higher molecular weight materials, larger particles, and/or higher density particles in the biomaterial waste stream. Such higher molecular weight materials, larger particles, and/or higher density particles may be removed from the heated biomaterial waste stream under natural gravity, or by means of a gravity induced by for example Coriolis, centrifugal, and/or centripetal forces applied to the heated biomaterial waste stream.

In one variation of this separation process, a separation unit is added in-line prior to sterilization unit. In another variation of the separation process, a separation unit is positioned partway or as an integral component of the sterilization unit. It is appreciated that the relative positioning of separation unit in sterilization unit may be advantageously optimized to achieve a balance between heating time and separation time. For example, separation unit may be placed near the end of sterilization unit to allow maximum heating of the biomaterial waste stream, allowing for maximum precipitation, polymerization, or aggregation of proteins, enzymes, peptides, and the like. It is understood that such an embodiment may require a longer sterilization time and/or higher sterilization temperatures due to the higher heat capacity of a biomaterial waste stream that still includes such proteins, enzymes, peptides, and the like. Alternatively, separation unit may be placed near the beginning of sterilization unit to allow early removal of precipitated, polymerized, or aggregated proteins, enzymes, peptides, and the like. It is understood that such an embodiment may require a higher initial heating temperature to accomplish the desired aggregation, but the sterilization time may be shorted due to the lower heat capacity of the pretreated biomaterial waste stream after removal of the proteins, enzymes, peptides, and the like. It is further appreciated that in this latter variation that early removal will allow either shorter duration or lower temperature sterilization steps. Such shorter duration or lower temperature sterilization steps may have the added benefit of decreasing overall costs of the processes described herein. In addition, such shorter duration or lower temperature sterilization steps may have the added benefit of preserving certain valuable nutrients useable by the fermenting organisms in systems that include fermentation processes, such as valuable organic molecules that might otherwise be degraded by longer duration or higher temperature sterilization steps. For example, certain vitamins and certain carbohydrates may be destroyed in sterilization procedures that include higher heat of sterilization and/or prolonged sterilization times. Illustratively, lower heats and/or shorter times may be used to preserve nutrients such as biotin, pantothenic acid, niacins, B vitamins, including Vitamin B₁, Vitamin B₃, Vitamin B₅, Vitamin B₆, and/or Vitamin B₁₂.

Similarly, the foregoing description is equally applicable to biomaterial waste streams that include vegetative cells, including live or dead bacterial cells. Such cells may tend to cause longer sterilization times and/or higher sterilization temperatures due to the higher heat capacity of biomaterial waste streams that include vegetative cells. It is appreciated that removal of such cells may shorten the time required, or lower the temperature required for sterilization. It is understood that the sterilization step desirably kills competing vegetative cells, and or spores that might compete for nutrients in the fermentation process and decrease overall yield or quality of product. However, the temperatures and or times required to kill cells are each typically greater than required to kill spores. Therefore, removal of vegetative cells either prior to or concurrent with sterilization will allow shorter times and/or lower temperatures to be used.

In another embodiment of the processes and apparatus described herein for fermentation, a sterilization step is included (see FIGS. 13A & 13B). In one illustrative aspect, the sterilization step illustratively reduces the spore count of the biomaterial waste stream entering the precipitating step by a factor of about 10⁶. In another illustrative aspect, the spore count is reduced to a value from about 10⁸ per mL or greater to a value of about 100 per mL or less. It is appreciated that such reductions of spore counts include the substantial removal of bacterial and other vegetative cells from the biomaterial waste stream entering the precipitating step as part of the pretreatment step. In another aspect, the particulate count, including the number of vegetative, bacterial cells, and the like present in the biomaterial waste stream entering the precipitating step is reduced by a precipitating process, such as the precipitating processes described herein, and illustratively shown in FIGS. 13B-13E.

It is understood that the sterilization rates of biomaterial waste streams may follow a logarithmic profile, namely that the rate of sterilization is first order with respect to the concentration of microorganisms present in the biomaterial waste streams entering the sterilization step. In one aspect, the process of sterilization proceeds over a time period t according to the following equation

$$t = \frac{2.303 \cdot log ( {N}_{i} / {N}_{t} )}{K}$$

where Nₜ is the number of organisms alive at time t, Nᵢ is the initial number of organisms, and K is the kinetic rate constant for particular organism destruction. Illustratively spores of Bacillus stearothermophilus are used as an indicator for successful steam sterilization because of their high resistance to this type of sterilization. Accordingly, sterilization processes described herein that are performed in a manner capable of achieving sterilization of Bacillus stearothermophilus are understood to be effective at sterilization of all or substantially all of other organisms present in biomaterial waste stream. The values of K for Bacillus stearothermophilus at different sterilization temperatures are listed in the Table 1

TABLE 1
Calculated rate constant K as a function of temperature for
Bacillus stearothermophilus.

| Temperature (° C.) | K (sec⁻¹) |
|---|---|
| 100 | 0.000235 |
| 103 | 0.000457 |
| 106 | 0.00103 |
| 109 | 0.00209 |
| 112 | 0.00408 |
| 115 | 0.00814 |
| 118 | 0.0162 |
| 121 | 0.0287 |
| 124 | 0.059 |
| 127 | 0.113 |
| 130 | 0.214 |
| 133 | 0.400 |
| 135 | 0.742 |
| 139 | 1.36 |

Illustratively, a biomaterial waste stream that has been pretreated using the precipitating step described herein will be sterilized at a faster rate and/or at lower temperature that would be required for the biomaterial waste stream entering the precipitating step. In one aspect, the biomaterial waste stream entering the precipitating step is barn waste having an Nᵢ=10⁶ spores/ml. In another aspect, the biomaterial waste stream exiting the precipitating step is barn waste having an Nᵢ=10² spores/ml. Illustratively, the predetermined maximum allowable spore count following sterilization is 1 spore per 1,000 gallons (3,800 liters) of fermentation media, and the working volume of the fermenter is about 180,000 gallons (about 680,000 L). According to this aspect, the target spore count (Nₜ) alive at time t is or 2.64×10⁻⁷ spores/mL. Using these values, the time required to achieve Nₜ from Nᵢ in this aspect as a function of temperature for biomaterial waste stream entering the precipitating step and for biomaterial waste stream exiting the precipitating step is shown in Table 2.

TABLE 2
Comparison of sterilization times at various temperatures for
biomaterial waste stream entering or exiting a precipitating step.

| | Sterilization time for | Sterilization time for |
|---|---|---|
| Temperature (° C.) | entering waste (min.) | exiting waste (min.) |
| 100 | 2054 | 1401 |
| 103 | 1056 | 720 |
| 106 | 469 | 320 |
| 109 | 231 | 158 |
| 112 | 118 | 80.7 |
| 115 | 59.3 | 40.4 |
| 118 | 29.8 | 20.3 |
| 121 | 16.8 | 11.5 |
| 124 | 8.18 | 5.58 |
| 127 | 4.27 | 2.91 |
| 130 | 2.26 | 1.54 |
| 133 | 1.21 | 0.82 |
| 135 | 0.65 | 0.44 |
| 139 | 0.36 | 0.24 |

Referring to Table 2, at each temperature, the time required for sterilizing the biomaterial waste stream exiting the precipitating step is decreased by nearly 32% form the time required for sterilizing the biomaterial waste stream entering the precipitating step.

It is appreciated that these processes may also convert a medium molecular weight material, such as proteins, enzymes, peptides, and the like in the range form about 10 kilo Daltons (kDa) to about 100 kDa into higher molecular weight components by heating that may be removed as described in separation unit. Subsequently, the removed high molecular weight components may be converted into low molecular weight components by acid degradation. Such low molecular weight components may be nutrients whereas the starting medium molecular weight components are not. Further, the resulting low molecular weight components may also not as readily precipitate, polymerize, or aggregate as the medium molecular weight components, and thus may be carried through sterilization steps and into subsequent fermentation processes. It is further appreciated that such medium molecular weight components may also include dangerous or undesirable materials such as proteinaceous infective agents (prions).

Prions (Prion protein, PrP) is a small glycosylated protein that is about 231 amino acids in length. The average molecular weight of the naturally occurring amino acids is about 136; therefore, prions are expected to have molecular weights in the range from about 20 kDa to about 40 kDa, or about 31 kDa. In particular, PrP has been found to be resistant to even extremes of pH, heat, chemical degradation, and protease degradation. Bovine Spongiform Encephalopathy (BSE) or Mad Cow Disease is theorized to be an abnormal misfolding of this normal protein to a highly β-sheet containing conformation. Therefore, the heat treating steps described herein are suitable for reducing the amount of, substantially removing, or in some cases completely removing such materials. As described, the removed materials may be discarded or alternatively recycled into the system via an acid hydrolysis step. It is understood that the acid hydrolysis step may not degrade prions to low molecular weight components and therefore such precipitates may be discarded. It is further understood that such components are desirably removed from certain products preparable from the processes described herein, such as animal feed and animal feed supplement products.

In one illustrative embodiment of these processes, a system for treating a biomaterial waste stream that includes a pretreatment step that involves the precipitating step of selected components in the biomaterial waste stream. In one variation of this precipitating step, other selected components remain part of the biomaterial waste stream following the precipitating step. In one aspect, the precipitating step provides the precipitation, agglomeration, and/or aggregation, collectively referred to as precipitation, of proteins, protein fragments, enzymes, enzyme fragments, and/or peptides, and the like. In one variation, the proteins, protein fragments, enzymes, enzyme fragments, and/or peptides having molecular weights of about 60 kDa or greater, or in the range of molecular weights from about 20 kDa to about 60 kDa, from about 20 kDa to about 40 kDa, from about 1 kDa to about 15 kDa, or molecular weights of about 1 kDa or less. In another variation, the proteins, protein fragments, enzymes, enzyme fragments, and/or peptides include prions. In another aspect, the precipitating step provides the precipitation, agglomeration, and/or aggregation of particulates, fine crystals, straw and bedding fragments, and the like.

In another aspect, the removed precipitated, polymerized, or aggregated proteins, enzymes, peptides, and the like, and/or the vegetative cells may be subsequently sent to acid hydrolysis units for degradation. Following degradation, it is appreciated that the subsequent material may be a nutrient for fermenting organisms in the fermentation processes described herein. Alternatively, the removed precipitated, polymerized, or aggregated proteins, enzymes, peptides, and the like, and/or the vegetative cells may be discarded, including those components that cannot be otherwise degraded into smaller components by conventional processes and apparatus, or by the processes and apparatus described herein.

In another aspect, the pretreatment includes the step of heating the biomaterial waste stream to cause the precipitation, polymerization, or aggregation of the proteins, enzymes, peptides, and the like, where the subsequently precipitated, polymerized, or aggregated material also traps additional material, such as suspended particles, including clay, cells, fine straw particulates, bedding particulates, lignin, and the like. The separation unit is configured to allow the aggregated material to be removed on the basis of density either under natural gravity or under an artificial gravity that is created by centrifugation, vortexing, or like process.

In another variation, metal salts are also added during the heating step to facilitate precipitation, polymerization, and/or aggregation of the suspended or dissolved material. Such metal salts include salts of aluminum, iron, other transition metals, divalent and trivalent metals, and like salts. Counter anions of such metal salts include hydroxide, carbonate, biocarbonate, sulfate, bisulfate, chloride, bromide, and the like.

In another variation, the removed aggregate material is recycled into other separation processes and/or degradation processes described herein, including acid hydrolysis processes. It is further appreciated that removing suspended or dissolved solids by precipitation as described herein may reduce or prevent the clogging of optional additional apparatus such as filters, centrifuges, ultracentrifuges, and the like.

In another embodiment, a system for treating a biomaterial waste stream that includes this separation process and associated apparatus described herein coupled to and feeding into a process and associated apparatus for precipitating dissolved solids from an aqueous solution as described herein. In one aspect of this embodiment, a fermentation step is also included. In another aspect of this embodiment, a fermentation step is not included.

In one embodiment of the precipitating step, the precipitated, agglomerated, and/or aggregated, collectively referred to as precipitated, components prepared in the precipitating step are removed by gravity settling. It is appreciated that in some configurations of the apparatus described herein, gravity settling may be unacceptably slow due a chimney effect in the settling tank. It is further appreciated that gravity settling may be impracticable in continuous flow apparatus. It is understood that the chimney effect may be used to facilitate settling of precipitated components prepared in the precipitating step in configurations that involve continuous flow by causing the precipitated components to collect and concentrate in a direction opposite to that of the clarified liquid component. In a vertical configuration, the precipitated component may be directed to the walls of a tank configured for performing the separating step creating thereby a downward flow. It is appreciated that in continuous flow configurations, the flow near the walls of the tank may be lower in velocity, allowing denser, or heavier particulate material to settle out of waste being treated, and leave a clarified liquid behind. It is further appreciated that due to heat loss at the walls of such tanks, settled particulates will tend to create a more pronounced downward flow due to the increased density of the cooler settled material.

It is appreciated that substantial buffering of the biomaterial waste stream exiting the precipitating-separating step is removed when components including proteins, bacterial cells, soluble fibers, and other components are removed from the biomaterial waste stream entering the precipitating-separating step. In system configurations that include both a precipitating-separation step and a post treatment step, it is appreciated that less base may be needed to raise the pH of the biomaterial waste stream entering the post treatment step.

Referring now to FIG. 13B, a schematic diagram of another illustrative embodiment of the sterilization unit 570′ and corresponding control system that forms part of the waste fermentation system 14 is shown. The sterilization unit 570′ and associated control system illustrated in FIG. 13B is identical in many respects to the sterilization unit 570 and associated control system illustrated in FIG. 13A, and like numbers are therefore used to identify like components. In the embodiment illustrated in FIG. 13B, a high pressure biomaterial waste settling tank 637 is interposed between the waste stream outlet of the heat exchanger HX1 and the waste stream inlet of the heat exchanger HX2. More particularly, the waste stream outlet of the heat exchanger HX1 is fluidly coupled to a waste stream inlet of the high pressure settling tank 637 via a conduit 623 coupled to a conduit 635, and a waste stream outlet of the high pressure settling tank 637 is fluidly coupled to the waste stream inlet of the heat exchanger HX2 via a conduit 639. A precipitation initiation tank 633 has an outlet fluidly coupled to an inlet of a conventional pump 627 via a conduit 631. An outlet of the pump 627 is fluidly coupled to an inlet of a mixer 625 having an outlet fluidly coupled to the junction of the conduits 623 and 635. A conventional pump driver 629 is electrically connected to the pump 627, and is electrically connected to another actuator output of the PLC circuit 120 via signal path 130_{A}. The precipitation initiation tank 633 contains a precipitation initiator fluid or mixture, as will be described in greater detail hereinafter, and the PLC circuit 120 is operable to control the pump 465 to controllably provide the precipitation initiator contained within the tank 633 to the waste inlet of the high pressure settling tank 637.

A waste outlet of the high pressure settling tank 637 is fluidly coupled to an inlet of another conventional pump 643 via a conduit 641, and the outlet of the pump 643 is fluidly coupled to an inlet of a control valve 647. A conventional pump driver 645 is electrically connected to the pump 643, and is electrically connected to another actuator output of the PLC circuit 120 via signal path 130_{B}. The control input of the control valve 647 is likewise electrically connected to another actuator output of the PLC circuit 120 via signal path 130_{C}. The outlet of the control valve 647 is fluidly coupled to a waste inlet of a low pressure settling tank 649 via a conduit 655, and a liquid outlet of the low pressure settling tank 649 is fluidly coupled to the residual liquid outlet 74 of the waste fermentation system 14. A waste outlet of the low pressure settling tank 649 is fluidly coupled to an inlet of another conventional pump 651 via a conduit 657, and an outlet of the pump 651 is fluidly coupled to the precipitated waste outlet conduit 80. Another conventional pump driver 653 is electrically connected to the pump 651, and is electrically connected to another actuator output of the PLC circuit 120 via signal path 130_{C}.

Referring now to FIG. 13C, a cross-sectional view of either of the settling tanks 637, 649 is shown. In the illustrated embodiment, the tank 637, 649 is cylindrically-shaped and has an outer wall 661 terminating at a top 687 at one end, and terminating at a bottom 683 at an opposite end. The top 687 defines a liquid outlet in fluid communication with the conduit 639, 74, and the bottom 683 defines a solid waste outlet 685 fluidly coupled to the conduit 641, 657. A number of inner cylinders 663₁-663_{N} are positioned inside of the tank 637, 649 and stacked one atop another, wherein N may be any positive integer. Referring to FIG. 13D, an illustrative embodiment of one of the inner cylinders 663 is shown. The inner cylinder 663 is hollow and has an open bottom end 665 and an opposite end having a truncated cone top 667. The truncated cone top defines an opening 669 therethrough, and the truncated cone top 667 slopes generally downwardly and away from the opening 669. A conical disk 671 is positioned approximately centrally within the inner cylinder 663, and is held in place by a suitable rod, plate or similar structure 673 secured to the wall of the inner cylinder 663 and the conical disk 671. The conical disk 671 is positioned within the inner cylinder 663 approximately mid way between the bottom 665 and the opening 669, with the tip of the cone extending generally toward a center of the opening 669.

Referring again to FIG. 13C, the inner cylinders 663₁-663_{N} are stacked one atop another, and the open bottom ends 665₁-665_{N} are sized relative to the truncated cone tops 667₁-667_{N} so that adjacent bottoms and tops of the inner cylinders 663₁-663_{N} form gaps 679 therebetween. It will be noted that the top-most inner cylinder 663_{N} does not have a truncated cone-top in the illustrated embodiment, and is instead open like the bottom end 665_{N}, although it will be understood that the top-most inner cylinder 663_{N} may alternatively include a truncated cone-top. A cone-shaped bottom member 681 is positioned adjacent to the bottom 683 of the tank 637, 649, and is sized to form a gap 679 between the bottom 665₁ of the bottom-most inner cylinder 663₁ and the bottom member 681 as illustrated in FIGS. 13C and 13D. The bottom-most inner cylinder 663₁ includes a second conical disk 675 inverted relative to the conical disk 671₁ with the conical disk juxtaposed over the conical disk 675. the distal end 635A, 655A of the waste inlet conduit 635, 655 is directed upwardly toward the tip of the conical disk 675.

In example one embodiment, the following dimensions apply to the inner cylinders 663₁-663_{N} and to the tank 637, 649, although it will be understood that the inner cylinders 663₁-663_{N} and tank 637, 649 may be constructed with other dimensions. Each of the inner cylinders 663₁-663_{N}, in this example, are 25 inches (64 cm) in height and 46 inches (117 cm) in diameter. The openings 669₁-669_{N} are 20 inches (51 cm) in diameter, and the conical disks 671₁-671_{N} are 22 inches (56 cm) in diameter. The tank 637, 649 is 12 feet (3.7 m) in height, and 4 feet (1.2 m) in diameter. The distance between the lowest edge of the conical disks 671₁-671_{N} and the openings 669₂-669_{N} above is 15.5 inches (39.4 cm), and the distance between the lowest edge of the conical disks 671₂-671_{N} and the openings 669₁-669_{N-1} below is 10 inches (25 cm). The size of the gaps 679 are ½ inch (1.3 cm), and the distance between the center of the conduit 635, 655 and the bottom 665₁ of the bottom-most inner cylinder 663₁ is 8 inches (20 cm). The distance between the distal end 635A, 655A of the conduit 635, 655 and the tip of the conical disk 675 is 5 inches (13 cm), the distance between the distal end 635A, 655A of the conduit 635, 655 and the tip of the conical disk 671₁ is 10.5 inches (26.7 m), and the distance between the distal end 635A, 655A of the conduit 635, 655 and the adjacent edges of the of the conical disks 671₁ and 675 is 7.5 inches (19.1 cm). The diameter of the waste outlet 685 is 1 foot (0.3 m), and the distance between the center of the conduit 641, 657 and the bottom of the waste outlet 685 is 3.5 inches (8.9 cm).

Referring now to FIGS. 13E and 13F, operation of the settling tanks 637, 649, as it relates to the flow of liquid biomaterial waste therethrough and extraction of solids, will now be described. As shown in FIG. 13E, liquid biomaterial waste, which is periodically mixed with a precipitation initiator from the precipitation initiator tank 633 as described herein, enters the waste inlet 635, 655 as illustrated by the directional arrow 689. This liquid mixture exits the distal end 635A, 655A of the waste inlet conduit 635, 655 and is directed toward the tip of the conical disk 675. As illustrated by the directional arrows 691, the conical disk 675 directs the liquid flow outwardly around the conical disks 675 and 671₁. Because the opening 669, is smaller in diameter than the conical disks 675 and 671₁, the flow of liquid is directed back toward the center of the inner cylinder 663₁ as shown. This process repeats as the liquid travels upwardly through the tank 635, 655. This liquid flow pattern causes the flow rate of liquid through the inner cylinders 663₁-663_{N} to be greater toward center of each of the inner cylinders 663₁-663_{N} and less near the outer walls of each of the inner cylinders 663₁-663_{N}. This is illustrated graphically in FIG. 13F by the directional arrows 693A and 693B, wherein the arrow 693B indicates a higher flow 30 rate and the arrow 693A indicates a relatively lesser flow rate. As a result of this liquid flow pattern, areas 695 of little or no liquid flow are created just above the truncated cone tops 667₁-667_{N} near the sidewalls of each of the inner cylinders 663₁-663_{N}. Heavier waste particles carried by the biomaterial waste tend to drop out of the liquid in the low or no flow areas 695, and begin to collect on the truncated conical tops 671₁-671_{N}. When sufficient amounts of waste particles have collected, the collective weight of the waste particles cause them to slide off the conical tops 671₁-671_{N}, through the gaps 679, and downwardly toward the bottom 683 of the tank 637, 649 as illustrated by the directional arrows 697 and 699. In addition, it is appreciated that due to heat loss through the outer walls of of tanks 637, 649, the liquid flowing in gap may be cooler than the bulk liquid entering and exiting tanks 637, 649. This cooler liquid will be more dense, and will facilitate movement of solid waste particles from low or no flow areas 695 into gaps 679, and downwardly toward the bottom 683 of the tank 637, 649. The resulting solid waste particles are collected in the waste outlet 685, as indicated by the directional arrows 701, and may be periodically pumped out by periodically activating the waste pumps 643 and 651. The periodic operation of the pumps 643 and/or 651 is, in one embodiment, time-based. Other pump control strategies may alternatively be used.

In one illustrative example, pre-fermentation barn flush liquid waste spiked with a 29 kDa protein was treated at pH 4, with added aluminum, and heated at 121° C. to remove proteins in the 20-40 kDa molecular weight range, and illustratively in the 27-32 kDa molecular weight range (understood to be the prion molecular weight range).

Operation of the sterilization unit 570 is controlled by the PLC circuit 120 based on information provided by one or more of the sensors associated with the sterilization unit 570. Referring now to FIGS. 14A-14C, a flowchart of one illustrative embodiment of a software algorithm 650 for controlling the sterilization unit 570 is shown. It will be understood that the software algorithm 650 represents one illustrative strategy for controlling the sterilization unit 570 during normal, continuous flow operation of the biomaterial waste processing system 10, and that the sterilization unit 570 may be controlled differently during other operational modes of the biomaterial waste processing system 10. Examples of other operational modes of the biomaterial waste processing system 10 may include, but are not limited to, off, power/air fail, power/air fail recovery, seeding, start-up, transition from start-up to normal, continuous flow operation, preparation for system sterilization and system sterilization. In any case, the software algorithm 650 is stored within, or programmed into, the PLC circuit 120, and the PLC circuit 120 is operable to execute algorithm 650 to control the operation of the sterilization unit 570.

The control algorithm 650 includes a number of different and independently executing control routines, and each of these different control routines will be described separately. For example, the control algorithm 650 includes a first control routine 652 for controlling the speed of the liquid waste pump 612 as a function of the inlet pressure signal on signal path 124₄ to maintain the pressure of the biomaterial waste stream entering the liquid waste pump 612 below a threshold inlet pressure, and also as a function of the flow rate signal on signal path 124₃ to maintain the flow rate of the biomaterial waste stream entering the liquid waste inlet port, LWI, of the sterilization unit 570 between upper and lower flow rate values. The control routine 652 begins at step 654 where the PLC circuit 120 is operable to sense the waste stream inlet pressure, P_{I}, by monitoring the inlet pressure signal on signal path 124₄. Thereafter at step 656, the PLC circuit 120 is operable to compare the waste stream inlet pressure, P_{I}, to an inlet pressure threshold, P_{ITH}. If P_{I} exceeds P_{ITH} at step 656, execution of the control routine 652 advances to step 658 where the PLC circuit 120 is operable to control the pump driver 616, by producing an appropriate actuator control on signal path 130₂, to reduce the pump speed of the liquid waste pump 612. If, on the other hand, the PLC circuit 120 determines at step 656 that P_{I} is less than or equal to P_{ITH}, execution of the control routine 652 advances to step 660 where the PLC circuit 120 is operable to sense the waste stream inlet flow rate, FRI, by monitoring the flow rate signal on signal path 124₃. Thereafter at step 662, the PLC circuit 120 is operable to compare the waste stream inlet flow rate, FRI, to a high flow rate threshold, FR_{HTH}. If FRI exceeds FR_{HTH} at step 662, execution of the control routine 654 advances to step 664 where the PLC circuit 120 is operable to control the pump driver 616, by producing an appropriate actuator control on signal path 130₂, to reduce the pump speed of the liquid waste pump 612. If, on the other hand, the PLC circuit 120 determines at step 662 that FRI is less than FR_{HTH}, execution of the control routine 652 advances to step 666 where the PLC circuit 120 is operable to compare the waste stream inlet flow rate, FRI, to a low flow rate threshold, FR_{LTH}, wherein FR_{LTH}<FR_{HTH}. If FRI is less than FR_{HTH} at step 666, execution of the control routine 652 advances to step 668 where the PLC circuit 120 is operable to control the pump driver 616, by producing an appropriate actuator control on signal path 130₂, to increase the pump speed of the liquid waste pump 612. If, on the other hand, the PLC circuit 120 determines at step 666 that FRI is greater than or equal to FR_{LTH}, execution of the control routine 652 loops back to step 654, as it also does following steps 658, 664 and 668. The PLC circuit 120 is thus operable, pursuant to control routine 652, to control the speed of the liquid waste pump 612 to maintain the liquid pump inlet pressure below a pressure threshold, P_{ITH}, and to maintain the flow rate of the incoming waste stream to the sterilization unit 570 within a flow rate window defined between lower and upper flow rate thresholds FR_{LTH} and FR_{HTH} respectively.

The sterilization unit control algorithm 650 further includes another control routine 670 for controlling the steam control valve 634 as a function of the heat exchanger outlet temperature signal on signal path 124₆ to maintain the temperature of the biomaterial waste stream exiting the pre-sterilization heat exchanger HX2 above a target sterilization temperature. The control routine 670 begins at step 672 where the PLC circuit 120 is operable to determine whether the sterilization unit 570 is enabled for operation. Generally, the PLC circuit 120 is operable to command sterilization operation under normal, continuous flow operation, and in such an operation mode sterilization is thus typically commanded. If, however, the PLC circuit 120 determines at step 672 that sterilization operation is not currently commanded or enabled, execution of the control routine 670 advances to step 674 where the PLC circuit 120 is operable to control the position of the steam control valve 634, by producing an appropriate actuator command signal on signal path 130₄, to a closed position. If, on the other hand, the PLC circuit 120 determines at step 672 that sterilization operation is currently commanded or enabled, the PLC circuit 120 is operable at step 676 to sense the temperature of the waste stream exiting the pre-sterilization heat exchanger HX2, T_{EX}, by monitoring the heat exchanger outlet temperature signal on signal path 124₆. Thereafter at step 678, the PLC circuit 120 is operable to compare T_{EX} to a target sterilization temperature, T_{ST}. If T_{EX} exceeds T_{ST} at step 678, execution of the control routine 670 advances to step 680 where the PLC circuit 120 is operable to control the position of the steam control valve 634, by producing an appropriate actuator control signal on signal path 130₄, to reduce T_{EX} by decreasing the flow area through the steam control valve 634. If, on the other hand, the PLC circuit 120 determines at step 678 that T_{EX} not greater than T_{ST}, execution of the control routine 670 advances to step 682 where the PLC circuit 120 is operable to again compare T_{EX} to the target sterilization temperature, T_{ST}. If T_{EX} is less than T_{ST} at step 682, execution of the control routine 670 advances to step 684 where the PLC circuit 120 is operable to control the position of the steam control valve 634, by producing an appropriate actuator control on signal path 130₄, to increase T_{EX} by increasing the flow area through the steam control valve 634. Execution of the control routine 672 loops from the “no” branch of step 682 back to step 672, as it also does following steps 674, 680 and 684. The PLC circuit 120 is thus operable, pursuant to control routine 670, to control the position of the steam control valve 634 to maintain the temperature of the waste stream exiting the pre-sterilization heat exchanger HX2 near a target sterilization temperature, T_{ST}.

The sterilization unit control algorithm 650 further includes another control routine 686 for controlling operation of the diverter valve 638 as a function of the sterilization outlet temperature signal on signal path 124₇ to ensure the temperature of the biomaterial waste stream within the sterilization loop 630 is maintained near the target sterilization temperature for a predefined sterilization time period. The control routine 686 begins at step 688 where the PLC circuit 120 is operable to determine whether the sterilization unit 570 is enabled for operation as described hereinabove. If the PLC circuit 120 determines at step 688 that sterilization operation is not currently commanded or enabled, execution of the control routine 686 advances to step 696 where the PLC circuit 120 is operable to control the position of the diverter valve 638, by producing an appropriate actuator command signal on signal path 130₅, to direct the biomaterial waste stream flowing through conduit 636 to conduit 640 to thereby recirculate the waste stream back through the sterilization unit 570. If, on the other hand, the PLC circuit 120 determines at step 688 that sterilization operation is currently commanded or enabled, the PLC circuit 120 is operable at step 690 to sense the temperature, TSLO, of the waste stream exiting the sterilization loop 630 by monitoring the sterilization loop outlet temperature signal on signal path 124₇. Thereafter at step 692, the PLC circuit 120 is operable to compare TSLO to the target sterilization temperature, T_{ST}. If TSLO is less than T_{ST} at step 692, execution of the control routine 686 advances to step 696. If, on the other hand, the PLC circuit 120 determines at step 692 that TSLO is greater than or equal to T_{ST}, execution of the control routine 686 advances to step 694 where the PLC circuit 120 is operable control the position of the diverter valve 638, by producing an appropriate actuator command signal on signal path 130₅, to direct the biomaterial waste stream flowing through conduit 636 to conduit 642 to thereby route the sterilized waste stream to the pressure control valve 644. Execution of the control routine 686 loops from either of steps 694 and 696 back to step 688. The PLC circuit 120 is thus operable, pursuant to control routine 686, to control the position of the diverter valve 638 to direct the waste stream exiting the sterilization loop 630 back through the sterilization unit 570 if the temperature of the waste stream is below the target sterilization temperature, T_{ST}, and to otherwise direct the waste stream exiting the sterilization loop 630 to the pressure control valve 644.

The sterilization unit control algorithm 650 further includes another control routine 698 for controlling operation of the pressure control valve 644 as a function of the pressure of the biomaterial waste stream within the sterilization unit 570 to maintain the pressure of the biomaterial waste stream within the sterilization unit 570 near a target pressure value. The control routine 698 begins at step 700 where the PLC circuit 120 is operable to sense the pressure, P_{S}, of the waste stream within the sterilization system 570 by monitoring the outlet pressure signal on signal path 124₈. Thereafter at step 702, the PLC circuit 120 is operable to compare the pressure, P_{S}, to a target pressure value or pressure set point, P_{SET}. If, P_{S} exceeds P_{SET} at step 702, execution of control routine 698 advances to step 704 where the PLC circuit 120 is operable to control the position of the pressure control valve 644, by producing an appropriate actuator command signal on signal path 130₆, to reduce P_{S} by decreasing the flow area through the pressure control valve 644. If, on the other hand, the PLC circuit 120 determines at step 702 that P_{S} is not greater than P_{SET}, execution of the control routine 698 advances to step 706 where the PLC circuit 120 is operable to compare P_{S} to a minimum waste stream pressure value, P_{MIN}. Generally, it is desirable to set P_{MIN} to a pressure value slightly above which the pressure of the waste stream within the sterilization unit 570 will drop if the safety pressure relief valve 619 opens to direct the biomaterial waste stream to the liquid waste return conduit 76. If, at step 706, the PLC circuit 120 determines that P_{S} is less than P_{MIN}, such as may occur if the safety pressure relief valve 619 opens, execution of the control routine 698 advances to step 708 where the PLC circuit 120 is operable to control the position of the pressure control valve 644, by producing an appropriate actuator control on signal path 130₆, to close the control valve 644 and thereby inhibit the flow of the biomaterial waste stream to the sterilized liquid waste outlet, SLWO, of the sterilization unit 570. If, on the other hand, the PLC circuit 120 determines at step 706 that P_{S} is not less than P_{MIN}, execution of the control routine 698 advances to step 710 where the PLC circuit 120 is operable to again compare P_{S} to P_{SET}. If, at step 710, the PLC circuit 120 determines that P_{S} is less than P_{SET}, execution of the control routine 698 advances to step 712 where the PLC circuit 120 is operable to control the position of the pressure control valve 644, by producing an appropriate actuator command signal on signal path 130₆, to raise the pressure, P_{S}, of the waste stream within the sterilization system 570. If, on the other hand, the PLC circuit 120 determines at step 710 that P_{S} is not less than P_{SET}, execution of the control routine 698 loops back to step 700, as it also does following steps 704, 708 and 712. The PLC circuit 120 is thus operable, pursuant to control routine 698, to control the position of the pressure control valve 644 to maintain the pressure of the waste stream within the sterilization unit 570 near a target or set pressure value, P_{SET}, and to close the pressure control valve 644 if the pressure of the waste stream within the sterilization system 570 drops below a minimum pressure value, P_{MIN}.

The sterilization unit control algorithm 650 further includes another control routine 714 for controlling operation of the control valve 622 between conduits 614 and 620 as a function of the pressure of the biomaterial waste stream within the sterilization unit 570, when the sterilization unit 570 is operating in a recirculation mode with the diverter valve 638 directing the waste stream flowing through conduit 636 to conduit 640, to prevent overpressure conditions. The control routine 714 begins at step 716 where the PLC circuit 120 is operable to determine whether the sterilization unit 570 is operating in recycle or recirculation mode. In the illustrated embodiment, the PLC circuit 120 is configured to execute step 716 by monitoring the status of the diverter valve 638. For example, if the diverter valve 638 is positioned to direct the liquid waste stream to conduit 640, the sterilization unit 570 is operating in recycle or recirculation mode, whereas if the diverter valve 638 is positioned to direct the liquid waste stream to conduit 642, the sterilization unit 570 is instead operating in the normal, continuous flow mode. If the PLC circuit 120 determines at step 716 that the sterilization unit 570 is not in recycle or recirculation mode, execution of the control routine 714 loops back for re-execution of step 716. If, on the other hand, the PLC circuit 120 determines at step 716 that the sterilization unit 570 is in recycle or recirculation mode, execution of the control routine 714 advances to step 718 where the PLC circuit 120 is operable to sense the pressure, P_{R}, of the waste stream within the sterilization system 570 by monitoring the outlet pressure signal on signal path 124₅. Thereafter at step 720, the PLC circuit 120 is operable to compare the pressure, P_{R}, to a threshold pressure value, P_{RTH}. If, P_{R} exceeds P_{RTH} at step 720, execution of control routine 714 advances to step 722 where the PLC circuit 120 is operable to control the position of the control valve 622, by producing an appropriate actuator command signal on signal path 130₃, to open the control valve 622. If, on the other hand, the PLC circuit 120 determines at step 722 that P_{R} is not greater than P_{RTH}, execution of the control routine 698 advances to step 724 where the PLC circuit 120 is operable to control the position of the control valve 622, by producing an appropriate actuator command signal on signal path 130₃, to close the control valve 622. Execution of the control routine 714 loops from either of steps 722 and 724 back to step 716. In any operating mode of the sterilization unit 570, the mechanical pressure relief valve 619 is configured to open if the pressure within conduit 614 exceeds a safe operating pressure, P_{SAFE}, to direct the flow of liquid waste through conduit 614 to the liquid waste return conduit 76. Valve 622 and control routine 714 provide some redundancy in this regard, and provide for more active control of the pressure of the liquid waste stream flowing through conduit 614.

### EXAMPLE 1

Sterilization of an animal waste stream within the sterilization unit 570 is achieved as a combination of time, temperature, and pH level of the waste stream. A relatively higher sterilization temperature will produce a relatively shorter sterilization time. It has been found that the quality of the resulting sterilized animal waste stream is higher with short duration sterilization times and concomitant higher sterilization temperatures. An example of settings found effective are summarized in Table 3:

TABLE 3

| | Design | | Design |
|---|---|---|---|
| | Temperature | | Pressure |
| Description | (° F.) | (° C.) | (psig) |
| Steam | 320 | 160 | 75 |
| Liquid entering sterilization loop 630 | 275 | 135 | 31 |
| Liquid exiting sterilization loop 630 | 270 | 132.222 | 27 |

| | | |
|---|---|---|
| Liquid after sterilization | ambient + 2-4 | 40 |
| Pump pressure required | | 55 |

Sterilization retention time (TIMING LOOP 630):

| | |
|---|---|
| Sterilization loop pipe diameter | 6 inches (15.24 centimeters) |
| Sterilization loop length | 173 feet (52.7304 meters) |
| Volume of loop | 254 gallons (961.494 liters) |
| Flow rate of Liquid | 125 gpm (473.177 lpm) |
| Retention time | 2.03 minutes |

The retention time at 100 gpm (379 lpm) is (125/100)*2.03=2.5 minutes

Referring now to FIG. 15, a schematic diagram of one illustrative embodiment of the steam unit 572 forming part of the waste fermentation system, 14 of FIG. 12 is shown. In the illustrated embodiment, the water inlet, WI, of the steam unit 572 is fluidly coupled to the water inlet conduit 66, and also to a water inlet conduit 730 coupled to an inlet of a boiler feed surge tank 732 via a conventional control valve 734 and a butterfly valve, BV. The control valve 734 represents one of the “M” actuators of the steam unit 572, and is electrically connected to an actuator output of the PLC circuit 120 via one of the “M” signal paths 130₇. The PLC circuit 120 is configured to control operation of the control valve 734 by providing an appropriate actuator control signal on signal path 130₇. One of the “N” sensors included within the steam unit 572 is a conventional pressure sensor 122₉ disposed in fluid communication with the boiler feed surge tank 732, and electrically connected to the PLC circuit 120 via one of the “N” signal paths 124₉. The pressure sensor 122₉ is operable to produce pressure signal on signal path 124₉ indicative of the water pressure within the boiler feed surge tank 732, and the PLC circuit 120 is configured to process the pressure signal in a known manner and determine a water level value corresponding to the level of water within the boiler feed surge tank 732.

The chemical inlet port, CHI, of the steam unit 572 is fluidly coupled to the chemical inlet conduit 54, and is coupled to a chemical inlet of the boiler feed surge tank 732 via a conventional control valve 738 and a butterfly valve, BV, disposed in-line with a conduit 736. The control valve 738 represents another one of the “M” actuators of the steam unit 572, and is electrically connected to an actuator output of the PLC circuit 120 via another one of the “M” signal paths 130₈. The PLC circuit 120 is configured to control operation of the control valve 738 by providing an appropriate actuator control signal on signal path 130₈. The drain outlet, D, of the steam unit 572 is fluidly coupled to the liquid waste return conduit, 76, of the waste fermentation system 14, and is fluidly coupled through a butterfly valve, BV, to a drain outlet of the boiler feed surge tank 732. Optionally, the butterfly valve, BV, may be replaced by a control valve that is electrically controlled by the PLC circuit 120. In this embodiment, the boiler feed surge tank 732 may thus be drained under the control of the PLC circuit 120. The boiler feed surge tank 732 is configured to store a quantity of pressurized water therein, and conventional water conditioning; e.g., water softening, chemicals may be provided to the boiler feed surge tank 732 via the chemical inlet, CHI, to condition/soften the water stored therein. It will be understood that in embodiments of the biomaterial waste processing system 10 that include a source of conditioned water, such as the water source 24 illustrated in FIG. 11, the water supplied to the boiler feed surge tank 732 via the water inlet, WI, of the steam unit 572 will be soft water. In such embodiments, the steam unit 572 need not include the chemical inlet, CHI, control valve 738 and associated butterfly valve, BV, and conduit 736, although these components may be included within the steam unit 572 to provide for farther water conditioning control.

A water outlet of the boiler feed surge tank 734 is fluidly coupled through a pair of butterfly valves, BV, to a fresh water inlet of a de-aeration tank 742 via a conduit 740. A water outlet of the de-aeration tank 742 is fluidly coupled through a pair of flow reducers in the form of globe valves, GV, to a water inlet of a conventional boiler 746. The de-aeration tank 742 is operable in a known manner to purge the water stored therein of air bubbles to minimize corrosion of the boiler 746 by oxygen carried by any such air bubbles. Although not shown in FIG. 15, the de-aeration tank 742 and boiler 746 include a conventional closed-loop feedback system therebetween that is not controlled by the PLC circuit 120, and that maintains the boiler 746 at a desired pressure/temperature.

A steam/water return inlet of the de-aeration tank 742 is fluidly coupled through a flow reducer in the form of a globe valve, GV, to an outlet of a conventional steam trap 750 via a steam return conduit 748, and an inlet of the steam trap 750 is fluidly coupled to an outlet of a conventional particle strainer 752. An inlet of the particle strainer 752 defines the sterilization steam inlet port, SSTI, of the steam unit 572 and is fluidly coupled to conduit 576. The steam return conduit 748 is further fluidly coupled via another globe valve, GV, and a check valve, CV, to an outlet of another conventional steam trap 756 via a conduit 754. An inlet of the steam trap 756 is fluidly coupled to an outlet of another conventional particle strainer 758 having an inlet defining the pasteurization steam inlet port, PSTI, of the steam unit 572 and is fluidly coupled to conduit 602.

The steam outlet conduit 760 fluidly coupled to the steam outlet of the boiler 746 defines the sterilization steam outlet, SSTO, the pasteurization steam outlet, PSTO, and the steam outlet, ST, to the air system 56, of the steam system 572, and is accordingly fluidly connected to conduits 574 and 604, and 64. The steam outlet conduit 760 is further fluidly coupled to a cleaning steam conduit 762 that is fluidly coupled to the cleaning steam outlet, CSO, of the steam unit 572, and therefore to conduit 578, through a pair of flow reducers in the form of globe valves, GV. The steam outlet conduit 760 is also fluidly coupled to a sample cleaning steam conduit 764 that is fluidly coupled to the sample cleaning steam outlet, SCSO, of the steam unit 572, and therefore to conduit 606, through another pair of flow reducers in the form of globe valves, GV. The boiler feed surge tank 732 is configured to supply water to the de-aeration and boiler tanks 742 and 746 respectively, and the boiler tank 746 is configured to heat the water supplied thereto to produce steam that is circulated through various other units of the waste fermentation system 14 and air system 56 and then returned to the de-aeration and boiler tanks 742 and 746 for reheating. A number of butterfly valves, BV, and globe valves, GV, are included within the steam unit 572 at various locations to allow for bypassing of, and maintenance or replacement of, various components of the steam unit 572. The globe valves, GV, also provide for predefined pressure or flow reductions of the steam or water across these valves.

Referring now to FIG. 16, a flowchart of one illustrative embodiment of a software algorithm 770 for controlling the steam unit 572 is shown. It will be understood that the software algorithm 770 represents one illustrative strategy for controlling the steam unit 572 during normal, continuous flow operation of the biomaterial waste processing system 10, and that the steam unit 572 may or may not be controlled differently during other operational modes of the biomaterial waste processing system 10. In any case, the software algorithm 770 is stored within, or programmed into, the PLC circuit 120, and the PLC circuit 120 is operable to execute algorithm 770 to control operation of the steam unit 572. The algorithm 770 begins at step 772 where the PLC circuit 120 is operable to determine the water level, L_{BF}, in the boiler feed surge tank 732. In the illustrated embodiment, the PLC circuit 120 is operable to execute step 772 by monitoring the signal produced by the pressure sensor 122₉ on signal path 124₉, and processing this signal in a known manner to determine L_{BF}. Thereafter at step 774, the PLC circuit 120 is operable to compare L_{BF} to a threshold water level, L_{TH}. If L_{BF} is less than L_{TH}, execution of the algorithm 770 advances to step 776 where the PLC circuit 120 is operable to control the water inlet valve 734, by producing an appropriate control signal on signal path 130₇, to open the water inlet valve 732. In one embodiment of the steam unit 572, water conditioning chemicals are automatically added whenever the water inlet valve 732 is opened. In this embodiment, algorithm 770 includes optional step 778 as shown in phantom in FIG. 16. If included, the PLC circuit 120 is operable at step 778 to control the chemical inlet valve 738, by producing an appropriate control signal on signal path 130₈, to open the chemical inlet valve 738. In alternative embodiments, water conditioning chemicals are added on a timed or other basis, or not at all, and in these embodiments the optional step 778 may be omitted. Algorithm execution loops from step 778, or from step 776 in embodiments where step 778 is not included in algorithm 770, back to step 772.

If, at step 774, the PLC circuit 120 determines that L_{BF} is greater than or equal to L_{TH}, algorithm execution advances to step 780 where the PLC circuit 120 is operable to control the water inlet valve 734, by producing an appropriate control signal on signal path 130₇ to close the water valve 734. In embodiments of the algorithm 770 including step 778, algorithm 770 further includes the optional step 782 shown in phantom. If included, the PLC circuit 120 is operable at step 782 to control the chemical inlet valve 738, by producing an appropriate control signal on signal path 130₈, to close the chemical inlet valve 738. Algorithm execution loops from step 782, or from step 780 in embodiments where step 782 is not included in algorithm 770, back to step 772.

Referring now to FIG. 17, a schematic diagram of one illustrative embodiment of the cooling tower unit 586 and corresponding control system that forms part of the waste fermentation system 14 of FIG. 12 is shown. In the illustrated embodiment, the cooling fluid inlet, CFI that is fluidly coupled to conduit 588 is also fluidly coupled through a butterfly valve, BV, to a cooling fluid inlet of a cooling tower 790. A conventional fan motor 792 drives a cooling fan associated with the cooling tower 790, and is electrically connected to a conventional motor driver 794. The motor driver 794 represents one of the “I” actuators of the cooling tower unit 586, and is electrically connected to another one of the actuator outputs of the PLC circuit 120 via one of the “I” signal paths 130₉. The cooling tower 790 is a conventional water cooling unit configured to cool water flowing therethrough via operation of its cooling fan, and the PLC circuit 120 is operable to control the rate of such cooling by controlling the fan motor 792 via the motor driver 794.

A fluid outlet of the cooling tower 790 is fluidly coupled to a cooling tower surge tank 798 via a conduit 796. One of the “J” sensors included within the cooling tower unit 586 is a conventional temperature sensor 122₁₁ disposed in fluid communication with the conduit 796 and electrically connected to the PLC circuit 120 via one of the “J” signal paths 124₁₁. The temperature sensor 122₁₁ is operable to produce a temperature signal on signal path 124₁₁ indicative of the temperature of the water flowing through the conduit 796. Another one of the “J” sensors included within the cooling tower unit 586 is a conventional pressure sensor 122₁₀ disposed in fluid communication with the cooling tower surge tank 798, and electrically connected to the PLC circuit 120 via one of the “J” signal paths 124₁₀. The pressure sensor 122₁₀ is operable to produce a pressure signal on signal path 124₁₀ indicative of the water pressure within the cooling tower surge tank 798, and the PLC circuit 120 is configured to process this pressure signal in a known manner and determine a water level value corresponding to the level of water within the cooling tower surge tank 798. The cooling tower surge tank 798 further includes a fresh water inlet coupled through an inlet control valve 800 to the water inlet, WI, of the cooling tower unit 586, which is fluidly coupled to the water inlet conduit 26. The inlet control valve 800 represents another one of the “I” actuators of the cooling tower unit 586, and is electrically connected to another one of the actuator outputs of the PLC circuit 120 via one of the “I” signal paths 130₁₀. The cooling tower surge tank 798 is a conventional water storage tank configured to store and controllably supply pressurized water.

The chemical inlet port, CHI, of the cooling tower unit 586 is fluidly coupled to the chemical inlet conduit 54, and is coupled to a chemical inlet of the cooling tower surge tank 798 via a conventional control valve 802 and a butterfly valve, BV. The control valve 802 represents another one of the “I” actuators of the cooling tower unit 598, and is electrically connected to another one of the actuator outputs of the PLC circuit 120 via one of the “I” signal paths 130₁₁. The PLC circuit 120 is configured to control operation of the control valve 802 by providing an appropriate actuator control signal on signal path 130₁₁. Conventional water conditioning; e.g., water softening, chemicals may be provided to the cooling tower surge tank 798 via the chemical inlet, CHI, to condition/soften the water stored therein. It will be understood that in embodiments of the biomaterial waste processing system 10 that include a source of conditioned water, such as the water source 24 illustrated in FIG. 11, the fresh water supplied to the cooling tower surge tank 798 via the water inlet, WI, of the cooling tower unit 586 will be soft water. In such embodiments, the cooling tower unit 586 need not include the chemical inlet, CHI, and control valve 802, although these components may be included within the cooling tower unit 586 to provide for further control of the condition of the water stored in the cooling tower surge tank 798. In embodiments of the biomaterial waste processing system 10 that do not include a source of fresh, conditioned water, and the fresh water supplied to the water inlet, WI, of the cooling tower unit 586 is therefore unconditioned water, the cooling tower unit 586 may further include conventional water conditioning components to condition the fresh water supplied to the cooling tower surge tank 798. In such embodiments, the water inlet, WI, of the cooling tower unit may be fluidly coupled to a conventional water conditioner, and the water conditioner fluidly coupled to the fresh water inlet of the cooling tower surge tank. The chemical inlet, CHI, in such embodiments may be coupled to a chemical inlet of the water conditioner and/or cooling tower surge tank 798.

The cooling tower surge tank 798 further includes an overflow outlet fluidly coupled to conduit 598 via the overflow outlet, OF, of the cooling tower unit 586. In embodiments of the biomaterial waste processing system 10 including a source of conditioned water, such as the water source 24 illustrated in FIG. 11, the overflow conduit 558 may be fluidly coupled to such a water source to recirculate overflow water through the water source 24. If, on the other hand, the biomaterial waste processing system 10 does not include a water source such as water source 24, but is instead configured to receive tap water from a conventional tap water source, the overflow conduit 558 may be fluidly coupled to a suitable container, another water processing system or vented to ground. Alternatively, in such embodiments wherein the cooling tower unit 586 includes water conditioning components as just described, the overflow outlet of the cooling tower surge tank 798 may be fluidly coupled to such water conditioning components.

The cooling tower surge tank 798 further includes a cooling fluid outlet fluidly coupled through a butterfly valve, BV, to the cooling fluid outlet, CFO, of the cooling tower unit 586 and also to conduit 590. Between the cooling fluid outlet of the cooling tower surge tank 798 and the butterfly valve, BV, another conduit 804 is coupled through an outlet control valve 806 to the drain outlet, D, of the cooling tower unit 586, which is fluidly coupled to conduit 592. The outlet control valve 806 represents another one of the “I” actuators of the cooling tower unit 586, and is electrically connected to another one of the actuator outputs of the PLC circuit 120 via another one of the “I” signal paths 13012. The conditioned water stored in the cooling tower surge tank 798 may, over time, become saturated with water conditioning chemicals, and the PLC circuit 120 is configured to control the outlet valve 806 to periodically drain some of the saturated water from the tank 798 so that appropriate water conditioning chemical levels may be restored.

Another one of the “J” sensors included within the cooling tower unit 586 is a conventional relative humidity sensor 122₁₂ disposed in fluid communication with the ambient air surrounding the cooling tower 790, and electrically connected to the PLC circuit 120 via one of the “J” signal paths 124₁₂. The relative humidity sensor 122₁₂ is operable to produce a signal on signal path 124₁₂ indicative of the relative humidity of the ambient air about the cooling tower 790. Yet another one of the “J” sensors included within the cooling tower unit 586 is another conventional temperature sensor 122₁₃ disposed in fluid communication with the ambient air about the cooling tower 790, and electrically connected to the PLC circuit 120 via one of the “J” signal paths 124₁₃. The temperature sensor 122₁₃ is operable to produce a temperature signal on signal path 124₁₃ indicative of the temperature of the ambient air surrounding the cooling tower 790. The PLC circuit 120 is configured to process the signals produced by the sensors 122₁₂ and 122₁₃ in a known manner to determine a dew point of the ambient air surrounding the cooling tower 790, and to control operation of the fan motor 792 as a function of the computed dew point.

The cooling tower unit 586 just described includes a number of manually actuated butterfly valves, BV, as illustrated in FIG. 17. Such valves are included within the cooling tower unit 586 at various locations to allow for bypassing of, and maintenance or replacement of, various components of the cooling tower unit 586.

Referring now to FIGS. 18A-18B, a flowchart of one illustrative embodiment of a software algorithm 810 for controlling the cooling tower unit of FIG. 17 is shown. It will be understood that the software algorithm 810 represents one illustrative strategy for controlling the cooling tower unit 586 during normal, continuous flow operation of the biomaterial waste processing system 10, and that the cooling tower unit 586 may be controlled differently during other operational modes of the biomaterial waste processing system 10. The software algorithm 810 includes a number of different and independently executing control routines, and each of these different control routines will be described separately. For example, the control algorithm 810 includes a first control routine 812 for controlling the level and condition of the water in the cooling tower surge tank 798. The control routine 812 begins at step 814 where the PLC circuit 120 is operable to determine the water level, L_{CTS}, in the cooling tower surge tank 798. In the illustrated embodiment, the PLC circuit 120 is operable to execute step 814 by monitoring the signal produced by the pressure sensor 122₁₀ on signal path 124₁₀, and processing this signal in a known manner to determine L_{CTS}. Thereafter at step 816, the PLC circuit 120 is operable to compare L_{CTS} to a threshold water level, L_{TH}. If L_{CTS} is less than L_{TH}, execution of the control routine 812 advances to step 818 where the PLC circuit 120 is operable to control the water inlet valve 800, by producing an appropriate control signal on signal path 130₁₀, to open the water inlet valve 800. Thereafter at step 820, the PLC circuit 120 is operable to control the chemical inlet valve 802, by producing an appropriate control signal on signal path 130₁₁, to open the chemical inlet valve 802. Alternatively, the PLC circuit 120 may be configured to control the chemical inlet valve 802 on a timed or other basis, in which case step 820 may be omitted from the control routine 812. Algorithm execution loops from step 820, or from step 818 in embodiments where step 820 is not included in control routine 812, back to step 814.

If, at step 816, the PLC circuit 120 determines that L_{CTS} is greater than or equal to L_{TH}, execution of the control routine 812 advances to step 822 where the PLC circuit 120 is operable to control the water inlet valve 800, by producing an appropriate control signal on signal path 130₁₀ to close the water valve 800. In embodiments of the control routine 812 including step 820, control routine 812 further includes step 824 where the PLC circuit 120 is operable to control the chemical inlet valve 802, by producing an appropriate control signal on signal path 130₁₁, to close the chemical inlet valve 802. Execution of the control routine 812 loops from step 824, or from step 822 in embodiments where step 824 is not included in control routine 812, back to step 814.

The cooling tower unit control algorithm 810 further includes another control routine 830 for controlling operation of the drain control valve 806. The control routine 830 begins at step 832 where the PLC circuit 120 is operable to monitor the status of a drain timer resident in the PLC circuit 120. Thereafter at step 834, the PLC circuit 120 is operable to determine whether the drain timer has timed out. If not, execution of the control routine loops back to step 832. If, however, the PLC circuit 120 determines at step 834 that the drain timer has timed out, execution of the control routine 830 advances to step 836 where the PLC circuit is operable to control the drain control valve 806 by opening the drain control valve 806 for a time period T_{D} to drain a desired quantity of water from the cooling tower surge tank 798, and then to close the drain control valve 806. Thereafter at step 838, the PLC circuit 120 is operable to reset the drain timer, and execution of the control routine 830 loops from step 838 back to step 832.

The cooling tower unit control algorithm 810 further includes another control routine 840 for controlling operation of the fan motor 792. The control routine 840 begins at step 842 where the PLC circuit 120 is operable to determine the relative humidity, RH, of the ambient air surrounding the cooling tower 790. In the illustrated embodiment, the PLC circuit 120 is operable to execute step 842 by monitoring the signal produced by the ambient relative humidity sensor 122₁₂ on signal path 124₁₂. Thereafter at step 844, the PLC circuit 120 is operable to determine the temperature, AT, of the ambient air surrounding the cooling tower 790. In the illustrated embodiment, the PLC circuit 120 is operable to execute step 844 by monitoring the signal produced by the ambient temperature sensor 122₁₃ on signal path 124₁₃. Following step 844, the PLC circuit 120 is operable at step 846 to calculate the dew point temperature, T_{DP}, as a known function of RH and AT.

Following step 846, the PLC circuit 120 is operable at step 848 to determine the temperature, T_{C}, of the water supplied by the cooling tower 790 to the cooling tower surge tank 798. In the illustrated embodiment, the PLC circuit 120 is operable to execute step 848 by monitoring the signal produced by the temperature sensor 122₁₁ on signal path 124₁₁. Thereafter at step 850, the PLC circuit 120 is operable to compare the temperature, T_{C}, of the water supplied by the cooling tower 790 to the cooling tower surge tank 798 with the dew point temperature, T_{DP}. If T_{C} is less than or equal to T_{DP} at step 850, then the cooling fan motor 792 is working harder than it needs to and execution of the control routine 840 advances to step 852 where the PLC circuit 120 is operable to control the motor driver 794 by producing an appropriate motor driver control signal on signal path 130₉, to decrease the speed of the fan motor 792 and therefore decrease the speed of the cooling tower fan. Execution of the control routine 840 loops from step 852 back to step 842.

If, at step 850, the PLC circuit determines that T_{C}>T_{DP}, execution of the control routine 840 advances to step 854 where the PLC circuit 120 is operable to monitor the signal produced by the temperature sensor 1221, on signal path 124₁₁ over a predefined time period to determine the change in T_{C}, or ΔT_{C}, over the predefined time period. Thereafter at step 856, the PLC circuit 120 is operable to compare ΔT_{C} to a threshold temperature, T_{TH}. If, at step 856, ΔT_{C}>T_{TH}, then the cooling fan motor is not working hard enough and execution of the control routine 840 advances to step 858 where the PLC circuit 120 is operable to control the motor driver 794 by producing an appropriate motor driver control signal on signal path 130₉, to increase the speed of the fan motor 792 and therefore increase the speed of the cooling tower fan. If, however, the PLC circuit 120 determines at step 856 that ΔT_{C} is less than or equal to T_{TH}, then any increase in the cooling tower fan speed will not correspondingly decrease T_{C} and execution of the control routine 840 advances to step 860 where the PLC circuit 120 is operable to control the motor driver 794 by producing an appropriate motor driver control signal on signal path 130₉, to maintain the current speed of the fan motor 792 and therefore maintain the current speed of the cooling tower fan. Execution of the control routine 840 loops from steps 858 and 860 back to step 842.

Referring now to FIG. 19, a diagrammatic representation of one illustrative embodiment of the fermentation unit 580 forming part of the waste fermentation system 14 of FIG. 12 is shown. In the illustrated embodiment, the fermentation unit 580 includes a first fermenter 870 having a reactor 872 in the form of an elongated, hollow cylinder, although other geometric shapes of the reactor 872 are contemplated. In the illustrated embodiment, an elongated, hollow bottom inner cylinder 874 is longitudinally received within the reactor 872 with a bottom end 876 positioned adjacent to a bottom end 878 of the reactor, and a top end 880, and with the sidewall of the bottom inner cylinder 874 positioned adjacent to and spaced apart from the sidewall of the reactor 872. A hollow top inner cylinder 884 is also received within the reactor 872 with a bottom end 882 positioned adjacent to and spaced apart from the top end 880 of the bottom inner cylinder 874, and a top end 886 positioned adjacent to a top end 888 of the reactor 872, and with the sidewall of the top inner cylinder 884 positioned adjacent to and spaced apart from the sidewall of the reactor 872. A liquid outlet conduit 900 is fluidly coupled to the reactor 872 adjacent to the sidewall of the top inner cylinder 884, and an air outlet conduit 902 is fluidly coupled to the reactor 872 through the top end 888 of the reactor 872.

The reactor 872 further includes a funnel-shaped cone 890 positioned adjacent to the bottom end 876 of the bottom inner cylinder 874 and fluidly coupled to a product outlet conduit 892 extending from the bottom of the cone 890, and extending outwardly from the bottom end 878 of the reactor 872. Fermenting organism formed in the first fermenter 870 is extracted via the product outlet conduit 892. A liquid waste inlet, LWI, is fluidly coupled via conduit 894 to the interior of the bottom inner cylinder 874 adjacent to the bottom end 876, and is configured to receive therein a continuous stream of liquid biomaterial waste. A primary air inlet, F1O, is fluidly coupled to an outer air sparger 896 configured to distribute incoming air evenly about the cone 890 within the bottom inner cylinder 874, and a secondary air inlet 898, F1I, is fluidly coupled to an inner air sparger 898 configured to distribute incoming air evenly within the interior of the cone 890.

The fermentation unit 580 further includes a second fermenter 910 fluidly coupled to the first fermenter 870. The second fermenter 910 is diagrammatically similar to the first fermenter 870 and includes a reactor 912 in the form of an elongated, hollow cylinder, although other geometric shapes and relative proportions of the reactor 912 than those illustrated are contemplated. In the illustrated embodiment, an elongated, hollow bottom inner cylinder 914 is longitudinally received within the reactor 912 with a bottom end 916 positioned adjacent to a bottom end 918 of the reactor, and a top end 920, and with the sidewall of the bottom inner cylinder 914 positioned adjacent to and spaced apart from the sidewall of the reactor 912. A hollow top inner cylinder 924 is also received within the reactor 912 with a bottom end 922 positioned adjacent to and spaced apart from the top end 920 of the bottom inner cylinder 914, and a top end 926 positioned adjacent to a top end 928 of the reactor 912, and with the sidewall of the top inner cylinder 924 positioned adjacent to and spaced apart from the sidewall of the reactor 912. A liquid outlet conduit 936 is fluidly coupled to the reactor 912 adjacent to the sidewall of the top inner cylinder 924, and is fluidly coupled to the residual liquid outlet, RLO, of the fermentation unit 580. An air outlet conduit 938 is fluidly coupled to the reactor 912 through the top end 928 of the reactor 912, and is fluidly coupled to the gas outlet, GO, of the fermentation unit 580.

The reactor 912 further includes a funnel-shaped cone 930 positioned adjacent to the bottom end 916 of the bottom inner cylinder 914 and fluidly coupled to a product outlet conduit 932 extending from the bottom of the cone 930, and extending outwardly from the bottom end 918 of the reactor 912. The product outlet conduit 932 is fluidly coupled to the product outlet, POF, of the fermentation unit 580. The product outlet conduit 892 of the first fermenter unit 870 is fluidly coupled to the lower portion of the cone 930 such that the fermenting organism extracted from the lower portion of the cone 890 of the fermenter 870 enters the lower portion of the cone 930 of the fermenter 910, below the inner air sparger 934. The liquid outlet conduit 900 of the first fermenter 870 is fluidly coupled to the interior of the bottom inner cylinder 914 adjacent to the bottom end 916, and conduit 900 thus forms the liquid waste inlet to the second fermenter 912 receiving therein a continuous stream of liquid exiting the first fermenter 870. The air outlet conduit 902 of the first fermenter 870 is fluidly coupled to an outer air sparger 904 configured to distribute incoming air evenly about the cone 930 within the bottom inner cylinder 916, conduit 902 thus forms the primary air inlet to the second fermenter 910. A secondary air inlet, F2I, is fluidly coupled to an inner air sparger 934 configured to distribute incoming air evenly within the interior of the cone 930.

The fermenters 870, 910 illustrated in FIG. 19 represent an “air-lift” design, wherein mixing is performed by the introduction of air into the inner bottom cylinders 874, 914 and taking advantage of the circulation created as the result of the expansion of the air as it rises in the reactor 872, 912. This air is introduced into the reactors 872, 912 via the outer air spargers 896, 904. Secondary air is selectively introduced within the cones 890, 930 via the inner air spargers 898, 934 to cause admixing of the cone contents with the reactor contents. Admixing is precluded when no secondary air flows through the inner air spargers 898, 934. Exhaust gases are constantly and controllably removed from the second fermenter 910 via conduit 939 to maintain a constant desired pressure within the fermenters 870, 910, and a constant volume of liquid is controllably removed via conduit 936 to maintain a constant desired liquid volume within the fermenters 870, 910. Flocculated fermenting organism is selectively removed from the first and second fermenters 870, 910 to adjust the fermenting organism content in the corresponding reactors 872, 912.

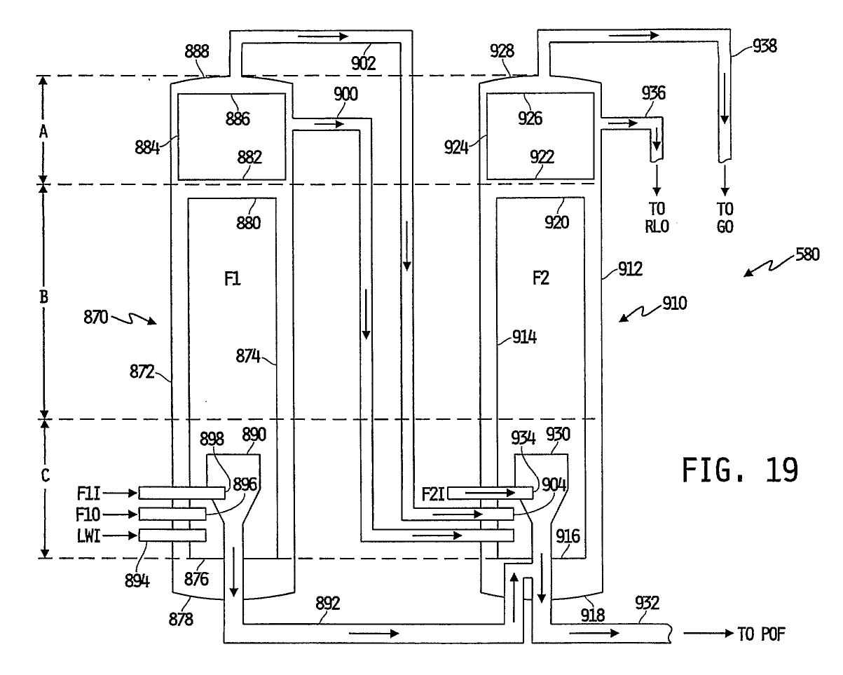

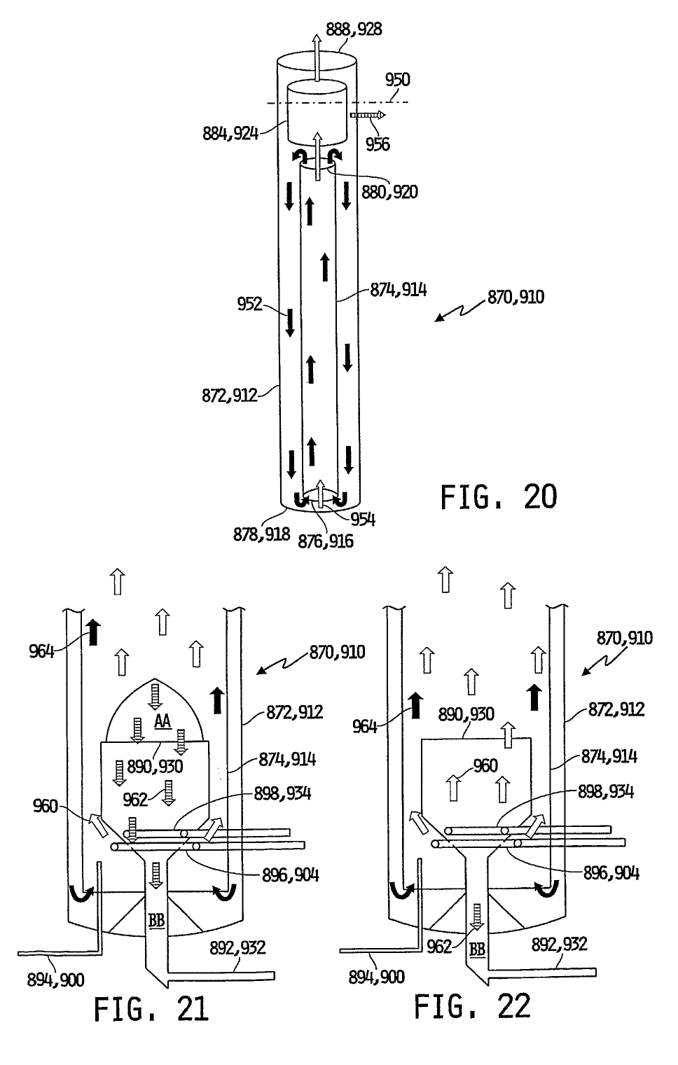

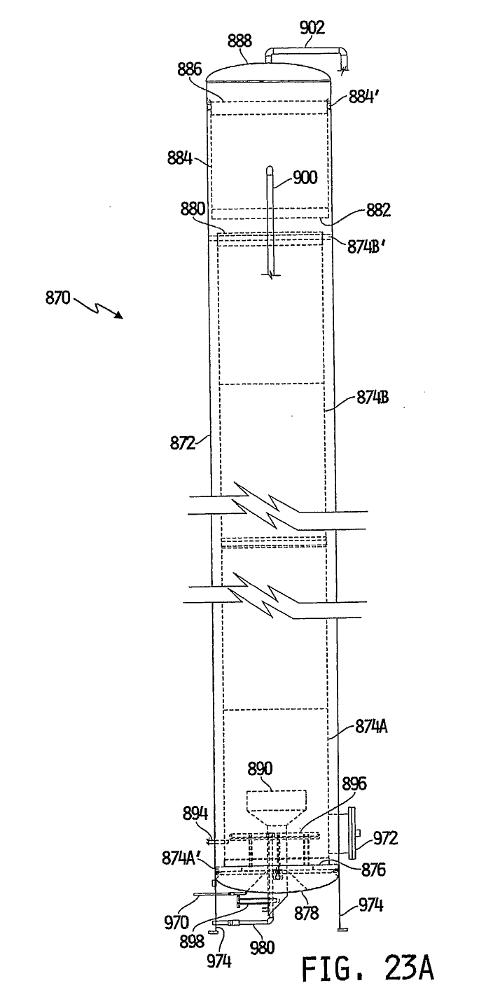

In general, region “A” illustrated in FIG. 19 represents the region of the fermenters 870, 910 where air and liquid are separated, region “B” represents the region where liquid/air mixing and fermenting organism growth occurs, and region “C” represents the region where fermenting organism reduction and separation is carried out. Details relating to each of these operational regions of the fermenters 870, 910 will now be described with respect to FIGS. 20-22, wherein FIG. 20 is a diagrammatic illustration of the general operation of either of the fermentation tanks 870, 910 in a normal, continuous flow operational mode, FIG. 21 is a diagrammatic illustration of the operation of the air spargers 896, 898 and 904,930 and fermenting organism collection cone 890, 930 in either of the fermentation tanks 870, 910 in a fermenting organism reduction operational mode, and FIG. 22 is a diagrammatic illustration of the operation of the air spargers 896, 898 and 904,930 and fermenting organism collection cone 890, 930 in either of the fermentation tanks 870, 910 in the normal, continuous flow operational mode. It will be understood that the concepts illustrated and described with respect to FIGS. 20-22 apply equally to the first and second fermenters 870 and 910, except where noted.

In the diagram of FIG. 20, the general operation of an “air lift” fermenter design is illustrated. Air is introduced via the outer air spargers 896, 904 adjacent to the bottom ends 876, 916 of the bottom inner cylinders 874, 914, and this air naturally rises to the top as illustrated by the arrows sharing a common design with arrow 954. The aspect ratios of the fermenters 870, 910; i.e., the height to diameter ratios of the fermenters 870, 910, are selected to create specified pressure differentials between the bottoms 878, 918 and the tops 888, 928 of each reactor 870, 910. Typically, at least the first fermenter 870 has a high aspect ratio; e.g., 5-6, and an example first fermenter having a height of 60 feet and a diameter of 9 feet will create a pressure differential of approximately 2 atmospheres or 29.4 pounds per square inch (2.08 kg per square centimeter) from bottom 878, 918 to top 888, 928. As the air rises within the reactors from the bottom inner cylinder 874, 914, it expands to multiples of its original volume, and this upward force and expansion displaces liquid which spills over the top 880, 920 of the bottom inner cylinder 874, 914 and falls downwardly through the space defined between the sidewall of the reactor 872, 912 and the sidewall of the bottom inner cylinder 874, 914, as illustrated by the arrows having a common pattern with arrow 952.

The top inner cylinder 884, 924 extends above the liquid level 950, and because the top inner cylinder 884, 924 is spaced apart from the bottom inner cylinder 874, 914 and the liquid is thereby spilled downwardly over the top 880, 920 of the bottom inner cylinder 874, 914, little or no net upward velocity is created in the top inner cylinder 884, 924. As a result, flocculated fermenting organism falls, along with the liquid, downwardly from the top of the bottom inner cylinder 880, 920 toward the bottom 876, 916 of the bottom inner cylinder 874, 914. Also as a result of little or no net upward velocity in the top inner cylinder 884, 924, a calm area is created in the area or gap between the sidewall of the reactor 872, 912 and the sidewall of the top inner cylinder 884, 924, allowing removal of liquid via liquid outlet conduit 900, 936 that is essentially free of flocculated fermenting organism as illustrated by arrow 956. Air escaping from the liquid above the liquid level 950 is directed out of the top 888, 928 of the fermenter 870, 910 via the air outlet conduit 902, 938. Via implementation of the multiple inner cylinder design of the fermenters 870, 910, as illustrated in FIGS. 19 and 20, air and liquid are separated within the region “A.”

In the bottom inner cylinder 874, 914, the rapid upward flow of the air supplied to the bottom inner cylinder 874, 914 is balanced with the rapid downward fall of liquid in the gap between the reactor 872, 912 and the bottom inner cylinder 874, 914. The upward flow of air into the bottom inner cylinder 874, 914 draws the rapidly falling liquid into the bottom 876, 916 of the bottom inner cylinder 874, 916, and circulation of the liquid about the bottom inner cylinder 874, 914, as illustrated by arrows having a common pattern with arrow 952, results in thorough mixing of the liquid and organisms within the mixing and growth region “B” illustrated in FIG. 19. Additionally, hyperbaric air at the bottom 876, 916 of the bottom inner cylinder 874, 914 causes rapid saturation of a high level of oxygen in the liquid. At maximum performance, air is rapidly removed; i.e., used, from the downward flow of liquid. The time of downward liquid travel is low because the net volume of the gap defined between the sidewall of the reactor 872, 912 and the sidewall of the bottom inner cylinder 874, 914 is small, thereby allowing organisms to rapidly reach the high oxygen zone within the bottom inner cylinder 874, 914.

FIGS. 21 and 22 illustrate the fermenting organism growth and separation region “C” illustrated in FIG. 19. FIG. 21 illustrates operation of the fermenter 870, 910 during times when fermenting organism concentration is being reduced. During such times, the inner air sparger 898, 934 is turned off so that no air flows from the inner air sparger 898, 934 to the interior of the cone 890, 930, and the outer air sparger 896, 904 stays on so that air flows from the outer air sparger 896, 904 about the cone 890, 930 and upwardly through the bottom inner cylinder 874, 914 as illustrated by arrows having a common pattern with arrow 960. Operation of the inner 898, 934 and outer 896, 904 air spargers in this manner results in a zone “AA” of low vertical upward velocity directly above the cone 890, 930, while normal mixing and aeration of the remainder of the fermenter 870, 910, and normal circulation up through the bottom inner cylinder 874, 914 and down through the gap between the reactor 872, 912 and the bottom inner cylinder 874, 914, is maintained, as illustrated by arrows having a common pattern with arrow 964. The zone “AA” of low vertical upward velocity allows flocculated (and some unflocculated) fermenting organism to settle into the cone 890, 930, as illustrated by arrows having a common pattern with arrow 962. Because the lower area of the cone 890, 930 and the fermenting organism exit port “BB” of the cone 890, 930 are substantially remote from turbulence, a high concentration of fermenting organism is allowed to accumulate therein for subsequent removal.

FIG. 22 illustrates operation of the fermenter 870, 910 during times when fermenting organism concentration is not being reduced. During such times, the inner air sparger 898, 934 is turned on so that air flows from the inner air sparger 898, 934 upwardly from the interior of the cone 890, 930, and the outer air sparger 896, 904 also stays on so that air flows from the outer air sparger 896, 904 about the cone 890, 930 and upwardly through the bottom inner cylinder 874, 914, all as illustrated by arrows having a common pattern with arrow 960. Normal mixing and aeration of the remainder of the fermenter 870, 910, and normal circulation of liquid up through the bottom inner cylinder 874, 914 and down through the gap between the reactor 872, 912 and the bottom inner cylinder 874, 914, is maintained, as illustrated by arrows having a common pattern with arrow 964. Operation of the inner 898, 934 and outer 896, 904 air spargers in this manner results in turbulence and admixing of flocculated and unflocculated fermenting organism inside of the cone 890, 930, precludes settling of fermenting organism in the cone 890, 930. Except for fermenting organism that has already entered the fermenting organism exit port “BB” of the cone, as illustrated by the arrow having a common pattern with arrow 962, all fermenting organism admixed by the operation of the inner air sparger 898, 934 resumes circulation through the fermenter 870, 910 as described hereinabove.

In a typical implementation of the first and second fermenters 870, 910 in the fermentation unit 580 illustrated in FIG. 19, the aspect ratio of the first fermenter 870 is much greater than that of the second fermenter 910. For example, the first fermenter 870 may have a height of approximately 60 feet and a diameter of approximately 9 feet, resulting in an aspect ratio of approximately 6.67, and the second fermenter 910 may have a height of approximately 17 feet and a diameter of approximately 12 feet, resulting in an aspect ratio of approximately 1.42. The fermenting organism collection cone 890 is therefore typically smaller in the first fermenter 870 than in the second fermenter 910. This type of configuration generally allows for high circulation velocities (low dwell time) and high oxygen-in-solution concentration in the first fermenter 870, which results in rapid fermentation of the biomaterial waste stream in the first fermenter 870. Because of the substantially lower aspect ratio, the second fermenter 910 has correspondingly lower circulation velocity (longer dwell time) and lower oxygen-in-solution concentration.

As described hereinabove, some unflocculated fermenting organism is collected along with flocculated fermenting organism in the fermenting organism exit port “BB” of the cone 890 as a result of the operation of the first fermenter 870. All of the collected fermenting organism in the first fermenter 870 is transferred to the lower portion of the cone 930 of the second fermenter 910, and the operation of the second fermenter 910, as generally described hereinabove, results in precipitation of most, if not all, of the unflocculated fermenting organism provided by the first fermenter 870, as well as growth and precipitation of additional fermenting organism. All such fermenting organism is collected in the comparatively larger cone 930 of the second fermenter 910 for subsequent removal as will be described in greater detail hereinafter.

Further details relating to the biomaterial waste stream fermentation and precipitation processes briefly described hereinabove are disclosed in detail in PCT/US2005/______, entitled FERMENTER AND FERMENTATION METHOD (attorney docket no. 35479-77851) which is assigned to the assignee of the present invention, and which is incorporated herein by reference. Further details relating to some of the structural details and to the operation of the inner and outer air spargers 898, 934 and 896, 904 respectively are disclosed in detail in PCT/US2005/______, entitled FLUID SPARGER AND DISSIPATER (attorney docket no. 35479-77856), which is assigned to the assignee of the present invention and is incorporated herein by reference.

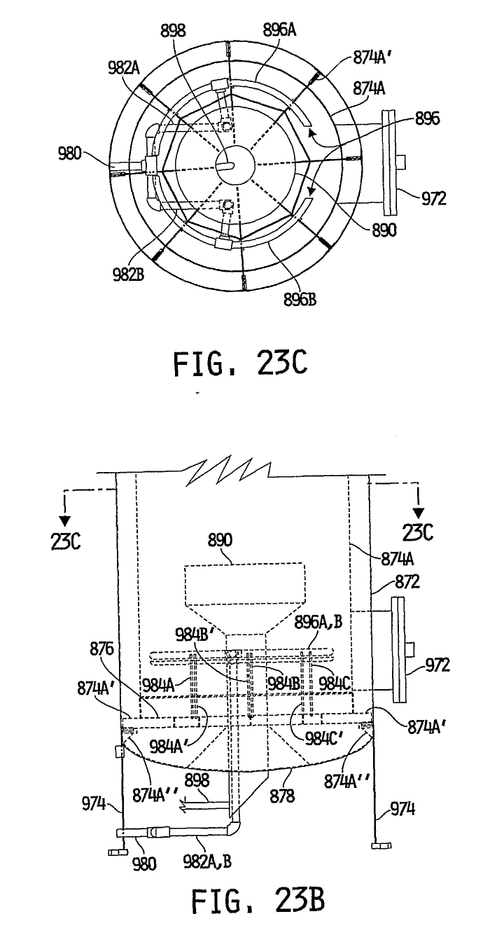

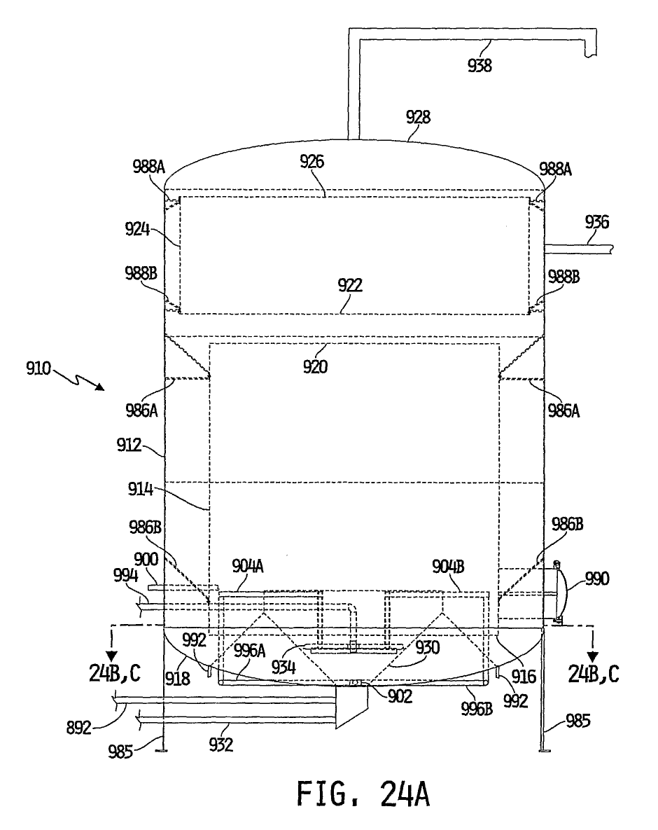

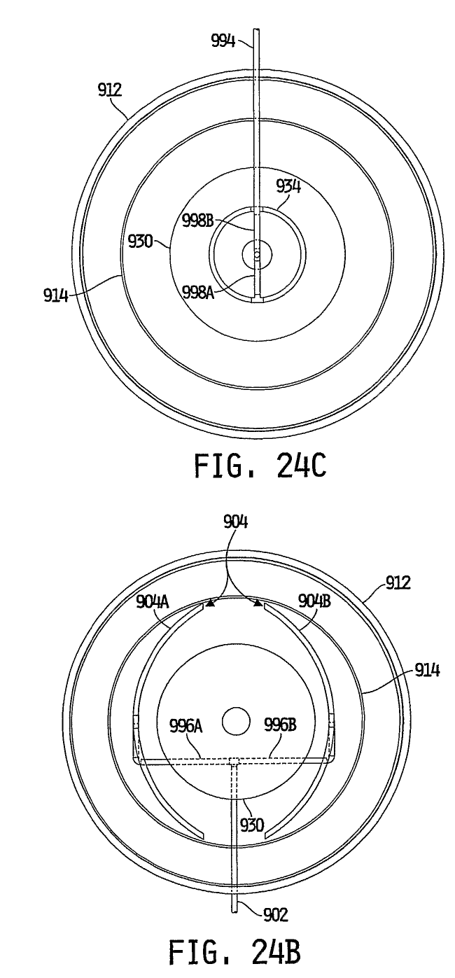

Referring now to FIGS. 23A-23C, one illustrative embodiment of the first fermentation tank 870 of FIG. 19 is shown. Referring to FIGS. 23A and 23B specifically, both of which show a front elevational view of the fermentation tank 870, the bottom inner cylinder 874 is formed of two inner cylinders 874A and 874B. The bottom end 876 of the lower bottom inner cylinder 874A is supported by a bottom support plate or grid 874A′, which is supported by one or more brackets 874A” mounted to the reactor 872 (see FIG. 23B) above the bottom 878 of the reactor 874. The upper bottom inner cylinder 874B is mounted to the reactor 872 via one or more brackets 874B′, and the lower and upper bottom inner cylinders 874A and 874B are joined together at adjacent ends via conventional joining techniques. The top inner cylinder 884 is positioned within the reactor 872 with the bottom end 882 positioned adjacent to and spaced apart from the top 880 of the upper bottom inner cylinder 874B, and the liquid outlet conduit 900 extends from the side of the reactor 872 adjacent to the top inner cylinder 884. The top end 886 of the top inner cylinder 884 is positioned adjacent to and spaced apart from the top 888 of the reactor 872, and the air outlet conduit 902 extends from the top 888 of the reactor 872. The fermenter 870 is supported in its vertical position by support legs 974, and a clean out/maintenance entrance 972 is provided through the reactor 872 and lower bottom inner cylinder 872 to allow access to the cone 890 and outer air sparger 896.

The liquid waste inlet conduit 894 extends through the reactor 872 and lower bottom inner cylinder 874A to allow the liquid biomaterial waste to enter the lower bottom inner cylinder 872 adjacent to the outer air sparger 896. A liquid drain conduit 970 extends upwardly through the bottom end 878 of the reactor 872 to provide for the draining of the fermenter 870 for maintenance or other purposes.

As most clearly shown in FIGS. 23B and 23C, the inner air sparger 898 extends through and into the lower end of the cone 890 to supply air internal to the cone 890 as described hereinabove. An outer air sparger air inlet conduit 980 extends under the bottom 878 of the reactor 872 and is split via a T-connection to air supply conduits 982A and 982B each extending laterally, then parallel via a 90° elbow toward the cone 890, then upwardly via another 90° elbow through the bottom 878 and continuing through the lower bottom inner cylinder 874A, then laterally and slightly back from the cone 890 via another 90° elbow. The air supply conduit 982A is fluidly coupled to a first outer air sparger ring 896A via another T-connection, and the air supply conduit 982B is fluidly coupled to a second outer air sparger ring 896B via yet another T-connection. The first and second outer air sparger rings 896A and 896B are each curved structures that generally follow the contour of the reactor 872 between the lower bottom inner cylinder 872 and the cone 890. The outer air sparger ring 896A is supported in its elevated position relative to the bottom end 876 of the lower bottom inner cylinder 874A by support members 984A, 984B and 984C, and the outer air sparger ring 896B is similarly supported in its elevated position by support members 984A′, 984B′ and 984C′. The outer air sparger 896 is operable, as described hereinabove, to supply air to the lower bottom inner cylinder 874A.

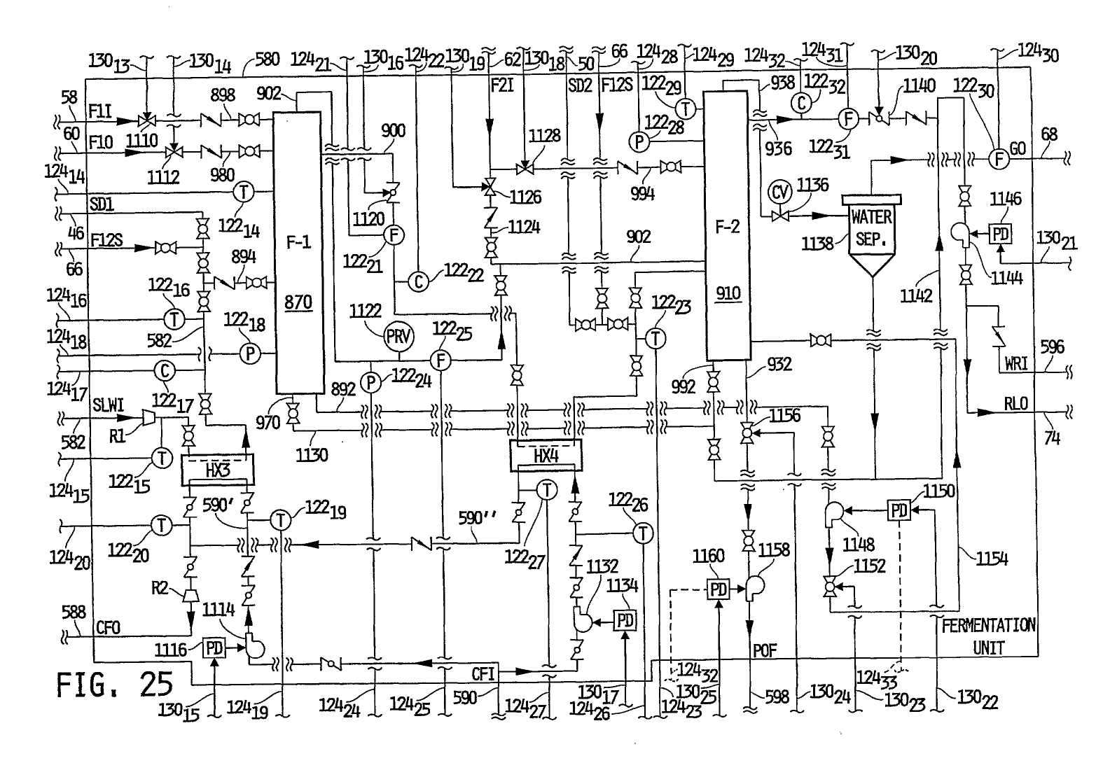

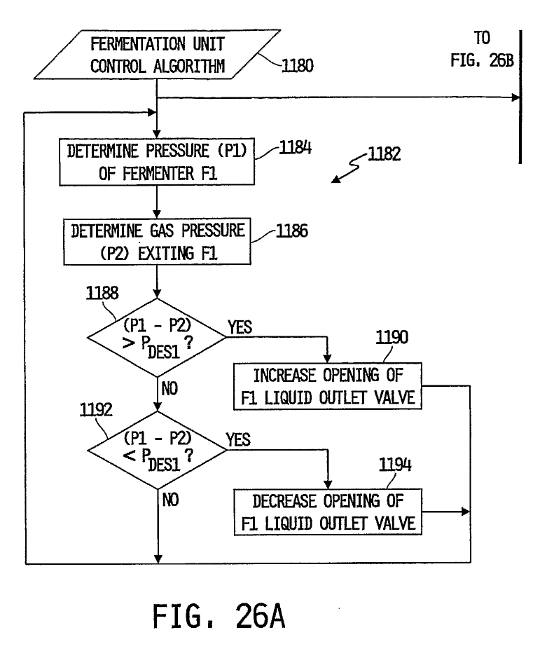

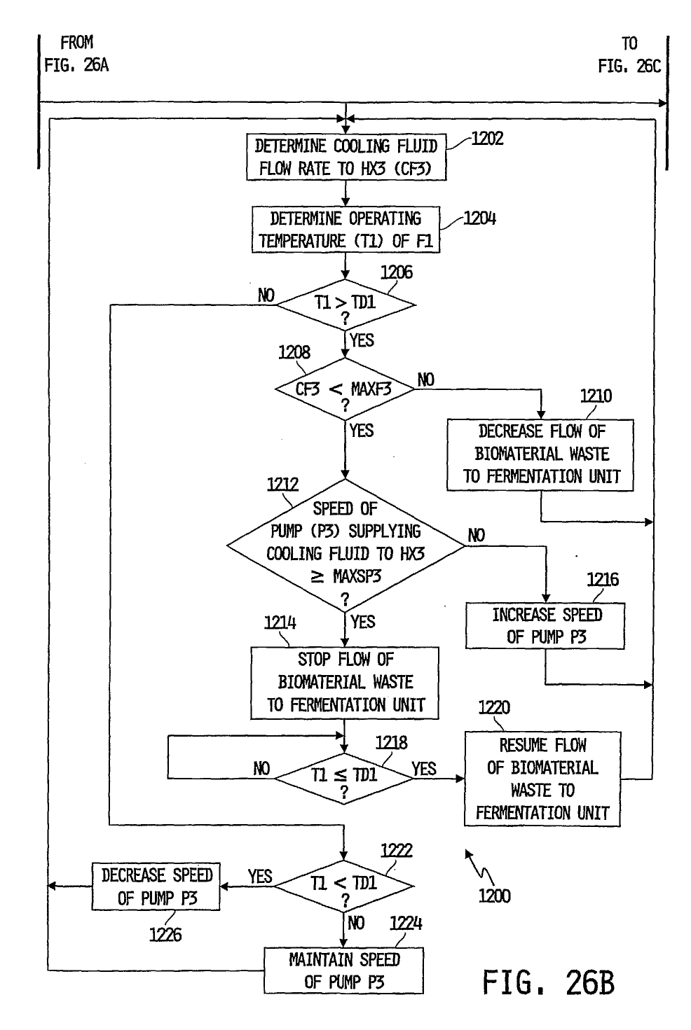

Referring now to FIGS. 24A-24C, one illustrative embodiment of the second fermentation tank 910 of FIG. 19 is shown. Referring to FIG. 24A specifically, which shows a front elevational view of the second fermentation tank 910, the upper end of the bottom inner cylinder 914 is mounted to the sidewall of the reactor 912 by one or more brackets 986A, and the lower end of the bottom inner cylinder 914 is likewise mounted to the sidewall of the reactor 912 by one or more brackets 986B. The top inner cylinder 924 is positioned within the reactor 912 with the bottom end 922 positioned adjacent to and spaced apart from the top end 920 of the bottom inner cylinder 914, and the liquid outlet conduit 936 extends from the side of the reactor 912 adjacent to the top inner cylinder 924. The top end 926 of the top inner cylinder 924 is positioned adjacent to and spaced apart from the top 928 of the reactor 912, and the air outlet conduit 938 extends from the top 928 of the reactor 912. The upper end of the top inner cylinder 914 is mounted to the sidewall of the reactor 912 by one or more brackets 988A, and the lower end of the top inner cylinder 914 is likewise mounted to the sidewall of the reactor 912 by one or more brackets 988B. The fermenter 910 is supported in its vertical position by support legs 985, and a clean out/maintenance entrance 990 is provided through the reactor 912 and lower bottom inner cylinder 912 to allow access to the cone 930 and air spargers 904 and 934.

The liquid waste inlet conduit 900 extends through the reactor 912 and bottom inner cylinder 914 to allow the liquid extracted from the first fermenter 870 to enter the bottom inner cylinder 914 adjacent to the top of the cone 930. A pair of liquid drain conduits 992 extend upwardly through the bottom end 918 of the reactor 912 to provide for the draining of the fermenter 910 for maintenance or other purposes. The product outlet conduit 892 of the first fermenter 870 is fluidly coupled to the lower portion of the cone 930 of the second fermenter 910, and the product outlet conduit 932 of the second fermenter 910 is also fluidly connected to a lower portion of the cone 930.

The outer air sparger inlet conduit 902 is fluidly connected to a pair of air conduits 996A and 996B that extend laterally via a T-connection, then upwardly in parallel and toward the cone 930 via a 90° elbow. The air conduits 996A and 996B continue through the bottom 918 of the reactor 910 and through the bottom 916 of the bottom inner cylinder 914 to a position approximately coplanar with the top of the cone 930. Via a T-connection, the air conduits 996A and 996B are in fluid communication with the outer air spargers 904A and 904B, respectively. In the illustrated embodiment, as most clearly shown in FIG. 24A, the air conduits 996A and 996B performs the dual functions of supplying inlet air to the outer air spargers 904A and 904B and mechanically supporting the outer air spargers 904A and 904B in their illustrated position. FIG. 24B shows a cross-sectional view through the second fermenter 910 that looks downwardly on the cone 930, and in FIG. 24B all details relating to the inner air sparger 934 have been omitted for clarity of illustration of the outer air sparger 904. In the illustrated embodiment, the outer air sparger 904 includes a first outer air sparger ring 904A fluidly coupled to the air conduit 996A, and a second outer air sparger ring 904B fluidly coupled to the air conduit 996B. The first and second outer air sparger rings 904A and 904B are opposing curved structures that generally follow the contour of the reactor 912 between the bottom inner cylinder 914 and the cone 930. The outer air sparger 904 is operable, as described hereinabove, to supply air to the bottom inner cylinder 914.

Referring again to FIG. 24A, an inner air sparger inlet conduit 994 extends laterally into the reactor 912 and bottom inner cylinder 914, and then extends downwardly into fluid communication with the inner air sparger 934 positioned within the cone 930. FIG. 24C shows a cross-sectional view through the second fermenter 910 that looks downwardly on the cone 930, and in FIG. 24C all details relating to the outer air sparger 934 have been omitted for clarity of illustration of the inner air sparger 934. In the illustrated embodiment, the air conduit 994 extends downwardly into the cone 930 and is fluidly connected to a pair of laterally opposing air conduits 998A and 998B by a T-connector. The pair of laterally opposing air conduits 998A and 998B are parallel to the air conduit 994. The inner air sparger 934 is provided in the form of a closed ring, and opposite ends of the air conduits 998A and 998B are fluidly connected to the inner air sparger 934 by T-connectors. The air inlet conduit 994 supports the inner air sparger 934 in its illustrated position, and the inner air sparger 934 is operable, as described hereinabove, to supply air to the interior of the cone 930.

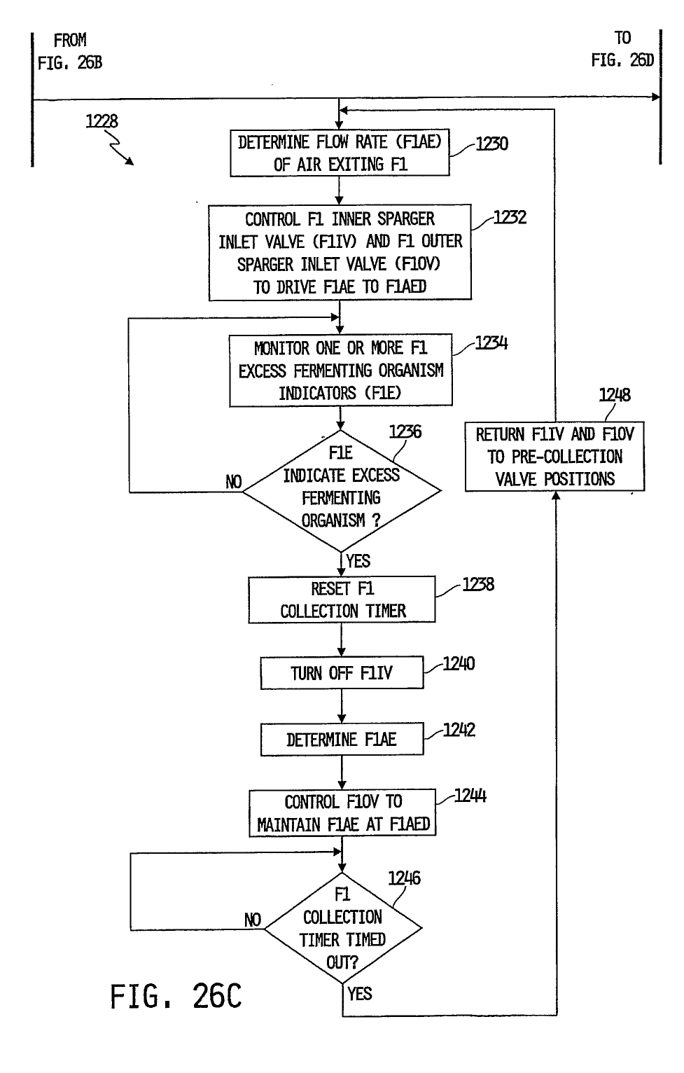

Referring now to FIG. 25, a schematic diagram of one illustrative embodiment of a control system for controlling the fermentation unit 580 of FIGS. 12 and 19 is shown. In the illustrated embodiment, the first fermenter inner air sparger inlet, F1I, is fluidly connected via conduit 58 to an inlet of an air control valve 1110 having an outlet fluidly coupled to the inner air sparger of the first fermenter 870 via the inner air sparger inlet conduit 898 having a check valve and ball valve disposed in-line therewith. The first fermenter outer air sparger inlet, F1O, is fluidly connected via conduit 60 to an inlet of another air control valve 1112 having an outlet fluidly coupled to the outer air sparger of the first fermenter 870 via the outer air sparger inlet conduit 980 having a check valve and ball valve disposed in-line therewith. The control valve 1110 represents one of the “O” actuators of the fermentation unit 580, and is electrically connected to one of the actuator outputs of the PLC circuit 120 via one of the “O” signal paths 130₁₃. The control valve 1112 represents another one of the “O” actuators of the fermentation unit 580, and is electrically connected to another one of the actuator outputs of the PLC circuit 120 via another one of the “O” signal paths 13014. The PLC circuit 120 is configured to control the operation of the air inlet valves 1110 and 1112 by producing appropriate signals on signal paths 130₁₃ and 13014 respectively. One of the “P” sensors included within the fermentation unit 580 is a conventional temperature sensor 122₁₄ disposed in fluid communication with the interior of the first fermenter 870 and electrically connected to the PLC circuit 120 via one of the “P” signal paths 12414. The temperature sensor 122₁₄ is operable to produce a temperature signal on signal path 12414 indicative of the temperature of the fluid within the first fermenter 870. Another one of the “P” sensors included within the fermentation unit 580 is a conventional pressure sensor 122₁₈ disposed in fluid communication with the interior of the first fermenter 870 and electrically connected to the PLC circuit 120 via one of the “P” signal paths 124₁₈. The pressure sensor 122₁₈ is operable to produce a pressure signal on signal path 124₁₈ indicative of the pressure within the first fermenter 870.

The sterilized liquid waste inlet, SLWI, is fluidly connected via conduit 582 through a first flow reducer, R1, through one side of a conventional heat exchanger HX3, and through a number of ball and check valves to the liquid inlet waste inlet conduit 894 of the first fermenter 870. Another one of the “P” sensors included within the fermentation unit 580 is another conventional temperature sensor 122₁₅ disposed in fluid communication with the sterilized waste inlet conduit 582 between the flow reducer, R1, and the inlet of the heat exchanger HX3, and electrically connected to the PLC circuit 120 via another one of the “P” signal paths 124₁₅. The temperature sensor 122₁₅ is operable to produce a temperature signal on signal path 124₁₅ indicative of the temperature of sterilized liquid waste entering the heat exchanger HX3. Yet another one of the “P” sensors included within the fermentation unit 580 is another conventional temperature sensor 122₁₆ disposed in fluid communication with the sterilized waste inlet conduit 582 between the outlet of the heat exchanger HX3 and liquid waste inlet conduit 894, and electrically connected to the PLC circuit 120 via another one of the “P” signal paths 124₁₆. The temperature sensor 122₁₆ is operable to produce a temperature signal on signal path 124₁₆ indicative of the temperature of sterilized liquid waste exiting the heat exchanger HX3. Still another one of the “P” sensors included within the fermentation unit 580 is a conventional conductivity sensor 122₁₇ disposed in fluid communication with the sterilized waste inlet conduit 582 between the outlet of the heat exchanger HX3 and liquid waste inlet conduit 894, and electrically connected to the PLC circuit 120 via another one of the “P” signal paths 124₁₇. The conductivity sensor 122₁₇ is operable to produce a conductivity signal on signal path 124₁₇ indicative of the electrical conductivity of sterilized liquid waste entering the first fermenter 870, and in this regard the conductivity sensor 122₁₇ may alternatively be disposed in fluid communication with the sterilized liquid waste stream anywhere along conduit 582 or conduit 894.

The first seed inlet, SD1, is fluidly connected via conduit 46 and through a pair of ball valves to the junction of conduits 582 and 894. The seed steam inlet, F12S, is fluidly connected through another ball valve to the seed inlet conduit 46 between the two ball valves in-line therewith. Organisms may be added to the first fermenter 870 via the SD1 inlet, and this inlet may be cleaned/sterilized via the steam inlet, F12S, via appropriate manual control of the various ball valves.

The coolant flow inlet, CFI, of the fermentation unit 580 is fluidly coupled via conduit 590 and butterfly valve to an inlet of a coolant fluid pump 1114 having a pump outlet fluidly coupled to a conduit 590′ that passes through a number of butterfly and check valves, and through the opposite side of the heat exchanger HX3 to the inlet of a flow expander, R2. The outlet of the flow expander, R2, is fluidly coupled to the coolant flow outlet, CFO, of the fermentation unit 580 via conduit 588. The pump 1114 is electrically connected to a conventional pump driver 1116, which also is electrically connected to one of the actuator outputs of the PLC circuit 120 via signal path 130₁₅. The pump driver 1116 represents another one of the “O” actuators, and the PLC circuit 120 is configured to control operation of the pump 1114, by producing an appropriate control signal on signal path 130₁₅, to thereby control the temperature of the sterilized liquid waste stream entering the first fermenter 870 by controlling the flow rate of coolant fluid through the heat exchanger HX3. Another one of the “P” sensors included within the fermentation unit 580 is another conventional temperature sensor 122₁₉ disposed in fluid communication with conduit 590′ between the outlet of the pump 1114 and the coolant fluid inlet of the heat exchanger HX3, and electrically connected to the PLC circuit 120 via another one of the “P” signal paths 124₁₉. The temperature sensor 122₁₉ is operable to produce a temperature signal on signal path 124₁₉ indicative of the temperature of the coolant fluid entering the heat exchanger HX3. Yet another one of the “P” sensors included within the fermentation unit 580 is another conventional temperature sensor 122₂₀ disposed in fluid communication with the conduit 590′ between the coolant fluid outlet of the heat exchanger HX3 and the flow expander, R2, and electrically connected to the PLC circuit 120 via another one of the “P” signal paths 124₂₀. The temperature sensor 122₂₀ is operable to produce a temperature signal on signal path 124₂₀ indicative of the temperature of coolant fluid exiting the heat exchanger HX3.

The liquid outlet conduit 900 extending from the liquid outlet of the first fermenter 870 is coupled through a control valve 1120, through one side of another conventional heat exchanger HX4, and through various ball valves to the liquid inlet of the second fermenter 910. The control valve 1120 represents another one of the “O” actuators of the fermentation unit 580, and is electrically connected to another one of the actuator outputs of the PLC circuit 120 via another one of the “O” signal paths 130₁₆. The PLC circuit 120 is configured to control the operation of the control valve 1120 by producing an appropriate signal on signal path 130₁₆. Another one of the “P” sensors included within the fermentation unit 580 is a conventional flow sensor or flow meter 122₂₁ disposed in fluid communication with the liquid outlet conduit 900 between the inlet control valve 1120 and the inlet of the heat exchanger HX4, and electrically connected to the PLC circuit 120 via one of the “P” signal paths 124₂₁. The flow sensor or flow meter 122₂₁ is operable to produce a signal on signal path 124₂₁ indicative of the flow rate of liquid flowing out of the first fermenter 870 and into the second fermenter 910, and as such may be alternatively positioned anywhere along conduit 900. Another one of the “P” sensors included within the fermentation unit 580 is another conventional conductivity sensor 122₂₂ disposed in fluid communication with the liquid outlet conduit 900 between the flow sensor 122₂₂ and the inlet of the heat exchanger HX4, and electrically connected to the PLC circuit 120 via one of the “P” signal paths 124₂₂. The conductivity sensor 122₂₁ is operable to produce a signal on signal path 124₂₂ indicative of the electrical conductivity of the liquid flowing out of the first fermenter 870, and as such may be alternatively positioned anywhere along conduit 900. Yet another one of the “P” sensors included within the fermentation unit 580 is another conventional temperature sensor 122₂₃ disposed in fluid communication with the liquid outlet conduit 900 between the outlet of the heat exchanger HX4 and the second fermenter 910, and electrically connected to the PLC circuit 120 via one of the “P” signal paths 124₂₃. The temperature sensor 122₂₃ is operable to produce a signal on signal path 124₂₃ indicative of the temperature of the liquid exiting the heat exchanger HX4.

The second seed inlet, SD2, is fluidly connected via conduit 50 and through a pair of ball valves to conduit 900. The seed steam inlet, F12S, is fluidly connected through another ball valve to the seed inlet conduit 50 between the two ball valves in-line therewith. Organisms may be added to the second fermenter 910 via the SD2 inlet, and this inlet may be cleaned/sterilized via the steam inlet, F12S, via appropriate manual control of the various ball valves.

The coolant flow inlet, CFI, of the fermentation unit 580 is also fluidly coupled via conduit 590 and butterfly valve to an inlet of another coolant fluid pump 1132 having a pump outlet fluidly coupled to a conduit 590″ that passes through a number of butterfly and check valves, through the opposite side of the heat exchanger HX4, and into fluid communication with the conduit 590′ between the heat exchanger HX3 and flow expander, R2, and downstream of the temperature sensor 122₁₅. The pump 1132 is electrically connected to another conventional pump driver 1134, which also is electrically connected to one of the actuator outputs of the PLC circuit 120 via signal path 130₁₇. The pump driver 1134 represents another one of the “O” actuators, and the PLC circuit 120 is configured to control operation of the pump 1132, by producing an appropriate control signal on signal path 130₁₇, to thereby control the temperature of the liquid stream entering the second fermenter 910 by controlling the flow rate of coolant fluid through the heat exchanger HX4. Another one of the “P” sensors included within the fermentation unit 580 is another conventional temperature sensor 122₂₆ disposed in fluid communication with conduit 590″ between the outlet of the pump 1132 and the coolant fluid inlet of the heat exchanger HX4, and electrically connected to the PLC circuit 120 via another one of the “P” signal paths 124₂₆. The temperature sensor 122₂₆ is operable to produce a temperature signal on signal path 124₂₆ indicative of the temperature of the coolant fluid entering the heat exchanger HX4. Yet another one of the “P” sensors included within the fermentation unit 580 is another conventional temperature sensor 122₂₇ disposed in fluid communication with the conduit 590″ between the coolant fluid outlet of the heat exchanger HX4 and conduit 590′, and electrically connected to the PLC circuit 120 via another one of the “P” signal paths 124₂₇. The temperature sensor 122₂₇ is operable to produce a temperature signal on signal path 124₂₇ indicative of the temperature of coolant fluid exiting the heat exchanger HX4.

The air outlet conduit 902 extending from the air outlet of the first fermenter 870 is coupled through a ball valve to the outer air sparger inlet of the second fermenter 910. Another one of the “P” sensors included within the fermentation unit 580 is another conventional pressure sensor 122₂₄ disposed in fluid communication with conduit 902 and electrically connected to the PLC circuit 120 via another one of the “P” signal paths 124₂₄. The pressure sensor 122₂₄ is operable to produce a pressure signal on signal path 124₂₄ indicative of the pressure of gas exiting the first fermenter 870 and entering the outer air sparger inlet of the second fermenter 910. Yet another one of the “P” sensors included within the fermentation unit 580 is a conventional mass flow sensor or mass flow meter 122₂₅ disposed in-line with conduit and electrically connected to the PLC circuit 120 via another one of the “P” signal paths 124₂₅. The mass flow sensor or mass flow meter 122₂₅ is operable to produce a signal on signal path 124₂₅ indicative of the mass flow rate of gas exiting the first fermenter 870 and entering the out air sparger inlet of the second fermenter 910. A pressure relief valve 1122 is also disposed in fluid communication with conduit 902. The pressure relief valve 1122 is a mechanical valve having an opening pressure that is set to prevent over-pressure and/or vacuum conditions within conduit 902.

The second fermenter inner air sparger inlet, F2I, is fluidly connected via conduit 62 to an inlet of an air control valve 1128 having an outlet fluidly coupled via a check valve and ball valve to the inner air sparger inlet conduit 994 of the second fermenter 910. The control valve 1128 represents another one of the “O” actuators of the fermentation unit 580, and is electrically connected to one of the actuator outputs of the PLC circuit 120 via one of the “O” signal paths 130₁₈. The PLC circuit 120 is configured to control the operation of the air inlet valve 1128 by producing an appropriate signal on signal path 130₁₈. The second fermenter inner air sparger inlet, F2I, is also coupled via conduit 62 to an inlet of another air control valve 1126 having an outlet fluidly coupled via a check valve and a ball valve to the air outlet conduit 902. The control valve 1126 represents yet another one of the “O” actuators of the fermentation unit 580, and is electrically connected to another one of the actuator outputs of the PLC circuit 120 via another one of the “O” signal paths 130₁₉. The PLC circuit 120 is configured to control the operation of the air inlet valve 1126 by producing an appropriate signal on signal path 130₁₉ to selectively supplement air provided to the outer air sparger of the second fermenter 910. Another one of the “P” sensors included within the fermentation unit 580 is a conventional pressure sensor 122₂₈ disposed in fluid communication with the interior of the second fermenter 910 and electrically connected to the PLC circuit 120 via another one of the “P” signal paths 124₂₈. The pressure sensor 122₂₈ is operable to produce a pressure signal on signal path 124₂₈ indicative of the pressure within the second fermenter 870. Another one of the “P” sensors included within the fermentation unit 580 is another conventional temperature sensor 122₂₉ disposed in fluid communication with the interior of the second fermenter 910 and electrically connected to the PLC circuit 120 via one of the “P” signal paths 124₂₉. The temperature sensor 122₂₉ is operable to produce a temperature signal on signal path 124₂₉ indicative of the temperature of the fluid within the second fermenter 910.

The product outlet conduit 892 fluidly connected to the outlet of the cone 890 of the first fermenter 870 is fluidly connected through a ball valve to an inlet of a product outlet pump 1148 having a pump outlet fluidly coupled to an inlet of a control valve 1152. An outlet of the control valve 1152 is fluidly coupled through another ball valve to the product inlet of the second fermenter 910 via conduit 1154. The pump 1148 is electrically connected to a conventional pump driver 1150 that is also electrically connected to an actuator output of the PLC circuit 120 via signal path 130₂₂. In some embodiments, the pump driver 1150 may also be electrically connected to a sensor input of the PLC circuit 120 via signal path 124₃₃ as shown in phantom in FIG. 25. The PLC circuit 120 is configured to control the speed of the pump 1148 in a known manner by producing an appropriate actuator control signal on signal path 130₂₂. The pump driver 1150 is responsive to the actuator control signal supplied by the PLC 120 on signal path 130₂₂ to drive the pump 1148. In the illustrated embodiment, the pump driver 1150 and/or pump 1148 further includes a “sensor” for determining and monitoring the operating torque of the pump 1148. Such a “sensor” may be a conventional strain-gauge type torque sensor operatively coupled to a rotating drive shaft of the pump 1148 and operable to produce a sensor signal corresponding to the operating torque of the pump 1148, or may alternatively be a so-called virtual sensor implemented in the form of one or more software algorithms resident within the PLC circuit 120 and responsive to one or more measurable operating parameters associated with the pump driver 1150 and/or pump 1148 to derive or infer the operating torque value. For example, the pump driver 1150 may include a current sensor producing a current sensor signal indicative of drive current being drawn by the pump driver 1148, and/or the pump 1150 may include a position and/or speed sensor producing a signal corresponding to the rotational speed and/or position of the pump 1148. The PLC circuit 120 may be responsive to any such sensor signals, and/or to other information relating to the operation of the pump driver 1150 and/or pump 1148, to estimate the operating torque of the pump 1148 as a known function thereof. In any case, the signal path 124₃₃ carries one or more torque feedback signals to the PLC circuit 120 from which the operating torque of the pump 1148 may be determined directly or estimated. The control valve 1152 is likewise electrically connected to another one of the actuator outputs of the PLC circuit 120 via signal path 130₂₃. The pump driver 1150 and control valve 1152 represent additional ones of the “O” actuators, and the PLC circuit 120 is configured to control operation of the pump 1150 and the control valve 1152, by producing appropriate control signals on signal paths 130₂₂ and 130₂₃ respectively, to control the timing and flow of fermenting organism from the first fermenter 870 to the second fermenter 910.

The drain outlet 970 of the first fermenter 780 is fluidly coupled through a ball valve to one end of a conduit 1130 having an opposite end fluidly coupled to a liquid outlet conduit 1142. The drain outlet 992 of the second fermenter 910 is fluidly coupled through another ball valve to the junction of conduits 1130 and 1142. The liquid outlet conduit 1142 is fluidly coupled through a pair of ball valves to an inlet of a liquid outlet pump 1144 having a pump outlet fluidly coupled through a ball valve to the residual liquid outlet, RLO, of the fermentation unit 580 and to the residual liquid outlet conduit 74. The waste return inlet, WRI, of the fermentation unit 580 that is fluidly coupled to conduit 596 is also fluidly coupled through a check valve to the residual liquid outlet, RLO. The liquid outlet conduit 936 that is fluidly coupled to the liquid outlet of the second fermenter 910 is coupled through a liquid outlet control valve 1140 and check valve to the liquid outlet conduit 1142. The liquid outlet pump 1144 is electrically connected to another conventional pump driver 1146, which also is electrically connected to another one of the actuator outputs of the PLC circuit 120 via signal path 130₂₁. The control valve 1140 is likewise electrically connected to another one of the actuator outputs of the PLC circuit 120 via signal path 130₂₀. The pump driver 1146 and control valve 1140 represent additional ones of the “O” actuators, and the PLC circuit 120 is configured to control operation of the pump 1144 and the control valve 1140, by producing appropriate control signals on signal paths 130₂₁ and 130₂₀ respectively, to control the timing and flow of liquid out of the second fermenter 910. Additionally, and independently of control valve 1140, the liquid outlet pump 1144 may be controlled by the PLC circuit 120 to drain liquid from the first and/or second fermenter 870, 910, via drain outlets 970 and 992 respectively, at a desired flow rate.

Another one of the “P” sensors included within the fermentation unit 580 is a conventional flow sensor or flow meter 122₃₁ disposed in fluid communication with the liquid outlet conduit 932 upstream of the control valve 1140, and electrically connected to the PLC circuit 120 via another one of the “P” signal paths 124₂₁. The flow sensor or flow meter 122₃₁ is operable to produce a signal on signal path 124₃₁ indicative of the flow rate of liquid flowing out of the second fermenter 910 via the liquid outlet conduit 936, and as such may be alternatively positioned anywhere along conduit 936. Yet another one of the “P” sensors included within the fermentation unit 580 is another conventional conductivity sensor 122₃₂ disposed in fluid communication with the liquid outlet conduit 936 upstream of the flow sensor or flow meter 122₃₁ and electrically connected to the PLC circuit 120 via another one of the “P” signal paths 124₃₂. The conductivity sensor 122₃₂ is operable to produce a signal on signal path 124₃₂ indicative of the conductivity of the liquid flowing out of the second fermenter 910 via conduit 936, and as such may be alternatively positioned anywhere along conduit 936.

The air outlet conduit 938 extending from the air outlet of the second fermenter 910 is coupled through a mechanical control valve 1136 to an inlet of a conventional water separation unit 1138. A water drain outlet of the water separation unit 1138 is fluidly coupled to the liquid outlet conduit 1142, and the air outlet of the water separation unit is fluidly coupled to the gas outlet, GO, of the fermentation unit 580 and also fluidly coupled to conduit 68. Optionally, a control valve (not shown) may be interposed between the water drain outlet of the water separation unit 1138 and the liquid outlet conduit 1142, which would be electrically controlled by the PLC circuit 120. In this embodiment, the water separation unit 1142 may thus be drained under the control of the PLC circuit 120. The control valve 1136 is a mechanical pressure control valve having a manually selectable set pressure value. The control valve 1136 is operable in a known manner to maintain the set air pressure within conduit 938. The water separation unit is operable in a known manner to condense water from the gas exiting the second fermenter 910 via conduit 938, and to direct the condensed water to the liquid outlet conduit 1142 while directing the remaining gas to the gas outlet, GO, of the fermentation unit 580. Another one of the “P” sensors included within the fermentation unit 580 is another conventional mass flow sensor or mass flow meter 122₃₀ disposed in fluid communication with the gas outlet, GO, and electrically connected to the PLC circuit 120 via another one of the “P” signal paths 124₃₀. The mass flow sensor or mass flow meter 122₃₀ is operable to produce a signal on signal path 124₃₀ indicative of the mass flow rate of gas exiting the second fermenter 910, and as such may be alternatively positioned anywhere along the air outlet conduit 936.

The product outlet conduit 932 fluidly coupled at one end to the outlet of the cone 930 of the second fermenter 910 is fluidly coupled at its opposite end through a control valve 1156 and a ball valve to the inlet of-a product outlet pump 1158 having a pump outlet fluidly coupled to the product outlet, POF, of the fermentation unit 580 and also to the conduit 598. The pump 1158 is electrically connected to another conventional pump driver 1160 that is also electrically connected to an actuator output of the PLC circuit 120 via signal path 130₂₅. In some embodiments, the pump driver 1160 may also be electrically connected to a sensor input of the PLC circuit 120 via signal path 124₃₂ as shown in phantom in FIG. 25. The PLC circuit 120 is configured to control the speed of the pump 1158 in a known manner by producing an appropriate actuator control signal on signal path 130₂₅. The pump driver 1160 is responsive to the actuator control signal supplied by the PLC 120 on signal path 130₂₅ to drive the pump 1158. In the illustrated embodiment, the pump driver 1160 and/or pump 1158 further includes a “sensor” for determining and monitoring the operating torque of the pump 1158, wherein such a “sensor” may be as described hereinabove with respect to the description of the pump driver 1150. The PLC circuit 120 may be responsive to any such sensor signals, and/or to other information relating to the operation of the pump driver 1160 and/or pump 1158, to estimate the operating torque of the pump 1158 as a known function thereof. In any case, the signal path 124₃₂ carries one or more torque feedback signals to the PLC circuit 120 from which the operating torque of the pump 1158 may be determined directly or estimated. The control valve 1156 is likewise electrically connected to another one of the actuator outputs of the PLC circuit 120 via signal path 130₂₄. The pump driver 1160 and control valve 1156 represent additional ones of the “O” actuators, and the PLC circuit 120 is configured to control operation of the pump 1158 and the control valve 1156, by producing appropriate control signals on signal paths 130₂₅ and 130₂₄ respectively, to control the timing and flow of fermenting organism from the second fermenter 870 to the pasteurization unit 595.

The fermentation unit 580 just described includes a number of manually actuated butterfly valves, ball valves and check valves as illustrated in FIG. 17. The ball valves and butterfly valves are included within the fermentation unit 580 at various locations to allow for bypassing of, and maintenance or replacement of, various components of the fermentation unit 580, and some are also used in relation to pre-start sterilization, cleaning and seeding operations. The check valves, on the other hand, are provided at various locations within the fermentation unit to ensure unidirectional flow therethrough of gas and/or liquid.

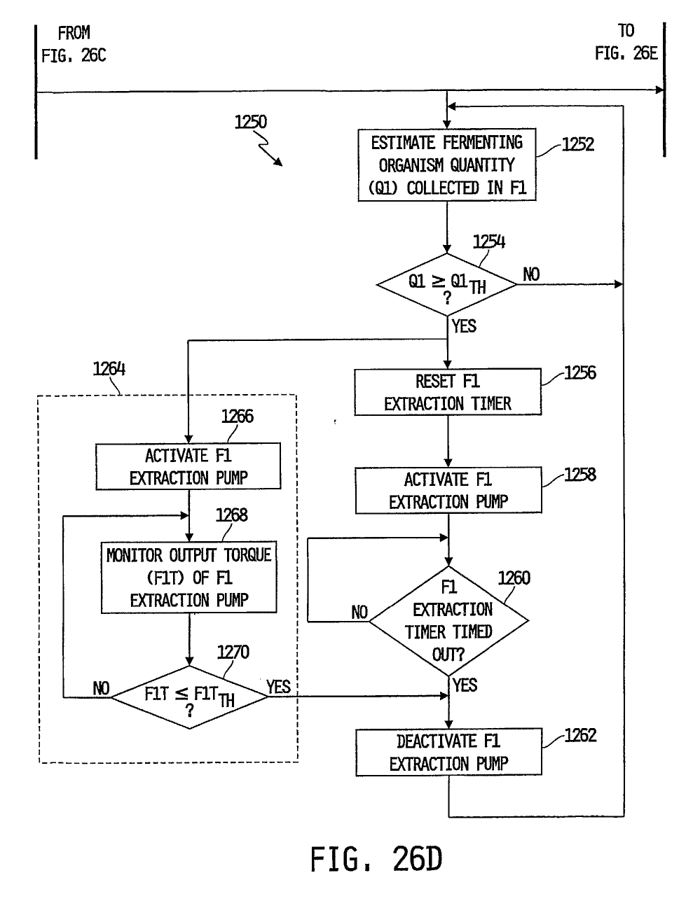

Referring now to FIGS. 26A-26H, a flowchart of one illustrative embodiment of a software control algorithm 1180 for controlling a fermentation unit of the type illustrated in FIGS. 12 and 19-24C via the control system of FIG. 25. It will be understood that the software control algorithm 1180 represents one illustrative strategy for controlling the fermentation unit 580 during normal, continuous flow operation of the biomaterial waste processing system 10, and that the fermentation unit 580 may be controlled differently during other operational modes of the biomaterial waste processing system 10. The software algorithm 1180 includes a number of different and independently executing control routines, and each of these different control routines will be described separately. For example, as illustrated in FIG. 26A, the control algorithm 1180 includes a first control routine 1182 for controlling the liquid level within the first fermenter 870. The control routine 1182 begins at step 1184 where the PLC circuit 120 is operable to determine the operating pressure, P1, of the first fermenter 870 by monitoring the pressure signal produced by the pressure sensor 122₁₈ on signal path 124₁₈. Thereafter at step 1186, the PLC circuit 120 is operable to determine the pressure, P2, of gas exiting the first fermenter 870 by monitoring the pressure signal produced by the pressure sensor 122₂₄ on signal path 124₂₄. Following step 1186, the PLC circuit 120 is operable at step 1188 to compare the difference between P1 and P2 to a design pressure, P_{DES1}, where P_{DES1} corresponds to a pressure equivalent of the desired liquid level within the first fermenter 870.

If, at step 1188, the PLC circuit 120 determines that (P1−P2) is greater than P_{DES1}, indicating that the liquid level within the first fermenter 870 is higher than desired, the PLC circuit 120 is operable thereafter at step 1190 to increase the opening of the liquid outlet valve 1120, by producing an appropriate actuator control signal on signal path 130₁₆, to increase the flow of liquid exiting the first fermenter 870. If, on the other hand, the PLC circuit 120 determines at step 1188 that (P1−P2) is not greater than P_{DES1}, execution of the control routine 1182 advances to step 1192 where the PLC circuit is again operable to compare the difference between P1 and P2 to the design pressure, P_{DES1}. If, at step 1192, the PLC circuit determines that (P1−P2) is less than P_{DES1}, indicating that the liquid level within the first fermenter 870 is lower than desired, the PLC circuit 120 is operable thereafter at step 1194 to decrease the opening of the liquid outlet valve 1120, by producing an appropriate actuator control signal on signal path 130₁₆, to decrease the flow of liquid exiting the first fermenter 870. If, on the other hand, the PLC circuit 120 determines at step 1192 that (P1−P2) is not less than P_{DES1}, execution of the control routine 1182 loops back to step 1184 as it also does following execution of steps 1190 and 1194.

The fermentation unit control algorithm 1180 further includes another control routine 1200, as illustrated in FIG. 26B, for controlling the operating temperature of the first fermenter 870 by controlling the temperature of the sterilized liquid waste entering the first fermenter 870 via control of coolant fluid flow through the heat exchanger HX3. The control routine 1200 begins at step 1202 where the PLC circuit 120 is operable to determine the flow rate of coolant fluid, CF3, from the cooling tower unit 586 to the heat exchanger HX3. In the illustrated embodiment, the PLC 120 is operable to execute step 1202 by computing CF3 as a function of the flow rate of the biomaterial waste entering the fermentation unit 580 via the sterilized liquid waste inlet conduit 582, the temperature difference between the biomaterial waste entering and exiting HX3 and the temperature difference between the cooling fluid entering and exiting HX3. In particular, the PLC 120 is operable at step 1202 to compute CF3 according to the equation CF3=F122₃*(T122₁₅31 T122₁₆)/(T122₁₉−T122₂₀), where F122₃ is the biomaterial waste flow rate signal produced by the flow sensor 122₃ comprising part of the sterilization unit 570 as illustrated in FIG. 13A, T122₁₅ is the temperature signal produced by the temperature sensor 122₁₅ on signal path 124₁₅ and represents the temperature of the biomaterial waste entering HX3, T122₁₆ is the temperature signal produced by the temperature sensor 122₁₆ on signal path 124₁₆ and represents the temperature of the biomaterial waste exiting HX3, T122₁₉ is the temperature signal produced by the temperature sensor 122₁₉ on signal path 124₁₉ and represents the temperature of the cooling fluid entering HX3 from the cooling tower unit 586, and T122₂₀ is the temperature signal produced by the temperature sensor 122₂₀ on signal path 124₂₀ and represents the temperature of the cooling fluid exiting HX3. Alternatively, the coolant flow path through HX3 may include a flow meter or sensor, and in this embodiment the PLC 120 may be operable to execute step 1202 by monitoring the flow signal produced by such a flow meter or sensor. In any case, the execution of routine 1200 advances from step 1202 to step 1204 where the PLC circuit 120 is operable to determine the operating temperature, T1, of the first fermenter 870 by monitoring the temperature signal produced by the temperature sensor 122₁₄ on signal path 124₁₄. Thereafter at step 1206, the PLC circuit 120 is operable to compare the temperature, T1, of the first fermenter 870 to a design temperature, T_{D1}, wherein T_{D1} corresponds to a desired operating or fermenting temperature of the first fermenter 870.

If, at step 1206, the PLC circuit 120 determines that T1 is greater than T_{D1}, indicating that the operating temperature of the first fermenter 870 is greater than the design temperature, T_{D1}, execution of the control routine 1200 advances to step 1208 where the PLC circuit 120 is operable to compare the cooling fluid flow rate, CF3, through HX3 to a maximum flow rate value, MAXF3, wherein MAXF3 corresponds to a desired maximum flow rate of cooling fluid from the cooling tower unit 586. If, at step 1208, the PLC circuit 120 determines that CF3 is greater than or equal to MAXF3, routine execution advances to step 1210 where the PLC circuit 120 is operable to decrease the flow of biomaterial waste to the fermentation unit 580. In one embodiment, the PLC circuit 120 is operable to execute step 1210 by decreasing the speed of the biomaterial waste pump 612 forming part of the sterilization unit 570 as illustrated in FIG. 13A. Alternatively or additionally, the PLC circuit 120 may be operable to execute step 1210 by controlling the diverter valve 638 of the sterilization unit 570 to divert at least some of the biomaterial waste stream exiting the sterilization loop 630 back through the sterilization unit 570 to thereby decrease the flow rate of biomaterial waste exiting the sterilization unit 570. Alternatively or additionally still, the PLC circuit 120 maybe operable to execute step 1210 by controlling the biomaterial waste return valve 622 to return at least some of the biomaterial waste stream flowing through the sterilization unit 570 back to the biomaterial waste source 20 (FIG. 1) to thereby decrease the flow rate of biomaterial waste exiting the sterilization unit 570. In any case, execution of the routine 1200 loops from step 1210 back to step 1202.

If, at step 1208, the PLC circuit 120 determines that the CF3 is less than MAXF3, routine execution advances to step 1212 where the PLC circuit 120 is operable to compare the speed of the pump 1114 (P3) supplying the cooling fluid from the cooling tower unit 586 to HX3 to a maximum pump speed, MAXSP3, wherein MAXSP3 corresponds to a maximum pump speed value that may be arbitrary or may be dictated by the physical properties of the pump 1114. In either case, if the PLC circuit 120 determines at step 1212 that the speed of the pump P3 is greater than or equal to MAXSP3, routine execution advances to step 1214 where the PLC circuit 120 is operable to stop the flow of biomaterial waste to the fermentation unit 580. In one embodiment, the PLC circuit 120 is operable to execute step 1214 by deactivating the biomaterial waste pump 612 forming part of the sterilization unit 570 as illustrated in FIG. 13A. Alternatively or additionally, the PLC circuit 120 may be operable to execute step 1214 by controlling the diverter valve 638 of the sterilization unit 570 to divert the biomaterial waste stream exiting the sterilization loop 630 back through the sterilization unit 570 to thereby stop the flow rate of biomaterial waste exiting the sterilization unit 570. Alternatively or additionally still, the PLC circuit 120 may be operable to execute step 1210 by controlling the biomaterial waste return valve 622 to return the biomaterial waste stream flowing through the sterilization unit 570 back to the biomaterial waste source 20 (FIG. 1) to thereby stop the flow rate of biomaterial waste exiting the sterilization unit 570. In any case, execution of the routine 1200 advances from step 1214 to step 1218 where the PLC circuit 120 is operable to pause until the temperature, T1, of the fermenter 870 is less than or equal to the design temperature, TD1. Thereafter, routine execution advances to step 1220 where the control circuit is operable to control the flow of the biomaterial waste stream entering the fermentation unit 580, using any of the techniques just described, to resume the flow of biomaterial waste into the fermentation unit 580. Thereafter, execution of the routine 1200 loops back to step 1202. If, at step 1212, the PLC circuit 120 determines that the speed of the cooling fluid pump P3 is less than MAXSP3, routine execution advances to step 1216 where the PLC circuit 120 is operable to increase the speed of the pump P3 by appropriately controlling the pump driver 1116. Thereafter, routine execution loops back to step 1202.

If, at step 1206, the PLC circuit 120 determines that the temperature, T1, of the fermenter 870 is greater than the design temperature, TD1, routine execution advances to step 1222 where the PLC circuit 120 is operable to determine whether T1 is less than TD1. If not, then T1=TD1 and routine execution advances to step 1224 where the PLC circuit 120 is operable to maintain the current speed of the pump P3 by appropriately controlling the pump driver 1116. If, at step 1222, the PLC circuit 120 determines that T1 is less than TD1, routine execution advances to step 1226 where the PLC circuit 120 is operable to decrease the speed of the pump P3 by appropriately controlling the pump driver 1116. Routine execution loops from either of steps 1224 and 1226 back to step 1202.

The PLC circuit 120 is operable, under the direction of the routine 1200, to control the temperature, T1, of the first fermenter 870 by comparing T1 to a design temperature, TD1, and increasing the speed of the pump 1114 (P3) supplying cooling fluid from the cooling tower unit 586 to the heat exchanger HX3 if T1 is greater than TD1, the flow rate of the cooling fluid through HX3 is less than a maximum cooling fluid flow rate, MAXF3, and the speed of the pump 1114 is not greater than or equal to a maximum pump speed, MAXSP3. If, however, T1 is greater than TD1 and the flow rate of the cooling fluid through HX3 is greater than or equal to MAXF3, then T1 cannot be lowered by increasing the speed of the pump 1114, and the flow rate of the biomaterial waste into the fermentation unit 580 is instead decreased. If T1 is greater than TD1 and the flow rate of the cooling fluid through HX3 is less than MAXF3 but the speed of the pump 1114 is greater than or equal to the maximum pump speed, MAXSP3, then no action of the cooling fluid pump 1114 will result in further cooling of the biomaterial waste stream, and in this case the flow of biomaterial waste into the fermentation unit 580 is stopped until T1 becomes less than or equal to TD1. If T1 is less than TD1, the speed of the pump 1114 is decreased, and if T1 is equal to TD1 the speed of the pump 1114 is maintained at its current pump speed.

The fermentation unit control algorithm 1180 further includes another control routine 1228, as illustrated in FIG. 26C, for controlling collection of the fermenting organism, e.g., yeast or other fermenting organism, within the lower portion of the cone 890 of the first fermenter 870 as illustrated in FIGS. 19 and 21-23C. It will be understood that the control routine 1228 represents one embodiment of a control routine for controlling fermenting organism collection within the fermenter 870 during normal, continuous flow operation, and that air flow into the outer and inner air spargers 896 and 898 respectively of the fermenter 870 will typically be established and controlled prior to normal, continuous flow operation by the PLC circuit 120. Prior to fermentation, e.g., prior to the normal, continuous flow operation of the first fermenter 870, the PLC circuit 120 is operable to determine a baseline exit gas mass flow rate as a known function of measured exit gas mass flow rate, e.g., from the mass flow rate signal produced by the mass flow sensor or meter 122₅, ambient air temperature, e.g., from the ambient air temperature signal produced by the ambient temperature sensor 122₁₂ associated with the cooling tower unit 586 (see FIG. 17), and relative humidity, e.g., from the relative humidity signal produced by the relative humidity sensor 122₁₃ forming part of the cooling tower unit 586, wherein the ambient temperature and relative humidity information are used to estimate or otherwise calculate a dew point value using known relationships therebetween. The control routine 1228 begins at step 1230 where the PLC circuit 120 is operable to determine the exit gas mass flow rate, e.g., mass flow rate of air exiting the fermenter 870, during the normal, continuous flow operating mode, e.g., during fermentation, by monitoring the mass flow signal produced by the mass flow meter or sensor 122₂₅ illustrated in FIG. 25. Thereafter at step 1232, the PLC circuit 120 is operable to control the inner sparger inlet valve 1110 and outer sparger inlet valve 1112 respectively of the fermenter 870 to drive the flow rate of air exiting the fermenter 870 to a design air flow exit value, F1AED, wherein F1AED represents a target air flow value that will depend, at least in part, on the physical dimensions of the fermenter 870, the flow rate of the biomaterial waste through the fermentation unit 580, the type of biomaterial waste and other factors.

Following step 1232, the PLC circuit 120 is operable at step 1234 to monitor one or more excess fermentation organism indicators, F1E. Following step 1234, the PLC circuit 120 is operable at step 1236 to determine whether the one or more excess fermentation organism indicators, F1E, indicate an excess of the fermentation organism within the fermenter 870. If not, the routine 1228 continually loops back to step 1234 until F1E indicates a fermentation organism excess. When the PLC circuit 120 determines at step 1236 that F1E indicates a fermentation organism excess, execution of the routine 1228 advances to step 1238. Details relating to some example strategies for determining when a fermentation organism excess condition exists according to steps 1234 and 1236 will be described hereinafter following the description of the general steps of the routine 1228.

At step 1238, the PLC circuit 120 is operable to reset a fermentation organism collection timer. Thereafter at step 1240, the PLC circuit 120 is operable to control the inner sparger inlet valve 1110 to a closed position to stop the flow of air to the inner sparger 898 of the fermenter 870. Thereafter at step 1242, the PLC circuit 120 is operable to determine the mass flow rate, F1AE, of gas exiting the fermenter 870 by monitoring the mass flow rate signal produced by the mass flow meter or sensor 122₂₅, and at the following step 1244 the PLC circuit 120 is operable to control the outer sparger inlet valve 1112 to increase the air flow to the outer sparger 896 to compensate for turning off the flow of air to the inner sparger 898 at step 1240. In the illustrative embodiment of routine 1228, it is desirable to maintain constant mass air flow through the fermenter 870, and the PLC circuit 120 is accordingly operable at step 1244 to control the outer sparger inlet valve 1112 to increase the air flow to the outer sparger 896 to an air flow level that maintains the mass air flow exiting the fermenter 870 at a constant level. Following step 1244, the PLC circuit 120 is operable at step 1246 to continually loop back to step 1246 until the F1 collection timer has timed out. Thereafter at step 1248, the PLC circuit is operable to return the positions of the outer and inner air spargers 896 and 898 respectively of the fermenter 870 to their pre-collection valve positions. Execution of the routine loops from step 1248 back to step 1230.

By stopping the flow of air to the inner sparger 898 of the fermenter 870 at step 1240 the fermenting organism present in and above the cone 890 settles, and is collected within, the lower portion of the cone 890, thereby reducing the total amount of the fermentation organism being circulated through the fermenter 870. In the illustrated embodiment, airflow to the inner sparger 898 of the fermenter 870 is turned off for a time period defined by the timeout duration of the F1 collection timer. The timeout duration of the F1 collection timer may be established according to any one or more of a number of timer strategies. For example, the timeout period of the F1 collection timer may be set to a constant value based on the physical dimensions of the fermenter 870, composition and flow rate of the biomaterial waste, type of fermenting organism and/or other factors. As another example, the timeout period of the F1 collection timer may be set as a function of the amount of time that has elapsed since the fermenting organism was last collected. As yet another example, the timeout period of the F1 collection timer may be set as a function of the change in conductivity of the biomaterial waste across the fermenter 870. In this embodiment, the routine 1228 will include a number of steps between steps 1244 and 1246 wherein the PLC circuit 120 is operable to determine the conductivity of the biomaterial waste entering the fermenter 870 by monitoring the output of the conductivity sensor 122₁₇, to determine the conductivity of the biomaterial waste exiting the fermenter 870 by monitoring the output of the conductivity sensor 122₂₂, and to determine the timeout period of the F1 collection timer, corresponding to the time that the inner sparger 898 is turned off, as a function of the corresponding input and output conductivity values. Those skilled in the art will recognize other strategies for determining an appropriate time out period of the F1 collection timer, and any such other strategies are intended to fall within the scope of the claims appended hereto. In any case, the fermenting organism is collected within the lower portion of the cone 890 such that when air flow to the inner sparger 898 is thereafter restored, the fermenting organism collected within the lower portion of the cone 890 remains in the lower portion of the cone 890 for subsequent extraction.

The PLC circuit 120 is generally operable to execute steps 1234 and 1236 to determine whether an excess amount of the fermenting organism exists in the fermenter 870 by monitoring and processing one or more operating parameters of the fermenter 870. Particular ones or combinations of the operating parameters of the fermenter 870 used to determine whether an excess of the fermenting organism exists in the fermenter 870 will depend on a number of factors including, but not limited to, the physical dimensions of the fermenter 870, the composition and flow rate of the biomaterial waste, the type of fermenting organism, and the like. As one illustrative example, the following list represents one or more parameters that may be monitored to determine whether an excess amount of the fermenting organism exists in the fermenter 870 in the case where the fermenter 870 has the physical dimensions given by example in reference to FIGS. 23A-23C, where the biomaterial waste is cattle waste having variable nutrient content, and where the flow rate of the cattle waste through the fermenter 870 is approximately 100 gallons (379 liters) per minute:
1. mass flow rate of gas (air) exiting the fermenter 870,
2. derivative of 1,
3. change, e.g., decrease, in conductivity across the fermenter 870,
4. derivative of 2,
5. BTU generated in the fermenter 870, and
6. ratios of one or more combinations of 1-5.

Those skilled in the art will recognize that the foregoing list may omit one or more items and/or include other operating parameters not specifically listed, and that any such alternate list will typically be dictated by the specific application of the biomaterial waste processing system 10.

In the illustrated example, the fermentation of cattle waste will generally replace oxygen with carbon dioxide, and the fermenter 870 is sized to allow no more fermentation than the amount of incoming air will support. Thus, if the mass flow rate of gas exiting the fermenter 870 increases beyond a predetermined ratio of the exit gas mass flow rate and the baseline exit gas mass flow rate determined prior to fermentation, or beyond a predetermined derivative of the baseline exit gas mass flow rate, this is an indication that the fermenter 870 does not have sufficient incoming airflow to support the amount of fermentation occurring in the fermenter 870. Subsequent reduction and collection of some of the fermenting organism circulating through the fermenter 870 will reduce the total amount of fermentation, thereby decreasing the mass flow rate of gas exiting the fermenter 870. In this example, the PLC circuit 120 may be operable at steps 1232 and 1234 to determine whether an excess of the fermentation organism exists in the fermenter 870 by monitoring the flow rate of gas (air) exiting the fermenter 870 and advancing to step 1238 if this exit gas mass flow rate increases above the aforementioned ratio or derivative value. The PLC circuit 120 may be operable to supplement the exit gas mass flow rate information with the derivative of the exit gas mass flow rate for more a more precise determination of an excess fermentation organism condition. In any case, the PLC circuit 120 is operable to maintain an array of such exit gas mass flow rate data, and to perform conventional regression analyses to track and predict behavior of this data. In the illustrated example, the exit gas mass flow rate data is a highly sensitive indicator of excess fermenting organism in the fermenter 870.

The heat (BTU) generated by the metabolic activity within the fermenter 870 is given by the equation BTU=F122₃*(TD1−T122₁₆), where F122₃ is the biomaterial waste flow rate signal produced by the flow sensor 122₃ comprising part of the sterilization unit 570 as illustrated in FIG. 13A, TD1 is the design fermentation temperature of the fermenter 870, and T122₁₆ is the temperature signal produced by the temperature sensor 122₁₆ on signal path 124₁₆ and represents the temperature of the biomaterial waste exiting HX3 and entering the fermenter 870. BTU is also the sum of the catabolic activity and the anabolic activity within the fermenter 870, where the difference in the conductivity across the fermenter 870 is a direct measure of the anabolic activity, e.g., anabolic activity=K*(C122₁₇−C122₂₂), where K is a constant, C122₁₇ is the conductivity signal produced by the conductivity sensor 122₁₇ and represents the conductivity of the biomaterial waste entering the fermenter 870, and C122₂₂ is the conductivity signal produced by the conductivity sensor 122₂₂ and represents the conductivity of the biomaterial waste exiting the fermenter 870. The catabolic activity, CA1, within the fermenter 870 is then the difference between the BTU value and the anabolic activity according to the equation CA1=F122₃*(TD1−T122₁₆)−K*(C122₁₇−C122₂₂). In this example, the PLC circuit 120 may be alternatively or additionally operable at steps 1232 and 1234 to determine whether an excess of the fermentation organism exists in the fermenter 870 by computing the catabolic activity, CA1, according to the above equation and advancing to step 1238 if CA1 falls below a threshold catabolic activity value. The PLC circuit 120 may be operable to supplement the CA1 information with the derivative of CA1 for more a more precise determination of an excess fermentation organism condition. In any case, the PLC circuit 120 is operable to maintain an array of such CA1 data, and to perform conventional regression analyses to track and predict behavior of this data.

Those skilled in the art will recognize that the foregoing examples are provided only for the purpose of illustration, and that any one or more, or any combination and/or ratio of, the fermenter 870 operating parameters in the above list may be monitored and processed by the PLC circuit 120 to determine whether an excess fermentation organism condition exists in the fermenter 870. Moreover, the above list may omit one or more of the enumerated items and/or may include one or more other fermenter 870 operating parameters that are not specifically enumerated, and any such alternative list is intended to fall within the scope of the claims appended hereto.

The fermentation unit control algorithm 1180 further includes another control routine 1250, as illustrated in FIG. 26D, for controlling extraction of the fermenting organism, e.g., yeast or other fermenting organism, from the first fermenter 870 by controlling operation of the fermenting organism extraction pump 1148. The control routine 1250 begins at step 1252 where the PLC circuit 120 is operable to estimate the quantity, Q1, of the fermenting organism collected within the lower portion of the cone 890. The PLC circuit 120 may be operable at step 1250 to estimate the quantity, Q1, of collected fermenting organism within the lower portion of the cone 980 according to any one or more of a number of estimation strategies. For example, Q1 may be estimated as a function of the amount of time that has elapsed since the fermenting organism was last extracted from the fermenter 870. As another example, Q1 may be estimated as a function of the change in conductivity of the biomaterial waste across the fermenter 870. In this embodiment, the routine 1250 will include a number of steps prior to step 1252 wherein the PLC circuit 120 is operable to determine the conductivity of the biomaterial waste entering the fermenter 870 by monitoring the output of the conductivity sensor 122₁₇, to determine the conductivity of the biomaterial waste exiting the fermenter 870 by monitoring the output of the conductivity sensor 122₂₂, and to estimate Q1 as a function of the corresponding input and output conductivity values. Those skilled in the art will recognize other strategies for estimating the quantity, Q1, of collected fermenting organism within the lower portion of the cone 890 of the fermenter 870, and any such other strategies are intended to fall within the scope of the claims appended hereto.

In any case, execution of the routine 1250 advances from step 1252 to step 1254 where the PLC circuit 120 is operable to compare Q1 to a threshold fermenting organism quantity, Q1_{TH}. If Q1 is greater than or equal to Q1_{TH}, algorithm execution advances to step 1256. If, however, the PLC circuit 120 determines at step 1254 that Q1 is less than Q1_{TH}, execution of the routine 1250 loops back to step 1252.

At step 1256, the PLC circuit 120 is operable to reset an F1 extraction timer. Thereafter at step 1258, the PLC circuit 120 is operable to activate the F1 extraction pump 1148 by appropriately controlling the corresponding pump driver 1150, and thereafter at step 1260 the PLC circuit 120 is operable to continually re-execute step 1260 until the F1 extraction timer has timed out. The timeout duration of the F1 extraction timer may be established according to any one or more of a number of timer strategies. For example, the timeout period of the F1 extraction timer may be set to a constant value based on the physical dimensions of the fermenter 870 and the cone 890, the composition and flow rate of the biomaterial waste, type of fermenting organism and/or other factors. As another example, the timeout period of the F1 extraction timer may be set as a function of the amount of time that has elapsed since the fermenting organism was last collected. As yet another example, the timeout period of the F1 extraction timer may be set as a function of the estimated quantity, Q1, of the fermenting organism collected within the lower portion of the cone 890. Those skilled in the art will recognize other strategies for determining an appropriate time out period of the F1 extraction timer, and any such other strategies are intended to fall within the scope of the claims appended hereto. In any case, execution of the routine 1250 advances from the “yes” branch of step 1260 to step 1262 where the PLC circuit 120 is operable to deactivate the F1 extraction pump 1148. Thereafter, execution of the routine 1250 loops back to step 1252.

As an alternative to steps 1256-1260, the routine 1250 may instead include steps 1266-1270 as shown encompassed within dashed-line box 1264 in FIG. 26D. In this embodiment execution of the routine 1250 advances from the “yes” branch of step 1254 to step 1266 where the PLC circuit 120 is operable to activate the F1 extraction pump 1148 by appropriately controlling the corresponding pump driver 1150. Thereafter at step 1268, the PLC circuit 120 is operable to determine an operating torque of the F1 extraction pump 1148. In this embodiment, the pump driver 1150 includes an output signal path 124₃₃ as shown in phantom in FIG. 25, and the pump driver 1150 is operable to determine an operating torque of the pump 1148 using any one or more of the techniques described herein, and produce a corresponding operating torque signal, F1T, on signal path 124₃₃. The PLC circuit 120 is operable at step 1268 to determine the operating torque of the F1 extraction pump 1148 by monitoring the output torque signal, F1T, on signal path 124₃₃, and execution of the routine 1250 advances therefrom to step 1270 where the PLC circuit 120 is operable to compare F1T to a torque threshold F1T_{TH}. As long as F1T is greater than F1T_{TH}, execution of the routine 1250 loops back to step 1268. If the PLC circuit 120 determines at step 1270 that F1T is less than or equal to F1T_{TH}, execution of the routine 1250 advances to step 1262. In the illustrated embodiment, F1T_{TH} is set at a torque value below which the quantity of fermenting organism collected in the lower portion of the cone 890 has been sufficiently extracted.

The PLC circuit 120 is operable, under the direction of the routine 1250, to selectively extract the fermenting organism collected within the lower portion of the cone 890 of the fermenter 870 by estimating the quantity of fermenting organism collected within the lower portion of the cone 890 and controlling the F1 extraction pump 1148 to extract the fermenting organism from the cone 890 when the estimated fermenting organism quantity is greater than or equal to a threshold quantity. In one embodiment, activation of the F1 extraction pump 1148 is controlled on a timed basis, and in an alternative embodiment activation of the F1 extraction pump 1148 is controlled as a function of the output torque of the pump 1148. In either case, collection and extraction of the fermenting organism within/from the fermenter 870 are asynchronous operations, and the routines 1228 and 1250 may accordingly be executed simultaneously or non-simultaneously.

The fermentation unit control algorithm 1180 further includes another control routine 1280, as illustrated in FIG. 26E, for controlling the liquid level within the second fermenter 910. The control routine 1280 begins at step 1282 where the PLC circuit 120 is operable to determine the operating pressure, P3, of the second fermenter 910 by monitoring the pressure signal produced by the pressure sensor 122₂₈ on signal path 124₂₈. Thereafter at step 1284, the PLC circuit 120 is operable to determine the pressure, P4, of gas exiting the second fermenter 910 by determining the set point of the mechanical pressure control valve 1136. Following step 1284, the PLC circuit 120 is operable at step 1286 to compare the difference between P3 and P4 to a design pressure, P_{DES2}, where P_{DES2} corresponds to a pressure equivalent of the desired liquid level within the second fermenter 910.

If, at step 1286, the PLC circuit 120 determines that (P3−P4) is greater than P_{DES2}, indicating that the liquid level within the second fermenter 910 is higher than desired, the PLC circuit 120 is operable thereafter at step 1288 to increase the speed of the residual liquid outlet pump 1144 by producing an appropriate actuator control signal on signal path 130₂₁, to increase the flow of liquid exiting the second fermenter 910. If, on the other hand, the PLC circuit 120 determines at step 1286 that (P3−P4) is not greater than P_{DES2}, execution of the control routine 1280 advances to step 1290 where the PLC circuit 120 is again operable to compare the difference between P3 and P4 to the design pressure, P_{DES2}. If, at step 1290, the PLC circuit determines that (P3−P4) is less than P_{DES2}, indicating that the liquid level within the second fermenter 910 is lower than desired, the PLC circuit 120 is operable thereafter at step 1292 to decrease the speed of the residual liquid outlet pump 1144 by producing an appropriate actuator control signal on signal path 130₂₁, to decrease the flow of liquid exiting the second fermenter 910. If, on the other hand, the PLC circuit 120 determines at step 1290 that (P3−P4) is not less than P_{DES2}, execution of the control routine 1280 loops back to step 1282 as it also does following execution of steps 1288 and 1292.

The fermentation unit control algorithm 1180 further includes another control routine 1300, as illustrated in FIG. 26F, for controlling the operating temperature of the second fermenter 910 by controlling the temperature of the liquid waste entering the second fermenter 910 from the first fermenter 870 via control of coolant fluid flow through the heat exchanger HX4. The control routine 1300 begins at step 1302 where the PLC circuit 120 is operable to determine the flow rate of coolant fluid, CF4, from the cooling tower unit 586 through the heat exchanger HX4. In the illustrated embodiment, the PLC 120 is operable to execute step 1302 by computing CF4 as a function of the flow rate of the biomaterial waste entering the second fermenter 910, via conduit 900, from the first fermenter 870, the temperature difference between the biomaterial waste entering and exiting HX4 and the temperature difference between the cooling fluid entering and exiting HX4. In particular, the PLC 120 is operable at step 1302 to compute CF4 according to the equation CF4=F122₂₁*(T122₁₄−T122₂₃)/(T122₂₆−T122₂₇), where F122₂₁ is the biomaterial waste flow rate signal produced by the flow sensor 122₂₁, T122₁₄ is the temperature signal produced by the temperature sensor 122₁₄ on signal path 124₁₄ and represents the operating temperature of the first fermenter 870 and thus the temperature of the biomaterial waste entering HX4, T122₂₃ is the temperature signal produced by the temperature sensor 122₂₃ on signal path 124₂₃ and represents the temperature of the biomaterial waste exiting HX4, T122₂₆ is the temperature signal produced by the temperature sensor 122₂₆ on signal path 124₂₆ and represents the temperature of the cooling fluid entering HX4 from the cooling tower unit 586, and T122₂₇ is the temperature signal produced by the temperature sensor 122₂₇ on signal path 124₂₇ and represents the temperature of the cooling fluid exiting HX4. Alternatively, the coolant flow path through HX4 may include a flow meter or sensor, and in this embodiment the PLC 120 may be operable to execute step 1302 by monitoring the flow signal produced by such a flow meter or sensor. In any case, the execution of routine 1300 advances from step 1302 to step 1304 where the PLC circuit 120 is operable to determine the operating temperature, T2, of the second fermenter 910 by monitoring the temperature signal produced by the temperature sensor 122₂₉ on signal path 124₂₉. Thereafter at step 1306, the PLC circuit 120 is operable to compare the temperature, T2, of the second fermenter 910 to a design temperature, T_{D2}, wherein T_{D2} corresponds to a desired operating or fermenting temperature of the second fermenter 910.

If, at step 1306, the PLC circuit 120 determines that T2 is greater than T_{D2}, indicating that the operating temperature of the second fermenter 910 is greater than the design temperature, T_{D2}, execution of the control routine 1300 advances to step 1308 where the PLC circuit 120 is operable to compare the cooling fluid flow rate, CF4, through HX4 to a maximum flow rate value, MAXF4, wherein MAXF4 corresponds to a desired maximum flow rate of cooling fluid from the cooling tower unit 586. If, at step 1308, the PLC circuit 120 determines that CF4 is greater than or equal to MAXF4, routine execution advances to step 1310 where the PLC circuit 120 is operable to decrease the flow of biomaterial waste to the fermentation unit 580. In one embodiment, the PLC circuit 120 is operable to execute step 1310 by decreasing the speed of the biomaterial waste pump 612 forming part of the sterilization unit 570 as illustrated in FIG. 13A. Alternatively or additionally, the PLC circuit 120 may be operable to execute step 1310 by controlling the diverter valve 638 of the sterilization unit 570 to divert at least some of the biomaterial waste stream exiting the sterilization loop 630 back through the sterilization unit 570 to thereby decrease the flow rate of biomaterial waste exiting the sterilization unit 570. Alternatively or additionally still, the PLC circuit 120 maybe operable to execute step 1310 by, controlling the biomaterial waste return valve 622 to return at least some of the biomaterial waste stream flowing through the sterilization unit 570 back to the biomaterial waste source 20 (FIG. 1) to thereby decrease the flow rate of biomaterial waste exiting the sterilization unit 570. In any case, execution of the routine 1300 loops from step 1310 back to step 1302.

If, at step 1308, the PLC circuit 120 determines that the CF4 is less than MAXF4, routine execution advances to step 1312 where the PLC circuit 120 is operable to compare the speed of the pump 1132 (P4) supplying the cooling fluid from the cooling tower unit 586 to HX4 to a maximum pump speed, MAXSP4, wherein MAXSP4 corresponds to a maximum pump speed value that may be arbitrary or may be dictated by the physical properties of the pump 1132. In either case, if the PLC circuit 120 determines at step 1312 that the speed of the pump P4 is greater than or equal to MAXSP4, routine execution advances to step 1314 where the PLC circuit 120 is operable to stop the flow of biomaterial waste to the fermentation unit 580. In one embodiment, the PLC circuit 120 is operable to execute step 1314 by deactivating the biomaterial waste pump 612 forming part of the sterilization unit 570 as illustrated in FIG. 13A. Alternatively or additionally, the PLC circuit 120 may be operable to execute step 1314 by controlling the diverter valve 638 of the sterilization unit 570 to divert the biomaterial waste stream exiting the sterilization loop 630 back through the sterilization unit 570 to thereby stop the flow rate of biomaterial waste exiting the sterilization unit 570. Alternatively or additionally still, the PLC circuit 120 may be operable to execute step 1310 by controlling the biomaterial waste return valve 622 to return the biomaterial waste stream flowing through the sterilization unit 570 back to the biomaterial waste source 20 (FIG. 1) to thereby stop the flow rate of biomaterial waste exiting the sterilization unit 570. In any case, execution of the routine 1300 advances from step 1314 to step 1318 where the PLC circuit 120 is operable to pause until the temperature, T2, of the fermenter 910 is less than or equal to the design temperature, TD2. Thereafter, routine execution advances to step 1320 where the control circuit is operable to control the flow of the biomaterial waste stream entering the fermentation unit 580, using any of the techniques just described, to resume the flow of biomaterial waste into the fermentation unit 580. Thereafter, execution of the routine 1300 loops back to step 1302. If, at step 1312, the PLC circuit 120 determines that the speed of the cooling fluid pump P4 is less than MAXSP4, routine execution advances to step 1316 where the PLC circuit 120 is operable to increase the speed of the pump P4 by appropriately controlling the pump driver 1134. Thereafter, routine execution loops back to step 1302.

If, at step 1306, the PLC circuit 120 determines that the temperature, T2, of the fermenter 910 is greater than the design temperature, TD2, routine execution advances to step 1322 where the PLC circuit 120 is operable to determine whether T2 is less than TD2. If not, then T2=TD2 and routine execution advances to step 1324 where the PLC circuit 120 is operable to maintain the current speed of the pump P4 by appropriately controlling the pump driver 1134. If, at step 1322, the PLC circuit 120 determines that T2 is less than TD2, routine execution advances to step 1326 where the PLC circuit 120 is operable to decrease the speed of the pump P4 by appropriately controlling the pump driver 1134. Routine execution loops from either of steps 1324 and 1326 back to step 1302.

The PLC circuit 120 is operable, under the direction of the routine 1300, to control the temperature, T2, of the second fermenter 910 by comparing T2 to a design temperature, TD2, and increasing the speed of the pump 1132 (P4) supplying cooling fluid from the cooling tower unit 586 to the heat exchanger HX4 if T2 is greater than TD2, the flow rate of the cooling fluid through HX4 is less than a maximum cooling fluid flow rate, MAXF4, and the speed of the pump 1132 is not greater than or equal to a maximum pump speed, MAXSP4. If, however, T2 is greater than TD2 and the flow rate of the cooling fluid through HX4 is greater than or equal to MAXF4, then T2 cannot be lowered by increasing the speed of the pump 1132, and the flow rate of the biomaterial waste into the fermentation unit 580 is instead decreased. If T2 is greater than TD2 and the flow rate of the cooling fluid through HX4 is less than MAXF4 but the speed of the pump 1132 is greater than or equal to the maximum pump speed, MAXSP4, then no action of the cooling fluid pump 1132 will result in further cooling of the biomaterial waste stream, and in this case the flow of biomaterial waste into the fermentation unit 580 is stopped until T2 becomes less than or equal to TD2. If T2 is less than TD2, the speed of the pump 1132 is decreased, and if T2 is equal to TD2 the speed of the pump 1132 is maintained at its current pump speed.

The fermentation unit control algorithm 1180 further includes another control routine 1328, as illustrated in FIG. 26G, for controlling collection of the fermenting organism, e.g., yeast or other fermenting organism, within the lower portion of the cone 930 of the second fermenter 910 as illustrated in FIGS. 19, 21-22 and 24A-24C. It will be understood that the control routine 1328 represents one embodiment of a control routine for controlling fermenting organism collection within the fermenter 910 during normal, continuous flow operation, and that air flow into the outer and inner air spargers 904 and 934 respectively of the fermenter 910 will typically be established and controlled prior to normal, continuous flow operation by the PLC circuit 120. The control routine 1328 begins at step 1330 where the PLC circuit 120 is operable to determine the flow rate of air exiting the fermenter 910 by monitoring the mass air flow signal produced by the mass flow meter or sensor 122₃₀ illustrated in FIG. 25. Thereafter at step 1332, the PLC circuit 120 is operable to control the inner sparger inlet valve 1128 and outer sparger inlet valve 1126 respectively of the fermenter 910 to drive the mass flow rate of air exiting the fermenter 910 to a design mass air flow exit value, F2AED, wherein F2AED represents a target mass air flow value that will depend, at least in part, on the physical dimensions of the fermenter 910, the flow rate of the biomaterial waste through the fermentation unit 580, the type of biomaterial waste and other factors.

Following step 1332, the PLC circuit 120 is operable at step 1334 to monitor one or more excess fermentation organism indicators, F2E. Following step 1334, the PLC circuit 120 is operable at step 1336 to determine whether the one or more excess fermentation organism indicators, F2E, indicate an excess of the fermentation organism within the fermenter 910. If not, the routine 1328 continually loops back to step 1334 until F2E indicates a fermentation organism excess. When the PLC circuit 120 determines at step 1336 that F2E indicates a fermentation organism excess, execution of the routine 1328 advances to step 1338. Details relating to some example strategies for determining when a fermentation organism excess condition exists according to steps 1334 and 1336 will be described hereinafter following the description of the general steps of the routine 1328.

At step 1338, the PLC circuit 120 is operable to reset a fermentation organism collection timer. Thereafter at step 1340, the PLC circuit 120 is operable to control the inner sparger inlet valve 1128 to a closed position to stop the flow of air to the inner sparger 934 of the fermenter 910. Thereafter at step 1342, the PLC circuit 120 is operable to determine the mass flow rate, F2AE, of air exiting the fermenter 910 by monitoring the mass flow rate signal produced by the mass flow meter or sensor 122₃₀, and at the following step 1344 the PLC circuit 120 is operable to control the outer sparger inlet valve 1126 to increase the air flow to the outer sparger 904 to compensate for turning off the flow of air to the inner sparger 934 at step 1340. In the illustrative embodiment of routine 1328, it is desirable to maintain constant mass air flow through the fermenter 910, and the PLC circuit 120 is accordingly operable at step 1344 to control the outer sparger inlet valve 1126 to increase the air flow to the outer sparger 904 to an air flow level that maintains the mass air flow exiting the fermenter 910 at a constant level. Following step 1344, the PLC circuit 120 is operable at step 1346 to continually loop back to step 1346 until the F2 collection timer has timed out. Thereafter at step 1348, the PLC circuit is operable to return the positions of the outer and inner air spargers 904 and 934 respectively of the fermenter 910 to their pre-collection valve positions. Execution of the routine loops from step 1348 back to step 1330.

By stopping the flow of air to the inner sparger 934 of the fermenter 910 at step 1340 the fermenting organism present in and above the cone 930 settles, and is collected within, the lower portion of the cone 930, thereby reducing the total amount of the fermentation organism being circulated through the fermenter 910. In the illustrated embodiment, airflow to the inner sparger 934 of the fermenter 910 is turned off for a time period defined by the timeout duration of the F2 collection timer. The timeout duration of the F2 collection timer may be established according to any one or more of a number of timer strategies. For example, the timeout period of the F2 collection timer may be set to a constant value based on the physical dimensions of the fermenter 910, composition and flow rate of the biomaterial waste, type of fermenting organism and/or other factors. As another example, the timeout period of the F2 collection timer may be set as a function of the amount of time that has elapsed since the fermenting organism was last collected. As yet another example, the timeout period of the F2 collection timer may be set as a function of the change in conductivity of the biomaterial waste across the fermenter 910. In this embodiment, the routine 1328 will include a number of steps between steps 1344 and 1346 wherein the PLC circuit 120 is operable to determine the conductivity of the biomaterial waste entering the fermenter 910 by monitoring the output of the conductivity sensor 122₂₂, to determine the conductivity of the biomaterial waste exiting the fermenter 910 by monitoring the output of the conductivity sensor 122₃₂, and to determine the timeout period of the F2 collection timer, corresponding to the time that the inner sparger 934 is turned off, as a function of the corresponding input and output conductivity values. Those skilled in the art will recognize other strategies for determining an appropriate time out period of the F2 collection timer, and any such other strategies are intended to fall within the scope of the claims appended hereto. In any case, the fermenting organism is collected within the lower portion of the cone 930 such that when air flow to the inner sparger 934 is thereafter restored, the fermenting organism collected within the lower portion of the cone 930 remains in the lower portion of the cone 930 for subsequent extraction.

The PLC circuit 120 is generally operable to execute steps 1334 and 1336 to determine whether an excess amount of the fermenting organism exists in the fermenter 910 by monitoring and processing one or more operating parameters of the fermenter 910. Particular ones or combinations of the operating parameters of the fermenter 910 used to determine whether an excess of the fermenting organism exists in the fermenter 910 will depend on a number of factors including, but not limited to, the physical dimensions of the fermenter 910, the composition and flow rate of the biomaterial waste, the type of fermenting organism, and the like. As one illustrative example, the following list represents one or more parameters that may be monitored to determine whether an excess amount of the fermenting organism exists in the fermenter 910 in the case where the fermenter 910 has the physical dimensions given by example in reference to FIGS. 24A-24C, where the biomaterial waste is cattle waste having variable nutrient content, and where the flow rate of the cattle waste through the fermenter 910 is approximately 100 gallons (379 liters) per minute:
1. mass flow rate of gas (air) exiting the fermenter 910,
2. derivative of 1,
3. change, e.g., decrease, in conductivity across the fermenter 910,
4. derivative of 2,
5. BTU generated in the fermenter 910, and
6. ratios of one or more combinations of 1-5.

Those skilled in the art will recognize that the foregoing list may omit one or more items and/or include other operating parameters not specifically listed, and that any such alternate list will typically be dictated by the specific application of the biomaterial waste processing system 10.

In the illustrated example, the fermenter 910 is sized to supply more incoming air than is required to support fermentation therein. Consequently, the fermenter 910 will typically not use all of the air supplied to it. As a result, the mass flow rate of gas (air) exiting the fermenter 910 will typically not be a highly sensitive indicator of excess fermenting organism in the fermenter 910. However, in other embodiments of the fermentation unit 580, the fermenter 910 may be sized and configured similarly as described hereinabove with respect to the fermenter 870, and in such cases the mass flow rate of gas (air) exiting the fermenter 910 may be a sensitive indicator of excess fermenting organism in the fermenter 910. In such cases, the PLC circuit 120 is operable as described hereinabove with respect to the control routine 1200 of FIG. 26C at steps 1332 and 1334 to determine whether an excess of the fermentation organism exists in the fermenter 910 by monitoring the mass flow rate of gas (air) exiting the fermenter 910 and advancing to step 1238 if the exit gas mass flow rate increases above a ratio of the exit gas mass flow rate and a baseline exit gas mass flow rate, which may be calculated prior to fermentation within the second fermenter 910 in a similar manner to that described hereinabove with respect to control routine 1228 of FIG. 26C, or above a derivative of the baseline exit gas mass flow rate value for the second fermenter 910.

Similarly as described hereinabove with respect to the fermenter 870, the heat (BTU) generated by the metabolic activity within the fermenter 910 is given by the equation BTU=F122₂₁*(TD2−T122₂₃), where F122₂₁ is the biomaterial waste flow rate signal produced by the flow sensor 122₂₁, TD2 is the design fermentation temperature of the fermenter 910, and T122₂₃ is the temperature signal produced by the temperature sensor 122₂₃ on signal path 124₂₃ and represents the temperature of the biomaterial waste exiting HX4 and entering the fermenter 910. BTU is also the sum of the catabolic activity and the anabolic activity within the fermenter 910, where the difference in the conductivity across the fermenter 910 is a direct measure of the anabolic activity, e.g., anabolic activity=K*(C122₂₂−C122₃₂), where K is a constant, C122₂₂ is the conductivity signal produced by the conductivity sensor 122₂₂ and represents the conductivity of the biomaterial waste entering the fermenter 910, and C122₃₂ is the conductivity signal produced by the conductivity sensor 122₃₂ and represents the conductivity of the biomaterial waste exiting the fermenter 910. The catabolic activity, CA2, within the fermenter 910 is then the difference between the BTU value and the anabolic activity according to the equation CA2=F122₂₁*(TD1−T122₂₃)−K*(C122₂₂−C122₃₂). In this example, the PLC circuit 120 is operable at steps 1332 and 1334 to determine whether an excess of the fermentation organism exists in the fermenter 910 by computing the catabolic activity, CA2, according to the above equation and advancing to step 1338 if CA2 falls below a threshold catabolic activity value. The PLC circuit 120 may be operable to supplement the CA2 information with the derivative of CA2 for more a more precise determination of an excess fermentation organism condition. In any case, the PLC circuit 120 is operable to maintain an array of such CA2 data, and to perform conventional regression analyses to track and predict behavior of this data. In the illustrated example, the catabolic activity data is a highly sensitive indicator of excess fermenting organism in the fermenter 910.

Those skilled in the art will recognize that the foregoing examples are provided only for the purpose of illustration, and that any one or more, or any combination and/or ratio of, the fermenter 910 operating parameters in the above list may be monitored and processed by the PLC circuit 120 to determine whether an excess fermentation organism condition exists in the fermenter 910. Moreover, the above list may omit one or more of the enumerated items and/or may include one or more other fermenter 910 operating parameters that are not specifically enumerated, and any such alternative list is intended to fall within the scope of the claims appended hereto.

The fermentation unit control algorithm 1180 further includes another control routine 1350, as illustrated in FIG. 26H, for controlling extraction of the fermenting organism, e.g., yeast or other fermenting organism, from the second fermenter 910 by controlling operation of the fermenting organism extraction pump 1158. The control routine 1350 begins at step 1352 where the PLC circuit 120 is operable to estimate the quantity, Q2, of the fermenting organism collected within the lower portion of the cone 930. The PLC circuit 120 may be operable at step 1350 to estimate the quantity, Q2, of collected fermenting organism within the lower portion of the cone 930 according to any one or more of a number of estimation strategies. For example, Q2 may be estimated as a function of the amount of time that has elapsed since the fermenting organism was last extracted from the fermenter 910. As another example, Q2 may be estimated as a function of the change in conductivity of the biomaterial waste across the fermenter 910. In this embodiment, the routine 1350 will include a number of steps prior to step 1352 wherein the PLC circuit 120 is operable to determine the conductivity of the biomaterial waste entering the fermenter 910 by monitoring the output of the conductivity sensor 122₂₂, to determine the conductivity of the biomaterial waste exiting the fermenter 910 by monitoring the output of the conductivity sensor 122₃₂, and to estimate Q2 as a function of the corresponding input and output conductivity values. Those skilled in the art will recognize other strategies for estimating the quantity, Q2, of collected fermenting organism within the lower portion of the cone 930 of the fermenter 910, and any such other strategies are intended to fall within the scope of the claims appended hereto.

In any case, execution of the routine 1350 advances from step 1352 to step 1354 where the PLC circuit 120 is operable to compare Q2 to a threshold fermenting organism quantity, Q2_{TH}. If Q2 is greater than or equal to Q2_{TH}, algorithm execution advances to step 1356. If, however, the PLC circuit 120 determines at step 1354 that Q2 is less than Q2_{TH}, execution of the routine 1350 loops back to step 1352.

At step 1356, the PLC circuit 120 is operable to reset an F2 extraction timer. Thereafter at step 1358, the PLC circuit 120 is operable to activate the F2 extraction pump 1158 by appropriately controlling the corresponding pump driver 1160, and thereafter at step 1360 the PLC circuit 120 is operable to continually re-execute step 1360 until the F2 extraction timer has timed out. The timeout duration of the F2 extraction timer may be established according to any one or more of a number of timer strategies. For example, the timeout period of the F2 extraction timer may be set to a constant value based on the physical dimensions of the fermenter 910 and the cone 930, the composition and flow rate of the biomaterial waste, type of fermenting organism and/or other factors. As another example, the timeout period of the F2 extraction timer may be set as a function of the amount of time that has elapsed since the fermenting organism was last collected. As yet another example, the timeout period of the F2 extraction timer may be set as a function of the estimated quantity, Q2, of the fermenting organism collected within the lower portion of the cone 930. Those skilled in the art will recognize other strategies for determining an appropriate time out period of the F2 extraction timer, and any such other strategies are intended to fall within the scope of the claims appended hereto. In any case, execution of the routine 1350 advances from the “yes” branch of step 1360 to step 1362 where the PLC circuit 120 is operable to deactivate the F2 extraction pump 1158. Thereafter, execution of the routine 1350 loops back to step 1352.

As an alternative to steps 1356-1360, the routine 1350 may instead include steps 1366-1370 as shown encompassed within dashed-line box 1364 in FIG. 26H. In this embodiment execution of the routine 1350 advances from the “yes” branch of step 1354 to step 1366 where the PLC circuit 120 is operable to activate the F2 extraction pump 1158 by appropriately controlling the corresponding pump driver 1160. Thereafter at step 1368, the PLC circuit 120 is operable to determine an operating torque of the F2 extraction pump 1158. In this embodiment, the pump driver 1160 includes an output signal path 124₃₂ as shown in phantom in FIG. 25, and the pump driver 1160 is operable to determine an operating torque of the pump 1158 using any one or more of the techniques described herein, and produce a corresponding operating torque signal, F2T, on signal path 124₃₂. The PLC circuit 120 is operable at step 1368 to determine the operating torque of the F2 extraction pump 1158 by monitoring the output torque signal, F2T, on signal path 124₃₂, and execution of the routine 1350 advances therefrom to step 1370 where the PLC circuit 120 is operable to compare F2T to a torque threshold F2T_{TH}. As long as F2T is greater than F2T_{TH}, execution of the routine 1350 loops back to step 1368. If the PLC circuit 120 determines at step 1370 that F2T is less than or equal to F2T_{TH}, execution of the routine 1350 advances to step 1362. In the illustrated embodiment, F2T_{TH} is set at a torque value below which the quantity of fermenting organism collected in the lower portion of the cone 930 has been sufficiently extracted.

The PLC circuit 120 is operable, under the direction of the routine 1350, to selectively extract the fermenting organism collected within the lower portion of the cone 930 of the fermenter 910 by estimating the quantity of fermenting organism collected within the lower portion of the cone 930 and controlling the F2 extraction pump 1158 to extract the fermenting organism from the cone 930 when the estimated fermenting organism quantity, Q2, is greater than or equal to a threshold quantity. In one embodiment, activation of the F2 extraction pump 1158 is controlled on a timed basis, and in an alternative embodiment activation of the F2 extraction pump 1158 is controlled as a function of the output torque of the pump 1158. In either case, collection and extraction of the fermenting organism within/from the fermenter 910 are asynchronous operations, and the routines 1328 and 1350 may accordingly be executed simultaneously or non-simultaneously.

Referring now to FIG. 27A, a schematic diagram of one illustrative embodiment of the pasteurization unit 594 and corresponding control system that forms part of the waste fermentation system of FIG. 12 is shown. In the illustrated embodiment, the conduit 598 that is fluidly coupled to the product inlet, PIP, of the pasteurization unit 594 passes through a first ball valve, BV, through a pasteurization heat exchanger HX6, through another pair of ball valves, BV, through a post-pasteurization heat exchanger HX7, and through another pair of ball valves, BV, to a fermenting organism product port of a fermenting organism product storage tank 1400. The fermenting organism product storage tank 1400 is, in the illustrated embodiment, an insulated tank of known construction and operable to maintain the temperature of the fermenting organism product supplied thereto near the temperature of the fermenting organism product exiting the post-pasteurization heat exchanger 1400. Alternatively, the fermenting organism product tank 1400 may include conventional temperature controls for controlling the temperature of the tank interior and its contents.

In any case, the pasteurization unit 594 farther includes a conventional agitator 1402 configured to agitate or stir the fermenting organism product stored in the tank 1400. The agitator 1402 represents one of the “Q” actuators of the pasteurization unit 594, and is electrically connected to one of the actuator outputs of the PLC circuit 120 via one of the “Q” signal paths 130₂₆. The PLC circuit 120 is operable to periodically control the agitator 1402 for a predefined time period by producing an appropriate signal on signal path 130₂₆ to periodically stir the fermenting organism product stored within the fermenting organism product storage tank 1400. One of the “R” sensors included within the pasteurization unit 594 is a conventional temperature sensor 122₃₄ disposed in fluid communication with the interior of the fermenting organism product storage tank 1400 and electrically connected to the PLC circuit 120 via one of the “R” signal paths 124₃₄. The temperature sensor 122₃₄ is operable to produce a temperature signal on signal path 124₃₄ indicative of the temperature of the fermenting organism product stored in the fermenting organism product storage tank 1400. In the illustrated embodiment, the PLC circuit 120 is configured to monitor the temperature signal produced by the temperature sensor 122₃₄ on signal path 124₃₄, and to activate a warning mechanism if the temperature within the fermenting organism product storage tank rises above a threshold temperature level. If this occurs, a technician may extract the fermenting organism product stored in the tank 1400 and suitably relocate the product. Alternatively, in embodiments wherein the fermenting organism product storage tank 1400 includes temperature controls, the PLC circuit 120 may be configured to adjust such temperature controls as a function of the temperature signal produced by the temperature sensor 122₃₄ on signal path 124₃₄ to maintain the fermenting organism product stored in the tank 1400 near a desired storage temperature.

The conduit 604 that is fluidly coupled to the pasteurization steam inlet, PSTI, is coupled through a steam control valve 1404, through a ball valve, BV, through a steam-to-water heat exchanger HX5, through another ball valve, BV, and then fluidly coupled to conduit 606 to define the pasteurization steam outlet, PSTO, of the pasteurization unit 594. The steam control valve 1404 represents another one of the “Q” actuators of the pasteurization unit 594, and is electrically connected to another one of the actuator outputs of the PLC circuit 120 via one of the “Q” signal paths 130₂₇. Another conduit 1414 passes through the opposite side of the steam-to-water heat exchanger HX5 and passes through a pair of ball valves, BV, to the pasteurization heat exchanger HX6, and from HX6 through another ball valve, BV, to an inlet of a water storage tank 1406 configured to store a quantity of water therein. An outlet of the water storage tank 1406 is coupled through a pair of ball valves, BV, to an inlet of a water pump 14010 having a pump outlet coupled through another pair of ball valves, BV, through HX5 via conduit 1414. A conventional pump driver 1412 is electrically connected to the water pump 1410. The pump driver 1412 represents another one of the “Q” actuators of the pasteurization unit 594, and is electrically connected to another one of the actuator outputs of the PLC circuit 120 via one of the “Q” signal paths 130₂₈. Another one of the “R” sensors included within the pasteurization unit 594 is another conventional-temperature sensor 122₃₅ disposed in fluid communication with the conduit 1414 between the heat exchangers HX5 and HX6 and electrically connected to the PLC circuit 120 via one of the “R” signal paths 124₃₅. The temperature sensor 122₃₅ is operable to produce a temperature signal on signal path 124₃₅ indicative of the temperature of the steam heated water exiting the steam-to-water heat exchanger HX5.

In the illustrated embodiment, the water conduit 26 fluidly coupled to the water inlet, WI, of the pasteurization unit 594 is coupled through an inlet control valve 1416 and a ball valve, BV, and passes through an opposite side of the post-pasteurization heat exchanger HX7 and another ball valve, BV, and then intersects the waste return outlet conduit 596 defining the waste return outlet, WRO, of the pasteurization unit 594. The inlet control valve 1416 represents another one of the “Q” actuators of the pasteurization unit 594, and is electrically connected to another one of the actuator outputs of the PLC circuit 120 via another one of the “Q” signal paths 130₂₉. Another one of the “R” sensors included within the pasteurization unit 594 is another conventional temperature sensor 122₃₆ disposed in fluid communication with the heat exchanger HX7 and electrically connected to the PLC circuit 120 via one of the “R” signal paths 124₃₆. The temperature sensor 122₃₆ is operable to produce a temperature signal on signal path 124₃₆ indicative of the temperature of the post-pasteurization heat exchanger HX7. In embodiments wherein the temperature of the water supplied by the conventional water system 24 to the post-pasteurization heat exchanger HX7 via conduit 26 is not low enough to sufficiently cool the pasteurized fermenting organism flowing through HX7, cooling fluid from the cooling tower unit 586 (see FIG. 17) may instead be circulated through the post-pasteurization heat exchanger HX7 via conduits 590 and 588. Alternatively still, the pasteurization unit 594 may include a dedicated cooling tower unit, similar or identical in operation to the cooling tower unit 586 illustrated in FIG. 17, to provide water or other cooling fluid to the post-pasteurization heat exchanger HX7 at a temperature low enough to sufficiently cool the pasteurized fermenting organism flowing through HX7.

The conduit 598 fluidly coupled to the fermenting organism product storage tank 1400 is fluidly connected to one end of another conduit 1418 between the two ball valves, BV, separating the fermenting organism product storage tank 1400 and the post-pasteurization heat exchanger HX7. The opposite end of the conduit 1418 is fluidly coupled to an inlet of a product extraction pump 1420 having an outlet fluidly coupled through another ball valve, BVV, to the product outlet, POP, of the pasteurization unit 594 and to the product outlet conduit 70. The outlet of the product outlet pump 1420 is also fluidly coupled through another ball valve, BVW, to the waste return outlet, WRO, of the pasteurization unit 594. The product outlet pump is electrically connected to a conventional pump driver 1422, which represents another of the “Q” actuators of the pasteurization unit 594, and the pump driver is electrically connected to another actuator outputs of the PLC 120 via another of the “Q” signal paths 130₃₀. The PLC circuit 120 is operable to activate the product outlet pump 1420 via an appropriate signal on signal path 130₃₀ whenever it is desirable to extract fermenting organism product from the fermenting organism product storage tank 1400. The fermenting organism product extracted by the product outlet pump 1420 is supplied to the product outlet, POP, when the ball valve BVW is closed and the ball valve BVV is open. If the ball valve BVV is closed and the ball valve BVW is open, the fermenting organism product extracted by the product outlet pump 1420 is instead directed to the waste return outlet, WRO, of the pasteurization unit 594. Under normal, fermenting organism collection operation, the product outlet pump is off and the ball valves BVV and BVW are closed.

The junction of conduits 598 and 1418 is also fluidly coupled through ball valves BVY and BVZ to the sample outlet, SMPL, of the pasteurization unit 594, which is fluidly connected to the product sample conduit 600. The outlet of the ball valve BVY and the inlet of the ball valve BVZ are fluidly coupled through another ball valve, BVX, to the sample clean steam inlet, SCSI, of the pasteurization unit 594, which is fluidly connected to the sample clean steam conduit 606. When it is desired to sample some of the fermenting organism product stored in the fermenting organism product storage tank 1400, the ball valves BVY and BVZ are opened while the ball valve BVX is closed. This allows the fermenting organism product to be drawn from the sample outlet, SMPL, of the pasteurization unit 594. The product sample passageway just described may be cleaned with steam provided by the steam unit 572 via conduit 606. When the ball valves BVX and BVZ are opened while the ball valve BVY is closed, steam entering the sample clean steam inlet, SCSI, is directed through valves BVX and BVZ to the sample outlet, SMPL, to clean and sterilize this passageway. Under normal, fermenting organism collection operation, the ball valves BVX, BVY and BVZ are closed.

The pasteurization unit 594 is operable, under the control of the PLC circuit 120, to pasteurize the fermenting organism product produced and supplied by the fermentation unit 580 via appropriate control of the heat exchangers HX5 and HX6, and to then cool the pasteurized fermenting organism product via appropriate control of HX7 prior to storage of the cooled and pasteurized fermenting organism product in the fermenting organism product storage tank 1400. The pasteurization unit 594 just described includes a number of additional manually activated ball valves, BV, as illustrated in FIG. 27A. Such valves are included within the pasteurization unit 594 at various locations to allow for bypassing of, and maintenance or replacement of, various components of the pasteurization unit 594.

Referring now to FIG. 27B, a schematic diagram of another illustrative embodiment of the pasteurization unit 594′ and corresponding control system that forms part of the waste fermentation system of FIG. 12 is shown. The pasteurization unit 594′ is identical in many respects to the pasteurization unit 594 of FIG. 27A, and like numbers are therefore used to identify like components. In the embodiment illustrated in FIG. 27B, another heat exchanger, HX8, is added to pre-heat the incoming product prior to entrance into the heat exchanger HX6 to thereby decrease the heating requirement of the heat exchanger HX6. In particular, a first product inlet of the heat exchanger HX8 is fluidly coupled to the product inlet port, PIP, of the pasteurization unit 594′ via conduit 598, and a first product outlet of the heat exchanger HX8 is fluidly coupled to the product inlet of the heat exchanger HX6 via a conduit 1413. The product outlet of the heat exchanger HX6 is fluidly coupled to a second product inlet of the heat exchanger HX8; and a second product outlet of the heat exchanger HX8 is fluidly coupled to the heat exchanger HX7 via conduit 1415. The heat exchanger HX8 effectively pre-heats the incoming product, using the heat in the product exiting the heat exchanger HX6, prior to entrance into the heat exchanger HX6, thereby decreasing the overall heating requirement of the heat exchanger HX6.

Referring now to FIG. 28, a flowchart of one illustrative embodiment of a software algorithm 1430 for controlling the pasteurization unit 594 of either of FIGS. 27A and 27B is shown. It will be understood that the software algorithm 1430 represents one illustrative strategy for controlling the pasteurization unit 594 during normal, continuous flow operation of the biomaterial waste processing system 10, and that the pasteurization unit 594 may be controlled differently during other operational modes of the biomaterial waste processing system 10. The software algorithm 1430 includes a number of different and independently executing control routines, and each of these different control routines will be described separately. For example, the control algorithm 1430 includes a first control routine 1432 for controlling the temperature of the pasteurization heat exchanger HX6. The control routine 1432 begins at step 1434 where the PLC circuit 120 is operable to determine the operating temperature, T₆, of the pasteurization heat exchanger HX6 by monitoring the temperature signal produced by the temperature sensor 122₃₃ on signal path 124₃₃. Thereafter at step 1436, the PLC circuit 120 is operable to compare T₆ to a target pasteurization temperature, T_{P}. If, at step 1436, the PLC circuit 120 determines that T₆ is greater than or equal to T_{P}, execution of the control routine 1432 advances to step 1438 where the PLC circuit 120 is operable to deactivate the water pump 1410 if it is currently activated. From step 1438, execution of the control routine 1430 loops back to step 1434.

If, at step 1436, the PLC circuit 120 determines that T₆ is less than T_{P}, then the PLC circuit 120 is operable thereafter at step 1440 to raise the temperature of the pasteurization heat exchanger HX6 by activating the water pump 1412, by producing an appropriate signal on signal path 130₂₈, to circulate water heated by the heat exchanger HX5 between the heat exchangers HX5 and HX6. Thereafter at step 1442, the PLC circuit 120 is operable to determine the temperature, T₅₆, of the water flowing through conduit 1414 between HX5 and HX6 by monitoring the temperature signal produced by the temperature sensor 122₃₅ on signal path 124₃₅. Following step 1442, the PLC circuit 120 is operable at step 1444 to compare T₅₆ to a threshold temperature T56_{TH}, wherein T56_{TH} corresponds to the temperature of the water flowing through pasteurization heat exchanger HX5 that is required to raise the temperature of the pasteurization heat exchanger HX5 to or above the target pasteurization temperature, T_{P}. If, at step 1444, T₅₆ is less than T56_{TH}, execution of the control routine 1434 advances to step 1446 where the PLC circuit 120 is operable to control the steam inlet valve 1404, by producing an appropriate signal on signal path 130₂₇, to increase the opening of the steam inlet valve 1404 to thereby supply more steam to the steam-to-water heat exchanger HX5 to raise the temperature T₅₆. Execution of the control routine 1432 loops from step 1446 back to step 1434.

If, at step 1444, the PLC circuit 120 determines that T₅₆ is greater than or equal to T56_{TH}, execution of the control routine 1432 advances to step 1448 where the PLC circuit 120 is again operable to compare T₅₆ to T56_{TH}. If, at step 1448, T₅₆ is greater than T56_{TH}, execution of the control routine 1434 advances to step 1450 where the PLC circuit 120 is operable to control the steam inlet valve 1404, by producing an appropriate signal on signal path 130₂₇, to decrease the opening of the steam inlet valve 1404 to thereby supply less steam to the steam-to-water heat exchanger HX5 to lower the temperature T₅₆. If, however, the PLC circuit 120 determines at step 1448 that T₅₆ is not greater than T56_{TH}, execution of the control routine 1432 advances to step 1452 where the PLC circuit 120 is operable to control the steam inlet valve 1404, by producing an appropriate signal on signal path 130₂₇, to maintain the current opening of the steam inlet valve 1404 to thereby maintain the current value of the temperature T₅₆. Execution of the control routine 1432 loops from either of steps 1450 and 1452 back to step 1434.

The pasteurization unit control algorithm 1430 further includes another control routine 1454 for controlling the temperature of the post-pasteurization heat exchanger HX7. The control routine 1454 begins at step 1456 where the PLC circuit 120 is operable to determine the operating temperature, T₇, of the post-pasteurization heat exchanger HX7 by monitoring the temperature signal produced by the temperature sensor 122₃₆ on signal path 124₃₆. Thereafter at step 1458, the PLC circuit 120 is operable to compare T₇ to a threshold temperature, T7_{TH}, wherein T7_{TH} corresponds to the temperature of the post-pasteurization heat exchanger HX7 that is required to cool the pasteurized fermenting organism product flowing therethrough to a suitable storage temperature. If, at step 1458, T₇ is greater than T7_{TH}, execution of the control routine 1454 advances to step 1460 where the PLC circuit 120 is operable to control the water inlet valve 1416, by producing an appropriate signal on signal path 130₂₉, to increase the opening of the water inlet valve 1416 to thereby supply more fresh water to the heat exchanger HX7 to lower the temperature T₇. Execution of the control routine 1454 loops from step 1460 back to step 1456.

If, at step 1458, the PLC circuit 120 determines that T₇ is greater than or equal to T7_{TH}, execution of the control routine 1454 advances to step 1462 where the PLC circuit 120 is again operable to compare T₇ to T7_{TH}. If, at step 1462, T₇ is less than T7_{TH}, execution of the control routine 1454 advances to step 1464 where the PLC circuit 120 is operable to control the water inlet valve 1416, by producing an appropriate signal on signal path 130₂₉, to decrease the opening of the water inlet valve 1416 to thereby supply less water to the heat exchanger HX5 to raise the temperature T₇. If, however, the PLC circuit 120 determines at step 1462 that T₇ is not less than T7_{TH}, execution of the control routine 1454 advances to step 1466 where the PLC circuit 120 is operable to control the water inlet valve 1416, by producing an appropriate signal on signal path 130₂₉, to maintain the current opening of the water inlet valve 1416 to thereby maintain the current value of the temperature T₇. Execution of the control routine 1454 loops from either of steps 1464 and 1466 back to step 1456.

Referring now to FIG. 29, a schematic diagram of one illustrative embodiment of the residual liquid processing unit 16 and corresponding control system that forms part of the biomaterial waste processing system 10 of FIG. 1 is shown. In the illustrated embodiment, an inlet diverter valve 1480 has an inlet fluidly coupled to the residual liquid inlet, RLI, of the residual liquid processing unit 16 and to the residual liquid inlet conduit 74. One outlet of the inlet diverter valve 1480 is fluidly coupled via a conduit 1482 to a residual liquid inlet of a first precipitation tank 1484, and another outlet of the inlet diverter valve 1480 is fluidly coupled via a conduit 1486 to a second precipitation tank 1488. The inlet diverter valve 1480 is electrically connected to an actuator output of the PLC circuit 140 via signal path 150₁, and the PLC circuit 140 is operable to control the diverter valve 1480, by producing an appropriate signal on signal path 150₁, between one position fluidly coupling the inlet of the inlet diverter to the inlet diverter valve outlet fluidly coupled to conduit 1482, and another position fluidly coupling the inlet of the inlet diverter valve to the inlet diverter valve outlet fluidly coupled to conduit 486. The precipitation tanks 1484 and 1488 are conventional tanks configured to receive and hold a quantity of liquid therein, and the first precipitation tank 1484 includes a level sensor producing a signal indicative of the level of liquid contained therein. Similarly, the second precipitation tank 1488 includes a level sensor producing a signal indicative of the level of liquid contained therein. In the illustrated embodiment, these level sensors are provided in the form of a pressure sensor 142₁ disposed in fluid communication with the interior of the first precipitation tank 1484 and electrically connected to a sensor input of the PLC circuit 140 via signal path 144₁, and a pressure sensor 142₂ disposed in fluid communication with the interior of the second precipitation tank 1488 and electrically connected to a sensor input of the PLC circuit 140 via signal path 144₂. The PLC circuit 140 is configured to process the signals produced by the pressure sensors 142₁ and 142₂ and determine corresponding levels of liquid in the precipitation tanks 1484 and 1488 respectively. Alternatively, one or more other known level sensors may be used with tanks 1484 and 1488 to produce one or more corresponding signals indicative of the liquid levels in the tanks 1486 and 1488.

A precipitation catalyst solution tank 1490 has a fluid outlet coupled through a control valve 1494 to an inlet of a conventional liquid pump 1496, and the outlet of the pump 1496 is fluidly coupled to the inlet of the inlet diverter valve 1480 via a conduit 1500. The pump 1496 is electrically connected to a conventional pump driver 1498 that is also electrically connected to an actuator output of the PLC circuit 140 via signal path 150₃. The PLC circuit 140 is configured to control the speed of the pump 1496 in a known manner by producing an appropriate actuator control signal on signal path 150₃. The control valve 1494 is electrically connected to another actuator output of the PLC circuit 140 via signal path 150₂, and the PLC circuit 140 is configured to control operation of the control valve by producing an appropriate actuator control signal on signal path 150₂. The precipitation catalyst solution tank 1490 is mechanically coupled to a conventional motor 1502, which is electrically connected to a conventional motor driver 1504. The motor driver 1504 is electrically connected to another actuator output of the PLC circuit 140 via signal path 1504. The PLC circuit 140 is configured to control the operation of the motor 1502 by producing an appropriate actuator control signal on signal path 150₄.

The precipitation catalyst solution tank 1490 is filled with a precipitation catalyst solution, and the PLC circuit 140 is configured to periodically activate the motor 1502 for a predefined time period to mix the precipitation catalyst solution within the tank 1490. In the illustrated embodiment, the PLC circuit 140 is further configured to maintain the control valve 1494 open and to control the speed of the pump 1496 to supply the precipitation catalyst solution to the inlet of the inlet diverter valve 1480 at a target precipitation catalyst solution flow rate. The precipitation catalyst solution thus mixes with the residual liquid supplied to the inlet of the inlet diverter valve 1480 via conduit 74, and this mixture is then supplied to the precipitation tanks 1484 and 1488 in alternating fashion via control of the inlet diverter valve. The precipitation catalyst solution is selected to modify the residual liquid supplied via conduit 74 in a manner that will facilitate precipitation of residual waste out of the residual liquid within the precipitation tanks 1484 and 1488. For example, residual liquids resulting from fermentation of biomaterial waste, such as animal waste, may have residual phosphorus-based components or nutrients. Suitable precipitation catalyst solutions may include, but are not limited to, clay, ferric-clay, limestone, ferric limestone, calcium carbonate, calcium carbonate-iron complexes, vermiculites, silica, aluminum silicates, bentonites, and the like, and combinations thereof.

The first precipitation tank 1484 further includes a pH adjustment solution inlet fluidly coupled to an outlet of a control valve 1512 via an inlet conduit 1510. The second precipitation tank 1488 also includes a pH adjustment solution inlet fluidly coupled to an outlet of another control valve 1518 via an inlet conduit 1516. The control valve 1512 is electrically connected to another actuator output of the PLC circuit 140 via signal path 150₅, and the control valve 1518 is electrically connected to yet another actuator output of the PLC circuit 140 via signal path 150₆. The PLC circuit 140 is operable to control the operation of each of the control valves 1512 and 1518 by producing appropriate actuator control signals on signal paths 150₅ and 150₆ respectively. The inlets of valves 1512 and 1518 are fluidly coupled to an outlet of a conventional liquid pump 1514 having a pump inlet fluidly connected to an outlet of another control valve 1524 via a conduit 1522. The pump 1514 is electrically connected to a conventional pump driver 1520 that is also electrically connected to an actuator output of the PLC circuit 140 via signal path 1507. The PLC circuit 140 is configured to control the speed of the pump 1514 in a known manner by producing an appropriate actuator control signal on signal path 150₇.

The outlet of the control valve 1524 is coupled to a fluid outlet of a pH adjustment solution tank 1526, and the control valve 1524 is electrically connected to another actuator output of the PLC circuit 140 via signal path 150₈. The PLC circuit 140 is configured to control operation of the control valve 1524 by producing an appropriate signal on signal path 150₈. The pH adjustment solution tank 1526 is mechanically coupled to a conventional motor 1528, which is electrically connected to a conventional motor driver 1530. The motor driver 1530 is electrically connected to another actuator output of the PLC circuit 140 via signal path 150₉. The PLC circuit 140 is configured to control the operation of the motor 1528 by producing an appropriate actuator control signal on signal path 150₉.

The pH adjustment solution tank 1526 is filled with a pH adjustment solution, and the PLC circuit 140 is configured to periodically activate the motor 1528 for a predefined time period to mix the pH adjustment solution within the tank 1526. In the illustrated embodiment, the PLC circuit 140 is further configured to maintain the control valve 1524 open and to control the speed of the pump 1514 to supply the pH adjustment solution to the inlets of the control valves 1512 and 1518 at a target pH adjustment solution flow rate. The PLC circuit 140 is further configured to control operation of the control valves 1512 and 1518 to selectively supply the pH adjustment agent to the precipitation tanks 1484 and 1488 in alternating fashion. The pH adjustment solution is selected to controllably change the pH level of the residual liquid and precipitation catalyst solution mixture in each of the precipitation tanks 1484 and 1488 to thereby precipitate residual waste out of the residual liquid to produce “cleaned” water that is substantially free of harmful organic or inorganic chemical substances and that can safely be released from the residual liquid processing unit 16 as ground water. For residual liquids resulting from fermentation of biomaterial waste in the form of animal waste, suitable pH adjustment solutions may include, but are not limited to, lime, calcium carbonate, iron-fortified calcium carbonate, and the like, and combinations thereof.

The first precipitation tank 1484 further includes a cleaned water outlet fluidly coupled to one inlet of an outlet diverter valve 1542 via a cleaned water outlet conduit 1544, and the second precipitation tank 1488 also has a cleaned water outlet fluidly coupled to another inlet of the outlet diverter valve 1542 via another cleaned water outlet conduit 1540. An outlet of the outlet diverter valve 1542 is fluidly coupled through a mechanical on/off valve, MV, and a butterfly valve, BV, to an inlet of another conventional liquid pump 1548 having a pump outlet fluidly coupled through additional mechanical on/off valves, MV, to the first and second liquid outlets, LO1 and LO2, of the residual liquid processing unit 594, and thus to the liquid outlet conduits 78 and 82 respectively. The mechanical valves, MV, at the liquid outlets LO1 and LO2 may be suitably manipulated to direct the flow of liquid from the pump 1548 out of the residual liquid processing unit 16 via conduit 82, or alternatively back to the liquefied waste source 20 via conduit 76. In any case, the outlet diverter valve 1542 is electrically connected to an actuator output of the PLC circuit 140 via signal path 150₁₀, and the PLC circuit 140 is configured to control operation of the outlet diverter valve 1542 by producing an appropriate signal on signal path 150₁₀. The pump 1548 is electrically connected to a conventional pump driver 1550 that is also electrically connected to an actuator output of the PLC circuit 140 via signal path 1501. The PLC circuit 140 is configured to control the speed of the pump 1548 in a known manner by producing an appropriate actuator control signal on signal path 150₁₁. In operation, the PLC circuit 140 is operable to control the position of the outlet diverter valve 1542 and the speed of the pump 1548 to selectively remove the cleaned water from the precipitation tanks 1484 and 1488 in alternating fashion.

The first precipitation tank 1484 further includes a precipitated waste outlet fluidly coupled to an inlet of a control valve 1572 via a conduit 1570. The second precipitation tank 1488 also includes a precipitated waste outlet fluidly coupled to an inlet of another control valve 1562 via a conduit 1560. The control valve 1562 is electrically connected to another actuator output of the PLC circuit 140 via signal path 150₁₂, and the control valve 1572 is electrically connected to yet another actuator output of the PLC circuit 140 via signal path 150₁₃. The PLC circuit 140 is operable to control the operation of each of the control valves 1562 and 1572 by producing appropriate actuator control signals on signal paths 150₁₁ and 150₁₂ respectively. The outlets of the control valves 1562 and 1572 are fluidly coupled to an inlet of another conventional pump 1564 via a conduit 1568, and an outlet of the pump 1564 is fluidly coupled to the precipitated waste outlet, PWO, of the residual liquid processing unit 16 and also to the precipitated waste outlet conduit 80. The pump 1564 is electrically connected to a conventional pump driver 1574 that is also electrically connected to an actuator output of the PLC circuit 140 via signal path 150₁₄ and also to a sensor input of the PLC circuit 140 via signal path 1443. The PLC circuit 140 is configured to control the speed of the pump 1564 in a known manner by producing an appropriate actuator control signal on signal path 150₁₄. The pump driver 1574 is responsive to an actuator control signal supplied by the PLC 140 on signal path 150₁₄ to drive the pump 1564, and the pump driver 1574 and/or pump 1564 further includes a “sensor” for determining and monitoring the operating torque of the pump 1564. Such a “sensor” may be a conventional strain-gauge type torque sensor operatively coupled to a rotating drive shaft of the pump 1564 and operable to produce a sensor signal corresponding to the operating torque of the pump 1564, or may alternatively be a so-called virtual sensor implemented in the form of one or more software algorithms resident within the PLC circuit 140 and responsive to one or more measurable operating parameters associated with the pump driver 1574 and/or pump 1564 to derive or infer the operating torque value. For example, the pump driver 1574 may include a current sensor producing a current sensor signal indicative of drive current being drawn by the pump driver 1574, and/or the pump 1564 may include a position and/or speed sensor producing a signal corresponding to the rotational speed and/or position of the pump 1564. The PLC circuit 140 may be responsive to any such sensor signals, and/or to other information relating to the operation of the pump driver 1574 and/or pump 1564, to estimate the operating torque of the pump 1564 as a known function thereof. In any case, the signal path 1443 carries one or more torque feedback signals to the PLC circuit 140 from which the operating torque of the pump 1564 may be determined directly or estimated. In operation, the PLC circuit 140 is operable to control operation of the control valves 1562 and 1572 and the speed of the pump 1564 to selectively remove precipitated waste from the precipitation tanks 1484 and 1488 in alternating fashion.

The residual liquid processing unit 16 is operable, under control of the PLC circuit 140, to fill one of the precipitation tanks 1484, 1488 with the residual liquid and precipitation catalyst solution mixture while the other tank 1484, 1488 is emptied of water. As either of the precipitation tanks 1484, 1488 is being filled, the pH adjustment solution is added to controllably change the pH level of the mixture and cause excess waste in the residual liquid to precipitate out. The timing of the inlet diverter valve 1480 and the outlet diverter valve 1542, and of the control valves 1512 and 1518, as well as the speed of the liquid outlet pump 1548, are controlled by the PLC circuit 140 so that while one of the precipitation tanks 1484, 1488 is being filled, the other tank 1484, 1488 is being emptied. Removal of the precipitated waste need only occur occasionally; e.g., every several days or weeks, and operation of the control valves 1562 and 1572 and of the solids outlet pump 1564 may therefore be independent and asynchronous with the remaining components of the residual liquid processing unit 16.

Referring now to FIG. 30, a flowchart of one illustrative embodiment of a software control algorithm 1600 for controlling the residual liquid processing unit 16 of FIG. 29 is shown. It will be understood that the software algorithm 1600 represents one illustrative strategy for controlling the residual liquid processing unit 16 during normal, continuous flow operation of the biomaterial waste processing system 10, and that the residual liquid processing unit 16 may be controlled differently during other operational modes of the biomaterial waste processing system 10. The software algorithm 1600 includes a number of different and independently executing control routines, and each of these different control routines will be described separately. For example, the control algorithm 1600 includes a first control routine 1602 for controlling the filling and emptying of the precipitation tanks 1484 and 1488. The control routine 1602 begins at step 1604 where the PLC circuit 140 is operable to control the precipitation catalyst pump 1496 to a target pump speed, S1. In the illustrated embodiment, the target pump speed, S1, is selected to provide a target flow rate of the precipitation catalyst solution from the precipitation catalyst solution tank 1490 to the inlet of the inlet diverter valve 1480, wherein this target flow rate is dependent on a number of factors including, but not limited to, the flow rate and flow volume of the residual liquid supplied to the inlet of the inlet diverter valve 1480, the desired ratio of residual liquid and precipitation catalyst solution, the chemical make up of the precipitation catalyst solution, and the like. In any case, for normal, continuous flow operation of the biomaterial waste processing system 10, the residual liquid flows into the residual liquid inlet, RLI, of the residual liquid processing unit 16 at a substantially constant rate, and the target speed, S1, of the precipitation catalyst pump 1496 will accordingly be a substantially constant pump speed.

Following step 1604, the PLC circuit 140 is operable at step 1606 to control the inlet diverter valve 1480 to fill one of the precipitation tanks 1484, 1488 with the residual liquid and precipitation catalyst solution mixture while the other precipitation tank 1484, 1488 is being emptied. In the illustrated embodiment, the PLC circuit 140 is configured to execute step 1606 by controlling the inlet diverter valve 1480, via an appropriate actuator control signal on signal path 1501, to fluidly couple the inlet of the inlet diverter valve 1480 to conduit 1482 to fill the first precipitation tank 1484 with the residual liquid and precipitation catalyst solution mixture, or to fluidly couple the inlet of the inlet diverter valve 1480 to conduit 1486 to fill the second precipitation tank 1488 with the residual liquid and precipitation catalyst solution mixture. Thereafter at step 1608, the PLC circuit 140 is operable to control the pH adjustment solution pump 1514 to a target pump speed, S2, and to control the pH adjustment solution inlet valves 1512 and 1518 to introduce the pH adjustment solution to the precipitation tank 1484, 1488 being filled. In one illustrative embodiment, as described hereinabove, the PLC circuit 140 is operable to continuously control the pH adjustment solution pump 1514 to the target pump speed, S2, wherein the target pump speed, S2, is selected to provide a continuous target flow rate of the pH adjustment solution from the pH adjustment solution tank 1526 to the pH adjustment solution inlet valves 1512 and 1518. In this embodiment, the PLC circuit 140 is operable to execute step 1608 by controlling the flow of the pH adjustment solution to the precipitation tanks 1484, 1488 via control of the inlet valves 1512 and 1518 in alternating fashion. Alternatively, the PLC circuit 140 may be configured to execute step 1608 by simultaneously opening an appropriate one of the inlet valves 1512, 1518 while closing the other inlet valve 1512, 1518 and activating the pH adjustment solution pump 1514 at the target pump speed, S2, and otherwise maintaining the inlet valves 1512 and 1518 closed and deactivating the pump 1514. In either case, the target pump speed, S2, is selected to provide a target flow rate of the pH adjustment solution from the pH adjustment tank 1526 to the pH adjustment solution inlet of the precipitation tanks 1484, 1488, wherein this target flow rate is dependent on a number of factors including, but not limited to, the mixture fill rate and fill volume of the precipitation tanks 1484, 1488, the timing, relative to the process of filling the precipitation tanks 1484, 1488 with the residual liquid and precipitation catalyst mixture, that the pH adjustment solution is added to the precipitation tanks 1484, 1488, the desired ratio of residual liquid and pH adjustment solution, the chemical make up of the pH adjustment solution, and the like.

Following step 1608, the PLC circuit 140 is operable at step 1610 to determine the level, L1, of the fluid in the precipitation tank 1484, 1488 being filled. In the illustrated embodiment, the PLC circuit 140 is configured to execute step 1610 by monitoring the signal produced by an appropriate one of the pressure sensors 142₁, 142₂ on a corresponding signal path 144₁, 144₂, and processing the pressure signal in a known manner to determine L1. It will be understood, however, that the PLC circuit 140 may be alternatively configured to determine the liquid level, L1, in accordance with any one or more other known liquid level determining techniques using any one or more other known sensors from which L1 may be determined directly or indirectly. In any case, execution of the control routine 1602 advances from step 1610 to step 1612 where the PLC circuit 140 is operable to compare L1 to a threshold level value, L1_{TH}, where L1_{TH} represents a level at which the precipitation tanks 1484, 1488 are considered to be full. If, at step 1612, L1 is less than L1_{TH}, execution of the control routine 1602 loops back to step 1610 to continue to determine and monitor L1. If, however, L1 is greater than or equal to L1_{TH} at step 1612, execution of the control routine 1602 advances to step 1614 where the PLC circuit 140 is operable to control the outlet diverter valve 1542, and operate the outlet pump 1548 at a target speed, S3, to begin emptying cleaned water from the now filled precipitation tank 1484, 1488.

In the illustrated embodiment, the PLC circuit 140 is operable to continuously control the outlet pump 1548 to the target pump speed, S3, wherein the target pump speed, S3, is selected to remove the cleaned water from the precipitation tanks 1484, 1488 at a continuous target flow rate. In this embodiment, the PLC circuit 140 is operable to execute step 1614 by controlling the outlet diverter valve 1542, in alternating fashion, to one position fluidly coupling the cleaned water outlet conduit 1544 to the outlet of the diverter valve 1542 to thereby remove cleaned water from the first precipitation tank 1484, or by controlling the outlet diverter valve 1542 to an opposite position coupling the cleaned water outlet conduit 1540 to the outlet of the diverter valve 1542 to thereby remove cleaned water from the second precipitation tank 1488. In either case, the target pump speed, S3, is selected to remove cleaned water from the precipitation tanks 1484, 1488 at a target flow rate, wherein the target flow rate is dependent on a number of factors including, but not limited to, the mixture fill rate and fill volume of the precipitation tanks 1484, 1488, the timing, relative to the process of filling the precipitation tanks 1484, 1488 with the residual liquid and precipitation catalyst mixture, that the pH adjustment solution is added to the precipitation tanks 1484, 1488, the rate of nutrient precipitation in the precipitation tanks 1484, 1488, and the like. Execution of the control routine 1602 loops from step 1614 back to step 1606.

Step 1606 of the control routine 1602 also advances to step 1616 where the PLC circuit 140 is operable to determine the level, L2, of the fluid in the precipitation tank 1484, 1488 being emptied. In the illustrated embodiment, the PLC circuit 140 is configured to execute step 1616 by monitoring the signal produced by an appropriate one of the pressure sensors 1421, 1422 on a corresponding signal path 144₁, 144₂, and processing the pressure signal in a known manner to determine L2. It will be understood, however, that the PLC circuit 140 may be alternatively configured to determine the liquid level, L2, in accordance with any one or more other known liquid level determining techniques using any one or more other known sensors from which L2 may be determined directly or indirectly. In any case, execution of the control routine 1602 advances from step 1616 to step 1618 where the PLC circuit 140 is operable to compare L2 to a threshold level value, L2_{TH}, where L2_{TH} represents a level at which the precipitation tanks 1484, 1488 are considered to be emptied of cleaned water. If, at step 1618, L1 is greater than L2_{TH}, execution of the control routine 1602 loops back to step 1616 to continue to determine and monitor L2. If, however, L2 is less than or equal to L2_{TH} at step 1618, execution of the control routine 1602 advances to step 1620 where the PLC circuit 140 is operable to control the outlet diverter valve 1542 to begin removing cleaned water from the opposite precipitation tank 1484, 1488. Execution of the control routine 1602 loops from step 1620 back to step 1606.

For normal, continuous flow operation of the residual liquid processing unit 16, control routine 1602 is coordinated in the timing of its various execution branches so that one precipitation tank 1484, 1488 is being filled with the residual liquid and precipitating catalyst solution mixture while the other precipitation tank 1484, 1488 is being simultaneously emptied of cleaned water. In such a continuous flow system, steps 1614 and 1620 thus loop directly back to step 1606 of control routine 1602. For non-continuous flow operation, control routine 1602 may require one or more delay steps to coordinate the filling of one precipitation tank 1484, 1488 with the emptying of the other precipitation tank 1484, 1488.

In any case, the residual liquid processing unit control algorithm 1600 includes another control routine 1622 that operates independently of control routine 1602 so that precipitated waste may be periodically extracted from the precipitation tanks 1484, 1488 independently from the liquid filling and emptying operations. Control routine 1622 begins at step 1624 where the PLC circuit 140 is operable to periodically control the precipitated waste outlet valves 1562 and 1572 and activate the precipitated waste extraction pump 1564 to extract precipitated waste from each of the precipitation tanks 1484, 1488. The PLC circuit 140 may be operable to control the precipitated waste outlet valves 1562 and 1572 to extract precipitated waste from both of the precipitation tanks 1484 and 1488 simultaneously, or may alternatively control the precipitated waste outlet valves 1562 and 1572 to extract precipitated waste from only one of the precipitation tanks 1484, 1488 at a time. In either case, execution of the control routine 1622 advances from step 1624 to step 1626 where the PLC circuit 140 is operable to determine the operating torque, TQ, of the precipitated waste extraction pump 1564. In the illustrated embodiment, the PLC circuit 140 is operable to execute step 1626 using any of the feedback torque monitoring techniques described hereinabove.

Following step 1626, the PLC circuit 140 is operable at step 1628 to compare the operating torque, TQ, of the precipitated waste extraction pump 1564 to a torque threshold, TQ_{TH}. As precipitated waste is extracted from either, or both, of the precipitation tanks 1484, 1488, the operating torque of the pump 1564 will decrease due to the diminishing quantity of the precipitated waste in either, or both, of the precipitation tanks 1484, 1488. The torque threshold TQ_{TH} corresponds to an operating torque of the pump 1564 below which either, or both, of the precipitation tanks 1484, 1488 may be considered to be sufficiently emptied of precipitated waste. Thus, if the PLC circuit 140 determines at step 1628 that TQ is greater than or equal to TQ_{TH}, either, or both, of the precipitation tanks 1484, 1488 still hold a quantity of precipitated waste that may be removed, and execution of the control routine 1622 thus loops back to step 1626. If, however, the PLC circuit 140 determines at step 1628 that TQ is less than TQ_{TH}, sufficient precipitated waste has been extracted from either, or both, of the precipitation tanks 1484, 1488 to consider it/them emptied of precipitated waste, and execution of the control routine 1622 advances to step 1630 where the PLC circuit 140 is operable to deactivate the precipitated waste extraction pump 1564 and close either, or both, of the outlet valves 1562, 1572. From step 1630, execution of the control algorithm 1622 loops back to step 1624.

Further details relating to the interaction between the residual liquid supplied to the residual liquid inlet, RLI, of the residual liquid processing unit 16 and the precipitation catalyst solution, as well as the interaction between the residual liquid and precipitation catalyst solution mixture and the pH adjustment solution, are disclosed in PCT/US2005/______, entitled SYSTEM FOR REMOVING SOLIDS FROM AQUEOUS SOLUTIONS (attorney docket no. 35479-77847) which is assigned to the assignee of the present invention and is incorporated herein by reference.

It will be understood that while many of the actuator drivers illustrated the drawings have been described as being controlled relative to maximum or minimum output torque values, at least some of such actuator drivers, particularly those driving some augers and pumps, may be conventional variable frequency drivers (VFD) capable of operating, at least for brief periods, at output torque values well above their rated maximum output torque values. When high starting torque or intermittent high torque loads are expected, e.g., as may be the case with one or more of the sand augers illustrated and described herein, such VFD's may be operated well above their rated maximum output torque values, e.g., 180% of the rated maximum, for brief time periods, e.g., 1-3 seconds, in order to “break” inertia or to overcome a high start-up load.

While the invention has been illustrated and described in detail in the foregoing drawings and description, the same is to be considered as illustrative and not restrictive in character, it being understood that only illustrative embodiments thereof have been shown and described and that all changes and modifications that come within the spirit of the invention are desired to be protected. For example, the various software algorithms and control structures described herein are provided to illustrate example operation of the biomaterial waste processing system 10 in a normal, continuous flow operating mode with the biomaterial waste being comprised of livestock waste. It will be appreciated that the biomaterial waste processing system 10 may include additional or alternative control algorithms when operating in modes other than normal, continuous flow operation and/or with biomaterial waste comprised of biomaterial waste other than livestock waste. In any case, such other software algorithms and control structures are intended to fall within the scope of the claims appended hereto.

### Illustrative Embodiments of a Process and Apparatus for Treatment of a Biomaterial Waste Stream

Illustrative biomaterial waste streams that can be treated with the treatment processes described herein include, but are not limited to, manure, cellulosistic solid waste, feathers, hair, whey broth from cheese production or biomaterial waste streams from other foodstuffs, broth remediation from alcohol or yeast production, tannery waste, slaughterhouse waste, tallow waste from rendering processes and including waste fats and oils, waste derived from plants, paper processing waste, land fill waste, and the like. The waste derived from plants can be, for example, waste from hay, leaves, weeds, sawdust, or wood and can be, for example, yard waste, landscaping waste, agricultural crop waste, forest waste, pasture waste, or grassland waste. The waste derived from foodstuffs can be fruit and vegetable processing waste, fish and meat processing wastes, bakery product waste, cheese whey, and the like. In embodiments where the waste is manure, the manure can be from an animal such as a human, a bovine animal, an equine animal, an ovine animal, a porcine animal, or poultry. In general, any organic waste containing proteins, simple carbohydrates, complex carbohydrates, lipids, and combinations thereof, can be pretreated as described herein.

In one embodiment, the processes described herein may be used for a wide variety of biomaterial waste streams for removing pollutants from the biomaterial waste stream, and alternatively converting the pollutants to a valuable product by fermentation. The treated biomaterial waste stream may be further processed using any number of additional apparatus or processes including those used to process biomaterial waste streams by fermentation, such as the systems, processes, and apparatus described herein and in PCT applications serial. nos. PCT/US2005/______, entitled SYSTEM FOR PROCESSING A BIOMATERIAL WASTE STREAM (attorney docket no. 35479-77858), PCT/US2005/______, entitled FERMENTER AND FERMENTATION METHOD (attorney docket no. 35479-77851), PCT/US2005/______, entitled SYSTEM FOR REMOVING SOLIDS FROM AQUEOUS SOLUTIONS (attorney docket no. 35479-77847) incorporated herein by reference. Further, the biomaterial waste stream may be directly derived from the source producing the waste, or may be the product of another process, method, system, or apparatus for treating biomaterial waste streams directly derived from the source producing the waste, including but not limited to the methods, processes, and apparatus described in PCT/US2005/______, entitled SAND AND ANIMAL WASTE SEPARATION SYSTEM (attorney docket no. 35479-77857) incorporated herein by reference.

In one embodiment, the biomaterial waste stream is a variable and dilute biomaterial waste stream derived from animal manure including waste from barn animals ruminants and partial ruminants, such as beef cattle, dairy cattle, and horses, and/or from swine, poultry, and the like.

In one embodiment, the treatment processes and apparatus described herein include a separating step. The separating step may be based on separating components having differing sizes, densities, or other distinguishing properties. In an embodiment where the biomaterial waste stream is derived from animal manure, the biomaterial waste stream can include higher density components such as sand, dirt, gravel, and the like, and combinations thereof; and lower density components such as fiber, hay, straw, bedding straw, sawdust, other cellulosistic material, hair, completely and incompletely digested feed, including protein and protein digestion residues, whole grain, spilled feed, and the like, and combinations thereof. Alternatively, these same components found in biomaterial waste streams derived from animal manure may be separated from each other according to relative size. In any case, separation of one component class from the other is contemplated in the processes and apparatus described herein. In one aspect, where the separation of components in the biomaterial waste stream is a density-based separation, the lower density components may have a high level of cellulose, hemicellulose, and cellulose-related components. The higher density material may be separated from the lower density material, and each separated from the liquefied waste; or the small particles may be separated from the large particles, and each separated from the liquefied waste; by any conventional solid/liquid separation process, or by introducing the biomaterial waste stream into the solid/liquid separation unit described herein and in PCT/US2005/______ entitled SYSTEM FOR PROCESSING A BIOMATERIAL WASTE STREAM (attorney docket no. 35479-77858).

In variations of the processes and apparatus described herein, the biomaterial waste stream from barn animals includes dissolved and undissolved components that may be precipitated by admixing with aggregation agents or catalysts, binding agents, and the like, by heat treatment, by adjusting the pH, and similar processes, and combinations thereof. Once precipitated, these additional components may be separated in a solid/liquid separation unit as described above.

In one aspect, biomaterial waste streams from barn animals include manure from full ruminants such as mature cattle, beef cattle, and dairy cattle. In another aspect, biomaterial waste streams from barn animals include manure from semi-ruminants or partial ruminants, such as horses. It is appreciated that biomaterial waste streams from semi-ruminants may include more or substantially more cellulose fiber, and/or less or substantially less completely digested material than biomaterial waste streams from fall ruminants. It is also appreciated that the treatment of biomaterial waste streams from semi-ruminants, using the processes and apparatus described herein, may include more vigorous or harsher conditions than included in comparable treatment of biomaterial waste streams from fall ruminants. Harsher and/or more vigorous conditions include higher temperatures, more extreme pH levels such as more acidic or more basic pH levels, higher acid concentrations, higher base concentrations, more aggressive enzymes, less selective enzymes, enzymes with higher turnover rates, more aggressive microorganism, and the like.

In another aspect of biomaterial waste streams derived from ruminant and partial or semi-ruminant animals, the waste stream may have a relatively high proportion of lignin. It is understood that ruminant and semi ruminant animals more efficiently remove useful nutrients, such as carbohydrates, from the fiber component of their feed than do other animals, such as swine and poultry. Therefore, it is appreciated that the lignin fraction is effectively concentrated and forms a relatively higher proportion in the waste from ruminant and semi-ruminant animals.

In another embodiment, the biomaterial waste stream is derived from animal manure, such as manure from swine, and includes higher density components such as sand, dirt, gravel, and the like, and combinations thereof; and lower density components such as fiber, hay, straw, bedding straw, sawdust, celluloses, hemicelluloses, cellulose related components, other cellulosistic material, incompletely digested feed such as grain residues, corn meal, soy meal, and the like, whole grain, spilled grain, hair, proteins, bile acids, starches, starch granules, and the like, and combinations thereof. In variations, the components in the biomaterial waste stream are distinguished and separated by particle size rather than density. It is appreciated that this biomaterial waste stream may be directly obtained from the animal, or may be the product of other processes and apparatus as described herein. In addition, dissolved and undissolved components including proteins, bile acids, starches, starch granules, and the like, may be precipitated or aggregated to increase the amount of lower density material. The lower density material, or certain sized components may be separated from other components as described herein.

In variations of the processes and apparatus described herein, the biomaterial waste stream from swine includes dissolved and undissolved components that may be precipitated by admixing with aggregation agents or catalysts, binding agents, and the like, by heat treatment, by adjusting the pH, and similar processes, and combinations thereof. Once precipitated, these additional components may be separated in a solid/liquid separation unit as described above. Such additional components include proteins, organic acids, bile acids, complex starches, and cellulose-related molecules, including cellulose and hemicellulose.

In one aspect of biomaterial waste streams from swine, undigested or incompletely digested grain, soy and/or corn meal, and complex starches may each be present. It is appreciated that a high proportion of the phosphorus in many grains is in the form complex organic molecules, such as phytic acid and other phosphoinositols, and is not well-digested, especially by nonruminants including swine. It is further appreciated that inorganic phosphate may be recovered from such complex organic molecules by fall or partial hydrolysis, generally at low pH, and/or by hydrolysis using enzymes including phytases.

In one aspect of treating biomaterial waste streams from swine, the treated waste is used as a liquid waste stream for a fermentation process, such as the fermentation processes described herein. It is understood that such a treated waste includes nutrients that are used by the fermenting organism. It is appreciated that the treatment steps described herein may be performed in a manner that maximizes the production of nutrients usable by the fermenting organism. Therefore, in some aspects, the pH of biomaterial waste stream from swine is adjusted to lower levels. Without being bound by theory, it is believed that such lower pH levels not only facilitate many of the treatment processes described herein, but also stabilize nutrients already present and those produced in the swine waste stream.

In another embodiment, the biomaterial waste stream is derived from animal manure, including manure from poultry, such as chickens, ducks, turkeys, and the like, and includes higher density components such as sand, dirt, gravel, and the like, and combinations thereof; and lower density components such as feathers, fiber, hay, straw, bedding straw, sawdust, other cellulosistic material, and the like, and combinations thereof. In variations, the components in the biomaterial waste stream are distinguished and separated by particle size rather than density. It is appreciated that this biomaterial waste stream may be directly obtained from the animal, or may be the product of other processes and apparatus as described herein. The lower density material, or certain sized components may be separated from other components as described herein.

In variations of the processes and apparatus described herein, the biomaterial waste stream from poultry includes dissolved and undissolved components that may be precipitated by admixing with aggregation agents or catalysts, binding agents, and the like, by heat treatment, by adjusting the pH, and similar processes, and combinations thereof. Once precipitated, these additional components may be separated in a solid/liquid separation unit as described above. Illustrative aggregation or precipitation catalysts for protein components includes sulfate salts such as sodium sulfate, ammonium sulfate, calcium salts, iron-calcium complexes, transition metals, metal complexes, and the like.

In one aspect of biomaterial waste streams from poultry, undigested or incompletely digested grain, corn meal and/or soy meal, and other complex starches may each be present. In addition, it is appreciated that a high proportion of the phosphorus in many grains is in the form of complex organic molecules, such as phytic acid, and these complex organic molecules are not always well-digested by poultry. It is further appreciated that inorganic phosphate may be recovered from such complex organic phosphate molecules by fall or partial hydrolysis, generally at low pH, and/or by hydrolysis using enzymes including phytases.

In embodiments of the processes and apparatus described herein that include fermentation, it is appreciated that many of the components in the animal waste are nutrients used by the fermenting organism, including urine, such as ammonia, amines, urea, indole, and other nitrogen compounds, phosphates and other salts, amino acids, and other organic acids, including acetic, butyric, valeric, and other acids.

It is appreciated that a solid component containing one or more fiber-like materials may be difficult to process in conventional fermentation systems until the solid component is treated, such as by solubization and/or degradation to smaller molecular weight, or more water soluble components. It is also appreciated that a solid component containing certain proteins, peptides, organic acids, organic phosphates, organic amines, and complex starches may be difficult to process in conventional fermentation systems until the solid component is treated, such as by solubization and/or degradation to smaller molecular weight, or more water soluble components. Pretreatment of the solid component in a chemical process, enzymatic process, or microbial process may convert portions of the component into a product that may be recombined with the liquefied waste prior to additional processing, including sterilization, fermentation, and the like.

In one aspect, prior to chemical, enzymatic, or microbial processing, the solid component is a lower density component including fiber-like materials. The fiber-like component may be dried, squeezed, drained, filtered, pressed, centrifuged, evaporated, and the like, and/or processed in a like manner to remove water. It is appreciated that removing water from the fiber-like component may decrease the quantities of chemicals, enzymes, and/or microorganisms needed for treatment or processing. It is also understood that removal of too much water from the fiber-like component may adversely affect mechanical processing, such as decreased ability to stir, and the like. In one aspect, the ratio of water to solids is in the range from about 2 to about 10, and is illustratively about 6. In another aspect, the ratio of water to solids is about 2.

In another aspect, the lower density component includes fiber, hay, straw, bedding straw, sawdust, other cellulosistic material, and the like, and combinations thereof. It is appreciated that the lower density component containing fiber-like material may represent as much as about 50% of the total solid content (dissolved and undissolved) present in the biomaterial waste stream. Illustratively, the fiber-like material represents about one-third of the total solid content. In another aspect, the lower density component includes feathers and/or hair. In another aspect, the lower density component includes proteins, polypeptides, peptides, organic acids, organic phosphates, organic amines, and the like, and combinations thereof that may be precipitated or otherwise aggregated.

In another aspect, prior to chemical, enzymatic, and/or microbial processing, the solid component includes proteins, peptides, organic acids, organic phosphates, organic amines, and complex starches, that may be optionally precipitated. The solid component may be in the form of a paste or sludge that is resuspended to form a liquid waste slurry suitable for chemical, enzymatic, or microbial processing. The liquid waste slurry illustratively has a solids content in the range from about 1% to about 10%, and is illustratively 4%, relative to moisture and an ash-free weight determination.

In one embodiment, chemical processing of the solid component containing fiber-like material, feathers, or precipitated material may be performed by treating the component with an acid including, but not limited to, inorganic or mineral acids such as hydrochloric acid, hydrobromic acid, sulfuric acid, and acidic salts thereof, phosphoric acid, and acidic salts thereof, and the like; and organic acids such as carbonic acid, formic acid, acetic acid, and the like; and combinations thereof. Acids may be used at high acidic pH or at low acidic pH, and at high concentration, and at low concentration. Illustrative pH levels include those in the range from about −1 to about 4, and in the range from about 0 to about 2. Illustrative concentrations include those in the range from about 0.01 M to about 5 M, and 0.1 M to about 1 M. In one aspect, concentrated sulfuric acid is added to the solid component, including sulfuric acid concentrations in the range from about 70% to about 95%. In another aspect, 78% or 72% sulfuric acid is added to the solid component. In another aspect, dilute sulfuric acid in the range from about 1% to about 10%, and illustratively 3% is added to the solid component.

In another embodiment, chemical processing of the solid component containing fiber-like material, feathers, or precipitated material may be performed by treating the component in a two-stage process, where the first stage includes treating the component with a high concentration of acid, such as a concentration in the range from about 60% to about 90%, and subsequently treating the component with a low concentration of acid, such as a concentration in the range from about 1% to about 30%. In the first stage, solubilization of the fiber-like material, feathers, or precipitated material may occur. It is appreciated that hydrolysis of the fiber-like material, feathers, or precipitated material may also occur. In the second stage, hydrolysis of the fiber-like material, feathers, or precipitated material may occur. It is understood that two-stage chemical processing of the solid component may be more efficient because the initial solubilization phase may facilitate the subsequent or concurrent hydrolysis phase. It is also understood that such a two-stage process may consume less acid overall, than the equivalent single-stage process to achieve the same level of solubilization and/or degradation of the fiber-like material, feathers, or precipitated material. It is also understood that such a two-stage process may decrease the number of unwanted side reactions, or the amount of unwanted side products formed during either solubilization or hydrolysis, such as decreasing the amount of either formic acid, levulinic acid, furfural, furfuryl alcohol, and the like that is produced. It is appreciated that in embodiments of the treatment processes and apparatus described herein that include a fermentation, such unwanted side reaction products may inhibit the growth or health of the fermenting organism.

In one aspect of chemical processing of the solid component, the solubilization and/or the hydrolysis step is conducted in a depleted oxygen or substantially oxygen-free environment. Oxygen can be removed from the solid components and/or liquid components alike. Oxygen may be removed by sparging the solid and/or liquid component with another gas capable of displacing or replacing the oxygen that is contained in or dissolved in the solid and/or liquid component. Illustrative gases include nitrogen, carbon dioxide, argon, helium, and the like. It is appreciated that the source of acid, water, acid solution, and the like used in the solubilization and/or the hydrolysis steps may also be depleted of or be substantially free of oxygen.

In one embodiment, enzymatic processing of the solid component containing fiber-like material, complex starches, feathers, or precipitated material may be accomplished by treating the component with one or more enzymes including, but not limited to, one or more cellulases, such as endocellulases, terminal cellulases, and the like, alpha amylase, beta amylase, gamma amylase, a proteolytic enzyme, a peptidase, a protease, a phytase, and the like, and combinations thereof. In variations of the processes described herein, one or more enzymes are used in succession, or a mixture of enzymes is used contemporaneously. It is appreciated that the conditions for the optimal conversion of the solid component containing waste stream may be adjusted to optimum levels for the enzyme or mixture of enzymes used, including optimum pH ranges, optimum temperatures, and the like. In aspects where a succession of enzymes or mixtures is used, conditions for optimal enzyme conversion for each enzyme may be included in the processes described. In aspects where a mixture of enzymes is used, the conditions may be optimized for the collection of enzymes in aggregate, or the conditions may be adjusted in a series of steps, such as for a multi-step enzymatic dwell, where optimum conditions are maintained for a predetermined period of time for a particular enzyme or mixture of enzymes, followed by changing the conditions to an optimum or optima for another enzyme or mixture of enzymes. In one aspect of the enzymatic processes described herein, the series of steps are similar to those used in the brewing industry, where for example, pH and temperature are stepped through a series of optimal levels to accommodate a series of enzymatic steps or processes. Sources of mixtures of enzymes include sprouted barley, malted barley, malted barley extract, sorghum extract, and the like. Such mixtures are understood to include phytases, cellulases, hemicellulases, amylases, and other enzymes capable of substantially or totally degrading fiber-like material to small molecular weight components, or solubilizing fiber-like material, to a material useable by fermenting organisms.

It is understood that many such sources of mixtures of enzymes may also include additional carbohydrate, such as complex starches from the barley, sorghum, and the like, as well as fiber-based components such as seed husks, and the like. In aspects of the treatment processes and apparatus described herein that form part of a fermentation system, such as the fermentation systems described herein, this additional carbohydrate may be used as a carbon or carbohydrate source by the fermenting organism. In aspects of the treatment processes and apparatus described herein that include a microbial process, for example in the degradation of proteins, cellulose, and the like, this additional carbohydrate may be used as a carbon or carbohydrate source by the microbes.

In one aspect, the mixture of enzymes includes gamma, beta, and alpha amylase, and proteolytic enzymes derived from malted barley, also referred to as sprouted barley, and the pH and the temperature are graduated to the optima of these enzymes. It is appreciated that the graduation may occur continuously at a predetermined rate, or may occur in a series of steps, each having a predetermined residence or dwell time. It is understood that an optimum step may be also included for the proteolytic enzyme in such processes described herein.

It is appreciated that though the addition of sprouted barley, malted barley, malted barley extract, sorghum extract, and the like mixtures of enzymes that are derived from vegetative matter increases the Chemical Oxygen Demand (COD) of the solid component, the fermenting organism will use the additional COD along with other nutrients such as nitrogen, potassium, and phosphate. In many cases, the limiting nutrient in fermentation processes is carbohydrate, and therefore the additional COD allows the fermenting organism to utilize more of the other nutrients than would be otherwise possible.

In another embodiment, chemical processing of the solid component containing fiber-like material, feathers, or precipitated material may be accomplished by treating the component with an inorganic or organic oxidizing agent. In one aspect, the oxidizing agent is added in a stoichiometric amount. In another aspect, the oxidizing agent is added catalytically along with an additional component capable of regenerating the oxidizing agent, such as oxygen gas. In another embodiment, microbial processing of the solid component containing fiber-like material, feathers, or precipitated material may be accomplished by treating the component with a microorganism.

In one aspect, chemical processing, enzymatic processing, microbial processing, and combinations thereof are performed for a time sufficient to degrade at least a portion of the fiber-like or precipitated material into smaller poly and oligosaccharides, or single sugars, smaller poly or oligopeptides, or single amino acids, and smaller poly phosphates, or inorganic phosphates. Such degradation products may be used by a microorganism in a fermentation process as a carbohydrate source, a nitrogen source, or a phosphorus source for its growth and/or proliferation. In another aspect, chemical processing, enzymatic processing, microbial processing, and combinations thereof are performed for a time sufficient to solubilize at least a portion of the fiber-like or precipitated material. Such solubilized products may be used by a microorganism in a fermentation process as a carbohydrate source, a nitrogen source, or a phosphorus source for its growth and/or proliferation.

In another aspect, chemical processing, enzymatic processing, microbial processing, and combinations thereof are performed at a predetermined temperature. Chemical processing involving acids, bases, oxidizing agents, and the like, may be performed at elevated temperatures to facilitate degradation. Enzymatic processing and/or microbial processing may be performed at elevated temperatures or temperatures below ambient depending upon the stability of the enzyme or enzymes, or microorganism or microorganisms used in the process.

In another aspect, the biomaterial waste stream includes phosphorus-containing organic molecules, such as phytic acid (myoinositolhexaphosphate) and/or other phosphoinositols. Illustratively, phytic acid may account for about 50% of the phosphorus-containing substances in the biomaterial waste stream, including inorganic phosphorus compounds. It is appreciated that biomaterial waste streams from ruminants and partial ruminants, such as mature dairy and beef cattle and horses, may not contain substantial amounts of phytic acid, or may contain much less phytic acid than other biomaterial waste streams, such as waste streams from non ruminants including swine and poultry.

In another embodiment, the treatment processes and apparatus described herein include combining biomaterial waste streams with one or more phytases, such as phytases from plant and grain sources including malted or sprouted grain. Phytases may also be obtained from the fermentation of yeast, or other microorganism that is capable of producing phytases capable of hydrolyzing phytic acid to inorganic phosphate among other things.

It is understood that phytic acid and other organic phosphates often arise in the waste because the animal feed is supplemented with grains, such as corn and soy meal. It is therefore appreciated that biomaterial waste streams coming from animals whose feed has been supplemented with other sources of phosphorus, including the yeast products described herein and in PCT applications serial nos. PCT/US2005/______, entitled SYSTEM FOR PROCESSING A BIOMATERIAL WASTE STREAM (attorney docket no. 35479-77858), PCT/US2005/______, entitled FERMENTER AND FERMENTATION METHOD (attorney docket no. 35479-77851), may have lower proportions of phytic acid.

In embodiments of the processes and apparatus described herein that will form part of a fermentation system, it is appreciated that as a proportion of total nutrient useable by a fermenting organism, the phosphorus component may be in excess. The fermenting organisms requirements for carbohydrate, nitrogen, potassium, and other nutrients may exceed their relative supply in most animal waste streams. Thus, at the end of fermentation, there may be excess nutrient as the limiting nutrient is exhausted. In some embodiments, the excess nutrients are inorganic phosphate salts, organophosphates, and other phosphorus-based compounds. Additional nutrients may be added to compensate for the relative abundance of phosphorus to assist its overall removal from the waste stream, and also to maximize the yield of fermenting organism produced, including, sucrose, corn syrup, molasses, ammonia, and the like. In addition, the treatment processes and apparatus described herein are illustrative of ways of increasing the relative amount of other nutrients, such as carbohydrates derived from fiber-based solids and amino acids derived from protein-based solids.

In variations of the processes described herein where additional nutrient is added, when carbohydrate is the limiting nutrient, simple carbohydrates may be added, such as glucose, sucrose, fructose, corn syrup, molasses, and the like, and combinations thereof. In variations of the processes described herein where additional nutrient is added, when nitrogen is the limiting nutrient, nitrogen sources may be added such as ammonia, ammonium hydroxide, ammonium chloride, and the like, and combinations thereof. Addition of these supplemental nutrients may be take place at any convenient step in the overall process. Illustratively, the supplemental nutrient is added before sterilization, or between sterilization and fermentation. It is appreciated that steps that include pH adjustment of the liquid waste stream may occur after the addition of some supplements, such as the nitrogen containing nutrients because of the possible pH change brought about by the addition. It is farther understood that when nitrogen containing supplements are added, the addition illustratively occurs in between sterilization and fermentation to minimize the production of complicated nitrogen-containing compounds occurring at the high sterilization temperatures, such as alkaloids that may adversely affect the fermentation step.

In another aspect, the biomaterial waste stream includes dissolved and undissolved solids such as lignans, lignins, chitin, and other substances. In some cases, lignin is present from incomplete ruminant digestion. Some dissolved and undissolved solids will also survive the treatment processes described herein, and may be optionally removed from the treated biomaterial waste stream. Illustratively, the dissolved and undissolved solids or surviving substances may be removed by conventional methods of removing suspended solids from liquids, such as by filtration or by collecting the fine fiber material on a vibrating screen.

In waste streams where lignin is present, the lignin may be present with a solid fraction that may be entrained on a shaker screen, such as fiber, bedding, straw, and other cellulosistic waste components. Alternatively, lignin may also be present in the liquid passing through the shaker screen. Lignins may also be present in a high density, small particle solid fraction that may be separated from a liquid fraction by allowing the solids to settle out of the liquid fraction, or by applying a force to separate higher density components, such as a centripetal or centrifugal force. Finally, in other waste streams, such as from non-ruminant animals, some lignin may still form part of the cellulose or fiber-based solid material as part of the matrix. It is generally understood that waste coming from ruminant animals will typically contain more free lignin than waste coming from non-ruminant, or partial or semi ruminant animals where the lignin may still form part of the cellulose-based matrix.

In embodiments of the processes and apparatus described herein that include fermentation of the treated waste stream, the lignin present in the liquid fraction may be removed by filtration before entry into fermentation and/or sterilization steps in the process. It is appreciated that precipitation of the lignin in larger aggregate particles may be facilitated by adjusting the pH or by heating to ease filtration and prevent filter clogging. In general, it is appreciated that the lignin removal may be accomplished by conventional techniques such as those used in the paper industry and in paper-pulp processing.

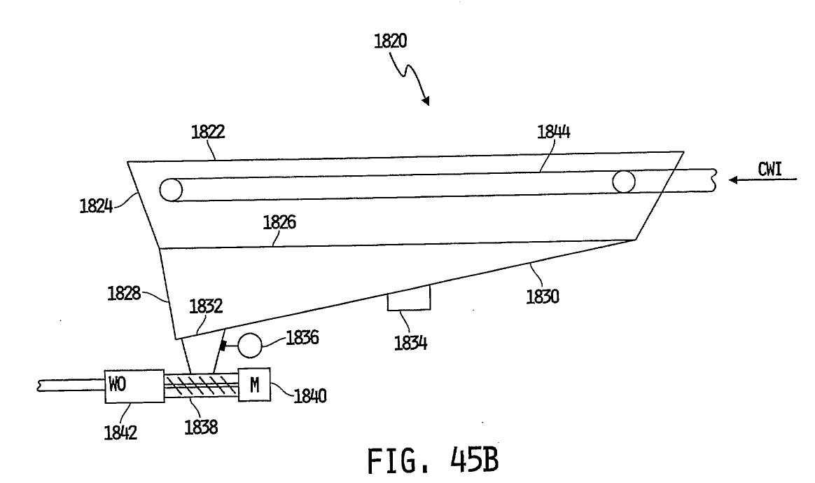

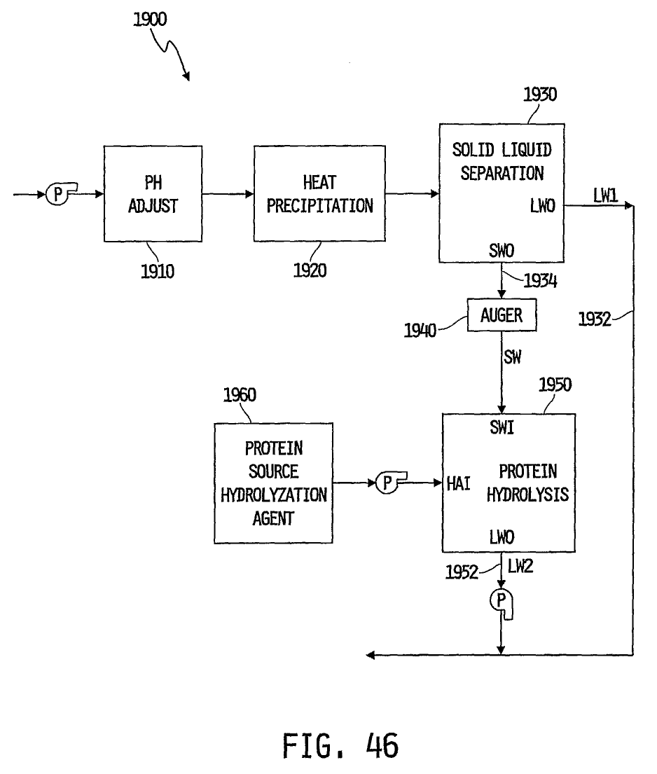

Lignin that is collected with the solid fraction in the solid/liquid separation processes described herein may be illustratively removed with the apparatus shown in FIG. 45 and with an associated process described herein. It is understood that certain processes used for treating the solid fraction may release additional lignin from the cellulose-based matrix. This additional lignin fraction released after any of the hydrolysis or mild hydrolysis processes described herein may be removed by filtration. In embodiments that form part of a fermentation process, the filtration may take place before or after the extract is reintroduced into the liquid waste stream, such as before sterilization, or before fermentation. Lignin that is not removed prior to fermentation may be removed as part of the fermenting organism fraction removed from fermentation systems, such as by flocculation. It is appreciated that lignin that is trapped with the fermenting organism during flocculation steps may be advantageous. In embodiments where the flocculated fermenting organism is subsequently used as a feed supplement, lignins may as act binding agents for ease of handling. Lignin may also be removed following fermentation using processes and apparatus described herein for removing dissolved and undissolved solids from aqueous solutions.

Solids that are separated from liquefied biomaterial waste streams may be treated by contacting the solid fraction with an acid to solubilize and/or hydrolyze at least a portion of the solid fraction. In one embodiment, the solid fraction is subjected to acid solubilization and hydrolysis. Acid solubilization and hydrolysis may be performed with an acid, such as a mineral acid including sulfuric acid, at a relative concentration in the range from about 60% to about 90%, illustratively in the range from about 70% to about 80%, and illustratively about 72% or at about 78%. Hydrolysis and solubilization may be performed for about 1 hour at ambient temperature, although the mixture may be optionally heated.

In another embodiment, the solid fraction is subjected to mild acid hydrolysis. Mild acid hydrolysis may be performed with an organic acid, a mineral acid, and combinations thereof, at an interdependent combination of acid concentration, temperature, and time. It is appreciated that lower temperatures and/or lower acid concentrations may require longer times for mild hydrolysis. Illustrative combinations of these three factors include: about 3% acid for about 1 h at 121° C. (autoclave temperature), about 1% to about 5% acid for about 1 h at about 100° C. or greater, about 5% to about 10% acid for about 1 h at about 90° C. or greater, about 10% to about 20% acid for about 1 h at about 60 to about 90° C., and about 20% to about 30% acid for about 1 h or greater at less than about 60° C. It is appreciated that temperatures above 100° C. may require a pressure vessel for conducting the mild acid hydrolysis. For example autoclave temperatures (121° C.) typically involve about 14 psi of pressure.

The solid fraction may be solubilized and/or hydrolyzed under stronger acid conditions as described herein, then subsequently hydrolyzed under milder acid conditions as described herein. In either case, the resulting treated waste stream may be subjected to an additional solid/liquid separation process to provide a treated liquid extract. The remaining solid fraction may be discarded or recycled into the solubilization and/or hydrolysis processes described herein.

In embodiments that include fermentation, the treated liquid waste stream or extract may be reintroduced to the liquid fraction removed at the solid/liquid separation step. Depending upon the relative volumes of each fraction, namely the original liquid fraction and the extract liquid fraction resulting from the treatment step described herein, the pH may be adjusted to levels for optimal sterilization and/or pH levels that are optimal for the health, growth, and/or proliferation of the fermenting organism. For example, when the fermenting organism is a yeast, the optimal pH is illustratively in the range from about 4.0 to about 4.5. If the pH is too high, additional acid, such as sulfuric acid may be added. If the pH is too low, additional base, such as calcium oxide, calcium hydroxide, calcium carbonate, lime, and the like may be added.

In some variations where a calcium containing base is added to adjust the pH, calcium sulfate may form a precipitate. This precipitate may be optionally removed before any sterilization or fermentation processes or apparatus. It is appreciated that in some situations, the precipitate is not removed until after the fermentation to avoid inadvertent removal of other nutrients that are useable by the fermenting organism, such as organic acids and nitrogen containing components.

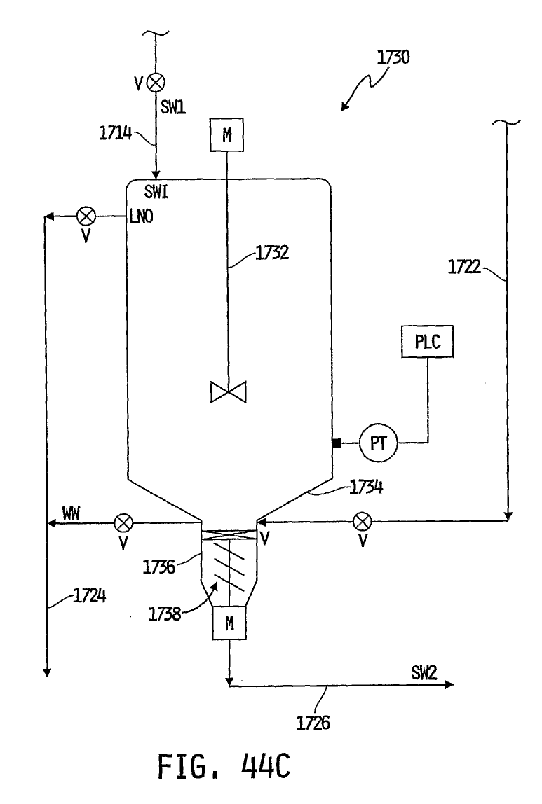

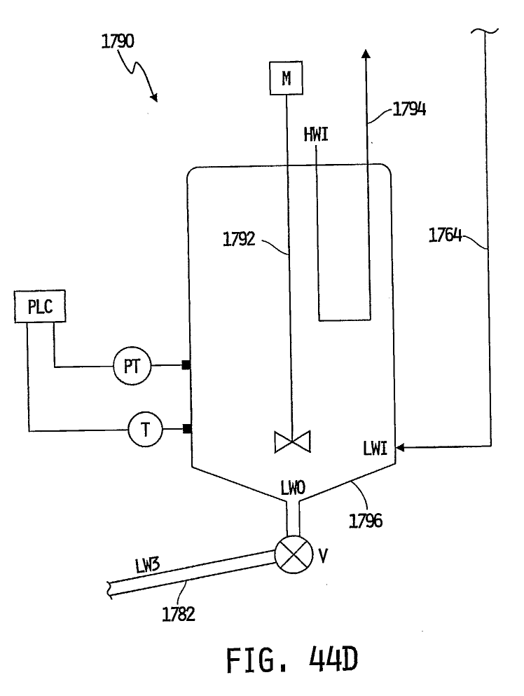

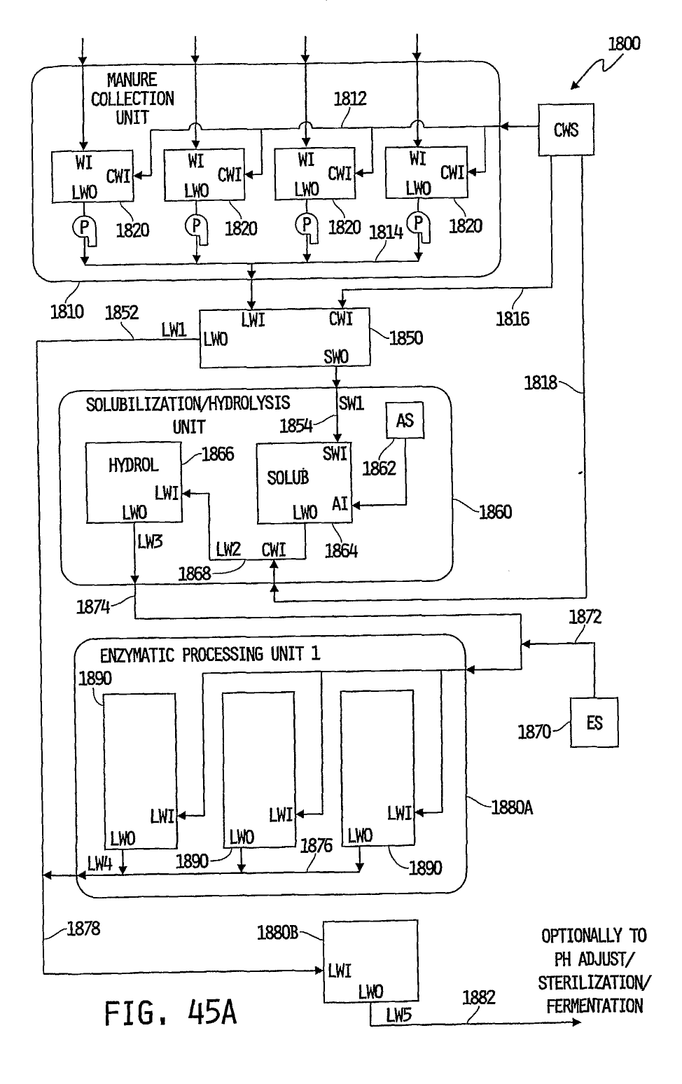

An illustrative embodiment of an apparatus 1700 and process for treating waste streams WS, including barn waste streams is shown in FIG. 44A. The apparatus includes a first solid/liquid separation unit 1710A, which may be any conventional solid/liquid separation system or the solid/liquid separation unit described in PCT/US2005/______, entitled SYSTEM FOR PROCESSING A BIOMATERIAL WASTE STREAM (attorney docket no. 35479-77858) to generate a first liquid waste stream LW1 and one or more solid waste streams SW. First solid/liquid separation unit 1710A includes a liquefied waste stream inlet LWI, a liquid outlet LO, and one or more solid outlets SO. Illustratively, waste stream WS, which has been optionally pre-processed using one or more processes described herein or in PCT/US2005/______, entitled SAND AND ANIMAL WASTE SEPARATION SYSTEM (attorney docket no. 35479-77857) and PCT/US2005/______, entitled SYSTEM FOR PROCESSING A BIOMATERIAL WASTE STREAM (attorney docket no. 35479-77858) enters first solid/liquid separation unit 1710A through inlet LWI. Waste stream inlet LWI is in fluid communication with waste steam conduit 1712, which is in fluid communication with a waste stream source WSS. First solid/liquid separation unit 1710A separates waste stream WS into a first liquid waste stream LW1 and one or more solid waste streams SW. First liquid waste stream LW1 exits separation unit 1710A through liquid outlet LO, which is in fluid communication with a liquid waste stream conduit 1716. First liquid waste stream LW1 is optionally further processed, such as by fermentation as described herein. At least one solid waste stream SW1 is a lower density and/or larger particle solid waste stream that includes fiber, hay, bedding, straw, and other cellulosistic components. First solid waste stream SW1 exits separation unit 1710A through solid outlet SO, which is coupled with a solid conveyer 1714. Solid conveyer 1714 feeds first solid waste stream SW1 into lignin removal unit 1720. Lignin removal unit 1720 includes one or more lignin removal tanks 1730 for removing lignin from first solid waste stream SW1 to provide a second solid waste stream SW2, such as washed fiber.

Referring to FIG. 44C, each lignin removal tank 1730 includes a solid waste stream inlet SWI, a clean water inlet CWI for supplying water to liquefy and wash first solid waste stream SW1, a lignin outlet LNO for removing the lignin suspension, a wash water out WWO for removing wash water after lignin removal, and a solid waste outlet SWO for removing second solid waste stream SW2 after lignin has been removed. Each lignin removal tank 1730 has a generally sloped bottom 1734 connected to solid waste outlet SWO. Outlet SWO is coupled to a collection chamber 1736 at the base of the removal tank 1730. An auger 1738, including a motor M, is coupled to collection chamber 1736 for removing first solid waste stream SW1 after lignin removal. Each lignin removal tank 1730 also includes, a stirring unit 1732, including a motor M, for mixing water and first solid waste stream SW1, and optionally a level or fill sensor for determining when lignin removal tanks 1730 are filled. Alternatively, the fill of lignin removal tanks 1730 may be determined by a known constant flow rate of solid waste SW and clean water CW, and the known capacity of tanks 1730. The optional level or fill sensor may illustratively be a pressure transducer which sends a signal to a programmable logic circuit controlling solid waste conveyer 1714. Upon receiving a signal from pressure transducer PT that a given tank 1730 is fall, conveyer 1714 is stopped or is diverted to move solid waste SW into a second or subsequent tank 1730.

In one illustrative embodiment, first solid waste stream SW1 from conveyer 1714 enters first lignin removal tank 1730 through inlet SWI. When first lignin removal tank 1730 is filled to capacity, conveyer 1714 diverts first solid waste stream SW1 to second lignin removal tank 1730. Lignin may be removed from first solid waste stream SW1 in first lignin removal tank 1730 by suspending the undissolved solids, allowing the undissolved solids to settle, floating off the fine fiber, filtering the fiber, and like processes. In one aspect, clean water enters first lignin removal tank 1730 through inlet CWI and the mixture is agitated or stirred with stirring unit 1732. Inlet CWI is in fluid communication with a clean water conduit 1722 coupled to a clean water source CWS. Fill levels may be determined by using a timing algorithm that includes a predetermined fill rate and volume of removal tanks 1730, by the appropriate placement of a fill level sensor such as a pressure transducer PT in removal tanks 1730, or by any other conventional method. After removal tank 1730 is filled and after an optional predetermined dwell time, the stirring or agitation of the contents in removal tanks 1730 is discontinued and the solid contents of the removal tank 1730 are allowed to settle. It is appreciated that the settling of the components making up first solid waste stream SW1 may follow standard Reynolds behavior where the smaller particles are concentrated toward the top of the settled material, and the larger particles are concentrated toward the bottom of the settled material. It is appreciated that such settling behavior is also dependent upon the relative density of the components making up the solid waste stream SW1, but where densities of various particles are similar, the settling rate will typically be determined by particle size as described herein. After settling, clean water is again introduced through inlet CWI in a countercurrent flow through the bottom of the settled material.

It is appreciated that in variations of removal tanks 1730 shown in FIG. 44C, the countercurrent flow of water may enter through a dedicated clean water inlet CWI, or through the same clean water inlet CWI used to fill removal tanks 1730. The water is introduced through clean water inlet CWI at a predetermined velocity capable of suspending the smaller particles, such as lignin particles, and leaving the larger particles, such as cellulose, hay, straw, and other cellulosistic material at the bottom of removal tanks 1730. It is understood that the smaller or finely divided particles are generally lignin particles or solids that may not be as useful for the subsequent hydrolysis steps than are the larger particles. Water flow is continued until a predetermined amount, illustratively a substantial amount, of the lignin is removed out the top of the removal tank 1730 through lignin outlet LNO, at which time water flow is discontinued. The remaining water in removal tanks 1730 is removed through wash water outlet WWO, which is in fluid communication with lignin conduit 1724, and the wash water is combined with the liquid exiting lignin outlet LNO. The second solid waste stream SW2 is removed from lignin removal tanks 1730 using auger 1738. In one illustrative embodiment, auger 1738 is vertically placed in collection chamber 1736. In another illustrative embodiment, auger 1738 is transverse to collection chamber 1736. In another illustrative embodiment, auger 1738 is fabricated from perfplate and allows water to through and around second solid waste stream SW2 as it is removed from tanks 1730. Second solid waste stream SW2 is moved onto conveyer 1726 and sent to solubilization unit 1760.

The removed lignin exits each lignin removal tank 1730 through lignin outlet LNO and is combined with wash water exiting wash water outlet WWO in lignin conduit 1724. Lignin conduit 1724 in fluid communication with a liquid waste inlet LWI on a second solid/liquid separation unit 1710B.

In aspects that include only one lignin removal tank 1730, the process is performed in a batch mode. In aspects that include more than one lignin removal tank 1730, the process is performed in a continuous mode, where one tank is filling while the remaining tank or tanks are in stirring phase, a settling phase, a washing phase, a draining phase, or a second solid waste stream SW2 removal phase. In either case, each lignin removal tank 1730 includes one or more valves V that may be each operated by a programmable logic circuit, controlling clean water entry into inlet CWI, wash water exit out of outlet WWO, lignin suspension exit out of outlet LNO, and the like. The algorithm controlling the dwell, filling, washing, settling, emptying, and fiber removal steps in the lignin removal process may include an elapsed time parameter, a parameter dependent on sensing a fill level in the tank, other comparable or conventional parameter for monitoring the lignin removal process, or a combination thereof.

Referring to FIG. 44A, illustratively, suspended lignin exiting through exit port LNO, including water removed from removal tank 1730 through wash water outlet WWO, enters second solid/liquid separation unit 1710B, where the removed lignin is separated from the liquid. Second solid/liquid separation unit 1710B includes a clean water inlet CWI in fluid communication with a clean water source via conduit 1728. Separation of the lignin may be accomplished by filtration, centrifugation, or by passing over a fine vibrating screen. The fine vibrating screen may be any conventional vibrating screen, including a vibrating screen assembly described herein. Illustratively, the separated lignin is removed for disposal, and the liquid is now clarified water and is sent to a lagoon, ordinary disposal streams, or to ground. Alternatively, the clarified water may be recycled into any of the processes or apparatus described herein.

Referring to FIG. 44B, upon completion of lignin removal, second solid waste stream SW2 enters solubilization unit 1760 via conveyer 1726. Solubilization unit 1760 includes one or more solubilization tanks 1770. In aspects that include only one solubilization tank 1770, the process is performed in a batch mode. In aspects that include more than one solubilization tank 1770, the process is performed in a continuous mode, where one tank is filling while the remaining tank or tanks are in a stirring phase, or a dwell phase. In either case, each tank 1770 includes a solid waste stream inlet SWI, a solublized or liquefied waste stream outlet LWO, an acid inlet AI, and a stirring unit 1772 including a motor M. Acid inlet AI is supplied by an acid source 1774, and both acid source 1774 and inlet AI are in fluid communication with an acid conduit 1762. Solubilization tanks 1770 optionally include a fill or level sensor, such as a pressure transducer. Alternatively, fill may be predetermined by operating the processes described herein at known flow rates using apparatus with known capacities. Each inlet and outlet of solubilization tanks 1770 is fitted with a valve V optionally coupled to and operated by a programmable logic circuit. The algorithm controlling the filling and emptying of each tank 1770, including acid inlet AI, second solid waste stream SW2, and the solubilized waste outlet LWO, in the solubilization process may include an elapsed time parameter, a parameter dependent on sensing a fill level in the tank, other comparable parameter, or a combination thereof. Each solubilization tank 1770 optionally includes a temperature sensor (not shown) and/or a heat exchanger (not shown). In such alternate embodiments, solubilzation may be performed at a higher than ambient temperature.

In one illustrative process, second solid waste stream SW2 enters the solubilization tanks 1770 through SWI, and acid is introduced into solubilization tanks 1770 through acid inlet AI. Illustratively, acid source AS contains about 95% sulfuric acid, and after addition, the concentration of sulfuric acid in solubilization tanks 1770 is about 72%. The contents are stirred with stirring unit 1772 for a predetermined period of time or until a predetermined measured parameter such as a predetermined conductivity, optical density, or like parameter of the bulk contents of the solubilization tanks 1770 is observed and indicates solubilization of second solid waste stream SW2 to provide a second liquefied waste stream LW2. At that time, second liquefied waste stream LW2 is removed through waste outlet LWO. Waste outlet LWO is in fluid communication with conduit 1764 which is also in fluid communication with acid hydrolysis unit 1780. It is appreciated that in certain variations of the solubilization process, some hydrolysis of second solid waste stream SW2, including hydrolysis of washed fiber, may also occur during the solubilization process.

In variations of the solubilization unit 1760 described herein, each tank 1770 is also fitted with a gas sparger (not shown) for removing oxygen from the solubilization process. It is understood that some acids used in the solubilization process may be incompatible with dissolved oxygen and may cause undesired side reactions, corrosion of the tanks, or other interfering events. Optional sparger is supplied by a gas capable of displacing or replacing the oxygen that is dissolved in the contents of solubilization tanks 1770. In other variations, acid source 1774 is also fitted with a gas sparger (not shown) for removing oxygen. In other variations, water supplied to the solubilization process has been sparged to remove dissolved oxygen. In other variations, second solid waste stream SW2 is also sparged to remove dissolved oxygen before introduction of the acid in solubilization unit 1760.

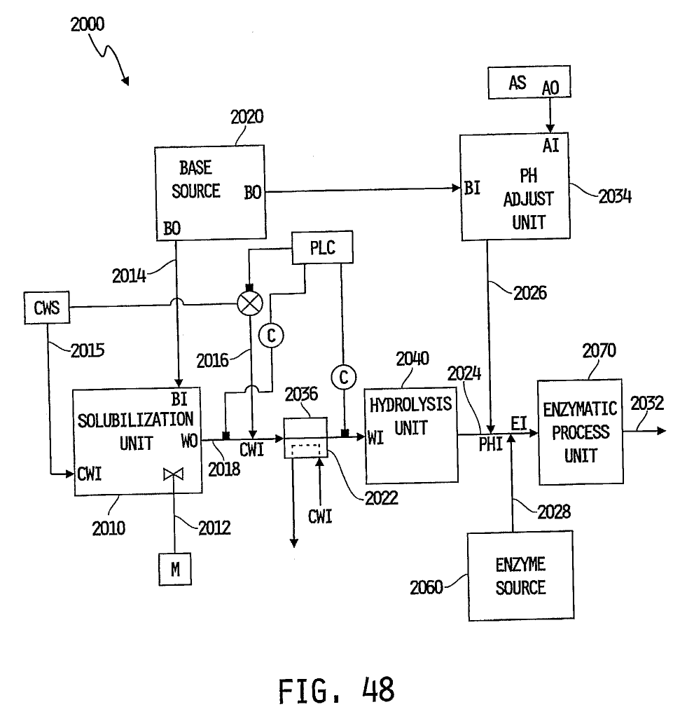

Conduit 1764 is fitted with a clean water inlet CWI in fluid communication with a clean water source via clean water conduit 1766. Second liquefied waste stream LW2 exiting liquefied waste outlet LWO is diluted with water supplied by clean water inlet CWI in conduit 1764. In variations of solubilization processes described herein, an optional heat exchanger 1768 is coupled to conduit 1764 after clean water inlet CWI and prior to acid hydrolysis unit 1780. It is appreciated that during dilution of the solubilized fiber with water, heat may be produced, and in some variations this heat is advantageously removed prior to entry into acid hydrolysis unit 1780. It is understood that this heat may be captured and removed, and optionally used for other steps or components of the processes or apparatus described herein that require heat. Conduit 1764 may also fitted with a series of sensors, such as pH sensors, conductivity sensors, concentration sensors, and the like, and combinations thereof. In one illustrative embodiment, a pair of conductivity sensors CS are coupled to conduit 1764 from which the pH of the second liquefied waste stream LW2 may be measured. A first conductivity sensor CS1 is placed upstream of clean water inlet CWI, and a second conductivity sensor CS2 is placed downstream of clean water inlet, and optionally downstream of heat exchanger 1768, and before hydrolysis unit 1780. Periodic measurements of the pair of conductivity sensors may be used to control the amount or rate of addition of clean water into inlet CWI. An illustrative relationship between conductivity and pH was determined in Example 2, and FIG. 47 shows an illustrative graphical representation of this relationship. Clean water inlet CWI used for diluting liquefied waste stream can be metered and controlled by an algorithm using the sensor data to introduce the appropriate amount of clean water into conduit 1764 to dilute second liquefied waste stream LW2 to achieve the predetermined acid concentration for entry into hydrolysis unit 1780.

Hydrolysis unit 1780 includes one or more hydrolysis tanks 1790. In aspects of hydrolysis unit 1780 that include only one hydrolysis tank 1790, the process is performed in a batch mode. In aspects that include more than one hydrolysis tank 1790, the process is performed in a continuous mode, where one tank is filling while the remaining tank or tanks are in a dwell phase, a stirring phase, a heating phase, or an emptying phase. In either case, each hydrolysis tank 1790 includes a liquid waste inlet LWI, and a liquid waste outlet LWO. Inlet LWI is in fluid communication with conduit 1764 and positioned after optional heat exchanger 1768. Second liquefied waste stream LW2 enters each hydrolysis tank 1790 through inlet LWI. After completion of the acid hydrolysis step, a third liquid waste stream LW3 is provided, which exits each hydrolysis tank 1790 through outlet LWO.

Referring to FIG. 44D, each hydrolysis tank 1790 includes a pair of valves V, optionally operated by a programmable logic circuit, that control flow into each hydrolysis tank 1790 through inlet LWI and flow out of each hydrolysis tank 1790 through outlet LWO. Each hydrolysis tank 1790 has a generally sloped bottom 1796, and also includes a stirring unit 1792, and an optional heating unit 1794. The algorithm controlling the filling, stirring, heating, and emptying of each tank 1790 in the hydrolysis process may include an elapsed time parameter, a parameter dependent on sensing a fill level in the tank, temperature sensor, other comparable parameter, or a combination thereof. Illustrative fill level sensors for use in the various apparatus described herein, including hydrolysis tanks 1790, include pressure sensitive components, pressure transducers, ratio frequency level sensors, weight sensitive components, and the like. Illustrative temperature sensors for use in the various apparatus described herein, including hydrolysis tanks 1790, include thermocouples such as J-type, K-type, E-type, or T-type thermocouples.

In variations of hydrolysis unit 1780 described herein, each tank 1790 is also fitted with a gas sparger (not shown) for removing oxygen from the hydrolysis process. It is understood that some acids used in the hydrolysis process may be incompatible with dissolved oxygen and may cause undesired side reactions, corrosion of the tanks, or other interfering events. The optional sparger is supplied by a gas capable of displacing or replacing the oxygen that is dissolved in the contents of hydrolysis tanks 1790. In other variations, water supplied to the hydrolysis process via inlet CWI in conduit 1764 has been sparged to remove or decrease the amount of dissolved oxygen.

In one illustrative embodiment, valve V to first hydrolysis tank 1790 is opened and second liquefied waste stream LW2 that has been diluted in conduit 1764 enters first hydrolysis tank 1790. Filling of first hydrolysis tank 1790 may be monitored by a fill or level sensor, such as a pressure transducer, first hydrolysis tank 1790. Alternatively, using a known tank capacity and a known flow rate, fill may be determined by elapsed time. After first tank 1790 is fall, valve V to first tank 1790 is closed, and valve V to second hydrolysis tank 1790 is opened and filling begins in the second tank. Stirrer 1792 is operated and if appropriate, heat exchanger 1794 is operated to raise the temperature of the contents of first hydrolysis tank 1790 to the predetermined temperature. A dwell phase ensues where hydrolysis proceeds to provide a third liquid waste stream LW3. After a predetermined period of time, or according to another algorithm used to assess the extent of hydrolysis, valve V controlling liquid waste outlet LWO of first hydrolysis tank 1790 is opened, and third liquid waste stream LW3 is emptied from first hydrolysis tank 1790. Similarly, after filling second hydrolysis tank 1790, a dwell phase for hydrolysis is started, and the filling subsequent hydrolysis tank 1790 begins. It is understood that after the last hydrolysis tank 1790 is filled, first hydrolysis tank 1790 reenters the processing cycle.

Liquid waste outlet LWO of hydrolysis tanks 1790 is in fluid communication with a conduit 1782, which is in fluid communication with a liquefied waste inlet LWI of a third liquid/solid separation unit 1710C. Third liquid/solid separation unit 1710C maybe any conventional solid/liquid separation unit, including a solid/liquid separation unit described herein, and may include a multiple-motor assembly capable of vibrating a screen shaker in two independent directions, and a clean water inlet CWI in fluid communication with a clean water source via conduit 1784, and used for washing the separated solids. Third liquid waste LW3 enters third solid/liquid separation unit 1710C, where solids are separated from third liquid waste LW3 to provide a fourth liquid waste stream LW4 and one or more solid waste streams SW. The one or more solid waste streams exit third solid/liquid separation unit 1710C via solid waste outlet SWO. These solid waste streams may be disposed of or discarded in standard sanitary landfills. Alternatively, one or more of the solid waste streams may be recycled into the processes and apparatus described herein for treating biomaterial waste streams. Fourth liquid waste stream LW4 exits third solid/liquid separation unit 1710C via liquid waste outlet LWO. Outlet LWO is in fluid communication with a liquid waste conduit 1718, which is in fluid communication with liquid waste conduit 1716 exiting liquid waste outlet LWO of first solid/liquid separation unit 1710A. Therefore, fourth liquid waste stream LW4 exiting third solid/liquid separation unit 1710C is admixed with first liquid waste stream LW1 exiting first solid/liquid separation unit 1710A. Combined liquid waste streams LW1 and LW4 may be further processed using any conventional biomaterial waste processing system or apparatus, including the systems described herein and in co-filed applications referenced herein, including additional processing by fermentation.

In embodiments where combined liquid waste streams LW1 and LW4 are further processed by fermentation, combined liquid waste streams LW1 and LW4 may enter a pH adjustment unit, then a sterilization unit, and then a fermentation unit. In processes and apparatus that include a pH adjustment step, that step may illustratively take place after the reintroduction of fourth liquid waste stream LW4 into first liquid waste stream LW1 to minimize the overall consumption of acid the process. It is understood that in such processes, the amount of acid added in the solubilization and hydrolysis steps is illustratively selected as a balance between efficient solubilization and hydrolysis of the components and the ultimate pH needed for processes that include fermentation. It is also appreciated that the overall volume of the fourth liquid waste stream LW4 may often be substantially lower than the overall volume of the first liquid waste streams LW1 exiting first solid/liquid separation unit 1710A. Therefore, even a mild acid hydrolysis solution, illustratively about 3% sulfuric acid, will have sufficient acid concentration to reduce the pH of the combined liquid waste streams LW1 and LW4 to about the level necessary for fermentation, illustratively about 4 to about 5.

A pH adjustment unit may include an acid source and a base source for adjusting the pH of the incoming combined liquid waste streams. Generally, a pH adjustment unit lowers the pH of the buffered alkaline waste, such as barn waste; however, in variations, a pH adjustment unit may raise the pH of the liquid waste stream due to a relatively large proportion of material coming from the processing of solid waste streams by solubilization and hydrolysis, and entering a pH adjustment unit prior to fermentation. Suitable acids include inorganic or mineral acids such as hydrochloric acid, hydrobromic acid, nitric acid, sulfuric acid, and acidic salts thereof, phosphoric acid, and acidic salts thereof, and the like. Suitable bases include inorganic bases such as carbonates, sulfates, phosphates, ammonia, and sodium, potassium, calcium, and other salts thereof, organic bases, and the like.

It is appreciated that the illustrative apparatus shown is FIGS. 44A, to 44B, 44C, and 44D are not restricted to treating barn waste, but is generally applicable to all animal-derived biomaterial waste streams. It is also appreciated that for non-ruminant animal-derived biomaterial waste streams, lignin removal unit 1720 forming part of the illustrative apparatus shown is FIG. 44A may be optional and bypassed.

An illustrative embodiment of an apparatus 1800 and process for treating swine waste streams is shown in FIG. 45A. Swine waste enters manure collection unit 1810. Manure collection unit 1810 includes one or more swine manure receptacles 1820 and a conveying unit 1814 for moving the combined waste to a central site. It is understood that swine waste can be concurrently collected or collected in batches by periodic collection. It is appreciated that cultural practices may suggest that the swine herd is segmented to minimize the transmission of disease. Therefore, the periodic batch collection of SW may also be conducted at a plurality of sites into a plurality of swine manure receptacles 1820, as depicted in FIG. 45A. In variations of the processes described herein where SW is collected concurrently or at a single site, it is understood that there may be only one receptacle 1820. In one aspect, each receptacle 1820 feeds into a pump P, such as a chopping pump, a progressive cavity pump, and the like. In variations of the apparatus shown in FIG. 45A, each receptacle 1820 may not be fitted with a pump P, and the waste collected from the plurality of receptacles 1820 is conveyed to a centralized site having one or more pumps P.

In an alternate embodiment, conveying unit 1814 moves the collected swine waste to a precipitation unit to precipitate proteins, including proteins, organic acids, such as bile acids, and the like that may not be metabolized or are otherwise unusable as nutrients by the fermenting organism in order to recover valuable dissolved solids from LW. A precipitation unit may employ any of a variety of treatments or components that facilitate the precipitation of dissolved solids or the aggregation of undissolved solids, including components such as aggregation catalysts, binders, binding agents, chelators, chelating agent, treatments such as heat, pH changes, and the like, or a combination thereof.

Liquefied waste LW derived directly from collection at one or receptacles 1820 or that exits a precipitation unit and enters solid/liquid separation unit 1850, which may be any conventional solid/liquid separation system or the solid/liquid separation unit described herein and in PCT/US2005/______, entitled SYSTEM FOR PROCESSING A BIOMATERIAL WASTE STREAM (attorney docket no. 35479-77858) to generate one more solid waste streams SWS and a liquid waste stream LWS. It is appreciated that SW may also be optionally preprocessed using one or more processes described in PCT/US2005/______, entitled SAND AND ANIMAL WASTE SEPARATION SYSTEM (attorney docket no. 35479-77857) and PCT/US2005/______, entitled SYSTEM FOR PROCESSING A BIOMATERIAL WASTE STREAM (attorney docket no. 35479-77858) before entering the solid/liquid separation unit 1850.

Solid/liquid separation unit 1850 includes a liquefied waste inlet LWI, a liquid waste stream outlet, LWO, and one or more solid waste stream outlets SWO. First liquid waste stream LW1 is separated from one or more solid waste streams SW in separation unit 1850, and exits separation unit 1850 via outlet LWO. Outlet LWO is in fluid communication with a conduit 1852. At least one solid waste stream outlet SWO is coupled with a conveyer 1854, and moves at least one solid waste stream, such as a first solid waste stream SW1 to solubilization unit/hydrolysis unit 1860. Solubilization unit/hydrolysis unit 1860 includes a solubilization unit 1864, a hydrolysis unit 1866, and acid source 1862. Acid source 1862 is coupled to solubilization unit 1864 via acid inlet AI. Solubilization unit 1864 and hydrolysis unit 1866 are in fluid communication via conduit 1868. Conduit 1868 is in fluid communication with clean water source CWI via conduit 1818. Illustratively, solubilization unit 1760, and hydrolysis unit 1780 shown in FIG. 44A and described above are such variations that may be used in the apparatus of FIG. 45A to solubilize first solid waste stream SW1 and provide second liquid waste stream LW2, dilute second liquid waste stream LW2, and hydrolyze diluted second liquid waste stream LW2 to provide third liquid waste stream LW3.

Following solubilization and hydrolysis, third liquid waste stream LW3 exits solubilization unit/hydrolysis unit 1860 via outlet LWO and enters pump P, which is in fluid communication with a conduit 1874. Conduit 1874 is in fluid communication with an enzyme source ES for supplying an enzyme or a mixture of enzymes to be admixed with third liquid waste stream LW3. Enzyme source ES may contain extracts of sprouted barley, malted barley, malted barley extract, sorghum extract, and the like. Conduit 1874 is also in fluid communication with a liquid waste inlet LWI on a first enzymatic processing unit 1880A. First enzymatic processing unit 1880A includes one or more enzymatic processing tanks 1890. If one enzymatic processing tank 1890 is included in the apparatus shown in FIG. 45A, the system is run in a batch mode. If more than one enzymatic processing tanks 1890 are included in the apparatus shown in FIG. 45A, the system is run in a continuous mode. The control of such a continuous mode parallels that described herein for multiple tank processing involving solubilization, hydrolysis, and the like. Each enzymatic processing tank includes a liquid waste inlet LWI, a liquid waste outlet LWO, a stirrer (not shown), an optional system for heating and/or cooling the contents of enzymatic processing tanks 1890, such as with heat exchangers, and optional temperature, conductivity, pH, fill or level sensors, pressure transducers, and the like for monitoring the enzymatic process performed in enzymatic processing tanks 1890.

Enzymatic processing results in a fourth liquid waste stream LW4 exiting each enzymatic processing tank 1890, and first enzymatic processing unit 1880A. Outlet LWO of enzymatic processing tanks 1890is in fluid communication with outlet LWO of solid/liquid separation unit 1850 via conduit 1852. Following enzymatic processing, fourth liquid waste stream LW4 exits first enzymatic processing unit 1880A and is admixed with first liquid waste stream LW1 in conduit 1852, and the mixture enters second enzymatic processing unit 1880B via conduit 1878. Second enzymatic processing unit 1880B is configured similarly to first enzymatic processing unit 1880A, and includes one or more enzymatic processing tanks 1890, which are configured similarly in both processing units 1880A and 1880B.

In variations of the processes and apparatus shown in FIG. 45A, first liquid waste stream LW1 is treated in a separate enzymatic processing unit 1880C, which is similarly configured. In other variations of the processes and apparatus described in FIG. 45A, third liquid waste stream LW3 exiting solubilization unit/hydrolysis unit 1860 is admixed with first liquid waste stream LW1 prior to enzyme source ES in conduit 1874. The combined first and third liquid waste streams LW1, LW3 enters enzymatic unit 1880A and treated as described above. In other variations of the processes and apparatus shown in FIG. 45A, first liquid waste stream LW1 is not treated in an enzymatic processing unit before or after first liquid waste stream LW1 is combined with fourth liquid waste stream LW4.

Combined liquid waste streams LW1 and LW4 may be further processed using any conventional biomaterial waste processing system or apparatus, including the systems described herein and in co-filed applications referenced herein, including additional processing by fermentation. In embodiments where combined liquid waste streams LW1 and LW4 are further processed by fermentation, combined liquid waste streams LW1 and LW4 may enter a pH adjustment unit, then a sterilization unit, and then a fermentation unit.

An illustrative embodiment of a swine waste receptacle 1820 is shown in FIG. 45B. Receptacle 1820 may be any of a variety of sloped pan designs, and illustratively has an upper bin portion 1822 having vertical sides 1824 each sloping inward, and a lower bin portion 1826 connected to upper bin portion 1822. Lower bin portion 1826 has vertical sides 1828 each sloping inward and connecting to pan floor 1830, which is sloped downward to outlet 1832.

In general, the one or more receptacles 1820 in the swine waste system shown in FIG. 45A are each optionally fitted with a grate (not shown), a clean water inlet CWI, a vibrator or vibrating motor 1834, a level sensor 1836, such as a ratio frequency (RF) level sensor capable of detecting liquid, at a low point in receptacle 1820, an auger feed 1838 in fluid communication with outlet 1832, and a motor 1840 operating auger feed 1838. The auger feed 1838 is in fluid communication with pump 1842. Clean water enters receptacles 1820 through CWI, which optionally includes a sprayer or sparger 1844, and vibrator 1834 encourages movement and mixing of the collected waste into auger feed 1838, and subsequently into pumps 1842, where it is further comminuted or pureed to provide liquefied waste stream LW. In general, the one or more receptacles 1820 are each constructed with a sloped pan to assist the movement of waste into pumps 1842. It is appreciated that clean water inlet CWI, sprayer 1844, vibrator 1834, auger feed 1838, and pump 1842 are coordinated and may be operated to generate liquefied waste stream LW continuously or non-continuously, and non-continuous operation may be periodic or intermittent according to an predetermined algorithm. The algorithm may take any or a variety of inputs including elapsed time, receptacle weight, receptacle fill level, pump torque profile, and the like to initiate a collection sequence as described herein. For example, after a predetermined elapsed time, or after a receptacle 1820 reaches a predetermined fill level or predetermined gross weight, clean water inlet CWI, sprayer 1844, vibrator 1834, auger feed 1838, and pump 1842 are coordinately actuated for collection and generation of LW. Timed sequences may be regularly spaced throughout a 24-hour period, spaced more frequently during daylight and less frequently at night, spaced more frequently in conjunction with feeding times, and the like. Emptying is illustratively continued for a period of time correlated with the volume of receptacle 1820, until a minimum fill level is reached, or when the torque profile of the pump falls below a predetermined threshold value.

In another embodiment, the biomaterial waste stream is a variable and dilute biomaterial waste stream derived from food processing including cheese processing, including whey, and the like. Whey is produced as a byproduct in cheese processing, and is primarily water and residual proteins, lactic acid, lactose, calcium, phosphorus, and other contaminants. Many of the residual proteins, and the lactic acid and lactose cannot be used by certain fermenting organisms and is therefore advantageously removed or degraded in processes and apparatus that include a fermentation process and/or apparatus, such as those described herein.

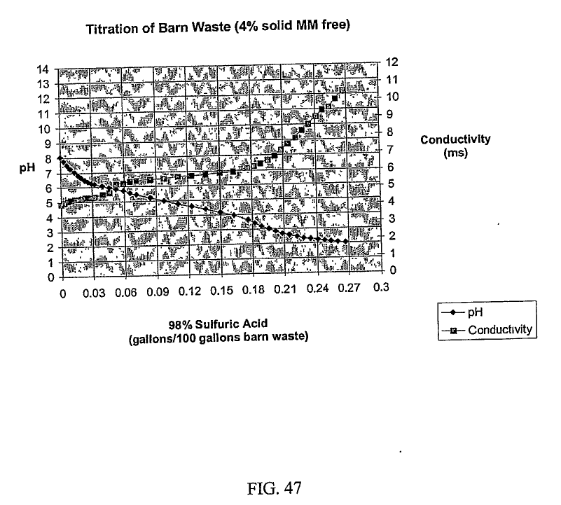

An illustrative embodiment of an apparatus 1900 and process for treating food processing waste streams, such as whey, is shown in FIG. 46. A food processing waste stream enters pH adjustment unit 1910 via pump P, and then into protein precipitation unit 1920. Protein precipitation unit 1920 may function by using aggregation catalysts, binding agents, complexing agents, chelating agents, or by using heat. After precipitation, the food processing waste stream enters a solid/liquid separation unit 1930, including any conventional solid/liquid separation system or the solid/liquid separation unit described herein and in PCT/US2005/______, entitled SYSTEM FOR PROCESSING A BIOMATERIAL WASTE STREAM (attorney docket no. 35479-77858) to generate one or more solid waste streams SW and a first liquid waste stream LW1. Liquid waste stream LW1 exits separation unit 1930 via outlet LWO into conduit 1932 in fluid communication with separation unit 1930. One or more solid waste streams SW exit separation unit 1930 via outlet SWO into conduit 1934 in fluid communication with separation unit 1930 and protein hydrolysis unit 1950. Solid waste streams SW enter protein hydrolysis unit 1950 via solid waste inlet SWI. An auger 1940 coupled to conduit 1934 may be included to move the one or more solid waste streams SW to protein hydrolysis unit 1950. Protein hydrolysis unit 1950 is in fluid communication with a protein hydrolyzation agent source 1960, which supplies a protein hydrolyzation agent via pump P in fluid communication with both source 1960 and unit 1950, and agent inlet HAI coupled to protein hydrolysis unit 1950. After protein hydrolysis has progressed to predetermined or otherwise acceptable levels, the hydrolyzed solid waste streams SW result in a second liquid waste stream LW2, which exits hydrolysis unit 1950 via outlet LWO and into conduit 1952. Liquid waste stream LW2 may be removed from hydrolysis unit 1950 using a pump P. First liquid waste stream LW1 exiting solid/liquid separation unit 1930 and entering conduit 1932 and second liquid waste stream LW2 exiting protein hydrolysis unit 1950 and entering conduit 1952 are admixed.

Combined liquid waste streams LW1 and LW2 may be further processed using any conventional biomaterial waste processing system or apparatus, including the systems described herein and in co-filed applications referenced herein, including additional processing by fermentation. In embodiments where combined liquid waste streams LW1 and LW2 are further processed by fermentation, combined liquid waste streams LW1 and LW2 may enter a pH adjustment unit, then a sterilization unit, and then a fermentation unit.

In another embodiment, the biomaterial waste stream is a variable biomaterial waste stream derived from food processing including waste oils and fats, such as cooking oils, deep frying fats, and the like. Waste oils and fats include glycerol-based fats, fatty acids, glycerols, and the like. Processes for treating such waste oils and fats include hydrolysis reactions, enzymatic degradations, and the like to degrade the fats and/or oils to components including glycerol and fatty or high molecular weight organic acids. Hydrolysis reactions may be performed at acidic pH or at basic pH. In aspects including acidic pH treatment, the waste oils and fats may be treated as described herein for treating cellulosistic materials, such as by treatment with mineral acids, including hydrochloric, hydrobromic, and sulfuric acids. In embodiments of the treatment processes described herein that include fermentation, the pH of the resulting treated waste may adjusted to the level required by the fermenting organism. In variations where the pH is either too low of too high for the fermenting organism, the pH may be increased or decreased in a pH adjustment step as described herein. It is appreciated that in some variations, the pH of the treated waste will be at or near that required by the fermenting organism, or may be pre-selected to match that required by the fermenting organism.

In aspects including basic pH treatment, the waste oils and fats may be treated with inorganic bases such as sodium hydroxide, potassium hydroxide, calcium oxide, calcium hydroxide, sodium and potassium salts of phosphate, sodium and potassium salts of carbonate, ammonium hydroxide, and the like. In addition, catalytic amounts of organic bases may be used, including amine bases such as DBU, DMAP, pyridine, lutidine, collidine, trialkylamines, and the like, in the presence of inorganic bases such as those described herein. In aspects including enzymatic treatment, the waste oils and fats may be treated with an enzyme capable of catalyzing the hydrolysis of esters, including esterases, and the like. In embodiments of the treatment processes described herein that include fermentation, the pH of the resulting treated waste may adjusted to the level required by the fermenting organism. In variations where the pH is either too low of too high for the fermenting organism, the pH may be increased or decreased in a pH adjustment step as described herein. It is appreciated that in some variations, the pH of the treated waste will be at or near that required by the fermenting organism, or may be pre-selected to match that required by the fermenting organism. It is appreciated that when ammonium hydroxide is used as the base, recovery of the base from the treated waste may be accomplished by evaporation. Similarly, if the pH is too high for a fermenting used in embodiments that include fermentation, the pH may be lowered by evaporation of ammonia from the treated waste stream. In variations that include other inorganic bases, it is understood that pH adjustment will produce salts such as sodium chloride, potassium chloride, and the like.

In variations of the processes described herein that include fermentation, it is appreciated that glycerol is often more readily used as a carbohydrate by a fermenting organism than is the parent fat or oil. Therefore, such fermentation processes may require less stringent or less harsh conditions to effect fermenting organism proliferation, or pollution removal than would otherwise be required if the nutrient source were not treated as described herein. It is further appreciated that two-stage fermentation processes, such as those described herein may be used to separately utilize the glycerol nutrient and the fatty acid nutrients. A fermenting organism that uses the glycerol nutrient may be used in one fermentation step, and a fermenting organism that uses the fatty acid nutrient may be used in the other fermentation step. It is further appreciated that the conditions for each nutrient use may be selected to optimize the growth of the fermenting organism, to optimize the utilization of the nutrient, and other desired end results. In one embodiment, the fermenting organism selected to use the fatty acid nutrient in one fermentation step is a Pichia species.

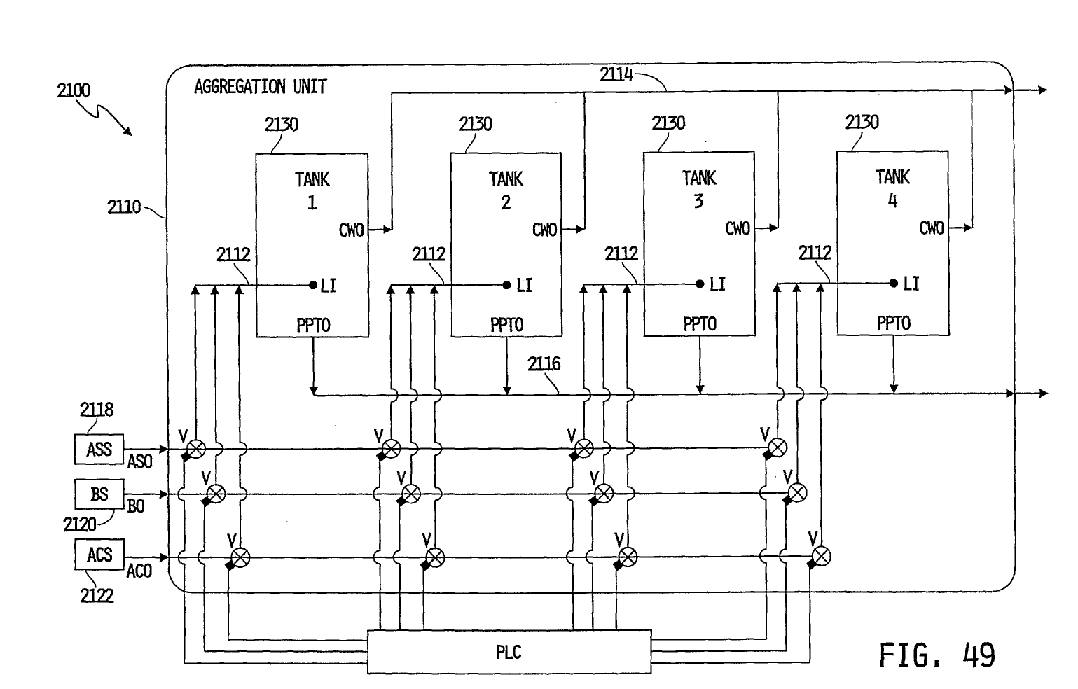

An illustrative embodiment of an apparatus 2000 and process for treating food processing waste streams, such as waste fats and oils, that includes a base hydrolysis or saponification process is shown in FIG. 48. A food processing waste stream enters base solubilization unit 2010, which is fitted with a stirrer 2012, a clean water inlet CWI supplied by clean water source CWS via conduit 2015, a base inlet BI in fluid communication with a base source 2020 supplied via conduit 2014, a solubilized waste outlet SWO, and an optional heating unit (not shown). Solubilization unit 2010 is configured to allow a continuous process where the material that has been the unit 2010 for the longest period of time is preferentially removed from unit 2010 through solubilized waste outlet SWO. In an alternate configuration, solubilization unit 2010 includes a plurality of tanks 2030, and the process is run in a serial batch mode that approximates a continuous operation, where while one tank 2030 is in a filling phase, the remaining tanks 2030 are in various stages of stirring, heating, dwell, or emptying phases. Solubilized waste exiting waste outlet WO enters conduit 2018, which is in fluid communication with waste inlet WI coupled with hydrolysis unit 2040. Hydrolysis unit 2040 may include one or more hydrolysis tanks 2050. Processes and apparatus similar to those described above and shown in FIGS. 44A, 44B, 44C, and 44D may be adapted to such embodiments for treating food processing waste streams in a solubilization unit and/or a hydrolysis unit. Conduit 2018 is also in fluid communication with clean water inlet CWI supplied by clean water source CWS via conduit 2016. Clean water is optionally admixed with waste exiting solubilization unit 2010 to dilute the waste stream for hydrolysis in hydrolysis unit 2040. Conduit 2018 is also optionally fitted with a heat exchanging system 2036. It is appreciated that admixing clean water via conduit 2016 and solubilized liquefied waste in conduit 2018 may produce heat, which is optionally dissipated or removed by heat exchanger 2036 prior to entry of diluted solubilized liquefied waste into hydrolysis unit 2040. Apparatus 2000 may also include a pair of conductivity sensors C coupled to conduit 2018. First conductivity sensor C is located upstream of clean water inlet CWI and second conductivity sensor C is located downstream of clean water inlet CWI. The pair of conductivity sensors C are connected to a programmable logic circuit PLC capable of receiving a signal from conductivity sensors C related to the conductivity of a waste stream in conduit 2018 and calculating a pH or concentration value. Depending upon the calculated pH or concentration value, programmable logic circuit PLC controls the amount of clean water entering conduit 2018 used to dilute a waste stream exiting solubilization unit 2040. It is appreciated that if optional heat exchanger 2036 is included in apparatus 2000, second conductivity sensor C is often located downstream of heat exchanger 2036 to decrease the impact of a temperature variable on the calculation of the pH or concentration value.

Following treatment in hydrolysis unit 2040, solubilized waste SW admixed with an enzyme, such a lipase, and the like, supplied by enzyme source 2060 and enters enzymatic processing unit 2070. Enzymatic processing unit 2070 is fitted with an hydrolyzed waste input LWI, and an enzyme-treated waste outlet LWO. Prior to contact with the enzyme in enzymatic processing unit 2070, the pH of the solubilized waste SW may be adjusted to a level optimum for the enzyme used in enzymatic processing unit 2070. Processes and apparatus similar to those described above and shown in FIG. 45 may be adapted to such embodiments for treating food processing waste streams in an enzymatic processing unit. In embodiments of the waste oil and fat treatment described herein that include a fermentation step, the material exiting enzymatic processing unit 2070 may enter a pH adjustment unit, a sterilization unit, and/or a fermentation unit.

Analogous to other apparatus described herein, in variations of the processes and apparatus described herein for treating waste fats and oils, solubilization unit 2010, hydrolysis unit 2040, and/or enzymatic processing unit 2070 may each include more than one tank, vessel, or container 2080 for performing the respective processing step. These alternate configurations allow the processes to be run in a serial batch mode that simulates a continuous process so that the supply of food waste FW is continuous to the apparatus shown in FIG. 48. In other variations, either hydrolysis unit 2040 or enzymatic processing unit 2070 is bypassed in the apparatus shown in FIG. 48.

### EXAMPLE 2

### Titration of Barn Waste with 98% H₂SO₄

A representative average sample of barn waste was adjusted to 4% solids by weight (MM free). A 100 gallon (379 liter) aliquot of the 4% barn waste slurry was titrated with 98% sulfurric acid, and the pH and the conductivity of the resulting mixture was measured as a function of added acid. The results of the titration are shown in FIG. 47. As the pH (diamonds) decreased with added acid, the conductivity (squares, millisiemens) increased. The barn waste began as an alkaline mixture. It was also observed that the components of the barn waste buffered the solution to pH change. As the pH of the mixture approached neutrality, the conductivity measurement formed a first plateau. As the pH changed through the pKₐ range of most organic acid components included in the barn waste (pH 5.5-3.5), the conductivity formed another plateau, indicative of buffering. As the pH decreased below about 3.5, the conductivity increased rapidly. The first plateau of observed conductivity may be used in algorithms described herein to halt pH adjustment at about pH neutrality, such as in the processes and apparatus described herein for precipitating salts from aqueous solutions. The second plateau of observed conductivity may be used in algorithms described herein to halt the pH adjustment at about 4 to about 4.5, such as in the processes and apparatus described herein for sterilization, fermentation, and the like where the pH is optimally adjusted in the range from about 4 to about 4.5. About 0.12 to about 0.15 gallon (0.4543 to about 0.5678 liter) of sulfuric acid was needed to reach this pH range.

### EXAMPLE 3

### Compositions of Illustrative Biomaterial Waste Streams

Table 4 illustrates representative compositions of horse, dairy, swine, and poultry waste streams.

TABLE 4
Manure and urine analysis per 1000 pounds of animal.^{(a)}

| | horse | dairy | beef | swine | layer | broiler | human |
|---|---|---|---|---|---|---|---|
| wet weight^{(b)} | 50 | 80 | 51.2 | 63.4 | 60.5 | 80 | 30 |
| % water | 78 | 87.5 | 88.4 | 90 | 75 | 75 | 89.1 |
| dry total | 11.0 | 10.0 | | 6.34 | 15.1 | | |
| solids^{(c)} | | | | | | | |
| COD^{(d)} | ND^{(e)} | 8.90 | | 6.06 | 13.7 | | |
| BOD(5)^{(f)} | ND | 1.60 | | 2.08 | 3.70 | | |
| N | 0.28 | 0.45 | 0.3 | 0.42 | 0.83 | 1.1 | 0.2 |
| P | 0.05 | 0.07 | 0.09 | 0.16 | 0.31 | 0.34 | 0.02 |
| K | 0.19 | 0.26 | | 0.22 | 0.34 | | |
| total dissolved | ND | 0.85 | | 1.29 | 2.89 | | |
| solids^{(g)} | | | | | | | |
| C/N^{(h)} | 19 | 10 | | 7 | 7 | | |
| AU⁽ⁱ⁾ | 1 | 0.74 | 1 | 9.09 | 250 | 455 | 8 |

^{(a)}Data from 40CFR., US Environmental Protection Agency; average human weight in US of 125 pounds; data on generation rates, moisture content, nitrogen, and phosphorus from Agricultural Waste Management Field Handbook, USDA Natural Resource Conservation Service, Chapter 4 (April 1992); dairy is lactating cow; beef on high energy diet; swine refers to growers; layers and broilers refer to poultry;
^{(b)}pounds/day/1000# animal;
^{(c)}determined by evaporation using standard EPA protocols;
^{(d)}chemical oxygen demand as determined using standard EPA protocols;
^{(e)}ND = not determined;
^{(f)}biological oxygen demand as determined using standard EPA protocols;
^{(g)}determined by passing the waste a 0.45 μm filter, and evaporating the filtrate; includes suspended solids smaller than 0.45 μm in size;
^{(h)}carbon/nitrogen ratio;
⁽ⁱ⁾number of animals per 100 pounds.

The data shown in Table 4 are illustrative, but it is appreciated that due to feed regimen, season, nutrition, animal location, animal lactation status, and many other variations, these data may vary substantially.

### EXAMPLE 4

### Predicted Results of Processing Illustrative Biomaterial Waste Streams

Table 5 illustrates the calculated nitrogen, phosphorus, and potassium required for conversion of horse, dairy, swine, and poultry waste streams.

TABLE 5^{(A)}

| | horse | dairy | swine | poultry |
|---|---|---|---|---|
| COD 1^{(b)} | 2.0 | 3.3 | 1.2 | 2.6 |
| COD 2^{(c)} | 5.7 | 3.3 | 4.8 | 11 |
| Total COD | 7.7 | 6.6 | 6.1 | 14 |
| required | 0.32 | 0.32 | 0.24 | 0.54 |
| nitrogen^{(d)} | | | | |
| excess (deficit) | (0.04) | 0.13 | 1.84 | 0.29 |
| nitrogen^{(e)} | | | | |
| nitrogen used | 100% | 72.0% | 57.6% | 65.3% |
| required | 0.050 | 0.050 | 0.038 | 0.084 |
| phosphorus^{(d)} | | | | |
| excess | 0 | 0.02 | 0.38 | 0.23 |
| phosphorous^{(e)} | | | | |
| phosphorus | 99.5% | 71.7% | 23.4% | 27.1% |
| used | | | | |
| required | 0.058 | 0.059 | 0.044 | 0.098 |
| potassium^{(d)} | | | | |
| excess | 0.13 | 0.20 | 0.12 | 0.24 |
| potassium^{(e)} | | | | |
| potassium used | 30.5% | 22.5% | 19.9% | 28.8% |

^{(a)}Dairy is lactating cow; swine refers to growers; poultry refers to layers; values given in pounds/day/1000# animal;
^{(b)}chemical oxygen demand of first extract diluted to 4% by weight solids content, as determined using standard EPA protocols;
^{(c)}chemical oxygen demand of second extract corrected to original 4% by weight solids content, as determined using standard EPA protocols;
^{(d)}values calculated based on complete conversion of total COD;
^{(e)}values calculated based on average amount in animal waste stream.

The horse, dairy, swine, and poultry waste streams were diluted to about 4% solids content. The first extract from each waste stream was obtained by settling the corresponding waste for 1 minute and decanting the supernatant liquid. It has been observed that this technique gives similar results to a shaker screen separation. In contrast, centrifugation removes a greater amount of solids, including bacteria. Chemical Oxygen Demand (COD) of the supernatant liquid was determined with standard EPA testing protocols. Fresh, representative scrapings are diluted to approximately 4% solid (total including suspended and dissolved, but excluding sand and minerals including NaCl, and other pure inorganic compounds, concentration relative to moisture and an ash free weight determination. The percentage of sand and un-dissolved solids is estimated by re-dissolving ash residues and decanting.

The first extract was obtained as in Example 3. Washed fiber from each was generated by overflow wash of settled fiber. Water was introduced into a cylinder such that the terminal velocity (settling speed) of ligneous and small cellulose containing fibers was exceeded by the upward velocity of the liquid, leaving only heavy or large fiber material in the flask. The second extract was obtained by exposing the heavy or large fiber to 72% sulfuric acid at ambient temperature for 60 minutes, then diluting to about 3% and heating to 121° C. (autoclave temperature) for 60 minutes. Residual ligneous material was removed. The second extract was added to the first extract and if necessary the pH was adjusted to 4.5 with calcium carbonate. Precipitated calcium sulfate was removed. Calcium carbonate in the form of lime rock facilitated the removal of the calcium sulfate and the ligneous material.

### EXAMPLE 5

### Predicted Results of Processing Illustrative Biomaterial Waste Streams with Added Malted Barley

Table 6 illustrates the calculated nitrogen, phosphorus, and potassium required for conversion of horse, dairy, swine, and poultry waste streams after addition of malted barley as an additional source of carbohydrate.

TABLE 6^{(A)}

| | horse | dairy | swine | poultry |
|---|---|---|---|---|
| COD 1^{(b)} | 2.0 | 3.3 | 1.2 | 2.6 |
| barley COD added^{(c)} | — | 3.2 | 6.0 | 6.0 |
| COD 2^{(d)} | 5.7 | 3.3 | 4.8 | 11 |
| Total COD | 7.7 | 9.8 | 12.1 | 19.7 |
| required nitrogen^{(e)} | 0.32 | 0.45 | 0.47 | 0.77 |
| excess (deficit) nitrogen^{(f)} | (0.04) | (0.04) | (0.13) | (0.02) |
| nitrogen used | 100% | 99.1% | 100% | 92.9% |
| required phosphorus^{(e)} | 0.050 | 0.069 | 0.073 | 0.12 |
| excess phosphorus^{(f)} | 0 | 0 | 0.09 | 0.19 |
| phosphorus used | 99.5% | 98.8% | 45.6% | 38.5% |
| required potassium^{(e)} | 0.058 | 0.081 | 0.085 | 0.14 |
| excess potassium^{(f)} | 0.13 | 0.18 | 0.13 | 0.20 |
| potassium used | 30.5% | 31.0% | 38.7% | 50.0% |

^{(a)}Dairy is lactating cow; swine refers to growers; poultry refers to layers; values given in pounds/day/1000# animal;
^{(b)}chemical oxygen demand of first extract diluted to 4% by weight solids content, as determined using standard EPA protocols;
^{(c)}barley also includes additional nitrogen, phosphorus, and potassium;
^{(d)}chemical oxygen demand of second extract corrected to original 4% by weight solids content, as determined using standard EPA protocols;
^{(e)}values calculated based on complete conversion of total COD;
^{(f)}values calculated based on average amount in animal waste stream.

As can be calculated from Table 5, 3.1 pounds of yeast would theoretically result from 1000 pounds of swine waste. However, only 23% of the available phosphorus is utilized. By addition of about 5 pounds of malt to the process, as illustrated in Table 6, 6.1 pounds of yeast would be theoretically produced, using 45.6% of the phosphorus (corrected for the additional phosphorus included by adding barley). It is appreciated that as much as 50% of the phosphorus may be in the form of phytic acid arising from the corn and soy feed. Corn and soy feed are often used as a replacement for inorganic phosphorus as a food supplement in dairy, beef, swine, and other animal feeds. It is understood that feeding animals a yeast, including a yeast produced in the fermentation processes described herein may eliminate other phosphorus supplementation, including corn and soy feed, and therefore less phosphorus may be in the form of phytic acid. Illustratively, 6.1 pounds of yeast replaces more than 6.1 pounds of soy protein and eliminates phosphate replacement as a feed supplement. Subsequent utilization of phosphate by swine may consequently move to levels higher than the observed 45.6%. The data in Table 6 show that nitrogen is the limiting nutrient, while the data in Table 5 show that COD is the limiting nutrient. Accordingly, it is understood that in order to increase phosphorus consumption, a suitable nitrogen source may be supplied to the fermenting organism, such as gaseous ammonia, ammonium hydroxide, and the like. It is further understood that increasing both the COD, such as by adding corn syrup, molasses, and the like, and nitrogen, such as by adding gaseous ammonia, ammonium hydroxide, and the like, supplied to the fermenting organism, increased consumption of phosphorus can be achieved.

### EXAMPLE 6

### Predicted Yeast Production and Removal of Nutrients/Pollutants

It is appreciated that yeast production is dependent upon the composition of nutrients available in the waste. A 4% w/w total solids (40 grams per liter) barn waste slurry was separated into a first liquid stream and a first solid stream. The COD of the first liquid stream (first extract) was 13.4 g/L. The first solid stream consisted primarily of cellulosistic waste including fiber. The cellulosistic waste was washed, and gave 15 g/original Liter of 4% material. The washed fiber was degraded with sulfuric acid in a two-stage process, and separated into a second liquid stream and a second solid stream. The COD of the second liquid stream (second extract) was 10.0 g/original L. In addition, the stream analyzed for phosphorus at 0.28 g/original L (0.007%), and nitrogen at 1.6 g/L (4%).

Yeast production from the first extract was 1 g yeast for each 1.1 g COD. The production rate of yeast from the first extract was calculated to be 4,187 g/min (9.2 pounds/min), based on a 379 L/min flow rate (100 gallon/min), 13.4 g COD, and 1.2 conversion rate (adjusted for inefficiency by 0.1). Yeast production from the second extract was 1 g yeast for each 2.2 g COD. The production rate of yeast from the second extract was calculated to be 1,630 g/min (3.6 pounds/min) based on a 379 L/min flow rate (100 gallon/min), 10 g COD, and 2.3 conversion rate (adjusted for inefficiency by 0.1). It is understood that the production rate from the first extract may be higher due to the presence of organic acids, urea, amino acids, lipids, and other valuable nutrients. In contrast, it is understood that the production rate from the second extract may be lower because the major nutrients are carbohydrate, as is consistent with conventional conversion rates for sugar, 1 g yeast for each 2.2 g sugar. It is further understood that production rates for the second extract may be improved in barn waste streams containing biomass bedding, such as straw, hay, sawdust, and the like. For example, hay contains significant protein that may improve yeast production.

### EXAMPLE 7

### Predicted Pollution/Nutrient Removal

An assay of a representative sample of yeast produced in 1 minute using the processes and with the apparatus described herein shows 7.5% nitrogen, 1.5% phosphorus, and 1.8% potassium. An assay of a representative sample of barn waste flowing at 379 L/min (100 gallons/min) shows 600 g of nitrogen, 150 grams of potassium, and 105 grams of phosphorus. The pollutant removed can be calculated, and is shown in Table 7.

TABLE 7^{(a)}

| nutrient source | Yeast | nitrogen | potassium | phosphorus |
|---|---|---|---|---|
| first extract | 4187 | 314 | 75.4 | 62.8 |
| second extract | 1630 | 122 | 29.3 | 24.5 |
| totals | 5817 | 436 | 105 | 87.3 |
| total nutrient^{(b)} | — | 600 | 150 | 105 |
| % removed | — | 73 | 70 | 83 |

^{(a)}Values given in grams;
^{(b)}amounts available in 100 gallons (379 liters) of barn waste.

### Illustrative Embodiments of a Fermenter and Fermentation Method

The method described herein is a method useful for treating a biomaterial waste stream to remove pollutants in the biomaterial waste stream by converting the pollutants to a valuable product. In one embodiment a biomaterial waste stream is subjected to oxidative fermentation in the presence of a microorganism (i.e., a fermenting organism) to convert the pollutants in the biomaterial waste stream to a valuable product. Accordingly, at least a portion of the pollutants (e.g., phosphorous, nitrogen, and potassium) is removed from the biomaterial waste stream and incorporated into the valuable product, for example, the microorganism, reducing environmental pollution. In one embodiment, fermentation of the biomaterial waste stream by the presently described method results in the production of a valuable protein product (e.g., a microorganism such as a yeast) that can be used, for example, as an animal feed additive, a feed supplement, a fertilizer, a fertilizer ingredient, or a soil conditioner.

As used in this application, “microorganism” and “fermenting organism” are interchangeable.

Exemplary biomaterial waste streams that can be treated in accordance with the method described herein include, but are not limited to, manure, cellulosistic solid waste, whey broth from cheese production or biomaterial waste streams from other foodstuffs, broth remediation from alcohol or yeast production, tannery waste, slaughterhouse waste, tallow waste from rendering processes, waste derived from plants, and land fill waste. The waste derived from plants can be, for example, waste from hay, leaves, weeds, or wood and can be, for example, yard waste, landscaping waste, agricultural crop waste, forest waste, pasture waste, or grassland waste. The waste derived from foodstuffs can be fruit and vegetable processing waste, fish and meat processing wastes, bakery product waste, and the like. In embodiments where the biomaterial waste stream is manure, the manure can be from an animal, for example, such as a human, a bovine animal, an equine animal, an ovine animal, a porcine animal, or poultry. In one embodiment the biomaterial waste stream is a variable and dilute biomaterial waste stream derived from animal manure or human waste. In general, any organic biomaterial waste stream containing proteins, simple or complex carbohydrates, or lipids, or a combination thereof, can be fermented by using the presently described method.

In one embodiment, the product generated is the microorganism (i.e., a fermenting organism) that contacts the biomaterial waste stream, and the microorganism utilizes the pollutants in the biomaterial waste stream (e.g., potassium, nitrogen, and phosphorus) as nutrients and removes the pollutants from the biomaterial waste stream. Illustratively, the product generated can be used as an animal feed, an animal feed supplement, a fertilizer, a fertilizer ingredient, or a soil conditioner.

An exemplary technique that can be used to estimate the potential capacity for removal of pollutants from the biomaterial waste stream is a chemical oxygen demand (COD) measurement. A COD measurement can be accomplished by estimating oxygen demand by oxygenation of compounds in the presence of an indicator of the oxygenation, and techniques for COD measurement are known in the art. A COD measurement provides an estimate of the quantity of compounds that may potentially be removed from the biomaterial waste stream by oxidative techniques. A COD measurement may be made, during, before, or after the fermentation process as a measurement of the extent of completion of removal of potential pollutants.

The microorganisms (i.e., fermenting organisms) that contact the biomaterial waste stream can be, for example, bacteria, yeast, fungi, mycoplasma, and combinations thereof, that utilize the pollutants in the biomaterial waste stream as nutrients. Yeast species that can be used in the presently described method include such yeast species as Saccharomyces species, Zygosaccharomyces species, Candida species, Hansenula species, Kluyveromyces species, Debaromyces species, Nadsonia species, Lipomyces species, Torulopsis species, Kloeckera species, Pichia species, Yersinia species, Schizosaccharomyces species, Trigonopsis species, Brettanomyces species, Cryptococcus species, Trichosporon species, Aureobasidium species, Phaffia species, Rhodotorula species, Yarrowia species, Schizosaccharomyces species, Karwinskia species, Torulospora species, Schwanniomyces species, or any other yeast species that is capable of fermenting organic waste. Various yeast species are described in N. J. W. Kreger-van Rij, Biology of Yeasts, Vol. 1, Chap. 2, A. H. Rose and J. S. Harrison, Eds. Academic Press, London, 1987, incorporated herein by reference.

Bacterial species that can be used in the presently described method include, for example, Proteus species, Klebsiella species, Providencia species, Yersinia species, Erwinia species, Enterobacter species, Salmonella species, Serratia species, Aerobacter species, Escherichia species, Pseudomonas species, Shigella species, Vibrio species, Aeromonas species, Campylobacter species, Streptococcus species, Staphylococcus species, Lactobacillus species, Micrococcus species, Moraxella species, Bacillus species, Bordetella species, Enterococcus species, Propionibacterium species, Streptomyces species, Clostridium species, Corynebacterium species, Eberthella species, Micrococcus species, Mycobacterium species, Neisseria species, Haemophilus species, Bacteroides species, Listeria species, Erysipelothrix species, Acinetobacter species, Brucella species, Pasteurella species, Vibrio species, Flavobacterium species, Fusobacterium species, Streptobacillus species, Calymmatobacterium species, Legionella species, Treponema species, Borrelia species, Leptospira species, Actinomyces species, Nocardia species, Rickettsia species, and any other bacterial species that is capable of fermenting organic waste.

Examples of fungi that can be used in the presently described fermentation method include, but are not limited to, fungi that grow as molds or are yeastlike, including, for example, fungi that cause diseases such as ringworm, histoplasmosis, blastomycosis, aspergillosis, cryptococcosis, sporotrichosis, coccidioidomycosis, paracoccidioidomycosis, mucormycosis, chromoblastomycosis, dermatophytosis, protothecosis, fusariosis, pityriasis, mycetoma, paracoccidioidomycosis, phaeohyphomycosis, pseudallescheriasis, sporotrichosis, trichosporosis, pneumocystis infection, and candidiasis.

In one embodiment, the microorganism (i.e., a fermenting organism) that contacts the biomaterial waste stream can be a thermophilic microorganism. In another embodiment, the microorganism can be a microorganism that is not thermophilic. The microorganism can be naturally present in the biomaterial waste stream or the biomaterial waste stream can be inoculated with the microorganism.

The microorganism can be partially or completely flocculated, and the microorganism can be artificially or naturally flocculated. In embodiments where the microorganism is artificially flocculated, a flocculating agent of a cationic type can be used in combination with a flocculating agent of an anionic type to catalyze flocculation. The flocculating agent of the cationic type can be selected from the group including ferrous chloride, ferrous sulphate, ferric chloride, ferric sulphate, chlorinated ferric sulphate, aluminium sulphates, chlorinated basic aluminium sulphates, magnesium chloride, magnesium sulphate, and combinations thereof, and the like, or other cationic flocculating agents described in more detail herein.

The flocculating agent of the anionic type can be selected from the group including an anionic polyacrylamide, a polyacrylate, a polymethacrylate, a polycarboxylate, a polysaccharide (e.g., xanthan gum, guar gum or alginate), chitosan, cellulose, and combinations thereof, and the like, or other anionic flocculating agents described in more detail herein. A mixture of flocculating agents of the cationic and/or the anionic type can also be used. In one embodiment, the microorganism is artificially flocculated using ferric chloride and xanthan gum. A method of catalyzing the flocculation of microorganisms is described more fully herein.

Illustratively, the fermentation unit 580 for use in the present method can be an air-lift fermenter and the fermentation method can be continuous flow fermentation where the fermentation is oxidative fermentation, and the fermentation is made oxidative by injecting sterilized air into the fermentation unit 580. In one embodiment, the fermentation unit 580 is cylindrical and the highest concentration of microorganisms is in the bottom half of the cylinder.

In another embodiment, the fermentation unit 580 can have an upwardly opening cone 890 at the bottom of the fermentation unit 580 for collection of the microorganism, and the lower portion of the upwardly opening cone 890 can be tapered for collection of the microorganism in the tapered region of the cone 890 for removal of the microorganism from the fermentation unit 580 through the product outlet port.

In one embodiment, the fermentation unit 580 can have a primary air inlet F10 to inject air into the fermentation unit 580 at a location outside of the cone 890 to circulate the microorganisms in the fermentation unit 580. In another embodiment, the cone 890 can have a secondary air inlet 898 to inject air into the cone 890. The injection of air into the cone 890 can remove at least a portion of the microorganisms that have collected in the cone 890 out of the cone 890 so that the concentration of microorganisms in the cone 890 is reduced. As a result, the amount of the microorganism that is removed from the fermentation unit 580 after collection in the cone 890 is reduced.

An exemplary system for the fermentation of a biomaterial waste stream, including the fermentation unit 580 that is part of the system, is described in detail herein and in PCT/US2005/______, entitled SYSTEM FOR PROCESSING A BIOMATERIAL WASTE STREAM (attorney docket no. 35479-77858) incorporated herein by reference.

One or more fermentation units 580 can be employed in the present method and, if more than one fermentation unit 580 is used, the fermentation units 580 are in fluid communication with each other. The fermentation unit 580 for use in the present method can be used directly on the site of an agricultural operation, if the system and method are used for the fermentation of animal manure, and can be adapted to any size animal feeding operation or to any size community, or to any type of biomaterial waste stream.

In one embodiment, the method includes the step of subjecting the biomaterial waste stream to conditions conducive to aerobic fermentation of the biomaterial waste stream. Illustratively, the conditions conducive to fermentation can include an oxygen level in the fermentation unit 580 that is hyperbaric in the region of the fermentation unit 580 containing the highest concentration of the microorganism (e.g., the bottom of the cylinder depicted in FIG. 20). In other embodiments, the conditions conducive to fermentation can include maintaining the biomaterial waste stream at a pH level of from about 2.0 to about 10.0 and/or maintaining the temperature of the biomaterial waste stream at a temperature of from about 15° C. to about 80° C. The conditions conducive to fermentation can be monitored by, for example, monitoring the conductivity, the temperature change (i.e. monitoring the amount of cooling required to maintain the temperature), or the gas volume/mass of the biomaterial waste stream. An exemplary system for the fermentation of a biomaterial waste stream, including the sensors and controls for monitoring the conductivity, the temperature change, or the gas volume/mass of the biomaterial waste stream, is described more fully herein and in PCT/US2005/______, entitled SYSTEM FOR PROCESSING A BIOMATERIAL WASTE STREAM (attorney docket no. 35479-77858) incorporated herein by reference.

The fermentation method can also be optimized by maintaining steady-state proliferation of the microorganisms resulting in efficient fermentation of the biomaterial waste stream. In embodiments where the biomaterial waste stream is variable and dilute, the steady-state proliferation of the microorganisms can be maintained by monitoring the conductivity, the temperature change (i.e., monitoring the amount of cooling required to maintain the temperature), or the gas volume/mass of the biomaterial waste stream, and combinations thereof, and by increasing or decreasing the amount of microorganisms in the fermentation unit 580. The amount of the microorganisms in the fermentation unit 580 can be adjusted, such as by removing a portion of the microorganisms from the fermentation unit 580 intermittently or continuously.

For example, in the embodiment where flocculated microorganisms are used, the flocculated microorganisms settle in the cone 890 and can be removed from the fermentation unit 580 through the product outlet port. In one embodiment, the flocculated microorganisms can be removed from the fermentation unit 580 through the product outlet port independently of the biomaterial waste stream due, in part, to settling and compression of the flocculated microorganisms in the cone 890 and the product outlet port. In this embodiment, if it is necessary to reduce the amount of flocculated microorganisms removed from the fermentation unit 580 and to allow the microorganisms to accumulate in the fermentation unit 580 to maintain steady-state proliferation, air can be injected into the secondary air inlet 898 to inject air into the cone 890. The injection of air into the cone 890 removes, out of the cone 890 and the product outlet port, at least a portion of the flocculated microorganisms that are settling and compressing in the cone 890 and the product outlet port so that the concentration of microorganisms in the cone 890 and the product outlet port is reduced. As a result, the amount of flocculated microorganisms removed from the fermentation unit 580 through the product outlet port is reduced and the amount of microorganisms that remain in the fermentation unit 580 is increased.

In another embodiment, if it is necessary to increase the amount of flocculated microorganisms removed from the fermentation unit 580, air injection into the secondary air inlet 898 can be stopped to allow the flocculated microorganisms to settle and compress in the cone 890 and the product outlet port. As a result, the amount of flocculated microorganisms removed from the fermentation unit 580 through the product outlet port is increased and the amount of microorganisms in the fermentation unit 580 is decreased.

In the embodiment of the presently described fermentation method where flocculated microorganisms are used, the capacity to control the amount of flocculated microorganisms removed from the fermentation unit 580, and to remove flocculated microorganisms from the fermentation unit 580 independently of the biomaterial waste stream, allows for steady-state proliferation to be maintained when the biomaterial waste stream being injected into the fermentation unit 580 has variable nutrient content. Because the flocculated microorganisms can be removed from the fermentation unit 580 independently of the biomaterial waste stream, steady-state proliferation of the microorganisms can be maintained in the fermentation unit 580 due to the ability to control the amount of microorganisms in the fermentation unit 580 relative to the amount of nutrient in the variable biomaterial waste stream present in the fermentation unit 580 at any one point in time. The ability to maintain steady-state proliferation of the microorganisms can result in efficient conversion (i.e., reproduction) of the microorganisms in the fermentation unit 580. In this embodiment, the steady-state proliferation of the microorganisms can also be maintained by monitoring the conductivity, the temperature change, and the gas volume/mass of the biomaterial waste stream, and combinations thereof, because these parameters are indicative of the state of proliferation of the microorganisms in the fermentation unit 580.

As discussed above, a valuable product (i.e., the microorganisms) is produced according to the presently described method. After removal of the microorganisms (i.e., the product) from the fermentation unit 580, the microorganisms can be preserved using any method known in the art for preventing degradation of microorganisms and/or their protein components. For example, the microorganisms can be pasteurized or the microorganisms can be refrigerated or frozen after removing the microorganisms from the fermentation unit 580. Alternatively, the microorganisms can be degraded or partially degraded.

The microorganisms can be used as a valuable product in the form of, for example, a paste, or another aqueous mixture, or a dry powder. The paste, aqueous mixture, or dry powder contains various nutrients and proteins that are suitable, for example, for use as an animal feed additive or an animal feed supplement or for use as a fertilizer, a fertilizer ingredient, or a soil conditioner. In an alternate embodiment, the wet product removed from the fermentation unit 580 can be used without further processing.

The system for processing a biomaterial waste stream according to the method described herein has a waste fermentation system 10, including, among other components, a fermentation unit 580, for converting the biomaterial waste stream to a valuable product. For a more detailed description of the fermentation system 10 and illustrative embodiments, including a more detailed description of the fermentation unit 580 which is a component of the system, see this application and PCT/US2005/______ entitled SYSTEM FOR PROCESSING A BIOMATERIAL WASTE STREAM (attorney docket no. 35479-77858).

Generally, the waste fermentation system 10 has a liquid waste inlet for receiving the biomaterial waste stream, a product outlet port for removing the microorganism and a liquid outlet for removing the residual biomaterial waste stream liquid (i.e., the treated biomaterial waste stream from which pollutants have been removed). A number of sensors can be provided to produce sensory information relating to operation of the waste fermentation system 10. A controller can be provided to monitor the sensory information, and the controller can be configured to control the waste fermentation system 10 based on the sensory information. The system can further include a number of actuators each responsive to a different actuator control signal to modify operation of the waste fermentation system 10, and the controller can be configured to produce the number of different actuator control signals based on the sensory information. The biomaterial waste stream can be provided in the form of a continuous flow of liquid biomaterial waste, and can have variable nutrient content. The system controller can accordingly be configured to control the waste fermentation system 10, based on the sensory information, to controllably remove the microorganism while the nutrient content in the continuous stream of biomaterial waste is varying.

The system can further include a waste pretreatment system having a liquid waste inlet for receiving biomaterial waste and a liquid waste outlet for producing the biomaterial waste stream, wherein the waste pretreatment system is operable to treat the biomaterial waste and supply the resulting biomaterial waste stream to the fermentation unit 580. The waste pretreatment system can include a separation unit 18 for separating waste solids from the biomaterial waste and producing a resulting liquid waste stream. The waste pretreatment system can include a pH adjustment unit 38 for modifying the pH level of the liquid waste stream to produce the biomaterial waste stream having a target pH.

The system can further include a waste post-treatment system having an inlet port for receiving the residual biomaterial waste stream liquid (i.e., the fermented biomaterial waste stream), a product outlet port and a liquid outlet port for producing a cleaned liquid stream, wherein the waste post-treatment system can be operable to precipitate excess nutrient from the residual biomaterial waste stream liquid, and produce a resulting product at the product outlet and the cleaned liquid stream at the liquid outlet.

In such a system, the waste fermentation system 10 can also include a sterilization unit 570 having a liquid waste inlet defining the liquid waste inlet of the waste fermentation system 10 and a liquid waste outlet, wherein the sterilization unit 570 can be operable to sterilize the biomaterial waste stream and produce a sterilized biomaterial waste stream at the liquid waste outlet of the sterilization unit 570.

Another one of the number of sensors of the biomaterial waste processing system can be a flow rate sensor 104₅ producing a flow rate signal indicative of a flow rate of the biomaterial waste stream entering the liquid waste inlet of the sterilization unit 570. A controller can be configured to control the flow rate of the biomaterial waste stream entering the liquid waste inlet of the sterilization unit 570 between upper and lower flow rate thresholds.

The sterilization unit 570 can further be fluidly coupled to an inlet of a sterilization loop 630, and a pre-sterilization heat exchanger HX2 having a fluid passageway having a temperature-controlled fluid passing therethrough. The pre-sterilization heat exchanger HX2 can be configured to control the temperature of the biomaterial waste stream to a target sterilization temperature as a function of the temperature of the temperature-controlled fluid. For example, the waste fermentation system 10 can further include a steam unit 572 supplying the temperature-controlled fluid to the pre-sterilization heat exchanger HX2 in the form of steam.

The sterilization unit 570 can further include a post-sterilization heat exchanger HX1 configured to transfer heat from the sterilized biomaterial waste stream exiting the sterilization unit 570 to the biomaterial waste stream entering the pre-sterilization heat exchanger.

The waste fermentation system 10 can further include a fermentation unit 580 having a sterilized waste stream inlet fluidly coupled to the waste stream outlet of the sterilization unit 570, a microorganism outlet defining the product outlet of the waste fermentation system 10 and a residual biomaterial waste stream liquid outlet fluidly coupled to the liquid outlet of the waste fermentation system 10. Such a fermentation unit 580 can be configured to aerobically ferment the sterilized biomaterial waste stream to produce the microorganism (i.e., a fermenting organism) and the residual biomaterial waste stream liquid. The fermentation unit 580 can further include a seed inlet SD1 and SD2 for receiving a microorganism, wherein contact of the microorganism with the sterilized biomaterial waste stream within the fermentation unit 580 can commence fermentation of the sterilized biomaterial waste stream. The waste fermentation system 10 can further include a cooling unit configured to control the temperature of the sterilized biomaterial waste stream entering the fermentation unit 580 to a target waste stream temperature.

In one embodiment, a method of treating a biomaterial waste stream to remove pollutants and to generate a product is provided. The method comprises the steps of injecting the biomaterial waste stream into a first fermentation unit 580, contacting the biomaterial waste stream with a first microorganism in the first fermentation unit 580, subjecting the biomaterial waste stream in the first fermentation unit 580 to conditions conducive to aerobic fermentation of the biomaterial waste stream, removing at least a portion of the biomaterial waste stream from the first fermentation unit 580, injecting the at least a portion of the biomaterial waste stream into a second fermentation unit 580 in fluid communication with the first fermentation unit 580, contacting the biomaterial waste stream with a second microorganism in the second fermentation unit 580, and subjecting the biomaterial waste stream in the second fermentation unit 580 to conditions conducive to aerobic fermentation of the biomaterial waste stream.

In this embodiment, the first and second microorganism can be the same species of microorganism or the first and second microorganism can be different species of microorganism. Further, in this embodiment, the first microorganism in the first fermentation unit can be selected from the group consisting of a non-flocculated organism, a naturally flocculating organism, and an artificially flocculating organism, and the second microorganism in the second fermentation unit can be selected from the group consisting of a non-flocculated organism, a naturally flocculating organism, and an artificially flocculating organism with the proviso that the first and the second microorganism cannot both be non-flocculating.

The system and fermentation method described above can be used to produce a valuable product. The microorganism removed from the fermentation unit 580, can be used, for example, as an animal feed additive, a feed supplement, a fertilizer, a fertilizer ingredient, or a soil conditioner.

### Illustrative Embodiments of a Flocculation Method and Flocculated Organism

The present invention is based, in part, on the discovery of a method useful for the catalyzed flocculation of microorganisms. The method comprises contacting the microorganisms with a cationic flocculating agent, contacting the microorganisms with an anionic flocculating agent, and flocculating the microorganisms. The rate and the extent of flocculation of microorganisms that are naturally flocculating, or are not naturally flocculating, can be controlled using this method. Thus, this method results in the catalyzed flocculation of microorganisms whereby the rate and extent of flocculation of naturally or non-naturally flocculating microorganisms can be controlled. The method can be used to separate naturally flocculating or non-flocculating microorganisms from bulk fluids by sedimentation, for example, when ultrafiltration or ultracentrifugation is impractical.

The microorganisms that can be flocculated using this method include, for example, bacteria, yeast, fungi, mycoplasma, and the like. Yeast species that can be used in the presently described method include such yeast species as Saccharomyces species, Zygosaccharomyces species, Candida species, Hansenula species, Kluyveromyces species, Debaromyces species, Nadsonia species, Lipomyces species, Torulopsis species, Kloeckera species, Pichia species, Yersinia species, Schizosaccharomyces species, Trigonopsis species, Brettanomyces species, Cryptococcus species, Trichosporon species, Aureobasidium species, Phaffia species, Rhodotorula species, Yarrowia species, or Schwanniomyces species, or any other yeast species that is capable of being flocculated using the method described herein. Various yeast species are described in N. J. W. Kreger-van Rij, Biology of Yeasts, Vol. 1, Chap. 2, A. H. Rose and J. S. Harrison, Eds. Academic Press, London, 1987, incorporated herein by reference.

Bacterial species that can be flocculated using the presently described method include gram positive and gram negative bacteria and include, for example, Proteus species, Klebsiella species, Providencia species, Yersinia species, Erwinia species, Enterobacter species, Salmonella species, Serratia species, Aerobacter species, Escherichia species, Pseudomonas species, Shigella species, Vibrio species, Aeromonas species, Campylobacter species, Streptococcus species, Staphylococcus species, Lactobacillus species, Micrococcus species, Moraxella species, Bacillus species, Bordetella species, Enterococcus species, Propionibacterium species, Streptomyces species, Clostridium species, Corynebacterium species, Eberthella species, Micrococcus species, Mycobacterium species, Neisseria species, Haemophilus species, Bacteroides species, Listeria species, Erysipelothrix species, Acinetobacter species, Brucella species, Pasteurella species, Vibrio species, Flavobacterium species, Fusobacterium species, Streptobacillus species, Calymmatobacterium species, Legionella species, Treponema species, Borrelia species, Leptospira species, Actinomyces species, Nocardia species, Rickettsia species, and any other bacterial species that is capable of being flocculated according to the method described herein.

Examples of fungi that can be flocculated using the presently described method include, but are not limited to, fungi that grow as molds or are yeastlike, including, for example, fungi that cause diseases such as ringworm, histoplasmosis, blastomycosis, aspergillosis, cryptococcosis, sporotrichosis, coccidioidomycosis, paracoccidioidomycosis, mucormycosis, chromoblastomycosis, dermatophytosis, protothecosis, fusariosis, pityriasis, mycetoma, paracoccidioidomycosis, phaeohyphomycosis, pseudallescheriasis, sporotrichosis, trichosporosis, pneumocystis infection, and candidiasis.

In one embodiment, the microorganisms flocculated in accordance with the presently described method are fermenting organisms. In one embodiment, the microorganisms flocculated in accordance with the presently described method can be thermophilic microorganisms. In another embodiment, the microorganisms can be microorganisms that are not thermophilic. The microorganisms can be naturally present in the sample in which the microorganisms are flocculated (e.g., bulk fluids) or the microorganisms can be flocculated and then inoculated into a sample (e.g., bulk fluids) in which the flocculated microorganisms are separated from the bulk fluids by sedimentation. The microorganisms can be partially or completely flocculated and the microorganisms can be non-flocculating or naturally flocculating.

In one embodiment, the microorganisms are microorganisms that have been previously isolated to obtain a single species of microorganism. In another embodiment, the microorganisms have not been previously isolated. In another embodiment, the microorganisms comprise a mixture of species of microorganisms, and that mixture of microorganisms can be a mixture of isolated microorganisms or can be a mixture of microorganisms that are naturally present in a sample. In yet another embodiment, the microorganisms can be flocculated and then inoculated into a sample. Alternatively, the microorganisms can be flocculated in a sample, or can be flocculated after removal from a sample.

In one embodiment, a method of sedimenting microorganisms is provided. The method can be used to separate the flocculated microorganisms from bulk fluids. The method comprises the steps of contacting the microorganisms with a cationic flocculating agent, contacting the microorganisms with an anionic flocculating agent, flocculating the microorganisms, and sedimenting the microorganisms, such as by, for example, allowing the flocculated microorganisms to settle. In one embodiment, the microorganisms can be flocculated, inoculated into bulk fluids, and then separated from the bulk fluids by sedimentation. In another embodiment, the microorganisms can be flocculated in the bulk fluids, and then separated from the bulk fluids by sedimentation.

Cationic flocculating agents useful in the compositions and methods described herein are positively charged molecules or molecules capable of carrying one or more positive charges under predetermined conditions, and include but are not limited to salt counterions, such as metal cations and salts thereof, including iron, chromium, cobalt, nickel, copper, manganese, and the like, and including multivalent metal cations, such as divalent and trivalent metal cations, and the like; small molecules such as di-, tri-, and tetraamines; polymeric materials, such as polyamines and salts thereof; and combinations thereof. Anionic flocculating agents useful in the compositions and processes described herein are negatively charged molecules or molecules capable of carrying one or more negative charges under predetermined conditions, and include but are not limited to salt counterions such as carbonates, sulfates, phosphates, and the like; small molecules such as di-, tri-, and tetracarboxylic acids, di-, tri-, and tetrasulfinic and sulfonic acids, di-, tri-, and tetraphosphinic and phosphonic acids; polymeric materials that carry or can carry a negative charge, such as polyols, polythiols, polyacids, polysulfonates, polycarboxylates, polyphosphonates, and salts thereof; and combinations thereof.

In one aspect, the metal cations have a “2+” or a “3+” charge.

In one embodiment, a flocculating agent of a cationic type can be used in combination with a flocculating agent of an anionic type to catalyze flocculation artificially. Any combination of cationic and anionic flocculating agents may be used to catalyze flocculation. It is appreciated that combinations of cationic and anionic flocculating agents that form higher levels of aggregated solids with microorganisms are more easily separated from the bulk fluids. Illustratively, the combinations of cationic and anionic flocculating agents used in the compositions and methods described herein include at least one agent that is a polymeric material.

Illustratively, the flocculating agent of the cationic type can be ferrous chloride, ferrous sulphate, ferric chloride, ferric sulphate, chlorinated ferric sulphate, aluminium sulphates, chlorinated basic aluminum sulphates, magnesium chloride, magnesium sulphate, and the like, and combinations thereof. Illustratively, the flocculating agent of the anionic type can be anionic polyacrylamides, polyacrylates, polymethacrylates, polycarboxylates, polysaccharides (e.g., xanthan gum, partially hydrolyzed guar gums, gum Arabic, or alginates and partially hydrolyzed alginates), chitosan, celluloses, and the like, and combinations thereof. A mixture of flocculating agents of the cationic and/or the anionic type can also be used. Any synthetic flocculating agent can also be used.

Without being bound by theory, it is believed that flocculation is accomplished by the interaction and aggregation of alternating anionic flocculating agents, cationic flocculating agents, and microorganisms. It is further believed that microorganisms generally present a surface having an overall negative charge. In one illustrative embodiment, flocculation including the following is described:

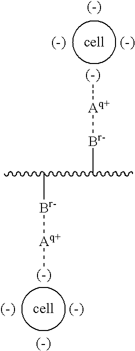

where B^{r-} represents an anionic flocculating agent and A^{q+} represents a cationic flocculating agent. In the above embodiment, the anionic flocculating agent is in the form of a polymeric compound. It is to be understood that other anionic and cationic flocculating agents may be involved in the alternating arrangement forming more complex aggregates.

In another illustrative embodiment, flocculation including the following is described:

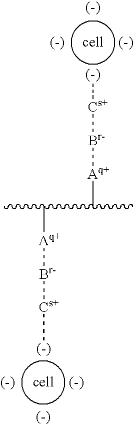

where A^{q+} represents a first cationic flocculating agent, B^{r-} represents an anionic flocculating agent, and C^{s+} represents a second cationic flocculating agent. In the above embodiment, the cationic flocculating agent is in the form of a polymeric compound.

In one aspect, where the anionic flocculating agent is a polymeric compound, the affinity of the anionic flocculating agent for the cationic flocculating agent is selected to be about competitive with the affinity of the cell for the cationic flocculating agent. It is appreciated that the relative affinities may be adjusted or modified by the conditions, such as by choice of solvent, ionic strength, pH, temperature, and the like.

In another aspect, the relative charge density on polymeric anionic and cationic flocculating agents is low. It is appreciated that low charge density may increase the aggregation of microorganisms by decreasing the amount of self-aggregation. In one aspect, the charges are separated on the polymeric anionic and/or cationic flocculating agents by more than about 50 or more than about 100 atoms. In variations where the entropy of the polymer is restricted, such as by the presence of branching, multiple bonds, and/or cyclic substructures, the charges are separated on the polymeric anionic and/or cationic flocculating agents by more than about 30 or more than about 40 atoms.

In another aspect, the relatively low charge density is understood in terms of molecular weight. Illustratively, the polymeric anionic and cationic flocculating agents include compounds having one charge per about 1000 or about 2000 atomic units. In variations where the entropy of the polymer is restricted, such as by the presence of branching, multiple bonds, and/or cyclic substructures, there is one charge per about 400 or about 600 atomic units.

Polymeric anionic and cationic flocculating agents include naturally occurring polymers, such as polysaccharides, xanthan gum, partially hydrolyzed guar gums or gum Arabic, alginates and partially hydrolyzed alginates, chitosan, celluloses, hemicelluloses, polypeptides and proteins, other emulsifying agents, and the like, and synthetic polymers, such as polyacrylamides, polyacrylates, polymethacrylates, polycarboxylates, partially hydrolyzed polyacrylamides, polyacrylates, polymethacrylates, and polycarboxylates, and the like. In one illustrative aspect, the polymeric anionic and cationic flocculating agents are food grade, such as xanthan gum and other emulsifying agents.

In one embodiment, the microorganism is artificially flocculated using ferric chloride and xanthan gum. In one embodiment, the concentration of the flocculating agent of the cationic type can range from about 0.01 ppm to about 300 ppm, and the concentration of the flocculating agent of the anionic type can range from about 0.001 g/L to about 10 g/L.

The bulk fluids can be of any volume. For example, the bulk fluids can range from a volume of about 0.1 ml to about 1000 liters. In other embodiments, the volume of the bulk fluids can be less than 0.1 ml or greater than 1000 liters. In one embodiment, the bulk fluids can be any fluids in which a microorganism is typically found. For example, the bulk fluids can be a biomaterial waste stream, a body fluid, a culture medium for microorganisms, or any fluid used to process microorganisms, such as fluids used for processing microorganisms in a research laboratory, or any other fluid in which microorganisms are typically present. In another embodiment, the microorganisms can be inoculated into the bulk fluids. The rate and extent of flocculation can be controlled by varying such conditions as pH, ion concentration (e.g., magnesium concentration), concentration of the cationic flocculating agent (e.g., iron), and by addition of organic molecules that bind to divalent ions (e.g., xylitol). Variation in such conditions can be used to flocculate a particular species of microorganism in a mixture if that microorganism flocculates under the particular conditions used and other microorganisms do not (see Examples 15-19). Thus, the method described herein can be used to separate a particular species of microorganism from another species of microorganism in a mixture of microorganisms. Accordingly, the method may be useful, for example, for separating microorganisms in a sample of body fluid for examination of the separated or isolated microorganisms employing techniques useful for diagnosis of disease states.

In one embodiment, the microorganisms flocculated by the method described herein are useful for treating a biomaterial waste stream to remove pollutants in the biomaterial waste stream by converting the pollutants to a valuable product. In this embodiment, the microorganism can be a fermenting organism. In one embodiment a biomaterial waste stream is subjected to oxidative fermentation in the presence of microorganisms flocculated by the method described herein (i.e., a fermenting organism) to convert the pollutants in the biomaterial waste stream to a valuable product. Accordingly, at least a portion of the pollutants (e.g., phosphorous, nitrogen, and potassium) is removed from the biomaterial waste stream and incorporated into the valuable product, for example, the microorganism (i.e., a fermenting organism), reducing environmental pollution. Fermentation of a biomaterial waste stream using flocculated microoganisms results in the production of a valuable protein product (e.g., a microorganism such as a yeast) that can be used, for example, as an animal feed additive, a feed supplement, a fertilizer, a fertilizer ingredient, or a soil conditioner.

Exemplary biomaterial waste streams that can be treated with microorganisms flocculated by the presently described method include, but are not limited to, manure, cellulosistic solid waste, whey broth from cheese production or biomaterial waste streams from other foodstuffs, broth remediation from alcohol or yeast production, tannery waste, slaughterhouse waste, tallow waste from rendering processes, waste derived from plants, and land fill waste. The waste derived from plants can be, for example, waste from hay, leaves, weeds, or wood and can be, for example, yard waste, landscaping waste, agricultural crop waste, forest waste, pasture waste, or grassland waste. The waste derived from foodstuffs can be fruit and vegetable processing waste, fish and meat processing wastes, bakery product waste, and the like. In embodiments where the waste is manure, the manure can be from an animal such as a human, a bovine animal, an equine animal, an ovine animal, a porcine animal, or poultry. In one embodiment the biomaterial waste stream is a variable and dilute biomaterial waste stream derived from animal manure or human waste. In general, any organic waste containing proteins, simple or complex carbohydrates, or lipids, or a combination thereof, can be treated with the microorganisms flocculated according to the method described herein. The use of microorganisms (i.e., a fermenting organism) flocculated according the presently described method allows for the extraction of nutrients from dilute biomaterial waste streams using, for example, dilution protocol fermentation. In embodiments where the flocculated microorganism is used to treat a biomaterial waste stream, the microorganism (i.e., a fermenting organism) can be any of those described above. In one embodiment, the flocculated microorganism that contacts the biomaterial waste stream can be a thermophilic microorganism. In another embodiment, the microorganism can be a microorganism that is not thermophilic. The microorganism (i.e., a fermenting organism) can be naturally present in the biomaterial waste stream and can be flocculated in the biomaterial waste stream, or the biomaterial waste stream can be inoculated with the flocculated microorganism. The microorganism can be partially or completely flocculated and the microorganism can be non-flocculating or naturally flocculating.

In embodiments where the flocculated microorganism is used to treat a biomaterial waste stream (i.e., a fermenting organism), a flocculating agent of a cationic type can be used in combination with a flocculating agent of an anionic type to induce flocculation artificially as described above. The flocculating agent of the cationic type can be any of those described above. For example, the flocculating agent of the cationic type can be selected from the group including ferrous chloride, ferrous sulphate, ferric chloride, ferric sulphate, chlorinated ferric sulphate, aluminium sulphates, chlorinated basic aluminum sulphates, magnesium chloride, magnesium sulphate, and the like. The flocculating agent of the anionic type can be any of those described above. For example, the flocculating agent of the anionic type can be selected from the group including an anionic polyacrylamide, a polyacrylate, a polymethacrylate, a polycarboxylate, a polysaccharide (e.g., xanthan gum, partially hydrolyzed guar gums or gum Arabic, or alginates or partially hydrolyzed alginates), chitosan, cellulose, and the like. A mixture of flocculating agents of the cationic and/or the anionic type can also be used. In one embodiment, the microorganism is artificially flocculated using ferric chloride and xanthan gum.

In one embodiment, the microorganism (i.e., a fermenting organism) flocculated according to the method described herein utilizes the pollutants in the biomaterial waste stream (e.g., potassium, nitrogen, and phosphorus) as nutrients, and the flocculated microorganisms produced during fermentation of the biomaterial waste stream can be used as an animal feed, an animal feed supplement, a fertilizer, a fertilizer ingredient, or a soil conditioner.

The flocculated microorganism can be preserved using any method known in the art for preventing degradation of a microorganism and/or its protein components. For example, the microorganism can be pasteurized or the microorganism can be refrigerated after removing the microorganism from the fermentation unit. The microorganism can be used as a valuable product in the form of, for example, a paste, or another aqueous mixture, or a dry powder. Alternatively, the wet product resulting from the fermentation can be used without further processing.

In one embodiment, a flocculated microorganism prepared according to the presently described method for flocculation of microorganisms is provided. In another embodiment, a feed composition is provided comprising an animal feed blend and a flocculated microorganism prepared in accordance with the presently described method. In still another embodiment an animal feed supplement comprising a flocculated microorganism prepared in accordance with the presently described method is provided.

In one embodiment, the flocculated microorganism is added to an animal feed blend to form a feed composition. Any animal feed blend known in the art can be used such as rapeseed meal, cottonseed meal, soybean meal, and cornmeal. Optional ingredients of the animal feed blend include sugars and complex carbohydrates such as both water-soluble and water-insoluble monosaccharides, disaccharides and polysaccharides. Optional amino acid ingredients that may be added to the feed blend are arginine, histidine, isoleucine, leucine, lysine, methionine, phenylalanine, threonine, tryptophan, valine, tyrosine ethyl HCl, alanine, aspartic acid, sodium glutamate, glycine, proline, serine, cysteine ethyl HCl, and analogs, and salts thereof. Vitamins that can optionally be added are thiamine HCl, riboflavin, pyridoxine HCl, niacin, niacinamide, inositol, choline chloride, calcium pantothenate, biotin, folic acid, ascorbic acid, and vitamins A, B, K, D, E, and the like. Protein ingredients can also be added and include protein obtained from meat meal, liquid or powdered egg, and the like. Any medicament ingredients known in the art can also be added to the animal feed blend such as antibiotics.

In one embodiment, the feed composition is supplemented with the flocculated microorganisms in amounts of about 0.025% to about 1% by weight of the feed composition. In another embodiment the feed composition is supplemented with the flocculated microorganisms in amounts of about 0.025% to about 2%. In yet another embodiment the feed composition is supplemented with the flocculated microorganisms in amounts of about 0.025% to about 5% by weight of the feed composition. In another embodiment the feed composition is supplemented with the flocculated microorganisms in amounts of about 0.025% to about 10% by weight of the feed composition. In each of these embodiments it is to be understood that the percentage of the flocculated microorganisms by weight of the feed composition refers to the final feed composition (i.e., the feed composition as a final mixture) containing the animal feed blend, the flocculated microorganisms, and any other optionally added ingredients.

An animal feed supplement comprising flocculated microorganisms is also provided. The animal feed supplement can be a wet or a dry product and the animal feed supplement can be processed so that it is in the form of a paste, an aqueous mixture, a dry powder, or in any other suitable form. The animal feed supplement can contain any of the components of the animal feed blend described above, and the animal feed supplement can be mixed with an animal feed blend to form a final mixture (i.e., a feed composition as a final mixture). The amounts of flocculated microorganisms by weight of the feed composition can be any of those described above.

### EXAMPLE 8

### Catalysis of Flocculation of Pichia Stipitis

Xanthan gum (0.25%; Sigma, St. Louis, Mo.) and ferric chloride solution (0.5%) were prepared. Pichia stipitis was grown in YPD medium (1% yeast extract, 2% peptone, and 2% glucose). A final yeast suspension of 4 g/L on a dry weight basis was used. Yeast suspension (40 ml) was poured into 50 ml plastic tubes, and 2 ml of 0.25% xanthan gum solution was added to achieve a final xanthan gum concentration in the yeast suspension of 0.125 g/L. Various amounts of ferric chloride solution were added to obtain an iron concentration of 20 to 90 ppm. The mixture was then gently shaken for 30 seconds, and the flocs of yeast were allowed to settle. After settling for 4 minutes, samples were taken from the top of the plastic tube and the samples contained a portion of the supernatant. Cell counts for the samples were determined by using a hemocytometer.

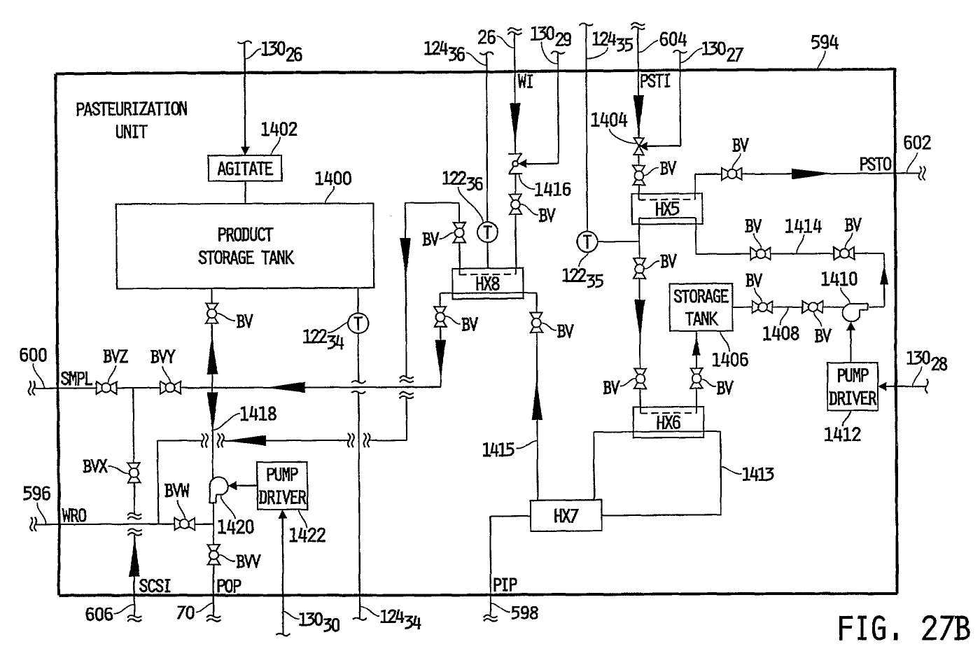

As shown in FIG. 31, iron caused a partial to a complete flocculation depending on the concentration of iron present (e.g., 70 ppm or greater for complete flocculation). The results depicted in FIG. 31 show not only that flocculation can be catalyzed, but that partial flocculation can be achieved by limiting the concentration of ferric chloride added. As the data in FIG. 31 show, at least a ten-fold variation in supernatant cell concentration can be obtained by varying the iron concentration.

Accordingly, catalyzed flocculation provides control of flocculation that is surprisingly better than that accomplished by using a naturally flocculating species of microorganism. The ratio of flocculated to unflocculated yeast cells using a naturally flocculating species, is typically about 2-3:1. When flocculation is catalyzed as shown in FIG. 31, the percentage of flocculated cells ranges from about 0 to about 100% depending on the ferric chloride concentration used.

### EXAMPLE 9

### Catalysis of Flocculation of Pinhia Stipitis

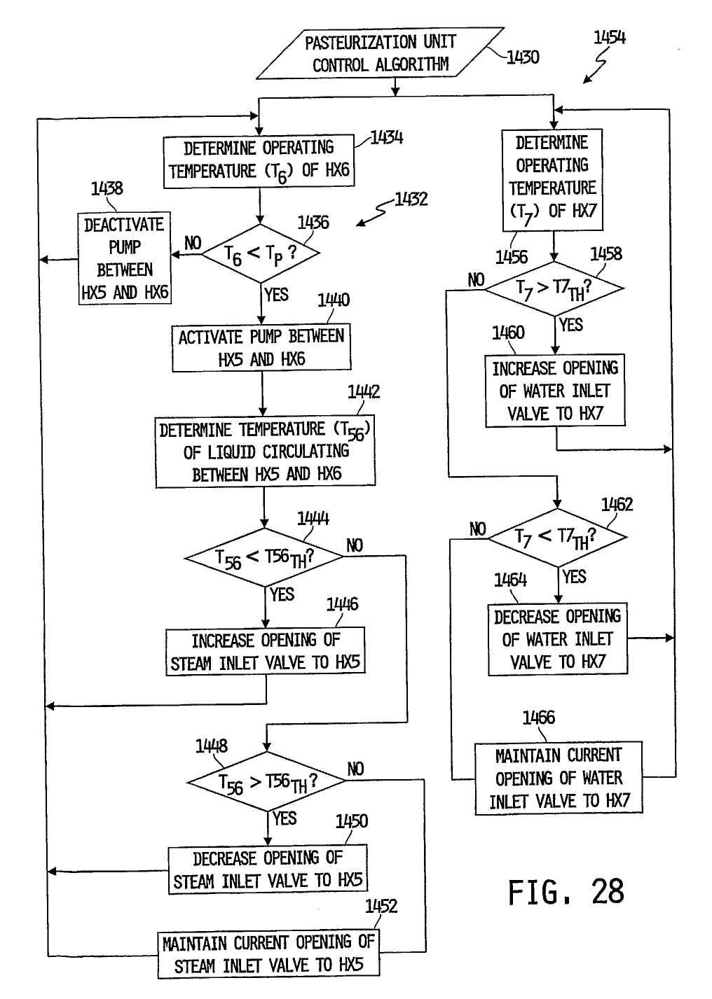

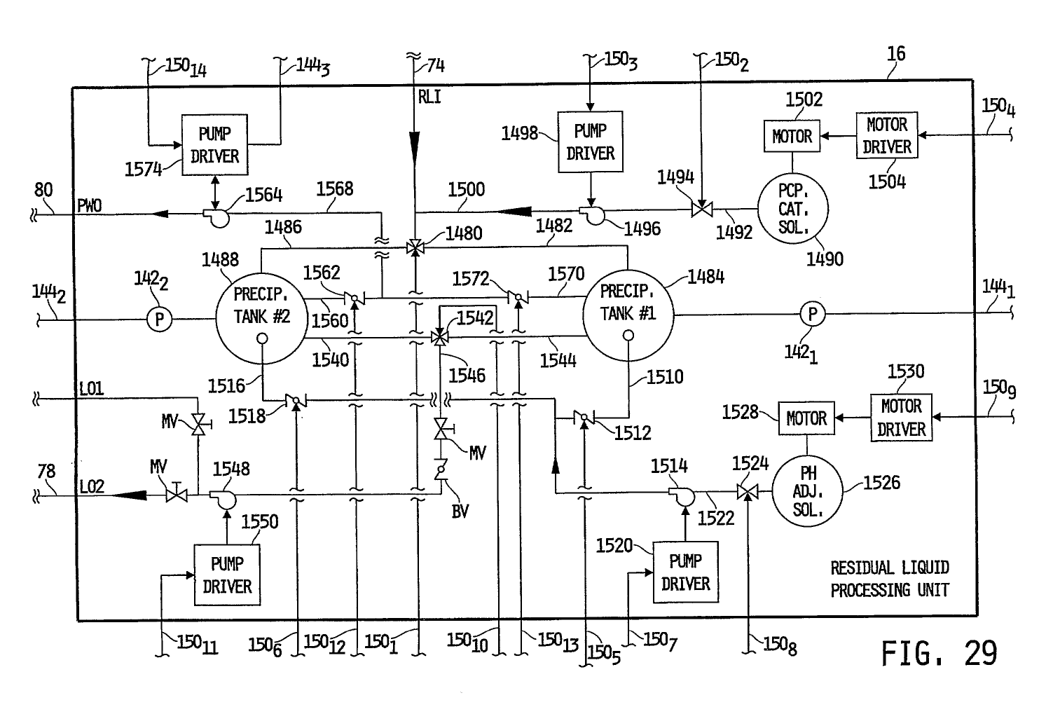

Xanthan gum (0.25%; Sigma St. Louis, Mo.) and ferric chloride solution (0.145%) were prepared. Pichia stipitis was grown in YPD medium (1% yeast extract, 2% peptone, and 2% glucose). A final yeast suspension of 4 g/L on a dry weight basis was used. Yeast suspension (10 ml) was poured into 15 ml plastic tubes, and various amounts of the xanthan gum and ferric chloride solutions were added to obtain a xanthan gum concentration of 0.00625 to 0.1 g/L, and an iron concentration of 20 to 90 ppm. The mixture was then gently shaken for 30 seconds, and the flocs of yeast were allowed to settle. After settling for 3 minutes, samples of the supernatant were taken from each tube. For each sample, optical density (O.D.) at 600 nm was measured (see FIG. 32 and Table 8), and the iron concentration was determined by using an iron diagnostic kit (Sigma, St. Louis, Mo.) based on the Persijn method (see FIG. 33 and Table 9). The results depicted in FIG. 32 show that flocculation can be catalyzed, that the concentration of iron that achieves complete flocculation is dependent on the concentration of xanthan gum, and that flocculation can be controlled (i.e., partial flocculation can be achieved) by varying the concentration of iron. Similar results were obtained using ferric sulfate in place of ferric chloride. With no yeast present, ferric ion and xanthan gum do not precipitate, and xanthan gum does not precipitate in the absence of ferric ion. Flocculation of Saccharomyces cerevisiae and Candida utilis were also catalyzed and controlled using this method.

TABLE 8

| | OD at 600 nm | | |
|---|---|---|---|
| Iron (ppm) | 0.1 g/L xanthan | 0.05 gL xanthan | 0.025 g/L xanthan |
| 0 | 4.96 | 4.99 | 5.02 |
| 5 | 4.41 | 4.26 | 4.67 |
| 10 | 3.47 | 3.26 | 2.83 |
| 15 | 2.37 | 1.08 | 0.227 |
| 20 | 0.417 | 0.074 | 0.101 |
| 25 | 0.095 | 0.05 | 0.077 |
| 30 | 0.066 | 0.05 | 0.077 |
| 35 | 0.066 | 0.05 | 0.077 |

TABLE 9

OD at 600 nm

| Iron (ppm) | 0.0125 g/L xanthan | 0.00625 gL xanthan |
|---|---|---|
| 0 | 5.4 | 5.37 |
| 2 | 5.29 | 5.44 |
| 4 | 4.95 | 5.32 |
| 6 | 4.6 | 4.86 |
| 8 | 3.99 | 4.53 |
| 10 | 2.94 | 3.82 |
| 12 | 1.67 | 2.63 |
| 14 | 0.744 | 1.45 |
| 16 | 0.134 | 0.312 |
| 18 | 0.134 | 0.215 |

### EXAMPLE 10

### Catalysis of Flocculation of Saccharomyces Cerevisiae

Xanthan gum (0.25%; Sigma St. Louis, Mo.), 0.145% ferric chloride, 0.56% magnesium sulphate, and 5% sodium chloride solutions were prepared. Saccharomyces cerevisiae was grown in YPD medium (1% yeast extract, 2% peptone, and 2% glucose). A final yeast suspension of 4 g/L on a dry weight basis was prepared at its natural pH of 4.8. Assays were performed to test the effects of magnesium, pH, and sodium chloride on flocculation.

In the first assay, 10 ml of yeast suspension was poured into 15 ml plastic tubes. Xanthan gum was added to obtain a final concentration of 0.025 g/L. Magnesium sulphate was added to each tube to obtain a final magnesium concentration of 0.5 g/L. Varying amounts of ferric chloride solution were added to obtain an iron concentration of 0 to 35 ppm.

In the second assay, the pH of the yeast suspension was adjusted to 7.11 by adding 4N sodium hydroxide. Yeast suspension (10 ml) was poured into 15 ml plastic tubes. Xanthan gum was added to achieve a final concentration of 0.025 g/L. Varying amounts of ferric chloride solution were added to obtain an iron concentration of 0 to 30 ppm.

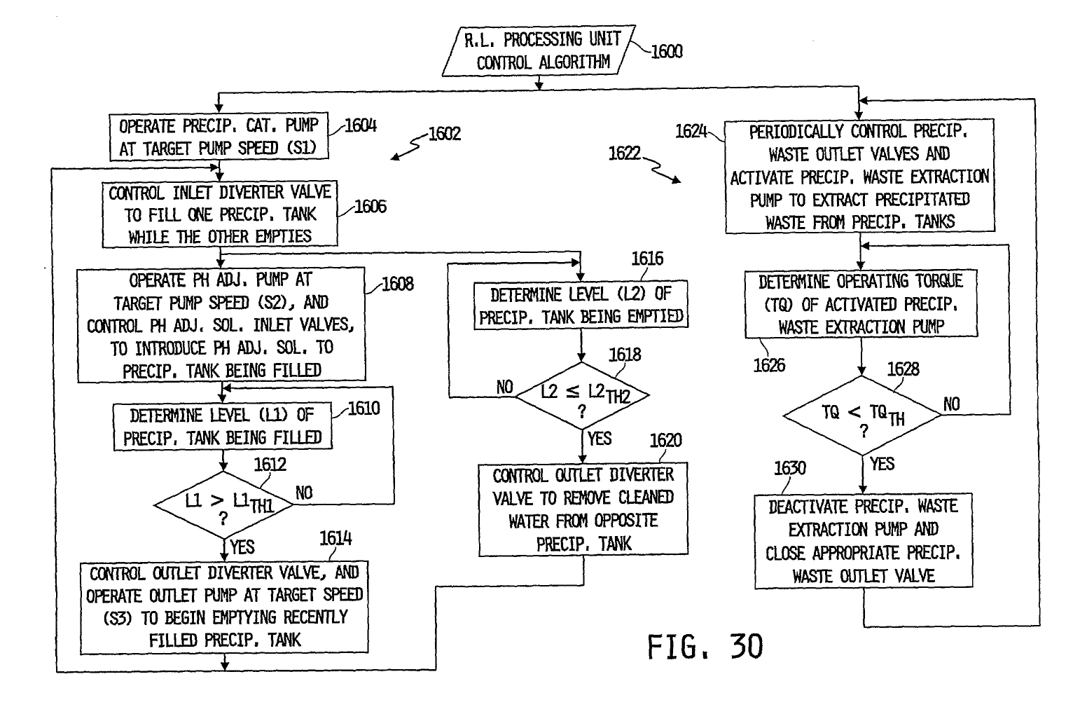

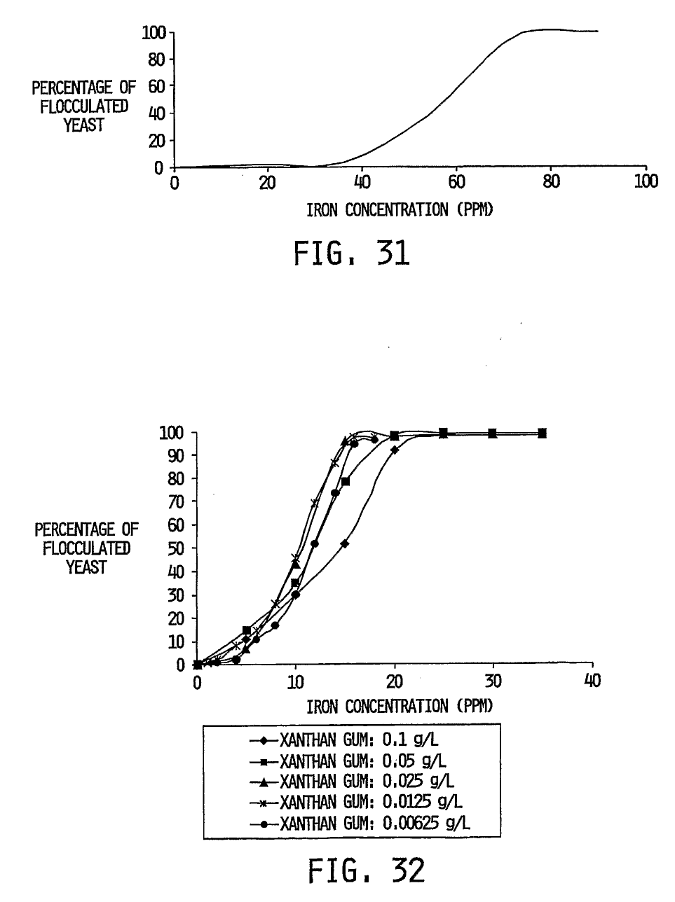

In the third assay, 10 ml of yeast suspension was poured into 15 ml plastic tubes. Xanthan gum was added to achieve a final concentration of 0.025 g/L. Sodium chloride was added to each tube to obtain a final concentration of 2.5 g/L, and varying amounts of ferric chloride solution were added to obtain an iron concentration of 0 to 20 ppm. A control assay was also included in which 10 ml of yeast suspension was poured into 15 ml plastic tubes and xanthan gum was added to achieve a final concentration of 0.025 g/L. Varying amounts of ferric chloride solution were added to obtain an iron concentration of 0 to 15 ppm. In all four assays, the flocs of yeast were allowed to settle for 2 minutes, and samples were taken from the supernatant in each tube. For each sample, optical density (O.D.) at 600 nm was measured (see FIG. 34 and Table 10), and the iron concentration was determined by using an iron diagnostic kit (Sigma, St. Louis, Mo.) based on the Persijn method (see FIG. 35 and Table 11).

Magnesium ion is expected to cause interference with ferric ion for binding to the complex containing yeast and xanthan gum. However, the results depicted in FIGS. 34 and 35 show that magnesium ion prevents the binding of excess ferric ion to the complex, but does not interfere with flocculation because flocculation proceeds in the presence of magnesium ion (see FIG. 34), but iron in the supernatant is increased in the presence of magnesium ion. Competing sodium ion and increased pH (i.e., a pH of about 7) have little effect.

TABLE 10

| | | | | | | | OD |
|---|---|---|---|---|---|---|---|
| | | | | | | | at |
| | | | | | | | 600 |
| pH = | OD at | pH = | OD at | | OD at | pH = | nm |
| 4.8 | 600 | 4.8 | 600 nm | | 600 nm | 4.8 | 2.5 |
| Iron | nm | Iron | 0.5 g/L | Iron | pH = | Iron | g/L |
| (ppm) | Control | (ppm) | Mg | (ppm) | 7.11 | (ppm) | NaCl |
| 2 | 4.75 | 0 | 4.31 | 0 | 4.54 | 0 | 4.69 |
| 4 | 4.76 | 5 | 4.32 | 5 | 4.39 | 5 | 4.56 |
| 6 | 3.82 | 10 | 2.95 | 10 | 3.93 | 9 | 2.32 |
| 7 | 3.27 | 12 | 2.2 | 15 | 2.95 | 12 | 1.37 |
| 8 | 2.38 | 15 | 1.29 | 20 | 1.14 | 15 | 0.497 |
| 9 | 1.61 | 20 | 0.91 | 25 | 0.273 | 18 | 0.322 |
| 10 | 1.39 | 25 | 0.76 | 30 | 0.032 | 20 | 0.184 |
| 12 | 0.51 | 30 | 0.6 | | | | |
| 15 | 0.018 | 35 | 0.62 | | | | |

TABLE 11

| | | | Iron in the | | Iron in the | | Iron in the |
|---|---|---|---|---|---|---|---|
| | Iron in the | | supernatant | | supernatant | | supernatant |
| pH = 4.8 | supernatant | pH = 4.8 | (ppm) | | (ppm) | pH = 4.8 | (ppm) |
| Iron | (ppm) | Iron | 0.5 | Iron | pH = | Iron | 2.5 g/L |
| (ppm) | Control | (ppm) | g/L Mg | (ppm) | 7.11 | (ppm) | NaCl |
| 2 | 1.5 | 0 | 0 | 0 | 0.8 | 0 | 0.2 |
| 4 | 2.6 | 5 | 3.3 | 5 | 2.8 | 5 | 2.9 |
| 6 | 2.8 | 10 | 4.2 | 10 | 5 | 9 | 3.4 |
| 7 | 2.8 | 12 | 4 | 15 | 3.5 | 12 | 3.4 |
| 8 | 2.5 | 15 | 4.6 | 20 | 2.4 | 15 | 4.4 |
| 9 | 2.5 | 20 | 5.6 | 25 | 1.3 | 18 | 6 |
| 10 | 2.6 | 25 | 7.4 | 30 | 2 | 20 | 7.4 |
| 12 | 3 | 30 | 8.7 | | | | |
| 15 | 4.2 | 35 | 9 | | | | |

### EXAMPLE 11

### Catalysis of Flocculation of Yeast Resists Dilution

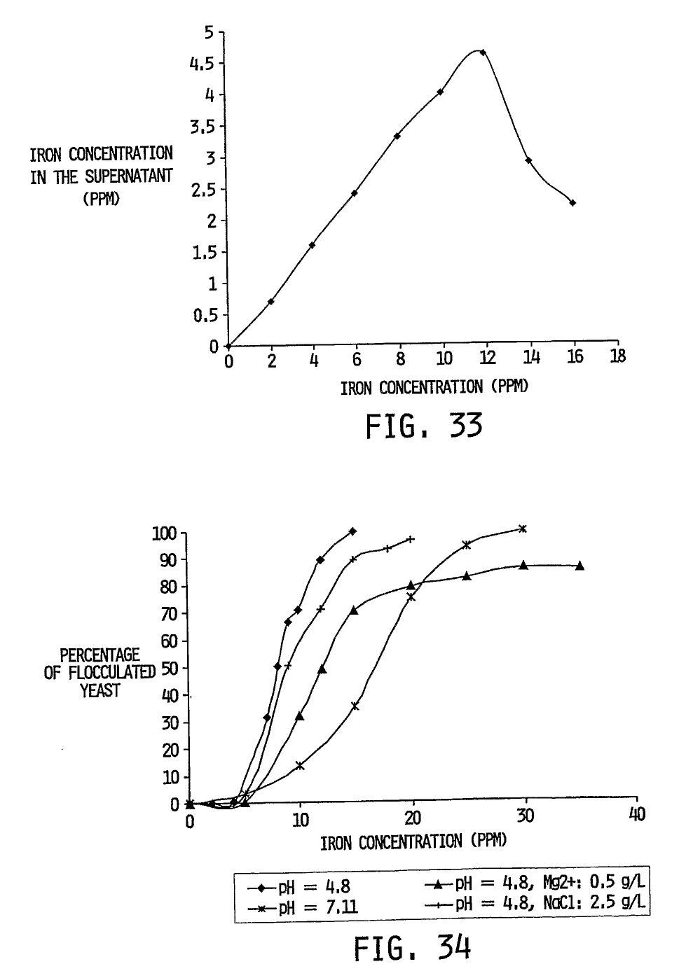

Saccharomyces cerevisiae, Pichia stipitis, and Candida utilis were used in the experiment shown in FIG. 36. All yeast types were grown in YPD medium. Yeast suspensions of approximately 4 g/L on a dry weight basis were prepared. Yeast suspensions of 10 ml each were placed in 15 ml tubes and 0.025 g/L xanthan gum and 15 ppm of iron (from ferric chloride) were added. Flocs of yeast were allowed to settle. After 3 minutes, 3 ml was taken from the supernatant, and the sample was replenished with 3 ml of distilled water. This dilution process was repeated as many times as necessary. For each sample, the optical density at 600 nm was measured to calculate the percentage of yeast in the flocculating form. The results depicted in FIG. 36 show that yeast flocculated with xanthan gum and ferric ion forms a stable complex that resists “wash-out” by dilution which is relevant to dilution protocol fermentation in which dilute substrates are used. The low concentration of xanthan gum (0.0025%) binds to yeast and resists washing under conditions of at least 100% dilution.

### EXAMPLE 12

### Settling Rate of Yeast after Catalyzed Flocculation

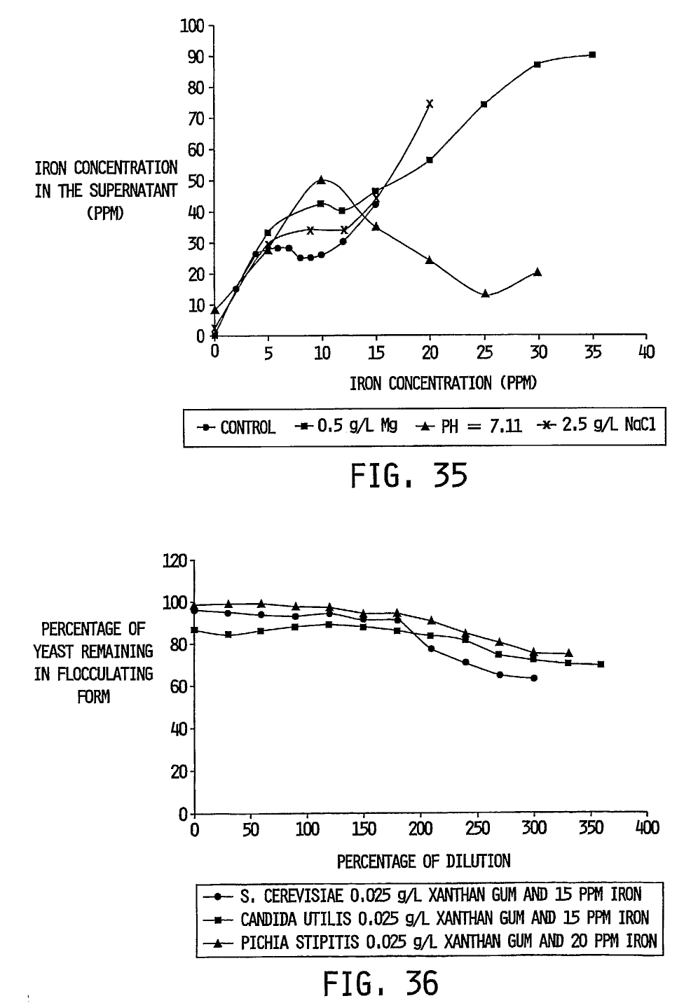

Saccharomyces cerevisiae, Kluyveromyces lactis, Pichia stipitis, and Candida utilis were used in the experiment shown in FIG. 37. All yeast were grown in YPD medium. Yeast suspensions of approximately 4 g/L on a dry weight basis were prepared. A 100 ml volume of yeast suspension with 0.025 g/L xanthan gum and 15 ppm iron from ferric chloride was poured into a 100 ml cylinder. The depth of the settled yeast flocs was measured against time to calculate the average settling rate of flocculated yeast (see FIG. 37). As shown in FIG. 37, the settling rate of yeast after flocculation is at least 0.1 inch/minute (0.254 centimeter/minute). In comparison, the settling rate of unflocculated yeast is about 0.008 inch/minute (0.020 centimeter/minute).

### EXAMPLE 13

### Effect of pH on the Catalysis of Flocculation of Saccharomyces Cerevisiae

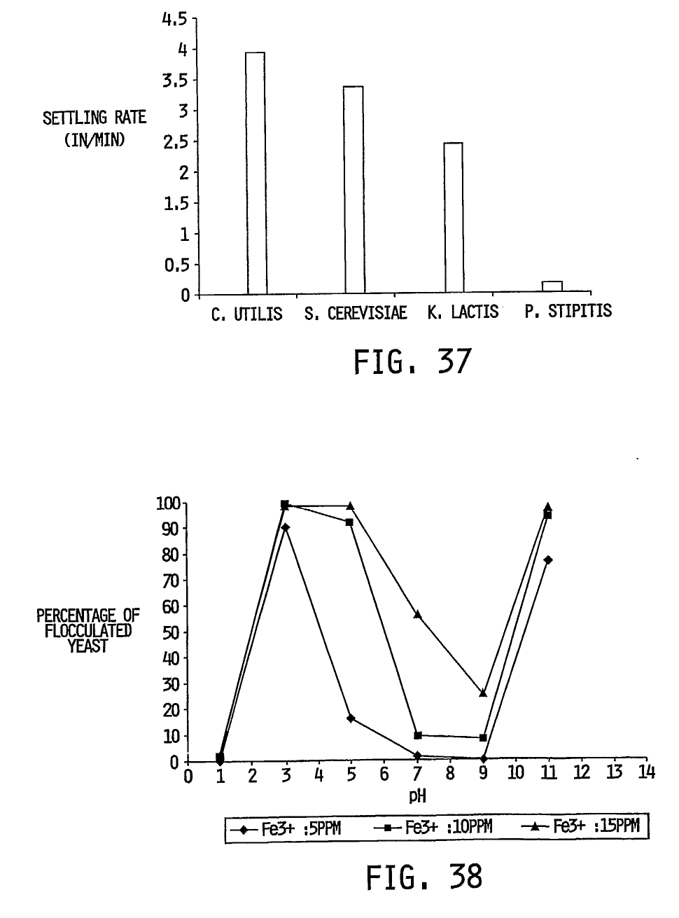

Xanthan gum (0.025 g/L; Sigma, St. Louis, Mo.) and 0.145% ferric chloride solutions were prepared. Saccharomyces cerevisiae was grown in YPD medium (1% yeast extract, 2% peptone, and 2% glucose). The yeast were harvested by centrifugation and were resuspended in deionized water. Final yeast suspensions of 4 g/L dry weight density were used. The pH's of the yeast suspensions were adjusted to pH 1, 3, 5, 7, 9, and 11 by adding appropriate amounts of 10% sulfuric acid and 10% sodium hydroxide. The yeast suspensions (10 ml) were poured into 15 ml tubes. Xanthan gum solution was added to obtain a xanthan gum concentration of 0.025 g/L, and ferric chloride solution was added to obtain an iron concentration of 5 ppm, 10 ppm, or 15ppm. The suspensions were mixed, settled for 3 minutes, and samples were taken from each tube from the supernatant. For each sample, optical density (O.D.) at 600 nm was measured, and the percentage of flocculated yeast was calculated (see FIG. 38). As shown in FIG. 38, variation in pH affects the catalysis of flocculation of yeast.

### EXAMPLE 14

### Effect of pH and Xylitol on the Catalysis of Flocculation of Saccharomyces Cerevisiae

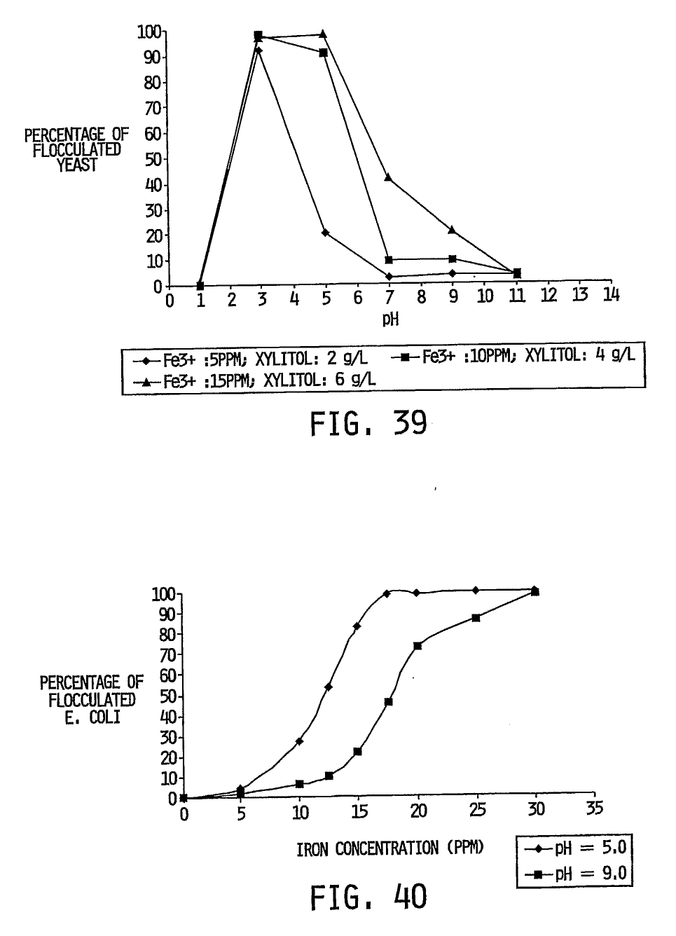

Xanthan gum (0.025 g/L; Sigma, St. Louis, Mo.), 20% xylitol (Sigma, St. Louis, Mo.) and 0.145% ferric chloride solutions were prepared. Saccharomyces cerevisiae was grown in YPD medium (1% yeast extract, 2% peptone, and 2% glucose). The yeast were harvested by centrifugation and resuspended in deionized water. Final yeast suspensions of 4 g/L dry weight density were used. The pH's of the yeast suspensions were adjusted to 1, 3, 5, 7, 9, and 11 by adding appropriate amounts of 10% sulfuric acid and 10% sodium hydroxide. The yeast suspensions (10 ml) were poured into 15 ml tubes. Xanthan gum solution was added to each suspension to obtain a xanthan gum concentration of 0.025 g/L. Ferric chloride solution was added to obtain an iron concentration of 5 ppm, 10 ppm, or 15 ppm and xylitol solution was added to obtain xylitol concentrations of 2 g/L, 4 g/L, or 6 g/L. The suspensions were mixed, settled for 3 minutes, and samples were taken from each tube from the supernatant. For each sample, optical density (O.D.) at 600 nm was measured, and the percentage of flocculated yeast was calculated (see FIG. 39). The results presented in FIG. 39 show that xylitol which binds divalent ions, blocks the recovery of flocculation that occurs at high pH (see FIG. 38) and increasing iron concentration tends to reverse this effect (see FIG. 39).

### EXAMPLE 15

### Catalysis of Flocculation of Gram Negative Bacteria

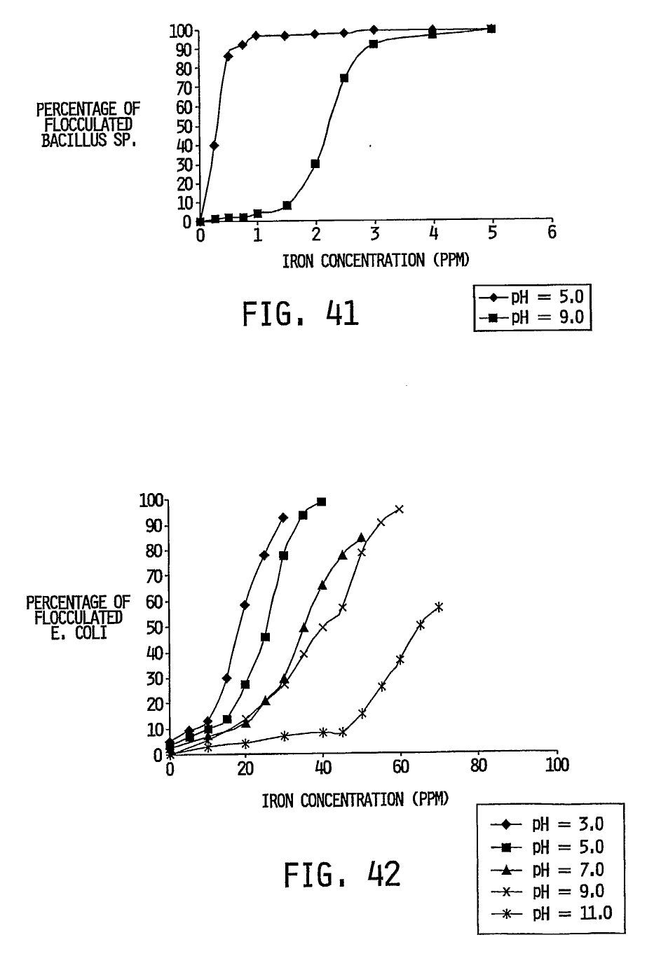

Xanthan gum (0.025 g/L; Sigma, St. Louis, Mo.) and 0.145% ferric chloride solutions were prepared. E. coli (gram negative bacteria) were grown in YPD medium (1% yeast extract, 2% peptone, and 2% glucose) under limited aeration. The E. coli were harvested by centrifugation and resuspended in deionized water. A final E. coli suspension of 4 g/L dry weight density was used. The pH's of E. coli suspensions were adjusted to 5 and 9 by adding appropriate amounts of 10% sulfurric acid and 10% sodium hydroxide. Aliquots of E. coli suspension were poured into 15 ml tubes. Xanthan gum solution was added to each tube to obtain a xanthan gum concentration of 0.025 g/L, and various amounts of ferric chloride solution were added to obtain iron concentrations of from 5 to 30 ppm. The solutions were mixed, settled for 3 minutes, and samples were taken from each tube from the supernatant. For each sample, optical density (O.D.) at 600 nm was measured, and the percentage of flocculated E. coli was calculated (see FIG. 40). The results depicted in FIG. 40 show that the flocculation of E. coli can be catalyzed and controlled and that the iron concentration for half-maximal flocculation is about 12 ppm at pH 5 and about 15 ppm at pH 9.

### EXAMPLE 16

### Catalysis of Flocculation of Gram Positive Bacteria

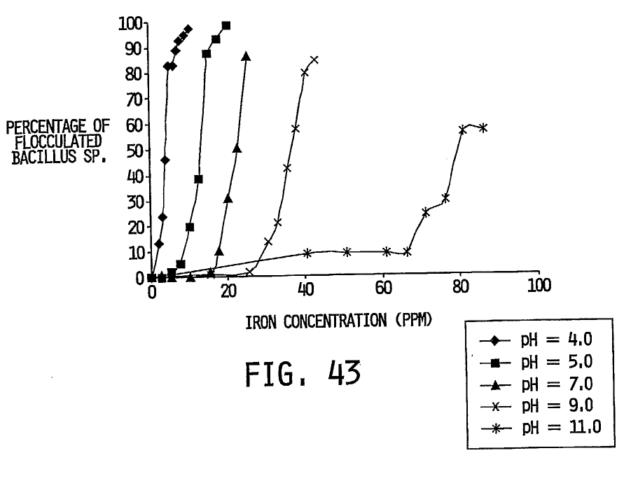

Xanthan gum (0.025 g/L; Sigma, St. Louis, Mo.) and 0.145% ferric chloride solutions were prepared. Bacillus sp. (gram positive bacteria) was grown in YPD medium (1% yeast extract, 2% peptone, and 2% glucose) under limited aeration. Bacillus sp. was harvested by centrifugation and resuspended in deionized water. A final Bacillus sp. suspension of 4 g/L dry weight density was used. The pH's of Bacillus sp. suspensions were adjusted to 5 and 9 by adding appropriate amounts of 10% sulfuric acid and 10% sodium hydroxide. Bacillus sp. suspensions were poured into 15 ml tubes. Xanthan gum solution was added to each tube to obtain a xanthan gum concentration of 0.025 g/L, and various amount of ferric chloride solution was added to obtain iron concentrations of from 0.2 to 5 ppm. The solutions were mixed, settled for 3 minutes, and samples were taken from each tube from the supernatant. For each sample, optical density (O.D.) at 600 nm was measured, and the percentage of flocculated Bacillus sp. was calculated. The results depicted in FIG. 41 show that the flocculation of Bacillus sp. can be catalyzed and controlled. In contrast to E. coli, the iron concentration for half-maximal flocculation is about 0.2 ppm at pH 5 and about 2 ppm at pH 9.

### EXAMPLE 17

### Catalysis of Flocculation of Gram Negative Bacteria

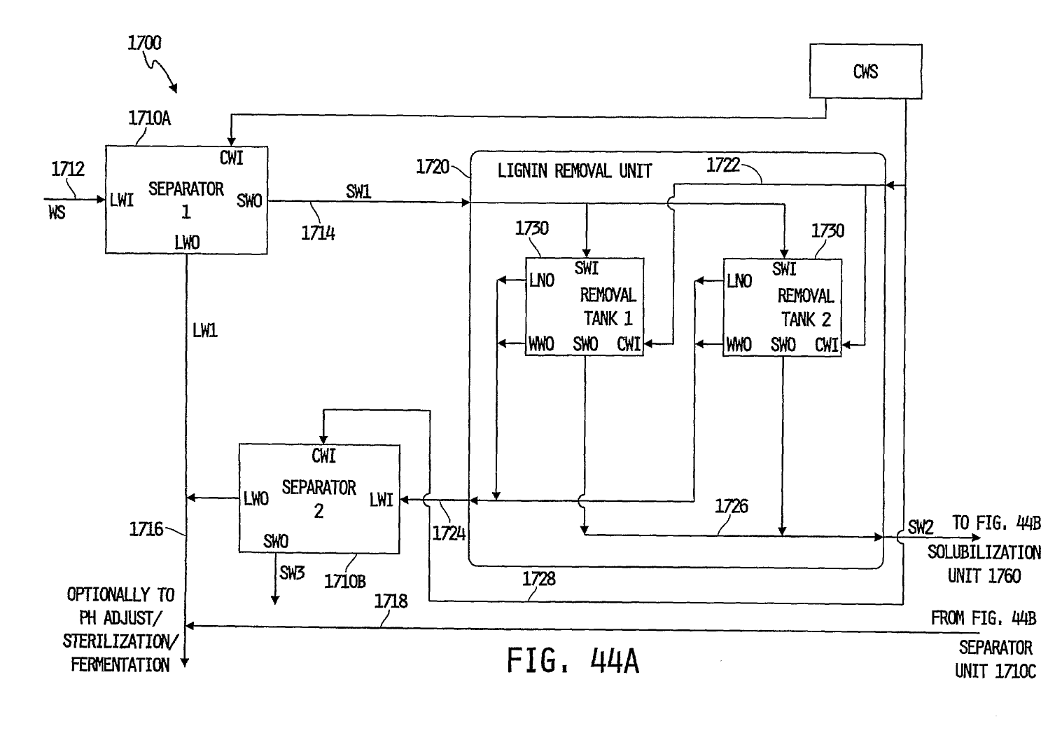

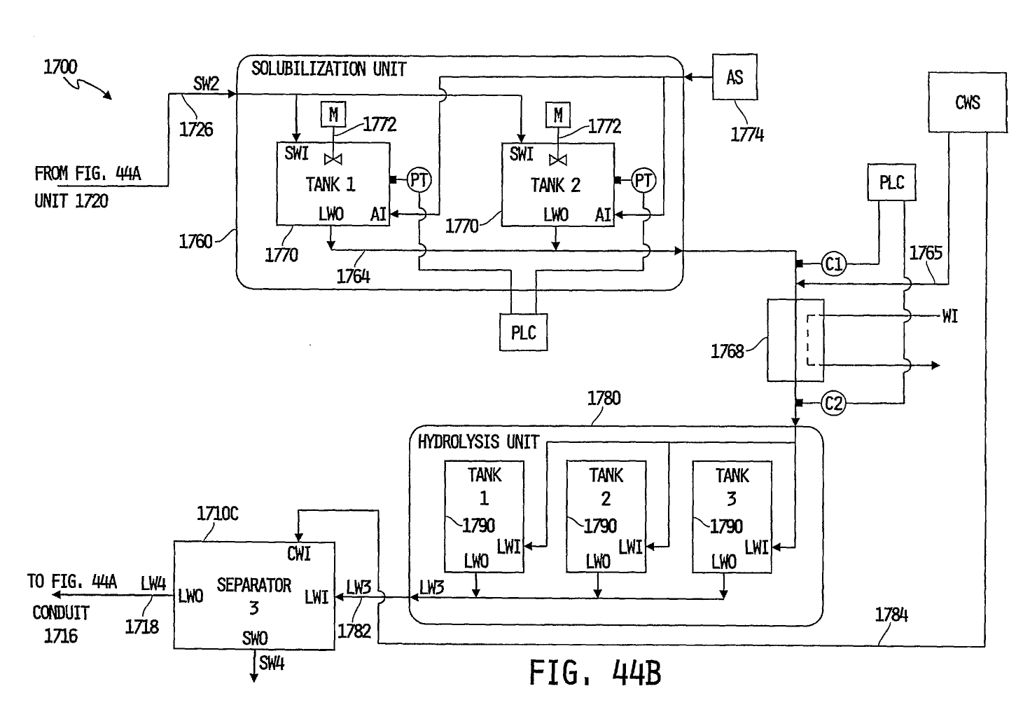

Xanthan gum (0.025 g/L; Sigma, St. Louis, Mo.) and 0.145% ferric chloride solutions were prepared. E. coli (gram negative bacteria) were grown in YPD medium (1% yeast extract, 2% peptone, and 2% glucose) under limited aeration. The E. coli were harvested by centrifugation and resuspended in deionized water. A final E. coli suspension of 4 g/L dry weight density was used. The pH's of E. coli suspensions were adjusted to 3, 5, 7, 9, and 11 by adding appropriate amounts of 10% sulfuric acid and 10% sodium hydroxide. Aliquots (10 ml) of E. coli suspension were poured into 15 ml tubes. Xanthan gum solution was added to each tube to obtain a xanthan gum concentration of 0.025 g/L, and various amounts of ferric chloride solution were added to obtain iron concentrations of from 5 to 70 ppm. The solutions were mixed, settled for 3 minutes, and samples were taken from each tube from the supernatant. For each sample, optical density (O.D.) at 600 nm was measured, and the percentage of flocculated E. coli was calculated (see FIG. 40). The results depicted in FIG. 42 show that the flocculation of E. coli can be catalyzed and controlled. In comparison to FIG. 43, the iron concentration for half-maximal flocculation of E. coli is at least 20 ppm at pH 3, and is about 2 ppm at pH 4 for Bacillus sp.

### EXAMPLE 18

### Catalysis of Flocculation of Gram Positive Bacteria

Xanthan gum (0.025 g/L; Sigma, St. Louis, Mo.) and 0.145% ferric chloride solutions were prepared. Bacillus sp. (gram positive bacteria) was grown in YPD medium (1% yeast extract, 2% peptone, and 2% glucose) under limited aeration. Bacillus sp. was harvested by centrifugation and resuspended in deionized water. A final Bacillus sp. suspension of 4 g/L dry weight density was used. The pH's of Bacillus sp. suspensions were adjusted to 4, 5, 7, 9, and 11 by adding appropriate amounts of 10% sulfuric acid and 10% sodium hydroxide. Bacillus sp. suspensions (10 ml) were poured into 15 ml tubes. Xanthan gum solution was added to each tube to obtain a xanthan gum concentration of 0.025 g/L, and various amounts of ferric chloride solution were added to obtain iron concentrations of from 2 to 85 ppm. The solutions were mixed, settled for 3 minutes, and samples were taken from each tube from the supernatant. For each sample, optical density (O.D.) at 600 nm was measured, and the percentage of flocculated Bacillus sp. was calculated. The results depicted in FIG. 43 show that the flocculation of Bacillus sp. can be catalyzed and controlled. In comparison to FIG. 42, the iron concentration for half-maximal flocculation of Bacillus sp. is about 2 ppm at pH 4 and is at least 20 ppm at pH 3 for E. coli. Thus, much lower iron concentration is needed for flocculation of gram-positive bacteria than for gram-negative bacteria, especially at low pH. Furthermore, in mixed cultures of gram-positive and negative bacteria, at appropriate iron and xanthan gum concentrations and at an appropriate pH, separation of gram positive and gram negative bacteria can be achieved (see Example 19).

### EXAMPLE 19

### Separation of Gram Negative and Gram Positive Bacteria

Xanthan gum (0.025 g/L; Sigma, St. Louis, Mo.) and 0.145% ferric chloride solutions were prepared. Bacillus sp. (gram positive bacteria) and E. coli (gram negative bacteria) were grown in YPD medium (1% yeast extract, 2% peptone, and 2% glucose) under limited aeration for 18 hours. For both types of microorganisms, cells were harvested by centrifugation and resuspended in deionized water. Final cell suspensions of 4 g/L dry weight density were used. The pH's of aliquots of the cell suspensions were adjusted to 4, 5, 7, 9, and 11 by adding appropriate amounts of 10% sulfuric acid and 10% sodium hydroxide. Bacillus sp. and E. coli suspensions (5 ml each) were mixed together in 15 ml tubes. Xanthan gum solution was added to each tube to obtain a xanthan gum concentration of 0.025 g/L, and ferric chloride solution was added to obtain an iron concentration of 10 ppm. The solutions were mixed, settled for 3 minutes, and samples were taken from each tube from the supernatant. An appropriate dilution of 10¹ to 10⁶ was made. A gram stain was performed on the sample that was diluted 1:10. The number of gram positive and gram negative cells were counted under the microscope, and 0.1 ml samples were spread on Bacto EMB agar plates and Bacto TSA blood agar plates. The plates were incubated at 37° C. for 24 hours.

The gram-stain showed that in the supernatant over 90% of cells were gram negative E. coli (30 gram-positive organisms versus 310 gram negative organisms per slide). The gram-positive Bacillus sp. causes a β-hemolytic reaction on TSA blood agar, and does not grow on EMB agar. On EMB agar, E. coli grows colonies with blue-black centers and a green metallic sheen. After 24 hours of incubation, the sample with the 1:106 dilution had 164 colonies on EMB agar, and no hemolytic reaction was observed on TSA blood agar. Thus, under appropriate conditions, gram negative E. coli can be enriched in the supernatant from the mixed cultures by flocculating most gram positive Bacillus sp.

### Illustrative Embodiments of a Process and Apparatus for Removing Solids from Aqueous Solutions

Processes and apparatus are described herein for removing dissolved, undissolved, and/or suspended solids from aqueous solutions. In one embodiment, processes and apparatus are described herein for removing dissolved, undissolved, or suspended solids from aqueous solutions by precipitation. In another embodiment, processes and apparatus are described herein for removing dissolved, undissolved, or suspended solids from aqueous solutions by crystallization. In another embodiment, processes and apparatus are described herein for removing dissolved, undissolved, or suspended solids from aqueous solutions by aggregation. In another embodiment, processes and apparatus are described herein for removing other dissolved, undissolved, or suspended solids from aqueous solutions by absorption and/or adsorption. As used herein, the term “aggregation” will generally refer to each of these processes and various combinations of these processes.

The aqueous solutions used in the processes and apparatus described herein may be derived from any source. In one embodiment, the aqueous solution is a dilute solution. In another embodiment, the aqueous solution is provided by or derived from a solution exiting a fermentation process, including a fermentation process described herein. It is understood that such a fermentation process may be used to remove certain components from an input stream, such as a biomaterial waste stream derived from animal waste, animal manure, including ruminant, semi-ruminant, swine, and poultry manure, cellulosistic waste, food processing waste, including whey broth from cheese production, broth remediation from alcohol production or yeast production, tannery waste, slaughterhouse waste, tallow waste, including waste from rendering processes, used fats and/or cooking oils, landscaping waste, including waste derived from plants, paper processing waste, land fill waste, and the like. The waste derived from animals that may be treated using the processes and apparatus described herein can be, for example, from ruminants, including semi, partial, and full ruminants, swine, including growers, beef cattle, dairy cattle, horses, poultry, including layers and broilers, and the like. The waste derived from plants can be, for example, waste from hay, leaves, weeds, sawdust, or wood and can be, for example, yard waste, landscaping waste, agricultural crop waste, forest waste, pasture waste, and/or grassland waste. The waste derived from foodstuffs can be fruit and vegetable processing waste, fish and meat processing wastes, bakery product waste, waste from cheese production such as whey, used fats and oils, and the like.

In one illustrative embodiment, the processes and apparatus described herein may be used to remove components from a biomaterial waste stream that are not removed by a fermentation process. In one aspect, a biomaterial waste stream is fed into a fermentation process, and the resulting fermented biomaterial waste stream is fed into a process or apparatus described herein for removing components from aqueous solutions. In one variation, the fermented biomaterial waste stream cannot be recycled, and/or discarded or disposed of in some manner because it contains a dissolved, undissolved, or suspended solid preventing disposal. In another variation, a biomaterial waste stream or a fermented biomaterial waste stream is fed into a process or apparatus described herein for removing components from aqueous solutions, and the resulting treated biomaterial waste stream is cleaned, purified, and/or clarified and may be discarded or disposed of in ordinary disposal streams, such as a sanitary landfill or as ground water, and/or is recycled as cleaned, clarified, or purified water into other processes or apparatus, such as processes or apparatus described herein.

Illustratively, dissolved, undissolved, and/or suspended solids or components that may be removed from aqueous solutions using the processes and apparatus described herein include metals, cations, and anions, including inorganic anions such as sulfate, phosphate, and the like. Other dissolved, undissolved, or suspended components that may be removed using the processes and apparatus described herein include organic molecules and natural polymers, such as organophosphates, lignins, peptides, proteins, and the like, as well as microorganisms such as bacteria, yeast, fermenting organisms used in a fermentation process, and the like.

In embodiments of the processes and apparatus described herein that include a fermentation step, it is appreciated that the fermenting organism used in a fermentation process may have specific requirements for using various nutrients including carbohydrate, protein, nitrogen, sodium, potassium, calcium, phosphate, and others. Because the input stream of waste fed to the fermenting organism may not have the identical ratio of such components that matches the requirements of the organism, after the fermentation, one or more nutrients may remain as the supply of limiting nutrients are exhausted. It is understood that nutrients also remain when the fermentation is performed at sub-optimal levels, and even the supply of limiting nutrient is not exhausted. Illustratively, in a fermentation processes, the limiting nutrient may be carbohydrate, and there may therefore be a relative abundance of other nutrients, such as phosphorus and nitrogen-containing compounds or components, in the aqueous solution exiting the fermentation step after the supply of carbohydrate is exhausted by the fermenting organism. When the absolute level of phosphorus, nitrogen, or some other component is higher than that which may be discarded as clarified, cleaned, or purified water, the processes and apparatus described herein may be used to remove a portion of this phosphorus, nitrogen, or other component sufficient to allow disposal of the aqueous solution as clarified, cleaned, or purified water.

In one embodiment, the aqueous solution includes phosphorus that may be removed. The phosphorus may be present in the aqueous solution as inorganic phosphates, or salts thereof, and/or as organic phosphates, including intermediates and metabolites of biochemical and biological processes, such as glucose phosphates, nucleotides, cyclic-AMP, ADP, and derivatives thereof, phytic acid and other phosphoinositols, and the like, and partial degradation products thereof. The phosphorus may also be present in aqueous solutions as components of microorganisms, bacteria, yeast, fermenting organisms, and the like. It is appreciated that in aqueous solutions containing phosphorus-containing components that exit a fermentation process, the major phosphorus-containing components may be organic phosphates. It is understood that some fermenting organisms will preferentially use inorganic phosphates present in the biomaterial waste stream before using organic phosphates. However, it is also understood that other fermenting organisms may use organic phosphates preferentially, or use inorganic or organic phosphates equally. Still other fermenting organisms may use phosphatase enzymes, such as phytase, nucleosidase enzymes, and the like to facilitate the use of organic phosphates.

It is understood that in embodiments that include a fermentation step, the aqueous solution provided to the processes and apparatus for removing solids described herein may also contain sodium, potassium, ferrous, ferric, chloride, hydroxide, carbonate, sulfate, and other ions, and salts. It is further understood, that if sufficient sodium and/or potassium are present, phosphate remains soluble. The addition of catalysts such as divalent metal ions, trivalent metal ions, transition metal ions, and/or polymeric components may allow complexes to form with the phosphate, and possibly other anions, including carbonate and sulfate. At predetermined pH levels, these complexes may not be soluble, or may not remain suspended in the aqueous solution.

Aggregation catalysts that may be used in the processes and apparatus described herein include divalent metal ions, trivalent metal ions, transition metal ions, and polymeric components. In one aspect, the divalent and trivalent metal ions include calcium and aluminum, and the like. In another aspect, the transition metal ions include iron, cobalt, nickel, copper, chromium, molybdenum, and the like. It is appreciated that transition metals such as iron and copper may provide more flexibility in disposal of precipitates, aggregates, absorption and/or adsorption complexes that are formed. In another aspect, aluminum ions may be added as aluminum sulfate, aluminum hydroxide, aluminum silicates, other silicates, silicas, BENTONITES, clays, vermiculites, and the like. It is appreciated that in embodiments of the processes and apparatus described herein where a fermentation of barn waste process is included, the aqueous solution may already include ample aluminum salts arising from the ingestion of soils, such as aluminum rich clay soils, by the barn animals generating the waste.

In one embodiment, inorganic phosphates and organophosphates are precipitated, aggregated, or otherwise removed from an aqueous solution using calcium ions, other divalent cations, or other Group IIA metal ions. In variations of this embodiment, ferric ions, aluminum ions, and/or anionic or non-ionic polymers are also included. In another embodiment, sulfate is precipitated from an aqueous solution using calcium ions, other divalent cations, or other Group IIA metal ions. In variations of this embodiment, ferric ions, aluminum ions, and/or non-ionic polymers are also included. Illustratively, the calcium ions derive from calcium hydroxide, calcium oxide, calcium chloride, and the like. Illustratively, the ferric ions derive from ferric hydroxide, ferric chloride, ferric sulfate, and the like. Illustratively, the aluminum ions derive from aluminum sulfate, aluminum hydroxide, aluminum chloride, and the like. Illustratively, the non-ionic polymer is a polyvinylpyrrolidone (PVP), including a PVP having an average molecular weight of about 300,000 or greater, or about 600,000 or greater. The non-ionic polymer may also be a partially hydrolyzed polyacrylamide, including a partially hydrolyzed polyacrylamide that has been hydrolyzed by about 30%. The non-ionic polymer may also be one or more polymerized BENTONITES, including polymerized BENTONITES that include a partially hydrolyzed polyacrylamide, vermiculite, silica, and the like. In the case of polymers that include BENTONITES, commercial sources may be in sodium and/or potassium forms. It is appreciated that such BENTONITES may be converted to other forms, including ferric forms, by washing the commercial material with a solution of the desired counterion, such as a solution of ferric sulfate, ferric chloride, and the like.

In another embodiment, the aqueous solution includes dissolved and undissolved solids, such as natural polymers, lignins, hemicelluloses, proteins, bacterial components, microorganisms, and the like. These solids may be removed using the processes and apparatus described herein. In one aspect, these solids are removed by treating the aqueous solution with ferric ions and non-ionic polymers as described herein. It is understood that the ferric ions and the non-ionic polymers may form aggregation, absorption, adsorption, or other complexes with these components and either remove them from the aqueous solution, or further aggregate to form larger aggregates or particles that may settle out of the aqueous solution. It is appreciated that the solubility of these natural polymers, including lignins, celluloses, and proteins, is dependent on their molecular weights, the pH of the aqueous solution, the ionic strength of the aqueous solution, and other physical parameters. In another aspect, the conditions that are substantially optimal for removing phosphorus-containing components may also effectively remove natural polymers, lignins, hemicelluloses, proteins, bacterial components, microorganisms, and the like.

The pH of the aqueous solution may be raised above acidic levels, to neutrality, to pH levels near or at the corresponding isoelectric point of the aqueous solution, or to a more alkaline pH by adding a base such as lime, slake lime, powdered limestone, calcium oxide, calcium chloride, sodium hydroxide, potassium hydroxide, carbonates and bicarbonates, including sodium, potassium, and calcium carbonates and bicarbonates, sulfates, including sodium, potassium, and calcium sulfates, and the like, and combinations thereof. In some variations, the various sources of lime, slake lime, and limestone may also include a percentage of iron salts. In other variations, a ferric form of clay is added to the aqueous solution. The choice of a pH level depends on the solubility characteristics of the dissolved or undissolved solids that are to be removed from the aqueous solution. For example, an aqueous solution that includes inorganic and/or organic phosphate components is treated with an iron salt, such as ferric sulfate, a non-ionic polymer, such as PVP, and a base, such as calcium oxide. The base is added to achieve a pH near or at the pH corresponding to the isoelectric point of the aqueous solution. Without being bound by theory, it is believed that the association of iron, PVP, and inorganic and/or organic phosphates is strongest at the isoelectric point. It is understood that such strong association contributes to large and/or dense particles, precipitates, aggregates, crystals, and absorption and adsorption complexes of iron, PVP, and inorganic and/or organic phosphates. Similar procedures may be used for aqueous solutions that include inorganic and/or organic sulfate components.

In variations of the processes described herein, the various precipitation or aggregation catalysts are added as separate components. In other variations, certain mixtures of precipitation or aggregation catalysts are added together either contemporaneously or as a prepared mixture. Illustratively, the non-ionic polymer and the transition metal ions are added as a mixture. In another aspect, the non-ionic polymer, the transition metal ions, and a portion or all of the calcium, other divalent cations, or other Group IIA metal ions are added contemporaneously, where the non-ionic polymer and the transition metal ions may optionally be added as a mixture.

The processes and apparatus described herein also use predetermined pH levels to facilitate the removal of dissolved and undissolved solids from aqueous solutions. Phosphate, sulfate, and other salts, and organophosphate compounds that might be removed from aqueous solutions have different solubilities at different pH levels. For example, both phosphate and sulfate salts of calcium are less soluble at higher pH levels than may be used during fermentation processes.

In addition, it is understood that gradual changes in pH may promote the formation of larger particles or crystals, where rapid changes in pH may lead to amorphous or finely divided solids. It is further understood that settling rates will generally follow the Reynolds equation, where the settling rate is inversely proportional to the square of the effective surface area of the particle. Therefore, particles of similar density will settle as a function of particle size, the larger of which tend to settle first. When particles are below a certain size, are finely divided, or amorphous, the settling rate may slow to an unusable rate. It is understood that in embodiments where particles settle by gravity, the settling rate is inversely proportional to the square of the effective surface area of the particle, and proportional to gravity.

Illustratively, the predetermined pH for precipitating and/or aggregating the dissolved and undissolved solids in the aqueous solutions is in the range from about 6 to about 8, in the range from about 6.5 to about 7.5, and is illustratively about 6.8. In some variations, the pH change may be performed in two steps, where the pH is changed rapidly to a point below the predetermined level, and then changed slowly to the predetermined level to maximize the size of particles precipitating from or aggregating in the aqueous solution. Illustratively, the pH may be changed rapidly to a level in the range from about 6.0 to about 6.5, or illustratively to about 6.4. After the rapid pH change, the pH is increased more slowly to the predetermined pH level.

The dissolved and undissolved solids removed from the aqueous solution may be periodically removed from the processes and apparatus described herein, such as in the form of a phosphorus rich clay. The resulting clarified water may also be periodically removed from the processes and apparatus described herein. It is understood that the phosphorus rich clay may contain calcium phosphate and other inorganic forms of phosphorus, as well as organic molecules containing phosphorus. In particular, it is understood that such inorganic and organic phosphates may form complexes with calcium, iron, and/or carbonate, that may be insoluble at high pH and subsequently form a precipitate or other aggregation that may be separated from the aqueous solution. The processes may be performed in a batch mode, a continuous mode, or in a series of batch cycles that may be run continuously.

In other embodiments, an excess of nitrogen-containing components is present in the aqueous solution. In one aspect where the aggregation processes described herein are used in conjunction with fermentation processes, such as those described herein, the fermentation process may cause most of the nitrogen-containing compounds to be an inorganic form of nitrogen due to enzymatic activity encountered during fermentation by fermenting organisms. In one embodiment, fermentation gases such as carbon dioxide that are recycled from the fermentation processes, may subsequently be contacted with the aqueous solution to facilitate the removal of nitrogen. The resulting ammonium carbonates may be removed from the aqueous solution by degassing, and collecting the nitrogen as ammonia. In addition, excess carbonate in the aqueous solution may also facilitate aggregation of other inorganic cations; it is understood that many carbonate salts are less soluble at alkaline pH than their corresponding sulfate salts, including carbonate salts of divalent metals.

An illustrative embodiment of the apparatus described herein for removing dissolved or undissolved solids by precipitation, aggregation, crystallization, absorption, and/or adsorption is shown in FIG. 49. Anions such as phosphates, sulfates, carbonates, and the like, cations such as calcium, potassium, iron, aluminum, and the like, organic molecules such as organophosphates, peptides, proteins, lignins, and the like, and organisms such as fermenting organisms, bacteria, yeast, and the like, may be illustratively removed using the system shown in FIG. 49. Referring to FIG. 49, aqueous solution AS enters aggregation unit 2110. It is understood that aqueous solution AS may be an aqueous solution exiting a fermentation process or apparatus, such as a fermentation process or apparatus described herein.

Aggregation unit 2110 includes one or more aggregation tanks 2130 each including a liquid inlet LI. Liquid inlet LI is in fluid communication with inlet conduit 2112. Inlet conduit 2112 is in fluid communication with aqueous solution outlet ASO for introducing aqueous solution AS, base outlet BO for introducing base, and one or more aggregation catalyst outlets ACO for introducing aggregation catalysts AC. Aggregation tanks 2130 also include a cleaned water outlet CWO in fluid communication with outlet conduit 2114, and an aggregate or precipitate outlet PPTO, optionally coupled with a solid conveyor unit 2116.

Aqueous solution outlet ASO is in fluid communication with an aqueous solution source 2118, such as the outlet of a fermentation system, a reservoir or lagoon containing an aqueous solution to be treated, and the like, and is in fluid communication with a pump P for pumping aqueous solution AS from source 2118 to outlet ASO. A valve V, optionally operated by a programmable logic circuit PLC is placed between outlet ASO and inlet conduit 2112. Base outlet BO is in fluid communication with a base source 2120 containing a base as described herein, and is in fluid communication with a pump P for pumping base B from source 2120 to outlet BO. A valve V, optionally operated by a programmable logic circuit PLC is placed between outlet BO and inlet conduit 2112. Each aggregation catalyst outlet ACO is in fluid communication with a corresponding aggregation catalyst source 2122, and is in fluid communication with a pump P for pumping aggregation catalyst AC from aggregation catalyst source 2122 to outlet ACO. A valve V, optionally operated by a programmable logic circuit PLC is placed between each outlet ACO and inlet conduit 2112. In an illustrative embodiment having three aggregation catalyst outlets ACO, first aggregation catalyst AC, is supplied by a first aggregation catalyst source 2122,₁ in fluid communication with outlet ACO₁, second aggregation catalyst AC₂ is supplied by a second aggregation catalyst source 2122₂ in fluid communication with outlet ACO₂, and third aggregation catalyst AC₃ is supplied by a third aggregation catalyst source 2122₃ in fluid communication with outlet ACO₃. It is understood that in variations of the apparatus, any of the first, second, third, or successive aggregation catalysts AC may be premixed with another one or more of the other aggregation catalysts AC, and the mixture is pumped into inlet conduit 2112 through one of aggregation catalyst outlets ACO.

As described herein, addition of base to aggregation tanks 2130 may take place in two steps or as a two-stage process. In the first step or stage, the majority of the base is added to adjust the pH of the aqueous solution to a pH level near the optimal pH level for aggregation or precipitation. In the second step or stage, a slower addition of base is made to adjust the pH of the aqueous solution to a pH level at the predetermined optimal pH level for aggregation or precipitation. The rapid addition of base may take place during of after filling aggregation tanks 2130. The slow addition of base may take place after filling aggregation tanks 2130. In one aspect, rapid addition of base is performed during the filling of a first aggregation tank 2130. After filling first aggregation tank 2130, a second aggregation tank 2130 begins to fill, and a slow addition of base starts in the first aggregation tank 2130. Valves V controlling base addition from base outlet BO to aggregation tanks 2130 may be operated in a time-share sense in that while second aggregation tank 2130 is filling and base is being added rapidly, base is intermittently added to first aggregation tank 2130 to effect the second stage of the base addition. In alternative embodiments, a separate base source BS (not shown) supplies base to aggregation tanks 2130 for the slow or fine pH adjustment step or stage.

In one aspect, aggregation unit 2110 includes one or more aggregation tanks 2130. In another aspect, aggregation unit 2110 includes two or more aggregation tanks 2130. In another aspect, aggregation unit 2110 includes three or more aggregation tanks 2130. In another aspect, aggregation unit 2110 includes four or more aggregation tanks 2130. It is appreciated that the number of aggregation tanks 2130 may depend upon the settling rate of aggregate or precipitate PPT, so that more aggregation tanks 2130 are used with slower settling aggregates or precipitates PPT, and fewer aggregation tanks 2130 are used with faster settling aggregates or precipitates PPT for a given volume processing rate. It is appreciated that in some embodiments, aqueous solution AS is a dilute solution of dissolved and/or undissolved solids; therefore, aggregates or precipitates PPT are removed from aggregation tanks 2130 through outlet PPTO only occasionally.

In an embodiment where aggregation unit 2110 includes one aggregation tank 2130, the system is run in a batch mode. In the embodiments where aggregation unit 2110 includes more than one aggregation tank 2130, the system is run in a continuous mode, where one tank 2130 is filling while the remaining tanks 2130 are in varying stages of settling or are being emptied of cleaned water CLW or aggregate or precipitate PPT.

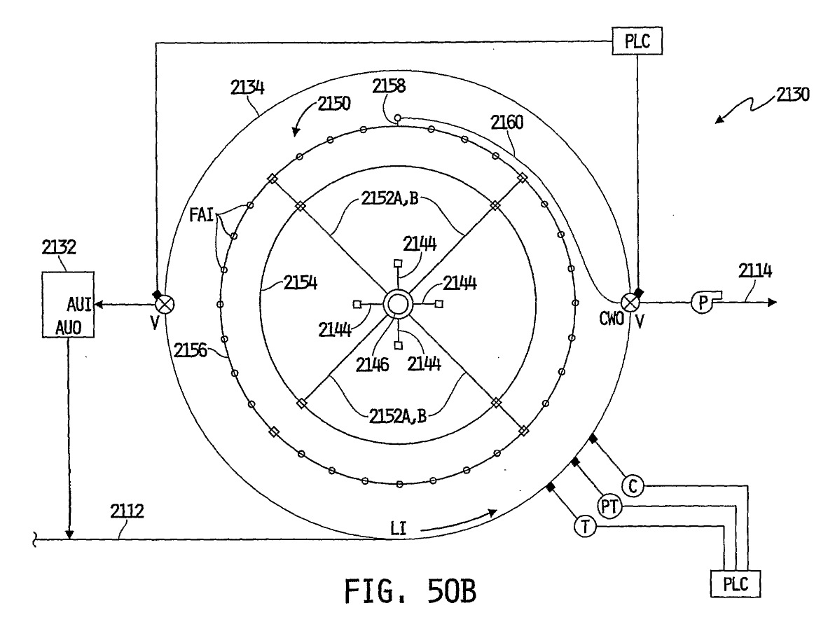

Referring to FIGS. 50A and 50B showing detail for each of the one or more aggregation tanks 2130, in one illustrative embodiment, aggregation tanks 2130 have a generally sloped bottom 2138 to facilitate the removal of aggregate or precipitate PPT through outlet PPTO located at the low point of sloped bottom 2138. Sloped bottom 2138 may have an arcuate, frustoconical, or linear profile, or a combination thereof. Aggregation tanks 2130 optionally have a roof or cover 2136. In embodiments including a roof or cover 2136, the roof or cover 2136 may also include one or more vents. Aggregation tanks 2130 are also optionally fitted with a clean water spraying unit (not shown) for facilitating the cleaning and/or maintenance of tanks 2130. Aggregation tanks 2130 are also optionally fitted with an agitation unit 2132, a level or volume sensor, such as a pressure transducer PT, a pH sensor, such as a conductivity sensing unit CS, and/or a temperature sensor TS. Each of the level or volume sensors, pH sensors, and/or temperature sensors TS are also optionally coupled to one or more programmable logic circuits PLC configured to operate one or more algorithms controlling the filling, emptying, mixing, dwell, and other phases of the processes used in the apparatus described herein, where the algorithms use the signal values obtained from these sensors. Agitation unit 2132 may be in the form of a recirculating system or pump that is fluid communication with an agitation unit outlet AUO on tank 2130, where the outlet is placed at a level L3. Liquid flow through outlet AUO is controlled by a valve V optionally coupled to a programmable logic circuit PLC. In one illustrative aspect, circuit PLC may operate valve V and agitation unit 2132 based on a signal obtained from pressure transducer PT indicating a fill level at or above level L3. Agitation unit 2132 is also in fluid communication with inlet conduit 2112, so that when the fill level is at or above L3, the liquid contents of tank 2130 are pumped through outlet AUO, and back into inlet conduit 2112. Therefore, the contents already present in aggregation tanks 2130 are admixed with the material introduced into aggregation tanks 2130 through inlet conduit 2112, including aqueous solution AS, base B, and one or more aggregation catalysts AC.

A valve V separates each liquid inlet LI into aggregation tanks 2130 from inlet conduit 2112, and is optionally controlled by a programmable logic circuit PLC. A valve V also separates each cleaned water outlet CWO from aggregation tanks 2130 from outlet conduit 2114, and is optionally controlled by a programmable logic circuit PLC. A pump P is also in fluid communication with cleaned water outlet CWO and outlet conduit 2114, and is operated to remove cleaned water from aggregation tanks 2130 as described below.

Aggregate or precipitate outlet PPTO is optionally fitted with an auger unit 2140 for removing aggregate or precipitate PPT. In embodiments that include auger 2140, auger 2140 is illustratively transverse to outlet PPTO, and may be in the form of a progressive cavity pump operated to periodically remove aggregate or precipitate PPT from aggregation tanks 2130. Auger 2140 also includes a motor M. In one illustrative aspect, motor M may include a torque sensing unit (not shown) that is capable of acting as a shutoff controller for auger 2140. For example, as the measured torque falls below a predetermined threshold level because of the removal of a desired or predetermined amount of aggregate or precipitate PPT, the torque sensing unit will shut down auger 2140. Aggregate or precipitate PPT that is removed by auger unit 2140 is moved to conveyor unit 2116.

The one or more aggregation tanks 2130 are also each fitted with a center post 2146 aligned with an axis of tank 2130 and supported at the top of tank 2130 by supports 2142, and at the bottom of tank 2130 by supports 2144. A hollow floating assembly 2150 is coupled with center post 2146 in a manner that allows floating assembly 2150 to slide up and down center post 2146 according to the level of the liquid in tanks 2130. Floating assembly 2150 may slide along center post 2146 between a bottom position L1, and a top position L2. Bottom position L1 is at or near the emptied level of aggregation tanks 2130, and top position L2 is a position at or near the filled level of aggregation tank 2130. It is understood that levels L1 and L2 do not necessarily define the complete capacity of aggregation tank 2130. Each floating assembly 2150 has one or more inlets FAI, which are in fluid communication with the hollow of floating assembly 2150, and the contents of aggregation tanks 2130. Floating assembly 2150 is coupled to a floating conduit 2160 in fluid communication with cleaned water outlet CWO. Cleaned water outlets CWO may be placed at about the vertical midpoint between levels L1 and L2 of aggregation tanks 2130. Cleaned water outlet CWO is also in fluid communication, controlled by valve V, with a pump P capable of pumping cleaned water through inlets FAI into hollow floating assembly 2150, subsequently into floating conduit 2160, and subsequently to cleaned water outlet CWO. As cleaned water exits aggregation tank 2130, and the level of liquid in aggregation tanks 2130 moves lower, and floating assembly 2150 moves downward along center post 2146 with the level of liquid continuing to release cleaned water into cleaned water outlet CWO until it reaches a predetermined location or level L1 above the bottom of aggregation tank 2130. Agitation unit 2132 is placed at a predetermined level L3 in aggregation tanks 2130 to minimize agitation of aggregate or precipitate PPT that has already settled in aggregation tanks 2130 and entered outlet PPTO. In addition, liquid inlet LI supplying aqueous solution AS, base B, precipitation catalysts AC, and recirculated contents from aggregation tank 2130 is place at a level L4. Level L4 is also selected so as to minimize agitation of aggregate or precipitate PPT that has already settled in aggregation tanks 2130 and entered outlet PPTO.

In one embodiment, floating assembly 2150 is in the form of a floating ring sparger that includes a hollow tube 2154 to provide buoyancy to floating assembly 2150, a ring sparger 2156 that includes one or more inlets FAI in fluid communication with the hollow space of sparger 2156, and a support structure for floating assembly 2150 that includes a series of upper horizontal supports 2152A and lower angled supports 2152B that are coupled with center post 2146. In one aspect, floating conduit 2160 is configured to spool onto floating assembly 2150, such that as floating assembly 2150 lowers to or rises to the level of cleaned water outlet CWO, floating conduit 2160 will spool onto floating assembly 2150. Conversely, as floating assembly 2150 rise above or lowers below the level of cleaned water outlet CWO, floating conduit 2160 will unspool from floating assembly 2150. It is understood that floating assembly 2150 is coupled to center post 2146 in a manner that allows floating assembly 2150 to rotate about the axis represented by center post 2146 and spool or unspool floating conduit 2160.

Referring to FIG. 50A, in one illustrative embodiment, aggregation tanks 2130 have a low aspect ratio, as defined by the ratio of a vertical dimension to a horizontal dimension. Low aspect ratios may decrease the overall elapsed time required for settling the particles aggregate, crystals, precipitate, absorption complex, or adsorption complex. Illustrative low aspect ratios include aspect ratios of about 2 or less, or aspect ratios of about 1 or less.

Referring to FIG. 50B, in one illustrative embodiment, aggregation tanks 2130 have a circular or elliptical cross-section. In this view, optional roof or cover 2136 is not shown for clarity. Such tanks may be generally spherical or generally cylindrical in overall shape. In another illustrative embodiment, liquid inlets LI are configured so that liquid entering aggregation tanks 2130 is directed along a side 2134 of aggregation tanks 2130, as indicated by arrow A in FIG. 50B. In variations where aggregation tanks 2130 have a circular or elliptical cross-section, liquid inlets LI enter aggregation tanks 2130 at a tangential point. In addition, liquid entering aggregation tanks 2130 may create a vortex in aggregation tanks 2130. It is appreciated that such a vortex may serve to mix the components of liquid entering aggregation tanks 2130 with each other as well as mix the liquid entering aggregation tanks 2130 with residual material already contained in aggregation tanks 2130. It is also appreciated that such a vortex may facilitate the movement of precipitate or aggregate away from the sides 2134 and down the sloped bottom 2138 of aggregation tanks 2130 toward outlet PPTO.

In variations of the apparatus, aggregation tanks 2130 may be fitted with additional liquid inlets LI that are in fluid communication with one or more aggregation catalyst outlets ACO, base outlet BO, or additional base outlets BO. It is understood that additional base outlets BO may be supplied by the same or by different base sources 2120. In variations where the same base source 2120 is used, an algorithm may be used to control the distribution of base as needed to any of base outlets BO ultimately in fluid communication with liquid inlets LI into aggregation tanks 2130. The algorithm may include parameters such as elapsed time, pH, conductivity, and like inputs or measurements taken from aggregation tanks 2130.

In variations of the apparatus where base outlet BO is in fluid communication with inlet conduit 2112, inlet conduit 2112 may be fitted with a pH sensing unit (not shown). The pH sensing unit may be in the form of two or more conductivity sensors CS, where at least one sensor CS is located upstream of base outlet BO, and at least one sensor CS is located downstream of base outlet BO. Conductivity sensors CS are capable of measuring a signal which may be sent to a programmable logic circuit capable of converting the conductivity of aqueous solution AS to a pH value that may be in turn used to control the addition of base through alternate base outlet BO coupled to inlet conduit 2112. In variations of the apparatus where alternate base outlet BO is coupled to inlet conduit 2112, inlet conduit 2112 optionally includes a heat exchanger (not shown) for cooling the aqueous solution as needed after the addition of base through alternate base outlet BO.

In one illustrative process using the apparatus shown in FIGS. 49, 50A, and 50B, aqueous solution AS enters inlet conduit 2112. In one illustrative aspect, a predetermined amount of one or more aggregation catalysts also enter inlet conduit 2112. The mixture then enters aggregation tank 2130 through liquid inlet LI. The conductivity of the contents of aggregation tank 2130 is measured with a conductivity sensor CS. The signal from sensor CS is sent to a programmable logic circuit that controls the addition of an appropriate amount of base entering inlet conduit 2112 through base outlet BO and mixing with aqueous solution AS. The conductivity of the contents of tank 2130 is continually or periodically measured to continually or periodically adjust the amount of base added to inlet conduit 2112. When a predetermined fill level is reaches, determined on the basis of elapsed time or using pressure transducer PT, agitation unit 2132 is operated to homogenize the contents of aggregation tank 2130 with the incoming stream entering liquid inlet LI so that conductivity measurements taken by conductivity sensor CS are representative of the bulk mixture rather than the mixture in the locale of conductivity sensor CS. An algorithm controls the amount of base entering conduit 2112 determined by evaluating the conductivity of the material in aggregation tank 2130, comparing that value with the difference between the desired value and the value predicted by the last addition or adjustment to the addition of base.

For example, the pH calculated from the reading taken by conductivity sensor CS of aqueous solution AS in aggregation tank 2130 is converted into a predetermined amount of base entering inlet conduit 2112 through base outlet BO. Subsequently, the pH calculated from the reading taken by conductivity sensor CS of the contents of aggregation tank 2130 is compared against a predicted value based on the predetermined amount of base entering inlet conduit 2112. If the predicted value matches the value measured by conductivity sensor CS of the contents of aggregation tank 2130, no change is made to the amount of base entering inlet conduit 2112. If the predicted value is higher than or lower than the value measured by conductivity sensor CS of the contents of aggregation tank 2130, a corresponding change to the amount of base entering inlet conduit 2112 is made. In one illustrative aspect, the predicted pH value of the contents of aggregation tank 2130 is a pH level slightly below the predetermined pH optimal for precipitate formation during a filling phase or step of a process described herein.

Aqueous solution AS, base B, and aggregation catalysts AC enter a first aggregation tank 2130 through inlet ASI at a level L4, or optionally at a point about level with the lowest possible location L1 of floating assembly 2150 to minimize agitation of the solution and aggregate or precipitate PPT remaining in the first aggregation tank 2130 from the last run. Aqueous solution AS, base B, and aggregation catalysts AC are added to first aggregation tank 2130 to a fill level that may be near the highest possible location L2 of floating assembly 2150. Filling of tank 2130 may be controlled by using a predetermined time based on the pumping rate and tank volume, or by using a level, volume, or pressure sensor PT that indicates the fill level of first tank 2130. When the fill level L3 is reached, agitation unit 2132 is optionally operated to homogenize the composition present in first aggregation tank 2130. After tank 2130 is full, valve V controlling the addition of aqueous solution AS, base B, and aggregation catalysts AC via inlet conduit 2112 to first aggregation tank 2130 is closed, and the corresponding valve V to second aggregation tank 2130 is opened. The filling process as described for first aggregation tank 2130 begins in second aggregation tank 2130.

After first aggregation tank 2130 is full, additional base is added through inlet conduit 2112, and the conductivity of the contents of first aggregation tank 2130 is measured with conductivity sensor CS. The corresponding pH of the contents of first aggregation tank 2130 is determined from the conductivity measurement and compared to a predetermined optimum pH value for crystallization, precipitation, aggregation, absorption, and/or adsorption. Base addition into first aggregation tank 2130 through inlet conduit 2112 is continued until the measured conductivity corresponds to a pH value at or near the predetermined optimum pH value as described herein. Agitation unit 2132 is optionally operated during this second stage addition of base into first aggregation tank 2130. In an alternate embodiment, aqueous solution AS and base B are added first, and then after the addition base, one or more aggregation catalysts are added to first aggregation tank 2130 through liquid inlet LI. In variations of this process, a mixture of a first aggregation catalyst and a second aggregation catalyst is added to first aggregation tank 2130 through liquid inlet LI to the contents of first aggregation tank 2130.

After the addition of the one or more aggregation catalysts, additional base is added through liquid inlet LI, and the conductivity of the contents of first aggregation tank 2130 is measured with conductivity sensor CS. The corresponding pH of the contents of first aggregation tank 2130 is determined from the conductivity measurement and compared to a predetermined optimum crystallization, precipitation, aggregation, absorption, and/or adsorption pH value. Base addition into first aggregation tank 2130 through liquid inlet LI is continued until the measured conductivity corresponds to a pH value at or near the predetermined optimum pH value as described herein. It is understood that agitation unit 2132 is optionally operated during this second stage addition of base into first aggregation tank 2130. In an alternate embodiment, the pH is adjusted to the optimum level before the addition of the one or more aggregation catalysts.

In one aspect, after first aggregation tank 2130 is full, the addition of base to raise the pH to the predetermined optimal pH level as described herein for crystallization, precipitation, aggregation, absorption, and/or adsorption is illustratively a slow rate of addition to facilitate the formation of larger particles, crystals, precipitates, aggregates, or absorption or adsorption complexes. When the optimal predetermined pH is reached, agitation unit 2132 is stopped, and aggregation and settling begins. After a predetermined settling wait period, pump P is operated to remove cleaned water from tank 2130 through outlet CWO via floating assembly 2150. The pumping rate is predetermined to be about less than or about equal to the settling rate of aggregate or precipitate PPT. Pumping is continued for a predetermined time based on the pumping rate and tank volume, or until a level or volume sensor PT indicates floating assembly 2150 has reached is lowest allowed position. Valve V located at outlet CWO is then closed to first aggregation tank 2130, and the corresponding valve is opened to second tank 2130, allowing the process to run in a continuous serial batch mode.

It is understood that several coordinated configurations are possible when more than one aggregation tank 2130 is used in aggregation unit 2110. In one embodiment, the elapsed time for settling and emptying of first tank 2130 may be selected to correspond with the filling and settling time in second tank 2130, such that upon completion of the emptying of first tank 2130, the emptying of second tank 2130 may begin. Correspondingly, the refilling of first filling tank 2130 or filling of third aggregation tank 2130 may begin. Other configurations are also possible where the filling, waiting, emptying, and idle times are coordinated to achieve a continuous processing of aqueous solution AS.

It is further understood that during times when a particular aggregation tank 2130 is idle after an emptying step, and awaiting the next filling cycle, the saturated precipitate solution remaining in tank 2130 may be continually recrystallizing or reaggregating, such that larger and larger crystals or particles are formed. Such a process may tend to minimize the amount of retained water in the aggregate or precipitate PPT slag that is periodically removed through outlet PPTO using auger 2140. It is further understood that such recrystallizing or reaggregating processes may tend to promote the formation of a more compact aggregate or precipitate PPT slag that is periodically removed. It is further understood that such a process may also tend to minimize the amount of agitation and or re-solution of aggregate or precipitate PPT that might occur during the next filling cycle.

In one aspect of the illustrative system shown in FIG. 49, the aqueous solution AS is a solution exiting a fermentation system, such as a fermentation system described herein. In another aspect, the first aggregation catalyst is a transition metal salt, such as a transition metal halide, hydroxide, or sulfate, including ferric chloride, ferric hydroxide, and ferric sulfate. In another aspect, the second aggregation catalyst is a Group IIA metal salt, such as a calcium salt including calcium chloride, calcium sulfate, calcium hydroxide, and the like, or a Group IIA metal oxide, such as calcium oxide.

In variations, additional aggregation catalysts are added, such as Group IIIA metal salts including aluminum sulfate, aluminum hydroxide, and the like. In other variations, one or more of the aggregation catalyst sources includes a pH adjustment unit (not shown) that includes an acid source, containing an inorganic or mineral acid such as hydrochloric acid, and a base source, containing an inorganic base such as a carbonic acid salt, including a sodium, potassium, or calcium salt thereof, an oxide, such as sodium, potassium, or calcium oxide, and the like.

In an illustrative embodiment having four aggregation tanks 2130, an algorithm using any number of a variety of signal inputs may be used to coordinate the filling of each aggregation tank 2130, the addition of base, the addition of any one of the one or more aggregation catalysts, the agitation, the dwell interval for settling, the emptying of aggregation tank 2130, and any idle interval. Signal inputs include, but are not limited to time, pH, conductivity, pressure, weight, temperature signal inputs, and the like. In one aspect, each of the four aggregation tanks 2130 has a known volume, and the filling phase, dwell phase, settling phase, emptying phase, and idle phase are each controlled by predetermining a filling rate, and determining an emptying rate corresponding to the settling rate of aggregate or precipitate PPT.

In one aspect, valve V to liquid inlet LI of first aggregation tank 2130 is opened and the tank is filled with aqueous solution AS. The first aggregation tank 2130 illustratively has a volume of 12,000 gallons (45,425 liters) and AS is pumped in at a rate of 100 gallons/min (379 liters/min). Valves V to liquid inlets LI of second, third, and fourth aggregation tanks 2130 are closed. Valve V controlling base outlet BO to inlet conduit 2112 of first aggregation tank 2130 is opened and a solution of calcium oxide (calcium hydroxide) is contemporaneously added. The amount of base added is controlled by measuring the conductivity of the contents of first aggregation tank 2130. The target conductivity of the contents of first aggregation tank 2130 is that conductivity corresponding to a pH in the range from about 6 to about 7. Agitation unit 2132 is operated throughout the filling of first aggregation tank 2130. The tank is filled in approximately 120 minutes. Valve V controlling outlet ASO through inlet conduit 2112 and liquid inlet LI to first aggregation tank 2130 is closed after a predetermined elapsed time or after a reading from pressure transducer indicates that first aggregation tank 2130 is filled to capacity. Simultaneously, valve V controlling outlet ASO through inlet conduit 2112 to inlet LI of second aggregation tank 2130 is opened and the tank is filled with aqueous solution AS. Second aggregation tank 2130 also illustratively has a volume of 12,000 gallons (45,425 liters), and AS is pumped in at a rate of 100 gallons/min (379 liters/min). In addition, valve V controlling base outlet BO to second aggregation tank 2130 is opened and a solution of calcium oxide (calcium hydroxide) is contemporaneously added. Base addition is controlled as described above for first aggregation tank 2130. Subsequent steps in the process using second aggregation tank 2130 proceed as described below. In addition, third and fourth aggregation tanks 2130 are used sequentially. It is understood that first aggregation tank 2130 reenters the process after fourth aggregation tank 2130 in a continuous cycle until the processing of aqueous solution AS is complete.

Base addition is continued into inlet conduit 2112 into first aggregation tank 2130, but at a slower or substantially slower addition rate, and agitation unit 2132 is continually operated. Base addition may continue into first aggregation tank 2130 by a time-share sequence where base is directed into both first and second aggregation tanks 2130 by appropriate operation of valves V controlling outlet BO into those tanks. Alternatively, an additional base source may be used to continue to add base to first aggregation tank 2130 after it is filled and while second aggregation tank 2130 is being filled. In one aspect, base addition is continued until a predetermined conductivity is detected by conductivity sensor CS. In another aspect, base addition is continued until a predetermined change in conductivity over time is detected. Valve V controlling aggregation catalyst outlet ACO to first aggregation tank 2130 is opened, and a mixture of a first aggregation catalyst, illustratively ferric sulfate, and a second aggregation catalyst, illustratively PVP, is added. Valves V to outlets ACO of second, third, and fourth aggregation tanks 2130 are closed. Illustratively, the mixture of the first and the second aggregation catalysts is continued for a predetermined length of time based on the addition rate of the mixture and the volume of first aggregation tank 2130. Valve V to aggregation catalyst inlet ACI of first aggregation tank 2130 is closed, agitation unit 2132 is stopped, and the contents of first aggregation tank 2130 are allowed to settle. After a predetermined period of time, valve V to outlet CWO of first aggregation tank 2130 is opened, pump P is operated, and cleaned water is removed from first aggregation tank 2130 through floating assembly inlets FAI into hollow floating assembly 2150. In variations, instead of an elapsed time parameter, an optical element may be included in aggregation tanks 2130 capable of measuring the optical density of the contents of the tank. The optical element may be used to determine that the settling of aggregate or precipitate PPT has progressed to or past a certain point in the tank and to initiate the removal of cleaned water from the tank. Cleaned water is pumped from the tank at a rate at or less than the continued settling rate of aggregate or precipitate PPT. After cleaned water has been removed from first aggregation tank 2130 to a predetermined lower level, pump P is stopped and valve V to outlet CWO of first aggregation tank 2130 is closed. The processes in second, third, and fourth aggregation tanks 2130 has continued simultaneously and is at various stages. In another embodiment of the processes described herein for precipitating dissolved solids from aqueous solutions, a process for precipitating solids from aqueous solutions using gaseous carbon dioxide is described. It is understood that this process may be accomplished with the apparatus described herein using modifications that allow for the introduction of a gas containing or consisting of carbon dioxide. Such introduction may be accomplished for example using any conventional sparger, or any of the sparger embodiments described herein, or incorporated herein by reference.

In one aspect of the process, an aqueous solution having any pH in the range from less than about 1 to less than about 10 or 11 is treated with a strong base or a strong base solution to raise the pH to about 10 or greater, or to about 11 or greater. In another aspect, the pH is raised to substantially above 11, including about 12 or about 13. In another aspect, the pH of the aqueous solution is raised as fast as is practicable. After a short dwell time, illustratively about 15 minutes, or about 30 minutes, the pH is illustratively at least greater than about 10, or greater than about 11. It is appreciated that a dwell time may be necessary for a pH equilibrium to be reached in embodiments where the pH is increased rapidly by the addition of base. slowly reduced by the addition of a source of gaseous carbon dioxide. In another aspect, the pH is subsequently reduced to a near neutral pH in the range from about 6.5 to less than about 8, and illustratively in the range from about 6.8 to about 7.5. In another aspect, the pH is subsequently reduced to a slightly basic final pH in the range from greater than about 7 to less than about 8, and illustratively in the range from greater than about 7 to less than about 7.5.

It is appreciated that depending upon the gaseous source of carbon dioxide, either a slightly basic pH or a nearly neutral pH may be the final pH. For example, if the source of carbon dioxide is that naturally occurring in atmospheric air, the final pH may only be slightly basic, such as less than about 8, or in the range from greater than about 7 to less than about 7.5. In contrast, if a more concentrated source of carbon dioxide, such as pure carbon dioxide is used to lower the pH, the final pH may be as low as about 6.5, or in the range from about 6.8 to about 7.5. It is understood that sources of carbon dioxide that are intermediate in concentration, such as gases collected from the fermentation processes described herein, may provide a either near neutral or slightly basic final pH.

It is further appreciated that in embodiments of the systems described herein than include fermentation processes, certain fermentations may provide more highly concentrated sources of carbon dioxide than others. For example, it is understood than fermentation processes that use ethanol generation waste or other alcohol production waste may provide relatively highly concentrated sources of carbon dioxide resulting from the fermentation thereof. In contrast, animal waste streams, or cheese and whey processing waste may provide relatively lower concentrations of carbon dioxide sources resulting from the fermentation thereof.

It has been observed that slow decreases in pH generally provides superior crystal quality, more highly organized, and/or more dense precipitates, agglomerates, and/or aggregates, that may also concomitantly trap less water or other solvent. These attributes of the resulting precipitates, agglomerates, and/or aggregates tend to decrease settling times and increase the overall purity of the clarified water produced in the processes described herein.

In another embodiment of this precipitating process, only carbon dioxide is added to the tank to cause precipitation of the remaining phosphorus compounds as carbonate salts. It is understood that within optimally selected pH ranges, carbonate salts of phosphate are less soluble than sulfate salts of phosphate, such as the sulfate salts produced in the acidifying steps of other processes described herein for treating biomaterial waste streams. It is understood that carbon dioxide is produced during the fermentation processes, and therefore that carbon dioxide may be trapped and used in the subsequent post processing steps to remove dissolved solids, such as phosphate solids. In this embodiment, the pH is adjusted higher by the addition of a base. It is appreciated that the addition of carbon dioxide will also alter the pH of the liquid being treated, and therefore subsequent addition of base is performed while taking account of the pH change causable by the carbon dioxide addition.

In another aspect, gaseous carbon dioxide is added via a sparger or other dispersing apparatus that decreases bubble size, allowing rapid mixing and minimizing the generation of local high concentrations of carbon dioxide in the aqueous solution being treated. It is therefore appreciated that both the rate and concentration of gaseous carbon dioxide in a gaseous input stream may be adjusted and modified to achieve a slow decrease in the pH of the aqueous solution being treated. For example, at one illustrative extreme, pure carbon dioxide gas may be added very slowly, optionally following a syncopated or metered profile, where small amounts of carbon dioxide are added, then a dwell period is included, followed by subsequent additional carbon dioxide gas. At another illustrative extreme, atmospheric air containing as little as about 0.03% to about 0.04% carbon dioxide may be added at a faster rate, with or without intermittent dwell periods. It is appreciated that below a certain threshold concentration and at a maximum addition rate, the addition time will be necessarily increased to ensure complete precipitation, or the obtention of a predetermined pH in the aqueous solution being treated. Other intermediate concentration sources of gaseous carbon dioxide include exhaust gases exiting the fermentation apparatus and processes described herein. The enrichment of carbon dioxide in those exhaust gases will likely depend upon the fermenting organism used and the nature of the components of the biomaterial waste stream being fermented. For example, biomaterial waste streams containing large amounts of ethanol, such as alcohol fermentation and production waste streams may tend to produce exhaust gases richer in carbon dioxide than may be produced by the fermentation of barn of swine waste. Regardless, at intermediate concentrations, the addition rate and addition time may be adjusted accordingly to achieve the predetermined rate of pH change. Further, the above sources of carbon dioxide, as well as other conventional sources, may each be further enriched by the addition of a purer source of carbon dioxide from an auxiliary tank or source, or be further diluted by the addition of atmospheric air.

In one illustrative variation, the pH may be monitored to determine when the final predetermined pH is achieved. In another variation, the pH is not monitored, rather estimates of sufficient time may be followed and the predetermined pH is achieved on the basis of an equilibrium being generated by the buffering supplied by the carbonic acid and salts thereof generated from the added carbon dioxide in conjunction with other non-precipitated components in the aqueous solution being treated, including carbonate and bicarbonate salts of calcium.

In one illustrative example, atmospheric air is used as the source of carbon dioxide, which is bubbled into about 180,000 to about 190,000 gallons (from about 680,000 to about 720,000 L) of an aqueous solution including phosphorus components among others. The air is introduced at a fast rate in the range from about 1500 to about 2000 ft³/min (from about 42 to about 57 m³/min), and illustratively at a rate of about 1700 ft³/min (about 48 m³/min), overnight for about 10-16 hr or for about 14 hr.

It is understood that like other processes described herein for removing dissolved solids from aqueous solutions, this embodiment may substantially reduce the amount of dissolved inorganic and organic phosphorus components, inorganic and organic nitrogen components, and the like, as well as other organic components that may contribute to chemical oxygen demand (COD) or biological oxygen demand (BOD), by precipitating the same as carbonate complexes. Phytic acid, a major component of animal waste-based biomaterial waste streams may also be specifically removed by this process. It is understood that phytic acid may generally not be decomposed by most fermenting organisms without the addition of phytases as described herein. Lignins and other colored organic components may also be specifically removed by this process.

Without being bound by theory, it is believed that in dilute aqueous solutions continuing dissolved solids, the initial rapid rise in pH causes the formation of insoluble salts of the components that are ultimately removed by precipitation. However, the dilute nature of the aqueous solution being treated may preclude the formation of large crystals for kinetic reasons, especially in large scale operations. It is therefore believed that such insoluble salts of the components that are ultimately removed by precipitation initially associate with calcium and subsequently with carbonate to either form complexes with each other or simply form larger and denser carbonate crystals, replacing the smaller hydroxide crystals. Settling times are therefore increased, along with the efficiency of dissolved solid removal. In any case, the clarified water exiting such dissolved solid precipitation processes and apparatus may be disposed as non-hazardous waste water. In particular, the processes described herein that use gaseous carbon dioxide generally achieve the near neutral pH range required for non-hazardous waste disposal.

### EXAMPLES

### EXAMPLE 20

### Illustrative Core Process with Optional Alternative Processes

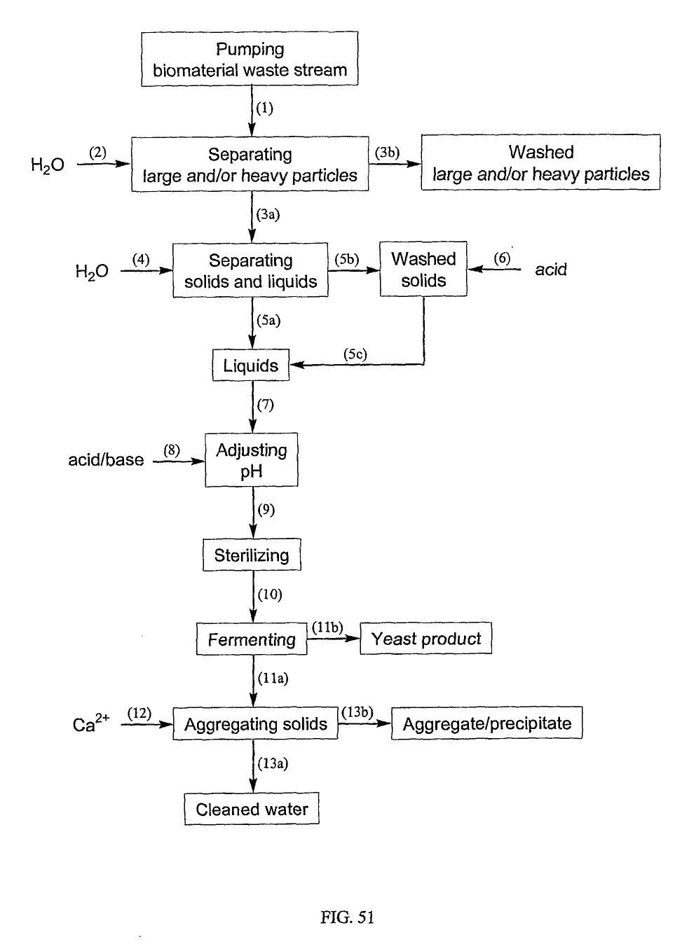

Steps of an illustrative process are shown FIG. 51. The process shown in FIG. 51 includes a core process and two separate and optional treatment steps, a pretreatment process and a post treatment process. The core process includes pumping (step 1), separating large and/or heavy particles, including sand (steps 2 & 3), separating solids, including fiber, from liquids (steps 4 & 5), adjusting the pH (steps 7 & 8), sterilizing (step 9), fermenting (step 10), and collecting yeast (step 11). In another illustrative embodiment, a pretreatment process may be included, and tailored to the particular biomaterial waste stream introduced into the process shown in FIG. 51. In one illustrative aspect, the pretreatment process includes collecting washed fibers from solid/liquid separation (steps 4 & 5), extracting the washed fibers, reintroducing the extract into the liquid stream (steps 5 & 6). Remaining solids from the extraction may be discarded, or alternatively, the extracted solids may be recycled into the process. In another illustrative embodiment, a post-treatment process may be include, and tailored to the particular biomaterial waste stream introduced into the process shown in FIG. 51. In one illustrative aspect, the post-treatment process includes collecting the liquid from fermentation (step 10), and aggregating or precipitating any dissolved or undissolved solids, including excess nutrients such as phosphorus-containing components, sulfate-containing components, and the like (steps 12 & 13). The resulting cleaned liquid may be discarded, or alternatively, the cleaned liquid may be used a source of clean water in any one or more of the clean water inlets CWI included in the processes and apparatus described herein.

In particular, step 1 includes pumping a biomaterial waste stream that may be a combination of solids and liquids. Waste material may be pumped from a suitable collection point by a sump pump designed to pump the waste material without clogging. Very large particles may be excluded by entry screens, grates, and the like. Separation of sand, rocks, and other large debris may be accomplished in step 3, where a solids entrainment and sand separation tank may receive the pumped material, and agitate the pumped material allowing the waste to be deposited on the bottom of the tank. Periodically this material may be allowed to discharge from the bottom of the tank into a reservoir, where it is washed with water (step 2), and then discharged.

The remaining liquid containing relatively lighter solids, is pumped to a liquid/solid separator (steps 4 & 5) where a vibratory filter screen may be used to separate the majority of solids from liquid. A water wash (step 4) may be used to dilute liquid saturating the solids, to enable higher recovery of nutrients and/or pollutants dissolved in the liquid. The solids, including fiber, cellulosistic, and other materials, may be directly discharged from the process at this point (step 5b), and may be optionally treated in a pretreatment process. Illustratively, the pretreatment process shown in FIG. 51 includes an acid solubilization/hydrolysis process as described herein. When a high content of cellulosistic material is present in the solid material, it is appreciated that a greater concentration of nutrients may be extracted by dilute sulfuric acid hydrolysis of the solid material. It is understood that other pretreatment processes described herein may be used to treat the separated solids. After treatment, the separated solids may be extracted, and the extract reintroduced to the process (step 5c) for pH adjustment (steps 7 & 8). Alternatively, the combined separated liquids are pumped to a dual, backwashing final filter, and then to pH adjustment (steps 7 & 8).

The pH adjustment may be accomplished by metering an acid as described herein, such as sulfuric acid into the liquid stream (step 8). Alternatively, if the pH is too low, a base as described herein, such as calcium hydroxide is added to the liquid stream to adjust the pH to a higher level. The pH may be determined in some embodiments by measuring the conductivity of the liquid. Following pH adjustment, the liquid may be sterilized (step 9), and fermented (step 10). Sterilization may be accomplished by heating the liquid, optionally under pressure, in an insulated loop of pipe. Illustratively, the liquid stream is heated for about 3 minutes, after which time the liquid is cooled. In alternate processes, the sterilized liquid may be heat exchanged with incoming liquid to both cool the sterilized liquid and recover a portion of the heat. The fermentation process (step 10) may include an air-lift design using sterile air that allows the fermenting organism, such as a yeast species, to convert carbon-containing compounds found in the liquid stream to a mass or population of fermenting organism. Population growth may also remove substantial amount of phosphorus, nitrogen, potassium, and other components from the liquid stream. Fermentation rates may be controlled by controlling the population of the fermenting organism as described herein. The progress of fermenting organism growth may be monitored, and excess fermenting organism may be removed from the system as it is detected (step 11b) to slow fermentation when necessary. Collected fermenting organism product may be subjected to pasteurization, cooled, and/or stored. Liquid exiting the fermentation process (step 11a) may be discarded or the process may include a post-treatment process for removing excess dissolved and undissolved solids remaining in the liquid stream following fermentation (steps 12 & 13). The liquid stream may be treated with a precipitating or other aggregating catalyst as described herein, such as calcium ions to remove dissolved and undissolved solids remaining in the liquid stream. It is understood that other precipitating or aggregating catalysts, including iron salts, and non-ionic polymeric components may be included in the excess solids aggregation process. Aggregated, precipitated, or adsorbed solids are removed (step 13b), and the cleaned liquid (step 13a) may be discarded, or alternatively used as a source of clean water for the processes and apparatus described herein.

It is understood that the process described in FIG. 51 may be used to remove components from a biomaterial waste stream where the components in the biomaterial waste stream are considered pollutants or contaminants, and/or used to grow a population of a fermenting organism where the components in the biomaterial waste stream serve as nutrients for the fermenting organism.

### EXAMPLE 21

### Process Mass Balance for Barn Waste from a Core Process

A sample of barn waste was diluted with water to prepare a slurry containing about 4% solids content. Large debris was removed by seftling for about 1 minute, and the supernatant was decanted away. It is understood that this settling technique performed on a smaller scale approximates results obtained when the solid/liquid separation is performed on a larger scale using the shaker screen process and apparatus described herein. It has been observed that centrifugation of the diluted. barn waste removed a greater percentage of the solids, including bacteria. A chemical oxygen demand (COD) determination was made on the supernatant according to standard EPA testing protocols, and the results are presented in Table 12. Fresh scrapings of the residue were diluted to about 4% solids content (by reference to the moisture and ash free weight determination). The percentages of sand and undissolved minerals was estimated by decanting redissolved ash residues.

Analysis for various components was performed at selected steps shown in FIG. 51, and the results are presented in Table 12. The data in Table 12 are representative of the performance of an illustrative embodiment of the invention, and are a compilation of data and results obtained from several batch and continuous flow experiments. Batch experiments were performed on a scale in the range from about 30 to about 250 mL, and continuous flow experiments were conducted using 2 L fermentation equipment. Fermentation was otherwise performed using conventional equipment and standard procedures. For example, batch fermentation was performed in flasks and the contents during fermentation were agitated on a shaker table. The values in Table 12 have been normalized to a 100 gallon (379 liter) per minute continuous flow process, as described herein.

TABLE 12
Analysis results from Example 21 at selected steps.

| Step^{(a)} | Stream | Water^{(b)} | Sand^{(c)} | Fiber^{(d)} | P^{(e)} | N^{(f)} | COD^{(g)} | SO₄^{2-(h)} | Ca²⁺⁽ⁱ⁾ | Yeast^{(j)} |
|---|---|---|---|---|---|---|---|---|---|---|
| 1 | BW^{(k)} | 363.3 | 5.0 | 9.9 | 104.4 | 596.4 | 4,994.5 | | | |
| 2 | water | 14.9 | | | | | | | | |
| 3b | sand | 2.3 | 4.5 | 0.1 | 1.3 | 7.4 | 61.9 | | | |
| 4 | water | 39.4 | | | | | | | | |
| 5a | liquid | 385.2 | | | 101.2 | 578.4 | 4,844.4 | | | |
| 5b | solid | 19.7 | 0.5 | 9.9 | 1.8 | 10.5 | 88.3 | | | |
| 8 | H₂SO₄ | | | | | | | 1.2 | | |
| 9 | liquid | 385.2 | | | 101.2 | 578.4 | 4,844.4 | 1.2 | | |
| 11a | liquid | 369.1 | | | 31.2 | 428.5 | | | | |
| 11b | yeast | 16.1 | | | 70.0 | 149.9 | | 0.1 | | 4.0 |
| % reduction | | | | | 70.1% | 28.2% | 100% | | | |

^{(a)}Referring to FIG. 51
^{(b)}Liters/min;
^{(c)}typical sand bedded dairies in kilograms/minute;
^{(d)}kilograms/minute of non-dissolved solids without sand;
^{(e)}grams/minute total organic and inorganic phosphorus;
^{(f)}grams/minute total organic and inorganic nitrogen;
^{(g)}Chemical Oxygen Demand, in grams/minute is a measurement that approximates the organic content of the stream;
^{(h)}Kilograms/minute, allows tracking of sulfuric acid added to the process;
⁽ⁱ⁾Kilograms/minute, allows tracking of calcium oxide (lime) added to the process,
^{(j)}Kilograms/minute;
^{(k)}raw dairy barn waste normalized to a continuous flow of 100 gallons/minute (379 liters/minute) of a 4% total solids concentration (excluding sand).

The data in Table 12 indicate that the core process alone removed 100% of the COD, and a substantial portion of the phosphorus-containing and nitrogen-containing components, 70% and 28%, respectively. Yeast production is high at a ratio of 1.2:1 for COD/yeast (kg/kg).

### EXAMPLE 22

### Process Mass Balance for Barn Waste from a Core Process and a Pretreatment of Washed Fiber

The procedure of Example 21 was followed to prepare the first extract. In addition, the fiber removed at step 5b was placed in a cylinder and washed with water in a countercurrent direction. The velocity of water flow was adjusted to exceed the settling speed of small fibers of cellulose and/or lignin particles. The wash water was allowed to flow over the upper edge of the cylinder and was discarded. After water flow was discontinued, the remaining water in the cylinder was drained away leaving the washed fiber behind, primarily the large and/or relatively heavy material. The washed fiber was treated with a minimum amount of 70-80%, or 72-78% H₂SO₄ at ambient temperature for about 30 minutes to 1 h. This mixture was diluted with water to 3% H₂SO₄, and the mixture was heated at 121° C. for 1 h in a pressure vessel (autoclave). After cooling, the mixture was filtered and a COD determination was made on the filtrate according to standard EPA testing protocols, and the results are presented in Table 12. The second extract was added the first extract and the pH adjusted within the range from about 4.0 to about 4.5 by accordingly adding the appropriate amount of H₂SO₄ or calcium carbonate. Precipitated calcium sulfate was optionally removed if present.

In variations, before the mixture was diluted with water to 3% H₂SO₄, the concentrated sulfuric acid was substantially removed. It was found that there was sufficient sulfuric acid remaining with the mixture to obtain a 3% H₂SO₄ solution capable of preparing the second extract. The removed sulfuric acid may be recycled into this or other processes described herein. In other variations, the mixture was diluted first to an intermediate concentration in the range from about 20% to about 50%, and illustratively about 30%. The first dilution to 30% caused the partially hydrolyzed or partially solubilized solids to gel. This intermediate dilution was optionally performed with cooling. The excess liquid was removed, and the gel was diluted with water to form the 3% H₂SO₄ solution. The removed sulfuric acid solution may be recycled into this or other processes described herein.

Analysis for various components was performed at selected steps shown in FIG. 51, and the results are presented in Table 13.

TABLE 13
Analysis results from Example 22 at selected steps.

| Step^{(a)} | Stream | Water^{(b)} | Sand^{(c)} | Fiber^{(d)} | P^{(e)} | N^{(f)} | COD^{(g)} | SO₄^{2-(h)} | Ca²⁺⁽ⁱ⁾ | Yeast^{(j)} |
|---|---|---|---|---|---|---|---|---|---|---|
| 1 | BW^{(k)} | 363.3 | 5.0 | 9.9 | 104.4 | 596.4 | 4,994.5 | | | |
| 2 | water | 14.9 | | | | | | | | |
| 3b | sand | 2.3 | 4.5 | 0.1 | 1.3 | 7.4 | 61.9 | | | |
| 4 | water | 39.4 | | | | | | | | |
| 5a | liquid | 385.2 | | | 101.2 | 578.4 | 4,844.4 | | | |
| 5b | solid | 19.7 | 0.5 | 9.9 | 1.8 | 10.5 | 88.3 | | | |
| 5c | liquid | | | | | | 1,970.9 | 1.2 | | |
| 6 | H₂SO₄ | 39.4 | | | | | | 1.2 | | |
| 9 | liquid | 365.5 | | | 101.2 | 578.4 | 6,815.2 | 1.2 | | |
| 11a | liquid | 365.7 | | | 17.1 | 398.1 | | | | |
| 11b | yeast | 19.6 | | | 84.1 | 180.4 | | 0.1 | | 4.9 |
| % reduction | | | | | 83.6% | 33.3% | 100% | | | |

^{(a)}See legend for Table 12.

The data in Table 13 indicate that pretreatment of the fiber collected from solid/liquid separation step 3 and reintroduction of the extract into the fermentation step 6 results in a higher removal of both phosphorus-containing and nitrogen-containing components compared to the process of Example 21. In addition, the yeast yield was increased over the process of Example 21.

### EXAMPLE 23

### Process Mass Balance for Barn Waster from a Core process and a Post Treatment

The procedure of Example 21 was followed to prepare the first extract. In addition, after fermentation, the resulting liquid was treated with a mixture of ferric sulfate and poly(vinylpyrrolidone) and the pH of the solution was rapidly adjusted to about 6.5 and slowly adjusted to 6.8. The pH was monitored with a pH meter. The mixture was allowed to settle, and the supernatant was analyzed.

Analysis for various components was performed at selected steps shown in FIG. 51, and the results are presented in Table 14.

TABLE 14
Analysis results from Example 23 at selected steps.

| Step^{(a)} | Stream | Water^{(b)} | Sand^{(c)} | Fiber^{(d)} | P^{(e)} | N^{(f)} | COD^{(g)} | SO₄^{2-(h)} | Ca²⁺⁽ⁱ⁾ | Yeast^{(j)} |
|---|---|---|---|---|---|---|---|---|---|---|
| 1 | BW^{(k)} | 363.3 | 5.0 | 9.9 | 104.4 | 596.4 | 4,994.5 | | | |
| 2 | water | 14.9 | | | | | | | | |
| 3b | sand | 2.3 | 4.5 | 0.1 | 1.3 | 7.4 | 61.9 | | | |
| 4 | water | 39.4 | | | | | | | | |
| 5a | liquid | 385.2 | | | 101.2 | 578.4 | 4,844.4 | | | |
| 5b | solid | 19.7 | 0.5 | 9.9 | 1.8 | 10.5 | 88.3 | | | |
| 8 | H₂SO₄ | | | | | | | 1.2 | | |
| 9 | liquid | 385.2 | | | 101.2 | 578.4 | 4,844.4 | 1.2 | | |
| 11a | liquid | 369.1 | | | 31.2 | 428.5 | | | | |
| 11b | yeast | 16.1 | | | 70.0 | 149.9 | | 0.1 | | 4.0 |
| 12 | CaO | | | | | | | | 1.5 | |
| 13a | water | 369.1 | | | 3.1 | 128.5 | | | 1.5 | |
| 13b | ppt | | | | | | | 1.2 | 1.2 | |
| % reduction | | | | | 97.0% | 78.4% | 100% | | | |

^{(a)}See legend for Table 12.

The data in Table 14 indicate that post-treatment of the liquid stream exiting fermentation step 6 increases the removal of both phosphorus-containing and nitrogen-containing components compared to the processes of Example 21 or 22.

### EXAMPLE 24

### Process Mass Balance for Barn Waste from a Core Process, Including a Pretreatment of Washed Fiber and a Post Treatment

The procedures of Examples 21 and 22, were followed to prepare the first extract and the second extract, and the post-treatment procedure of Example 23 was followed. Analysis for various components was performed at selected steps shown in FIG. 51, and the results are presented in Table 15.

TABLE 15
Analysis results from Example 24 at selected steps.

| Step^{(a)} | Stream | Water^{(b)} | Sand^{(c)} | Fiber^{(d)} | P^{(e)} | N^{(f)} | COD^{(g)} | SO₄^{2-(h)} | Ca²⁺⁽ⁱ⁾ | Yeast^{(j)} |
|---|---|---|---|---|---|---|---|---|---|---|
| 1 | BW^{(k)} | 363.3 | 5.0 | 9.9 | 104.4 | 596.4 | 4,994.5 | | | |
| 2 | water | 14.9 | | | | | | | | |
| 3b | sand | 2.3 | 4.5 | 0.1 | 1.3 | 7.4 | 61.9 | | | |
| 4 | water | 39.4 | | | | | | | | |
| 5a | liquid | 385.2 | | | 101.2 | 578.4 | 4,844.4 | | | |
| 5b | solid | 19.7 | 0.5 | 9.9 | 1.8 | 10.5 | 88.3 | | | |
| 5c | liquid | | | | | | 1,970.9 | 1.2 | | |
| 6 | H₂SO₄ | 39.4 | | | | | | 1.2 | | |
| 9 | liquid | 365.5 | | | 101.2 | 578.4 | 6,815.2 | | | |
| 11a | liquid | 365.7 | | | 17.1 | 398.1 | | | | |
| 11b | yeast | 19.6 | | | 84.1 | 180.4 | | | | 4.9 |
| 12 | CaO | | | | | | | | 1.5 | |
| 13a | water | 365.7 | | | 1.7 | 119.4 | | | 1.5 | |
| 13b | ppt | | | | | | | 1.2 | 1.2 | |
| % reduction | | | | | 98.4% | 80.0% | 100% | | | |

^{(a)}See legend for Table 12.

The data in Table 15 indicate that the process including pretreatment of the fiber collected from solid/liquid separation step 3 and reintroduction of the extract into the fermentation step 6, and post-treatment of the combined liquid stream exiting fermentation step 6 results in an even higher removal of both phosphorus-containing and nitrogen-containing components compared to any of the processes of Examples 21, 22, or 23. In addition, the yeast yield was increased over the process of Examples 21 or 23.

### EXAMPLE 25

### Process Mass Balance for Barn Waste/Bedding Combination from a Core Process, Including a Pretreatment of Washed Fiber and a Post Treatment

The procedures of Example 24 were followed, except that the barn waste included fiber bedding material (sawdust, straw, etc.). Analysis for various components was performed at selected steps shown in FIG. 51, and the results are presented in Table 16.

TABLE 16
Analysis results from Example 25 at selected steps.

| Step^{(a)} | Stream | Water^{(b)} | Sand^{(c)} | Fiber^{(d)} | P^{(e)} | N^{(f)} | COD^{(g)} | SO₄^{2-(h)} | Ca²⁺⁽ⁱ⁾ | Yeast^{(j)} |
|---|---|---|---|---|---|---|---|---|---|---|
| 1 | BW^{(k)} | 356.3 | 5.0 | 19.9 | 104.4 | 596.4 | 4,994.5 | | | |
| 2 | water | 14.9 | | | | | | | | |
| 3b | sand | 2.3 | 4.5 | 0.1 | 1.3 | 7.5 | 63.1 | | | |
| 4 | water | 79.4 | | | | | | | | |
| 5a | liquid | 398.2 | | | 99.3 | 567.2 | 4,750.1 | | | |
| 5b | solid | 39.7 | 0.5 | 19.9 | 3.8 | 21.7 | 181.4 | | | |
| 5c | liquid | | | | | | 6,949.0 | 1.2 | | |
| 6 | H₂SO₄ | 79.4 | | | | | | 1.2 | | |
| 9 | liquid | 358.5 | | | 99.3 | 567.2 | 11,699.1 | | | |
| 11a | liquid | 370.3 | | | | 316.5 | | | | |
| 11b | yeast | 27.9 | | | 117.3 | 250.7 | | | | 7.0 |
| 12 | CaO | | | | | | | | 1.5 | |
| 13a | water | 370.3 | | | | 31.6 | | | 1.5 | |
| 13b | ppt | | | | | | | 1.2 | 1.2 | |
| % reduction | | | | | 100% | 94.7% | 100% | | | |

^{(a)}See legend for Table 12.

The increased carbohydrate from the bedding material resulted in a higher removal of phosphorus-containing and nitrogen-containing components than Examples, 21, 22, 23, or 24. In addition, the yield of yeast was higher than Examples, 21, 22, 23, or 24.

It is appreciated that the relative improvements in phosphorus-containing and nitrogen-containing component reduction and yeast yield attributable to the optional pretreatment and/or post-treatment processes may be better or worse depending upon each batch of barn waste, including the amount of bedding material contained therein. It is further appreciated that the relative improvements in phosphorus-containing and nitrogen-containing component reduction and yeast yield attributable to the optional pretreatment and/or post-treatment processes may be better or worse depending upon the source of biomaterial waste.

### EXAMPLE 26

### Removal of a 29 kDa Protein Spiked into Barn Waste

Samples of barn flush waste were spiked with Bovine Carbonic Anhydrase (BCA, MW 29 kDa) at 1.25 mg/mL. The pH of each was adjusted to 4.0 with 30% w/w H₂SO₄ and 100 ppm Al added (aluminum source was aluminum sulfate) and was autoclaved at 121° C. for 10 min. The samples were filtered through 0.45 μm filter material to remove larger particles, then were fractionated on a Sephadex G-100 gel filtration column according to the following: 2 mL sample on 1.5 cm×˜45 cm column (79 mL column volume) at 2.5 rpm (15 mL/hr). Each fraction was tested for protein, and the molecular weight (range) determined by a modification of the micro Lowry method.

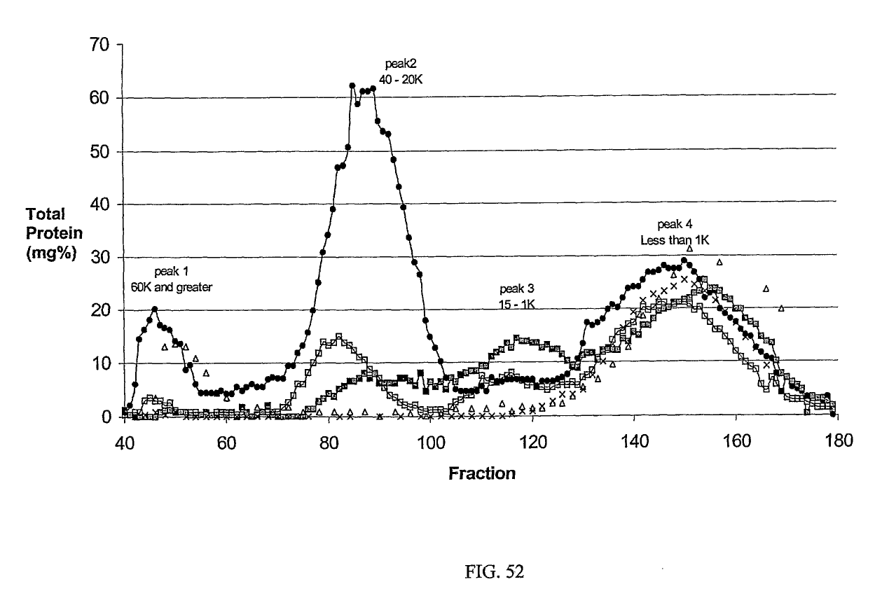

Analysis of the fractions showed that the barn flush waste had 4 protein/polypeptide peaks of interest when separated on the Sephadex G-100. The first peak corresponded to the void volume at fractions 42-56 and consisted of proteins 60 kDa and higher. The second peak at fractions 73-102 consisted of proteins having 20-40 kDa. This second peak included the spiked in 29 kDa protein, BCA, and was not present in unspiked barn flush waste at detectable levels. The third peak at fractions 106-125 consisted of proteins and polypeptides from 15-1 kDa, and this third peak also was not present in the unspiked barn flush waste at detectable levels. The last peak at fractions 130-170 contained polypeptides with molecular weights below 1 kDa that react with the Lowry protein method, as shown in Table 17, and in FIG. 52. Referring to FIG. 52, Trace a (●) refers to pH 8, with spiked 29 kDa protein; Trace b (▪) refers to pH 4, 100 ppm Al, heating for 10 min. at 121 ° C., with spiked 29 kDa protein; Trace c (□) refers to pH 4, 100 ppm Al, no heat, with spiked 29 kDa protein; Trace d (Δ) refers to pH 8, without spiked 29 kDa protein; and Trace e (×) refers to pH 4, 100 ppm Al, heating at 95° C., without spiked 29 kDa protein.

When the protein spiked barn flush was adjusted to pH 4 with sulfuric acid, and aluminum in the form of aluminum sulfate was added, proteins over 20 kDa were reduced by 80% to 86%. In this example, the 20 kDa proteins were not totally removed. The reduction in protein was determined to be due in part to precipitation; however, some of that protein fraction also was determined to be degraded or broken down to smaller polypeptides. Peaks 1 and 2 were reduced with the lowering of pH to 4 and the addition of aluminum, but peak 3 increased approximately 90%, as shown by Samples (b) and (c) in Table 17, and in FIG. 52. Heating increased the amount of 20 kDa proteins removed by about an additional 5% to 10%. Heating also increased the amount of higher molecular weight proteins removed by similar amounts. However, heating increased the amount of lower molecular weight 15-1 kDa proteins, suggesting some degradation of higher molecular weight proteins.

TABLE 17
Protein summary of eluted peaks.

| | | | | | % Change from | |
|---|---|---|---|---|---|---|
| Proteins | | Protein in Fractions (mg) | | | Trace a | |
| (kDa) | Fractions | Trace a | Trace b | Trace c | Trace b | Trace c |
| ≧60 | 42-56 | 1.00 | 0.05 | 0.14 | −96% | −86% |
| | (peak 1) | | | | | |
| 20-40 | 73-102 | 5.87 | 0.83 | 1.16 | −86% | −80% |
| | (peak 2) | | | | | |
| 15-1 | 106-125 | 0.65 | 1.26 | 0.63 | +93% | −3% |
| | (peak 3) | | | | | |
| <1 | 130-170 | 4.40 | 3.75 | 3.12 | −15% | −29% |
| | (peak 4) | | | | | |

In addition, lowering the pH and heat the barn flush waste appeared to increase the solubility of other components, as shown by the inorganic and organic nitrogen results in Table 18. When solids were removed from the pH 8 sample, the inorganic and organic nitrogen decreased by 7% and 26%, respectively. When acid was added to adjust the sample to pH 4, the inorganic nitrogen was observed to be the same as that of the original sample before solids were removed, even though the solids were removed from the pH 4 sample. When the pH 4+aluminum sample was heated, the organic nitrogen was reduced 62%, but it was reduced 70% without heating. It was observed that although organic nitrogen was decreased more without heating, the solids required longer settling times.

TABLE 18
Nitrogen summary of barn flush waste samples.

| | | Inorganic | | Organic |
|---|---|---|---|---|
| | Inorganic | Nitrogen | Organic | Nitrogen |
| | Nitrogen | (% difference | Nitrogen | (% difference |
| Sample | (mg %) | from Trace a) | (mg %) | from Trace a) |
| Trace a | 30 | — | 36 | — |
| (before | | | | |
| filtration) | | | | |
| Trace a (after | 25 | −7% | 27 | −26% |
| filtration) | | | | |
| Trace b | 30 | 0% | 14 | −62% |
| (before | | | | |
| filtration) | | | | |
| Trace b (after | 30 | 0% | 15 | −59% |
| filtration) | | | | |
| Trace c (after | 30 | 0% | 11 | −70% |
| filtration) | | | | |

The foregoing description, illustrative embodiments, and exemplary embodiments are intended to illustrate the invention. It is to be understood that nothing in the foregoing should be construed to limit the invention.

I claim:

1-41. (canceled)

42. A method of treating a biomaterial waste stream to remove pollutants and to generate a product comprising the steps of contacting microorganisms with a cationic flocculating agent, contacting the microorganisms with an anionic flocculating agent, flocculating the microorganisms, contacting the flocculated microorganisms with the biomaterial waste stream, and removing a pollutant.

43. The method of claim 42 wherein the microorganisms are selected from the group consisting of yeast, bacteria, fungi, mycoplasma, and combinations thereof.

44. The method of claim 42 wherein the microorganisms are yeast.

45. The method of claim 42 wherein the microorganisms are bacteria.

46. The method of claim 42 wherein the microorganisms are inoculated into a sample.

47. The method of claim 42 wherein the flocculating agent of the cationic type is selected from the group consisting of ferrous chloride, ferrous sulphate, ferric chloride, ferric sulphate, chlorinated ferric sulphate, aluminum sulphates, chlorinated basic aluminum sulphates, magnesium chloride, magnesium sulphate, and combinations thereof.

48. The method of claim 42 wherein the flocculating agent of the anionic type is selected from the group consisting of an anionic polyacrylamide, a polyacrylate, a polymethacrylate, a polycarboxylate, a polysaccharide including xanthan gum, partially hydrolyzed guar gums, gum Arabic, alginates or partially hydrolyzed alginates, chitosan, cellulose, and combinations thereof.

49. The method of claim 42 wherein the flocculating agent of the cationic type is ferric chloride.

50. The method of a claim 42 wherein the flocculating agent of the anionic type is xanthan gum.

51. The method of claim 42 wherein the flocculating agent of the cationic type is ferric chloride and the flocculating agent of the anionic type is xanthan gum.

52. The method of claim 42 wherein the microorganism is selected from the group consisting of Sacchromyces species, Candida species, Kluyveromyces species, Pichia species, and combinations thereof.

53. The method of claim 42 wherein the concentration of the flocculating agent of the cationic type is from about 0.01 ppm to about 300 ppm.

54. The method of claim 42 wherein the concentration of the flocculating agent of the anionic type is from about 0.001 g/L to about 10 g/L.

55. A composition comprising a cationic flocculating agent, an anionic flocculating agent, and a flocculated microorganism.

56. The composition of claim 55 wherein the flocculating agent of the cationic type is selected from the group consisting of ferrous chloride, ferrous sulphate, ferric chloride, ferric sulphate, chlorinated ferric sulphate, aluminum sulphates, chlorinated basic aluminum sulphates, magnesium chloride, magnesium sulphate, and combinations thereof.

57. The composition of claim 55 wherein the flocculating agent of the anionic type is selected from the group consisting of an anionic polyacrylamide, a polyacrylate, a polymethacrylate, a polycarboxylate, a polysaccharide including xanthan gum, partially hydrolyzed guar gums, gum Arabic, alginates or partially hydrolyzed alginates, chitosan, cellulose, and combinations thereof.

58. The composition of claim 55 wherein the flocculating agent of the cationic type is ferric chloride and the flocculating agent of the anionic type is xanthan gum.

59. The composition of claim 55 wherein the concentration of the flocculating agent of the cationic type is from about 0.01 ppm to about 300 ppm.

60. The composition of claim 55 wherein the concentration of the flocculating agent of the anionic type is from about 0.001 g/L to about 10 g/L.

61. A method of flocculating microorganisms, the method comprising the steps of contacting the microorganisms with a cationic flocculating agent, contacting the microorganisms with an anionic flocculating agent, and flocculating the microorganisms.
